(19) United States
(12) Patent Application Publication
(10) Pub. No.: US 2013/0012506 A1
(43) Pub. Date: Jan. 10, 2013

(54) ANTI-INFECTIVE PYRIDO (1,2-A) PYRIMIDINES

(76) Inventors: Zaesung No, Gyeonggido (KR); Jaeseung Kim, Seoul (KR); Priscille Brodin, Paris (FR); Min Jung Seo, Gyeonggi-do (KR); Eunjung Park, Seoul (KR); Jonathan Cechetto, Seoul (KR); Heekyoung Jeon, Gyeonggi-do (KR); EunHye Kim, Gyeonggi-do (KR); Jamung Heo, Gyeonggi-do (KR); Ji Youn Nam, Yongin (KR); Denis Philippe Cedric Fenistein, Amsterdam (NL); Thierry Christophe, Pontarlier (FR); Monica Contreras Dominguez, Saint Louis (FR); Auguste Genovesio, Paris (FR); Saeyeon Lee, Gyeonggi-do (KR); Sunhee Kang, Gyeonggi-do (KR); Fanny Anne Ewann, Haramont (FR)

(21) Appl. No.: 13/518,292

(22) PCT Filed: Jan. 13, 2011
(86) PCT No.: PCT/EP2011/000135

§ 371 Date: Sep. 5, 2012

Related U.S. Application Data
(60) Provisional application No. 61/294,552, filed on Jan. 13, 2010.

(51) Int. Cl. C07D 239/70; A61P 31/06; A61K 31/5377; A61P 31/04; A61K 31/519; C07D 413/14

(52) U.S. Cl. 514/233.2; 544/282; 514/259.41; 514/259.4; 514/252.16; 544/116

(57) ABSTRACT

The present invention relates to small molecule compounds and their use in the treatment of bacterial infections, in particular Tuberculosis.

The present invention relates to small molecule compounds and their use in the treatment of bacterial infections, in particular Tuberculosis.

### BACKGROUND OF THE INVENTION

Tuberculosis (TB) as a disease continues to result in millions of deaths each year. Inadequate use of chemotherapy has led to an increasing number of drug resistant cases. This situation is likely to worsen with the emergence of extremely resistant strains to all currently known drugs (Van Rie and Enarson, 2006). The internationally recommended TB control strategy, also referred to as directly observed short-course chemotherapy (DOTS), relies on a combination of five antibacterial agents to be taken for a protracted period of more than six months (http://www.who.int/tb/dots/en/). With the use of a mathematical model, taking into consideration treatment duration and TB dynamics, benefits of reduced treatment length were predicted to be substantial and likely to greatly contribute to a reduced global TB burden (Salomon et al., 2006).

Current chemotherapy consists of compounds that directly target Mycobacterium tuberculosis bacillus, either by neutralizing general information pathways and critical processes such as RNA polymerization and protein synthesis inhibition or by interfering with mycobacterial specific cell envelope synthesis. The most widely used dedicated anti-tubercular drugs isoniazid, ethionamide and pyrazinamide are pro-drugs that first require activation. As active forms, they demonstrate inhibitory activity on a wide range of mycobacterial targets, which have not yet been fully characterized. As for other chronic infectious diseases like human immunodeficiency virus, a multi-therapy approach, including drugs that target a wide range of critical features of M. tuberculosis, proved to be the most successful strategy to date. It is, thus, likely that a combination of current drug inhibitors, having different mechanisms of action against M. tuberculosis, will be the solution for the control of the disease.

The most challenging approaches for discovering new anti-TB drugs rely on screening for active compounds that target critical features essential for the survival of the bacillus. Although there is still a lack of understanding of the biological mechanisms behind tubercle bacillus persistence, i.e. the location and state of latent bacteria, in humans, M. tuberculosis is thought to reside in primary granulomas under hypoxic conditions (Lenaerts et al., 2007) as well as to hide within various types of cells (Houben et al., 2006; Neyrolles et al., 2006). The bacillus mainly localizes inside phagocytic cells, such as macrophages and dendritic cells, and it has clearly been established that the tubercle bacillus adopts a different phenotype in the host macrophage's phagosome compared to growth in extracellular conditions (Rohde et al., 2007; Schnappinger et al., 2003). Upon infection, an inflammatory response is induced, thereby initiating recruitment of T lymphocytes that release interleukins and cytokines, which in turn activate the infected macrophages to enable the destruction of the pathogen. Upon the appropriate trigger, the host macrophage is, thus, able to eliminate the invading bacillus. This is further supported by the fact that of the people that inhale M. tuberculosis, more than 95% percent do not develop the disease, suggesting that the human host response is sufficient in most cases to thwart M. tuberculosis induced pathogenesis. This gives rise to the hypothesis that small molecular compounds could mimic the immune cell response signals and induce the host cells to clear the mycobacteria.

Accordingly, a phenotypic cell-based assay, suitable for high throughput screening, that allows for the search of compounds that would prevent M. tuberculosis multiplication inside the host macrophage was utilized.

Up to now, this type of investigation of the tubercle bacillus growth within host cells relied on colony forming units (CFUs) determination after host cell lysis followed by serial dilutions and a 3-week incubation period required for bacterial growth on agar plates. Luciferase-expressing mycobacteria have been shown to be efficient in reducing the experiment duration, although cell lysis and luciferin substrate addition steps are still required (Arain et al., 1996). Also, these types of experiments are not easily amenable to large scale screening.

It was an object of the present invention to identify compounds effective against bacterial infections, in particular compounds that would prevent M. tuberculosis multiplication inside the host macrophage.

### DESCRIPTION OF THE INVENTION

In one aspect, the present invention relates to compounds having the general formula I:

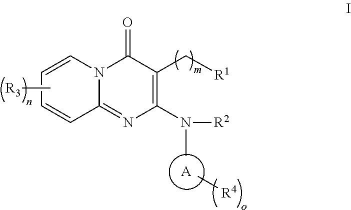

wherein
m is 0, 1, 2, or 3;
n is 1, 2, 3, or, 4;
o is 1, 2, 3, or, 4;
A is C₅-C₁₂ heteroaryl;
R¹ is selected from the group consisting of hydrogen, halogen, C₁-C₁₀ alkyl, C₃-C₁₀ cycloalkyl, C₂-C₁₀ alkenyl, C₃-C₁₀ cycloalkenyl, C₃-C₁₅ cycloalkylalkoxy, C₃-C₁₅ cycloalkylalkyl, hydroxyl, haloalkyl, oxo, —OR⁵, —OC(O)R⁵, —OC(O)N(R⁵)₂, —C(O)OR⁵, —C(O)R⁵, —C(O)N(R⁵)₂, —CN, —NO₂, —NH₂, —N(R⁵)₂, —N(R⁵)C(O)R⁵, —N(R⁵)C(O)N(R⁵)₂, —OR⁵HetA, —OR⁵N(R⁵)₂, —C(O)N(R⁵)R⁵HetA, —C(O)N(R⁵)HetA, —C(O)HetA, —C(O)N(R⁵)R⁵S(O)₂R⁵; SH, C(S)H, —S(O)₂N(R⁵)₂, —S(O)₂R⁵, —N(R⁵)C(O)R⁵SR⁵, —N(R⁵)R⁵S(O)₂R⁴, or —N(R⁵)S(O)₂R⁵, aryl, benzyl, heteroaryl, or heterocyclyl, any of which is optionally substituted;
R² is selected from the group consisting of hydrogen, halogen, C₁-C₁₀ alkyl, C₃-C₁₀ cycloalkyl, C₂-C₁₀ alkenyl, C₃-C₁₀ cycloalkenyl, C₃-C₁₅ cycloalkylalkyl, —NH₂, —N(R⁶)₂, —C(O)R⁶, —C(O)OR⁶, —C(O)N(R⁶)₂, —S(O)R⁶, —S(O)₂R⁶, —S(O)₂N(R⁶)₂, aryl, benzyl, heteroaryl, or heterocyclyl, or two groups of R¹ and R² are connected with each other to make a five or six membered cyclic or heterocyclic ring, any of which is optionally substituted;
R³ is selected from the group consisting of hydrogen, halogen, C₁-C₁₀ alkyl, C₃-C₁₀ cycloalkyl, hydroxyl, —OR⁶, —CN, —NO₂, —NH₂, —N(R⁶)C(O)R⁶, —C(O)R⁶, —C(O)OR⁶, —C(O)N(R⁶)₂, —S(O)R⁶, —S(O)₂R⁶, —S(O)₂N(R⁶)₂, aryl, benzyl, heteroaryl, heterocyclyl, or two groups of R³ are connected with each other to make a five or six membered cyclic or heterocyclic ring, any of which is optionally substituted;
R⁴ is independently, at each occurrence, selected from the group consisting of hydrogen, halogen, C₁-C₁₀ alkyl, C₃-C₁₀ cycloalkyl, hydroxyl, —OR⁶, —CN, —NO₂, —NH₂, —N(R⁶)C(O)R⁶, —C(O)R⁶, —C(O)OR⁶, —C(O)N(R⁶)₂, —S(O)R⁶, —S(O)₂R⁶, —S(O)₂N(R⁶)₂, aryl, benzyl, heteroaryl, heterocyclyl, or two groups of R⁴ are connected with each other to make five or six membered cyclic or heterocyclic ring, any of which is optionally substituted;
R⁵ and R⁶ are independently, at each occurrence, selected from the group consisting from hydrogen, C₁-C₁₀ alkyl, C₃-C₁₀ cycloalkyl, C₂-C₁₀ alkenyl, C₃-C₁₀ cycloalkenyl, C₂-C₁₀ alkynyl, C₁-C₁₀ haloalkyl, aryl, benzyl, heteroaryl, or heterocyclyl, any of which is optionally substituted; The term “HetA” refers to “heteroaryl”;
The term “optionally substituted” as used herein is meant to indicate that a hydrogen atom attached to a member atom within a group is possibly replaced by group, such as halogen including fluorine, C₁-C₁₀ alkyl, C₁-C₃ haloalkyl, C₃-C₇ cycloalkyl, oxo, —OH, —OR⁷, —OC(O)R⁷, —CN, —NO₂, —N(R⁷)₂, —N(R⁷)C(O)R⁷, —C(O)R⁷, —C(O)OR⁷, —C(O)N(R⁷)₂, —S(O)R⁷, —S(O)₂R⁷, —S(O)₂N(R⁷)₂, phenyl, benzyl, heteroaryl, or heterocyclyl;
R⁷ is independently, at each occurrence, selected from the group consisting of hydrogen, aryl, benzyl, heteroaryl, heterocyclyl, C₁-C₈ alkyl, or C₃-C₇ cycloalkyl;

The term “alkyl” refers to a monovalent straight or branched chain, saturated aliphatic hydrocarbon radical having a number of carbon atoms in the specified range. Thus, for example, “C₁-C₆ alkyl” refers to any of the hexyl alkyl and pentyl alkyl isomers as well as n-, iso-, sec-, and t-butyl, n- and isopropyl, ethyl and methyl.

The term “alkoxy” means a group having the formula —O-alkyl, in which an alkyl group, as defined above, is attached to the parent molecule via an oxygen atom. The alkyl portion of an alkoxy group can have 1 to 20 carbon atoms (i.e., C₁-C₂₀ alkoxy), 1 to 12 carbon atoms (i.e., C₁-C₁₂ alkoxy), or 1 to 6 carbon atoms (i.e., C₁-C₆ alkoxy). Examples of suitable alkoxy groups include, but are not limited to, methoxy (—O—CH₃ or OMe), ethoxy (—OCH₂CH₃ or -OEt), t-butoxy (—O—C(CH₃)₃ or -OtBu) and the like.

The term “alkenyl” refers to a monovalent straight or branched chain aliphatic hydrocarbon radical containing one carbon-carbon double bond and having a number of carbon atoms in the specified range. Thus, for example, “C₂-C₆ alkenyl” refers to all of the hexenyl and pentenyl isomers as well as 1-butenyl, 2-butenyl, 3-butenyl, isobutenyl, 1-propenyl, 2-propenyl, and ethenyl (or vinyl).

The term “alkynyl” refers to a monovalent straight or branched chain aliphatic hydrocarbon radical containing one carbon-carbon triple bond and having a number of carbon atoms in the specified range. Thus, for example, “C₂-C₆ alkynyl” refers to all of the hexynyl and pentynyl isomers as well as 1-butynyl, 2-butynyl, 3-butynyl, 1-propynyl, 2-propynyl, and ethynyl.

The term “alkylene” refers to a saturated, branched or straight chain or cyclic hydrocarbon radical having two monovalent radical centers derived by the removal of two hydrogen atoms from the same or two different carbon atoms, 1 to 10 carbon atoms, or 1 to 6 carbon atoms. Typical alkylene radicals include, but are not limited to, methylene (—CH₂—), 1,1-ethyl (—CH(CH₃)—), 1,2-ethyl (—CH₂CH₂—), 1,1-propyl (—CH(CH₂CH₃)—), 1,2-propyl (—CH₂CH(CH₃)—), 1,3-propyl (—CH₂CH₂CH₂—), 1,4-butyl (—CH₂CH₂CH₂CH₂—), and the like.

The term “alkenylene” refers to an unsaturated, branched or straight chain or cyclic hydrocarbon radical having two monovalent radical centers derived by the removal of two hydrogen atoms from the same or two different carbon atoms of parent alkene. For example, an alkenylene group can have 1 to 20 carbon atoms, 1 to 10 carbon atoms, or 1 to 6 carbon atoms. Typical alkenylene radicals include, but are not limited to, 1,2-ethenyl (—CH═CH—).

The term “alkynylene” refers to an unsaturated, branched or straight chain or cyclic hydrocarbon radical having two monovalent radical centers derived by the removal of two hydrogen atoms from the same or two different carbon atoms of parent alkyne. For example, an alkynylene group can have 1 to 20 carbon atoms, 1 to 10 carbon atoms or 1 to 6 carbon atoms. Typical alkynylene radicals include, but are not limited to, acetylene (—C≡C—), propargyl (—CH₂C≡C—), and 4-pentynyl (—CH₂CH₂CH₂C≡CH—).

The term “cycloalkyl”, alone or in combination with any other term, refers to a group, such as optionally substituted or non-substituted cyclic hydrocarbon, having from three to eight carbon atoms, unless otherwise defined. Thus, for example, “C₃-C₈ cycloalkyl” refers to cyclopropyl, cyclobutyl, cyclopentyl, cyclohexyl, cycloheptyl, and cyclooctyl.

The term “haloalkyl” refers to an alkyl group, as defined herein that is substituted with at least one halogen. Examples of straight or branched chained “haloalkyl” groups useful in the present invention include, but are not limited to, methyl, ethyl, propyl, isopropyl, n-butyl, and t-butyl substituted independently with one or more halogens. The term “haloalkyl” should be interpreted to include such substituents such as —CHF₂, —CF₃, —CH₂—CH₂—F, —CH₂—CF₃, and the like.

The term “heteroalkyl” refers to an alkyl group where one or more carbon atoms have been replaced with a heteroatom, such as, O, N, or S. For example, if the carbon atom of alkyl group which is attached to the parent molecule is replaced with a heteroatom (e.g., O, N, or S) the resulting heteroalkyl groups are, respectively, an alkoxy group (e.g., —OCH₃, etc.), an amine (e.g., —NHCH₃, —N(CH₃)₂, etc.), or thioalkyl group (e.g., —SCH₃, etc.). If a non-terminal carbon atom of the alkyl group which is not attached to the parent molecule is replaced with a heteroatom (e.g., O, N, or S) and the resulting heteroalkyl groups are, respectively, an alkyl ether (e.g., —CH₂CH₂—O—CH₃, etc.), alkyl amine (e.g., —CH₂NHCH₃, —CH₂N(CH₃)₂, etc.), or thioalkyl ether (e.g., —CH₂—S—CH₃).

The term “halogen” refers to fluorine, chlorine, bromine, or iodine.

The term “aryl” refers to (i) optionally substituted phenyl, (ii) optionally substituted 9- or 10 membered bicyclic, fused carbocyclic ring systems in which at least one ring is aromatic, and (iii) optionally substituted 11- to 14-membered tricyclic, fused carbocyclic ring systems in which at least one ring is aromatic. Suitable aryls include, for example, phenyl, biphenyl, naphthyl, tetrahydronaphthyl (tetralinyl), indenyl, anthracenyl, and fluorenyl.

The term “phenyl” as used herein is meant to indicate that optionally substituted or non-substituted phenyl group.

The term “benzyl” as used herein is meant to indicate that optionally substituted or non-substituted benzyl group.

The term “heteroaryl” refers to (i) optionally substituted 5- and 6-membered heteroaromatic rings and (ii) optionally substituted 9- and 10-membered bicyclic, fused ring systems in which at least one ring is aromatic, wherein the heteroaromatic ring or the bicyclic, fused ring system contains from 1 to 4 heteroatoms independently selected from N, O, and S, where each N is optionally in the form of an oxide and each S in a ring which is not aromatic is optionally S(O) or S(O)₂. Suitable 5- and 6-membered heteroaromatic rings include, for example, pyridyl, pyrrolyl, pyrazinyl, pyrimidinyl, pyridazinyl, triazinyl, thienyl, furanyl, imidazolyl, pyrazolyl, triazolyl, tetrazolyl, oxazolyl, isooxazolyl, oxadiazolyl, thiazolyl, isothiazolyl, and thiadiazolyl. Suitable 9- and 10-membered heterobicyclic, fused ring systems include, for example, benzofuranyl, indolyl, indazolyl, naphthyridinyl, isobenzofuranyl, benzopiperidinyl, benzisoxazolyl, benzoxazolyl, chromenyl, quinolinyl, isoquinolinyl, cinnolinyl, quinazolinyl, tetrahydroquinolinyl, tetrahydroisoquinolinyl, isoindolyl, benzodioxolyl, benzofuranyl, imidazo[1,2-a]pyridinyl, benzotriazolyl, dihydroindolyl, dihydroisoindolyl, indazolyl, indolinyl, isoindolinyl, quinoxalinyl, quinazolinyl, 2,3-dihydrobenzofuranyl, and 2,3-dihydrobenzo-1,4-dioxinyl.

The term “heterocyclyl” refers to (i) optionally substituted 4- to 8-membered, saturated and unsaturated but non-aromatic monocyclic rings containing at least one carbon atom and from 1 to 4 heteroatoms, (ii) optionally substituted bicyclic ring systems containing from 1 to 6 heteroatoms, and (iii) optionally substituted tricyclic ring systems, wherein each ring in (ii) or (iii) is independent of fused to, or bridged with the other ring or rings and each ring is saturated or unsaturated but nonaromatic, and wherein each heteroatom in (i), (ii), and (iii) is independently selected from N, O, and S, wherein each N is optionally in the form of an oxide and each S is optionally oxidized to S(O) or S(O)₂. Suitable 4- to 8-membered saturated heterocyclyls include, for example, azetidinyl, piperidinyl, morpholinyl, thiomorpholinyl, thiazolidinyl, isothiazolidinyl, oxazolidinyl, oxazolidonyl, isoxazolidinyl, pyrrolidinyl, imidazolidinyl, piperazinyl, tetrahydrofuranyl, tetrahydrothienyl, pyrazolidinyl, hexahydropyrimidinyl, thiazinanyl, thiazepanyl, azepanyl, diazepanyl, tetrahydropyranyl, tetrahydrothiopyranyl, dioxanyl, and azacyclooctyl. Suitable unsaturated heterocyclic rings include those corresponding to the saturated heterocyclic rings listed in the above sentence in which a single bond is replaced with a double bond. It is understood that the specific rings and ring systems suitable for use in the present invention are not limited to those listed in this and the preceding paragraphs. These rings and ring systems are merely representative.

According to another aspect, the present invention relates to compounds having the general formula II:

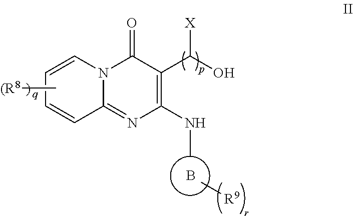

wherein
p is 0, 1, 2, or 3;
q is 1, 2, 3, or, 4;
r is 1, 2, 3, or, 4;
X is alkyl or aryl;
B is C₅-C₁₂ aryl;
R⁸ is selected from the group consisting of hydrogen, halogen, C₁-C₁₀ alkyl, C₃-C₁₀ cycloalkyl, hydroxyl, —OR¹⁰, —CN, —NO₂, —NH₂, —N(R¹⁰)C(O)R¹⁰, —C(O)R¹⁰, —C(O)—OR¹⁰, —C(O)N(R¹⁰)₂, —S(O)R¹⁰, —S(O)₂R¹⁰), —S(O)₂N(R¹⁰)₂, aryl, benzyl, heteroaryl, hetero-cyclyl, or two groups of R⁸ are connected with each other to make a five or six membered cyclic or heterocyclic ring, any of which is optionally substituted;
R⁹ is selected from the group consisting of hydrogen, halogen, C₁-C₁₀ alkyl, C₃-C₁₀ cycloalkyl, C₂-C₁₀ alkenyl, C₃-C₁₀ cycloalkenyl, C₃-C₁₅ cycloalkylalkoxy, C₃-C₁₅ cycloalkylalkyl, hydroxyl, oxo, —OR¹¹, —OC(O)R¹¹, —OC(O)N(R¹¹)₂, —C(O)OR¹¹, —C(O)R¹¹, —C(O)N(R¹¹)₂, —CN, —NO₂, —NH₂, —N(R¹¹)₂, —N(R¹¹)C(O)R¹¹, —N(R¹¹)C(O)N(R¹¹)₂, —OR¹¹HetA, —OR¹¹N(R¹¹)₂, —C(O)N(R¹¹)R¹¹HetA, —C(O)N(R¹¹)HetA, —C(O)HetA, —C(O)N(R¹¹)R¹¹—S(O)₂R¹¹, —S(O)₂N(R¹¹)₂, —S(O)₂R¹¹, —N(R¹¹)C(O)R¹¹SR¹¹, —N(R¹¹)R¹¹S(O)₂R¹¹, or —N(R¹¹)—S(O)₂R¹¹, —R¹¹P(O)(OR¹¹)₂, aryl, benzyl, heteroaryl, heterocyclyl, or two groups of R⁹ are connected with each other to make a five or six membered cyclic or heterocyclic ring, any of which is optionally substituted;
R¹⁰ and R¹¹ are independently, at each occurrence, selected from the group consisting from hydrogen, C₁-C₁₀ alkyl, C₃-C₁₀ cycloalkyl, C₂-C₁₀ alkenyl, C₃-C₁₀ cycloalkenyl, C₂-C₁₀ alkynyl, C₁-C₁₀ haloalkyl, aryl, benzyl, heteroaryl, or heterocyclyl, any of which is optionally substituted;
The term “optionally substituted” as used herein is meant to indicate that a hydrogen atom attached to a member atom within a group is possibly replaced by group, such as halogen including fluorine, C₁-C₁₀ alkyl, C₁-C₃ haloalkyl, C₃-C₇ cycloalkyl, oxo, —OH, —OR¹², —OC(O)R¹², —CN, —NO₂, —N(R¹²)₂, —N(R¹²)C(O)R¹², —C(O)R¹², —C(O)OR¹², —C(O)N(R¹²)₂, —S(O)R¹², —S(O)₂R¹², —S(O)₂N(R¹²)₂, phenyl, benzyl, heteroaryl, or heterocyclyl, as also defined further above;
R¹² is independently, at each occurrence, selected from the group consisting of hydrogen, aryl, benzyl, heteroaryl, heterocyclyl, C₁-C₈ alkyl, or C₃-C₇ cycloalkyl;
The term “HetA” refers to “heteroaryl”;

The term “optionally substituted” has the same meaning as defined above.

The term “alkyl” has the same meaning as defined above.

The term “alkoxy” has the same meaning as defined above.

The term “alkenyl” has the same meaning as defined above.

The term “alkynyl” has the same meaning as defined above.

The term “alkylene” has the same meaning as defined above.

The term “alkenylene” has the same meaning as defined above.

The term “alkynylene” has the same meaning as defined above.

The term “cycloalkyl”, alone or in combination with any other term, has the same meaning as defined above.

The term “haloalkyl” has the same meaning as defined above.

The term “heteroalkyl” has the same meaning as defined above.

The term “halogen” has the same meaning as defined above.

The term “aryl” has the same meaning as defined above.

The term “phenyl” has the same meaning as defined above.

The term “benzyl” has the same meaning as defined above.

The term “heteroaryl” has the same meaning as defined above.

The term “heterocyclyl” has the same meaning as defined above.

Another embodiment of the present invention is compounds of general formula I and II, or pharmaceutically acceptable salts thereof.

In another aspect, the present invention relates to compounds having the general formula VIII:

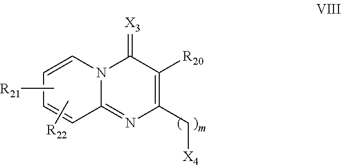

wherein
m is 0, 1, 2, or 3;
X₃ is selected from the group comprising CH₂, O, S and NH;
X₄ is selected from the group comprising halide, alkyl, OR₂₃, SR₂₄ and NR₂₅R₂₆;
R₂₀ is selected from the group comprising acyl, alkoxy, alkyl, alkylamino, alkylcarboxylic acid, arylcarboxylic acid, alkylcarboxylic alkylester, alkylene, alkylether, alkylhydroxy, alkylthio, alkynyl, amido, amino, aryl, arylalkoxy, arylamino, arylthio, carboxylic acid, cyano, cycloalkyl, carboxylic acid, ester, halo, haloalkoxy, haloalkyl, haloalkylether, heteroaryl, heteroarylamino, heterocycloalkyl and hydrogen, any of which is optionally substituted;
R₂₁ and R₂₂ are each independently selected from the group comprising alkoxy, alkyl, alkylamino, alkylene, alkylether, alkylthio, alkynyl, amido, amino, aryl, arylether, arylalkoxy, arylamino, arylthio, carboxy, cyano, cycloalkyl, ester, halo, haloalkoxy, haloalkyl, heteroaryl, heteroarylamino, heterocycloalkyl, hydroxyl, hydrogen, nitro, thio, sulfonate, sulfonyl and sulfonylamino, any of which is optionally substituted;
R₂₃ is selected from the group comprising acyl, alkyl, alkylamino, alkylene, alkynyl, aryl, arylalkoxy, arylamino, arylthio, carboxy, cycloalkyl, ester, ether, haloalkyl, heteroaryl, heteroarylamino, heterocycloalkyl, hydrogen, thio, sulfonate, and sulfonylamino, any of which is optionally substituted;
R₂₄ is selected from the group comprising alkyl, alkylaryl, alkylene, alkynyl, aryl, cycloalkyl, ester, halo, haloalkyl, heteroaryl, heterocycloalkyl, and hydrogen, any of which is optionally substituted; and
R₂₅ and R₂₆ are each independently selected from the group comprising acyl, alkyl, aminoalkyl, alkylene, alkylthio, alkynyl, aryl, arylalkoxy, arylamino, arylthio, carboxy, cycloalkyl, ester, ether, halo, haloalkoxy, haloalkyl, haloalkylether, heteroaryl, heteroarylamino, heterocycloalkyl and hydrogen, any of which is optionally substituted.

In general, the term “optionally substituted” as used herein is meant to indicate that a group, such as alkyl, alkylene, alkynyl, aryl, cycloalkyl, heterocycloalkyl, or heteroaryl, may be unsubstituted or substituted with one or more substituents as also defined further above.

“Substituted” in reference to a group indicates that a hydrogen atom attached to a member atom within a group is replaced as also defined further above.

In another aspect, the present invention relates to compounds having the general formula VIIIa:

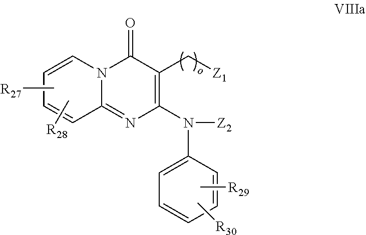

wherein
o is 0, 1, 2, or 3;
Z₁ and Z₂ are each independently selected from the group comprising hydrogen, halogen, C₁-C₁₀ alkyl, C₃-C₁₀ cycloalkyl, C₂-C₁₀ alkenyl, C₃-C₁₀ cycloalkenyl, C₃-C₁₅ cycloalkylalkoxy, C₃-C₁₅ cycloalkylalkyl, hydroxyl, haloalkyl, oxo, —OR³¹, —OC(O)R³¹, —OC(O)N(R³¹)₂, —C(O)OR³¹, —C(O)R³¹, —C(O)N(R³¹)₂, —CN, —NO₂, —NH₂, —N(R³¹)₂, —N(R³¹)C(O)R³¹, —N(R³¹)C(O)N(R³¹)₂, —OR³¹HetA, —OR³¹N(R³¹)₂, —C(O)N(R³¹)R³¹HetA, —C(O)N(R³¹)HetA, —C(O)HetA, —C(O)N(R³¹)R³¹S(O)₂R³¹; SH, C(S)H, —S(O)₂N(R³¹)₂, —S(O)₂R³¹, —N(R³¹)C(O)R³¹SR³¹, —N(R³¹)R³¹S(O)₂R³¹, or N(R³¹)S(O)₂R³¹, aryl, benzyl, heteroaryl, heterocyclyl, or two groups of Z₁ and Z₂ are connected with each other to make a five or six membered cyclic, heterocyclic or heteroaryl ring, any of which is optionally substituted;
R₂₇ and R₂₈ are each independently selected from the group comprising alkoxy, alkyl, alkylamino, alkylene, alkylether, alkylthio, alkynyl, amido, amino, aryl, arylether, arylalkoxy, arylamino, arylthio, carboxy, cyano, cycloalkyl, ester, halo, haloalkoxy, haloalkyl, heteroaryl, heteroarylamino, heterocycloalkyl, hydroxyl, hydrogen, nitro, thio, sulfonate, sulfonyl and sulfonylamino, any of which is optionally substituted;
R₂₉ and R₃₀ are each independently selected from the group comprising alkoxy, alkyl, alkylamino, alkylene, alkylether, alkylthio, alkynyl, amido, amino, aryl, arylether, arylalkoxy, arylamino, arylthio, carboxy, cyano, cycloalkyl, ester, halo, haloalkoxy, haloalkyl, heteroaryl, heteroarylamino, heterocycloalkyl, hydroxyl, hydrogen, nitro, thio, sulfonate, sulfonyl and sulfonylamino, or two groups of R₂₉ and R₃₀ are connected with each other to make a five or six membered cyclic, heterocyclic, aryl, or heteroaryl ring, any of which is optionally substituted;
R³¹ is independently, at each occurrence, selected from the group consisting from hydrogen, C₁-C₁₀ alkyl, C₃-C₁₀ cycloalkyl, C₂-C₁₀ alkenyl, C₃-C₁₀ cycloalkenyl, C₂-C₁₀ alkynyl, C₁-C₁₀ haloalkyl, aryl, benzyl, heteroaryl, or heterocyclyl, any of which is optionally substituted.

The term “alkyl” as used herein is meant to indicate that a group, such as substituted or non-substituted C₁-C₁₀ alkyl group which has the straight or branched chain.

The term “cycloalkyl” as used herein is meant to indicate that a group, such as substituted or non-substituted cyclic compound of C₃-C₈ ring structure.

The term “heteroaryl” as used herein is meant to indicate that a group, such as substituted or non-substituted 5- to 9-membered aromatic compounds which have more than one heteroatom of N, O, and S in the ring structure itself.

The term “optionally substituted” as used herein is meant to indicate that a hydrogen atom attached to a member atom within a group is possibly replaced by group, such as C₁-C₁₀ alkyl, halogen including fluorine, OH, NO₂, OR₃₁, CN, NR₃₁R₃₂, COR₃₁, SOR₃₂, SO₂R₃₁, SO₂NR₃₁, CR₃₁═CR₃₁R₃₂, CR₃₁═NR₃₂, aryl, aryloxy, C₄-C₁₀ heteroaryl group, or —NR₃₁—COR₃₂, —O—COR₃₁ as also defined further above

R₃₁ and R₃₂ are each independently selected from the group comprising hydrogen, alkyl, alkyloxy, alkylamino, alkylcarbonyl, alkylcarbonylamino, alkylcarbonyloxy, alkylaminocarbonyl, alkyloxycarbonyl, cycloalkyl, cycloalkyloxy, cycloalkylamino, cycloalkylcarbonyl, cycloalkylcarbonylamino, cycloalkylcarbonyloxy, cycloalkylaminocarbonyl, cycloalkyloxycarbonyl, heteroaryl, heteroaryloxy, heteroaryl amino, heteroaryl carbonyl, heteroaryl carbonylamino, heteroaryl carbonyloxy, heteroaryl aminocarbonyl, heteroaryl oxycarbonyl, heteroaryl alkyl, heteroaryl alkyloxy, heteroaryl alkylamino, heteroaryl alkylcarbonyl, heteroaryl alkylcarbonylamino, heteroaryl alkylcarbonyloxy, heteroaryl alkylaminocarbonyl, heteroaryl alkyloxycarbonyl, phenyl, phenyloxy, phenylamino, phenylcarbonyl, phenylcarbonylamino, phenylcarbonyloxy, phenylaminocarbonyl, and phenyloxycarbonyl, any of which is optionally substituted.

In another aspect, the present invention relates to compounds having one of the formulas 1-120, as shown in Example 6, 125-359 as shown in Example 7, preferably 4, 5, 13, 61, 65, 71, 74, 78, 97, 102-105, 117,132-135, 137, 139-140, 147, 151-152, 160, 163, 173, 180, 184-185, 193, 195, 199-201, 204, 206-222, 224, 226, 229, 231-243, 245-278, 280-286, 290-305, 316, 324, 337, 340, 341, 355 and 356 as shown in Tables 1 or 2. Particularly preferred compounds are compounds having one of the formulas 4, 5, 13, 61, 65, 71, 74, 78, 97, 102-105, 117, 133, 206-210, 220, 231, 232, 235, 236, 257-259, 261, 264, 265, 267, 270, 273, 278, 295, 299-305, 337, 340 and 356 as shown in Tables 1-4.

Preferably, the compounds as defined above have an inhibitory activity, preferably an inhibitory activity above 65%, on bacterial growth, preferably on the growth of M. tuberculosis, inside a host cell, preferably a macrophage, at a concentration between 5-20 μM, preferably less than 5 μM.

Pharmaceutically acceptable salts of compounds in accordance with the present invention are also contemplated herein. For example such pharmaceutically acceptable salts may be acid addition salts. Thus, a compound in accordance with the present invention treated with an inorganic acid such as hydrochloric, hydrobromic, sulfuric, nitric, phosphoric, and the like, or an organic acid such as an acetic, propionic, glycolic, pyruvic, oxalic, malic, malonic, succinic, maleic, fumaric, tataric, citric, benzoic, cinnamic, mandelic, methanesulfonic, ethanesulfonic, p-toluenesulfonic, salicyclic and the like, to provide a water soluble salt of the compound is suitable for use in the invention.

In one aspect, the present invention relates to compounds as defined above for use in the treatment of bacterial infections.

In one aspect, the present invention relates to compounds as defined above for use in the treatment of Tuberculosis.

In one aspect, the present invention relates to a pharmaceutical composition comprising a compound as defined above.

In one aspect, the present invention relates to a method of treatment of Tuberculosis, comprising the application of a pharmaceutically suitable amount of a compound as defined above to a person in need thereof.

In another aspect, the present invention relates to compounds having one of the general formulas/scaffolds I, II, VIII and VIIIa, or pharmaceutically acceptable salts thereof, as defined further above.

In one aspect, the present invention relates to a compound listed in Table 1.

In another aspect, the present invention relates to a compound listed in Table 2.

In one aspect, the present invention relates to compounds as defined above for use in the treatment of bacterial infections.

In one aspect, the present invention relates to compounds as defined above for use in the treatment of Tuberculosis.

In one aspect, the present invention relates to a pharmaceutical composition comprising a compound as defined above.

In one aspect, the present invention relates to a method of treatment of a bacterial infection, in particular tuberculosis, said method comprising the application of a pharmaceutically suitable amount of a compound as defined above to a patient in need thereof.

In one embodiment, the patient is a non-human animal, in another embodiment, the patient is a human.

Pharmaceutical compositions of the invention are suitable for use in a variety of drug delivery systems. Suitable formulations for use in the present invention are found in Remington's Pharmaceutical Sciences, Mace Publishing Company, Philadelphia, Pa., 17th ed. (1985). For a brief review of methods for drug delivery, see, Langer, Science 249:1527-1533 (1990).

The pharmaceutical compositions may be formulated for any appropriate manner of administration, including for example, topical, oral, nasal, intravenous, intracranial, intraperitoneal, subcutaneous or intramuscular administration. For parenteral administration, such as subcutaneous injection, the carrier preferably comprises water, saline, alcohol, a fat, a wax or a buffer. For oral administration, any of the above carriers or a solid carrier, such as mannitol, lactose, starch, magnesium stearate, sodium saccharine, talcum, cellulose, glucose, sucrose, and magnesium carbonate, may be employed. Biodegradable microspheres (e.g., polylactate polyglycolate) may also be employed as carriers for the pharmaceutical compositions of this invention. Suitable biodegradable microspheres are disclosed, for example, in U.S. Pat. Nos. 4,897,268 and 5,075,109.

Commonly, the pharmaceutical compositions are administered parenterally, e.g., intravenously. Thus, the invention provides compositions for parenteral administration which comprise the compound dissolved or suspended in an acceptable carrier, preferably an aqueous carrier, e.g., water, buffered water, saline, PBS and the like. The compositions may contain pharmaceutically acceptable auxiliary substances as required to approximate physiological conditions, such as pH adjusting and buffering agents, tonicity adjusting agents, wetting agents, detergents and the like.

These compositions may be sterilized by conventional sterilization techniques, or may be sterile filtered. The resulting aqueous solutions may be packaged for use as is, or lyophilized, the lyophilized preparation being combined with a sterile aqueous carrier prior to administration. The pH of the preparations typically will be between 3 and 11, more preferably from 5 to 9 and most preferably from 7 to 8.

In some embodiments the compounds of the invention can be incorporated into liposomes formed from standard vesicle-forming lipids. A variety of methods are available for preparing liposomes, as described in, e.g., Szoka et al., Ann. Rev. Biophys. Bioeng. 9: 467 (1980), U.S. Pat. Nos. 4,235,871, 4,501,728 and 4,837,028. The targeting of liposomes using a variety of targeting agents is well known in the art (see, e.g., U.S. Pat. Nos. 4,957,773 and 4,603,044).

The dosage ranges for the administration of the compounds of the invention are those large enough to produce the desired anti-infective effect. The dosage should not be so large as to cause adverse side effects. Generally, the dosage will vary with the age, condition, sex and extent of the disease in the animal/patient and can be determined by one of skill in the art. The dosage can be adjusted by the individual physician in the event of any counterindications.

Additional pharmaceutical methods may be employed to control the duration of action. Controlled release preparations may be achieved by the use of polymers to conjugate, complex or adsorb the compound. The controlled delivery may be exercised by selecting appropriate macromolecules (for example, polyesters, polyamino carboxymethylcellulose, and protamine sulfate) and the concentration of macromolecules as well as the methods of incorporation in order to control release. Another possible method to control the duration of action by controlled release preparations is to incorporate the compound into particles of a polymeric material such as polyesters, polyamino acids, hydrogels, poly (lactic acid) or ethylene vinylacetate copolymers.

### FIGURES AND TABLES

Reference is now made to the figures and tables, wherein

FIG. 1 shows the monitoring of tubercle bacillus intracellular growth inside macrophages by automated confocal microscopy: (a) Representative pictures of Raw264.7 cells infected with M. tuberculosis H37Rv-GFP at different time points after infection. (b) Image analysis: 1: Typical 2-color image; 2: Circled object corresponds to detected cells; 3: Circled object corresponds to bacterial aggregates; 4: Filled purple cells correspond to infected cells. (c,d,e) Image-based quantification of the percentage of infected cells and the mean number of cells from 2 hours to day 7 after infection with H37Rv-GFP at a multiplicity of infection of 0.5 (gray square), 1 (black circle) and 2 (dark gray triangle). Non-infected cells (black diamonds) were used as the negative control;

FIG. 2 shows the pharmacological validation and MIC (minimal inhibitory concentration) comparison of the reference drugs in the in vitro growth fluorescence assay and the phenotypic cell-based assay: (a) Representative pictures of infected cells in presence of INH at 1 μg/mL or DMSO control. (b,c,d) Dose-response of INH, rifampin and ethionamide; black square and line corresponds to growth inhibition in cell-based assay; gray circle and line correspond to in vitro growth inhibition; shown is a representative data set;

FIG. 3 shows assay automation validation of the phenotypic cell-based assay: (a) Percent of M. tuberculosis infected cells relative to 384-plate well-index. Black square, dark gray square, gray square and open square correspond to INH 1 μg/mL, rifampin 5 μg/mL, PBS and DMSO control respectively. (b,c) Percent of M. tuberculosis infected cells relative to INH and rifampin concentration. Experiments were performed on four different plates on two independent days;

FIG. 4 shows primary screening results for the phenotypic cell-based assay and the in vitro growth assay for 26500 compounds: (a) Percent inhibition based on infection ratio relative to each compound and distribution. (b) Percent inhibition based on RFU relative to each compound and distribution. (c) Comparison of inhibition percentage for the phenotypic cell-based assay and the in vitro growth assay for each compound;

FIG. 5 shows serial dilution results from the in vitro growth fluorescence assay and the phenotypic cell-based assay: Typical curves for compounds inhibiting (a,b,c) in vitro bacterial growth (d,e,f) both in vitro and intracellular growth and (g,h,i) intracellular growth only. (a,d,g) Infection ratio relative to compound concentration. (b,e,h) Cell number relative to compound concentration. (c,f,i) Relative fluorescence intensity relative to compound concentration. Compound concentration is given in M;

FIG. 6 shows (a) a scheme of assay automation. (b) a 384-plate format description; (c) a 384-plate dose-response curve description, A to P and a to b correspond to 2-fold serial dilution of INH and Rifampin respectively with a starting concentration of 20 mg/mL in well A or a; RIF: Rifampin 5 μg/mL, Cpd: compound, INH100 1 μg/mL, INH50 0.05 μg/mL;

FIG. 7 illustrates the colony forming units (CFUs) recovered from macrophages at different time points after infection with M. tuberculosis H37Rv. Either Raw264.7 cells (a) or murine BMDM (b) were infected at an MOI of 1:1 and treated with the indicated amount of pyridopyrimidione compound 232 (20 μM) with DMSO, INH (10 μM) and RIF (10 μM) as controls;

FIG. 8 illustrates the colony forming units (CFUs) recovered from macrophages at different time points after infection with M. tuberculosis H37Rv. Cells were infected and treated with the indicated amount of pyridopyrimidione compound 71 (4 to 20 μM);

Table 1 pyridopyrimidinone derivatives (general scaffold I and II, respectively) with their respective inhibitory activities, wherein the numbers in bold print refer to the compounds listed in Example 6;

Table 2 pyridopyrimidinone derivatives (general scaffold VIII and VIIIa) with their respective inhibitory activities, wherein the numbers in bold print refer to the compounds listed in Example 7;

Table 3 shows the cytotoxicity and antibacterial spectrum of pyridopyrimidinone compound 133 (see Table 2); and

Table 4 shows the frequency of spontaneous resistance for representative pyridopyrimidinone compound 264 (see Table 2).

### EXAMPLES

The invention is now further described by reference to the following examples which are intended to illustrate, not to limit the scope of the invention.

### Materials and Methods

### Genetic Constructs and Mycobacterial Strains

A recombinant strain of M. tuberculosis H37Rv expressing the green fluorescent protein (H37Rv-GFP) was obtained by transformation of an integrative plasmid (Abadie et al., 2005; Cremer et al., 2002). Within this plasmid, which is derived from the Ms6 mycobacteriophage, the gfp gene is cloned and constitutively expressed under the strong mycobacterial promoter pBlaF. Electrocompetent cells for M. tuberculosis H37Rv-GFP were prepared from 400 mL of a 15 days old Middlebrook 7H9 culture (Difco, Sparks Md., USA) supplemented with albumin-dextrose-catalase (ADC, Difco, Sparks Md., USA), glycerol and 0.05% Tween 80. Bacilli were harvested by centrifugation at 3000 g for 20 min, washed twice with H₂O at room temperature, and resuspended in 1-2 mL of 10% glycerol at room temperature after recentrifugation. 250 μl of bacilli were mixed with green fluorescent protein encoding plasmid and electroporated using a Biorad Gene Pulser (Biorad). After electroporation, bacilli were resuspended in medium and left one day at 37° C. Transformants were selected on Middlebrook 7H11 medium (Difco, Sparks Md., USA) supplemented with oleic acid-albumin-dextrose-catalase (OADC, Difco, Sparks Md., USA) and 50 μg/mL hygromycin (Invitrogen, Carlsbad, Calif. USA). The selected hygromycin-resistant and green fluorescent colonies appeared after 3 weeks. A 100 mL culture of the H37Rv-GFP strain was grown in Middlebrook 7H9-ADC medium supplemented with 0.05% Tween 80 and 50 μg/mL of hygromycin. Bacteria were harvested, washed twice and suspended in 50 mM sodium phosphate buffer (pH 7.5). The bacteria were then sonicated and allowed to stand for 1 hour to allow residual aggregates to settle. The bacterial suspensions were then aliquoted and frozen at −80° C. A single defrosted aliquot was used to quantify the CFUs (colony forming units) prior to inoculation and typical stock concentrations were about 2 to 5×10⁸ CFU/mL.

### Host Cells

Mouse macrophage cell lines Raw 264.7 (ATCC #TIB-71), J774A.1 (ATCC #TIB-67) or human monocytes (ATCC #TIB-202) differentiated with 50 ng/mL PMA (Sigma) were grown in RPMI 1640 (Gibco) with 10% heat-inactivated fetal calf serum (Gibco).

### Chemical Compounds

The small synthetic molecules from the screening libraries were suspended in pure DMSO (Sigma, D5879-500 mL) at a concentration of 10 mM (Master plates) in Corning 96 well clear V-bottom polypropylene plates (Corning, #3956). The compounds were then reformatted in Greiner 384 well V-shape polypropylene plates (Greiner, #781280) and diluted to a final concentration of 2 mM in pure DMSO. The compounds were kept frozen until use. For screening, compound plates were incubated at room temperature until thawed. The compounds were directly added into the assay plates from the DMSO stock using an EVObird liquid handler (Evotec Technologies), which transfers 250 nl of compound twice to reach a final dilution of 1:100. This one-step dilution reduces the risk of compound precipitation in intermediate plates and allows for a low final DMSO concentration (1%).

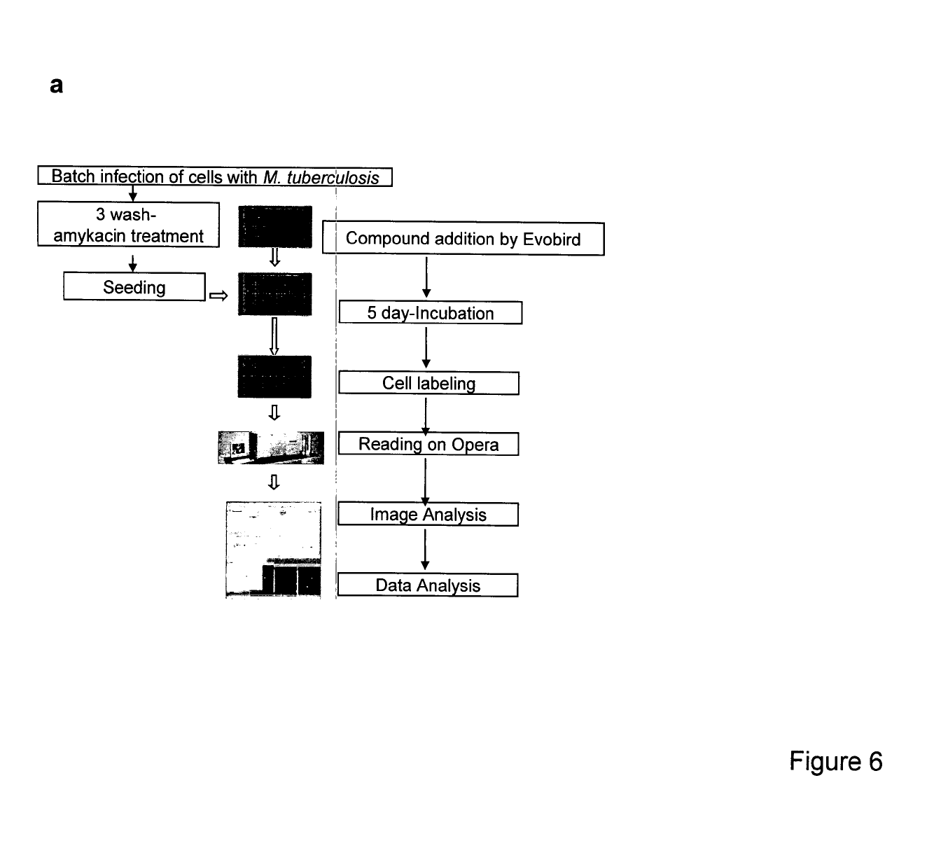

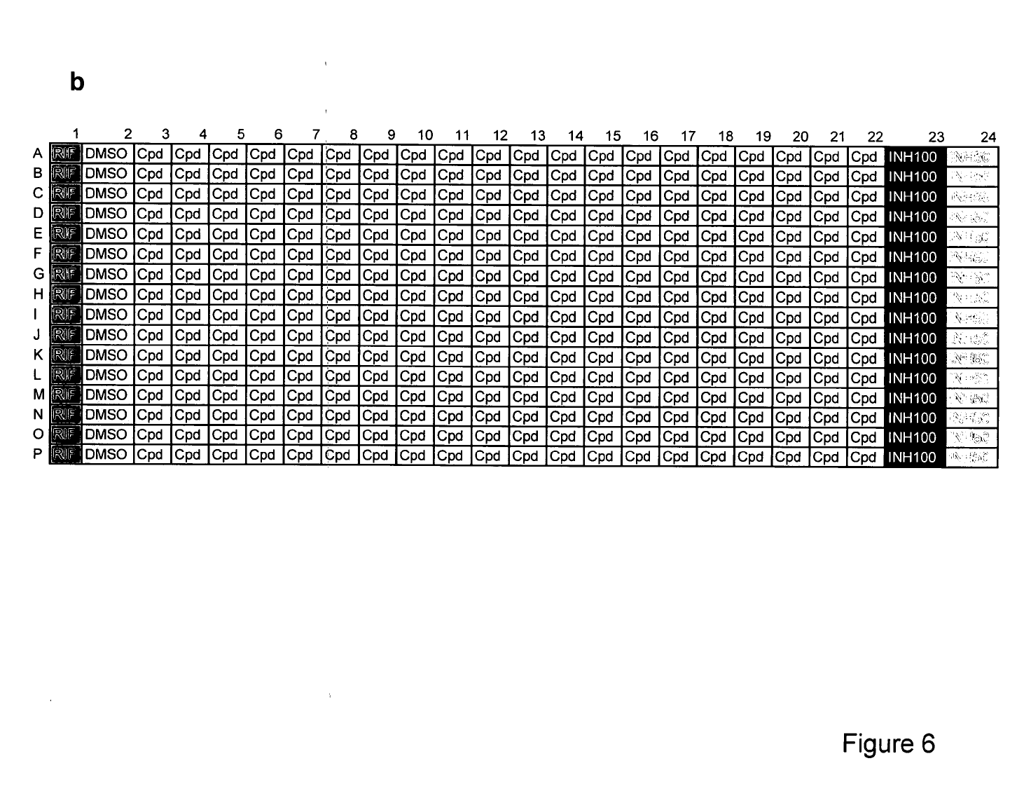

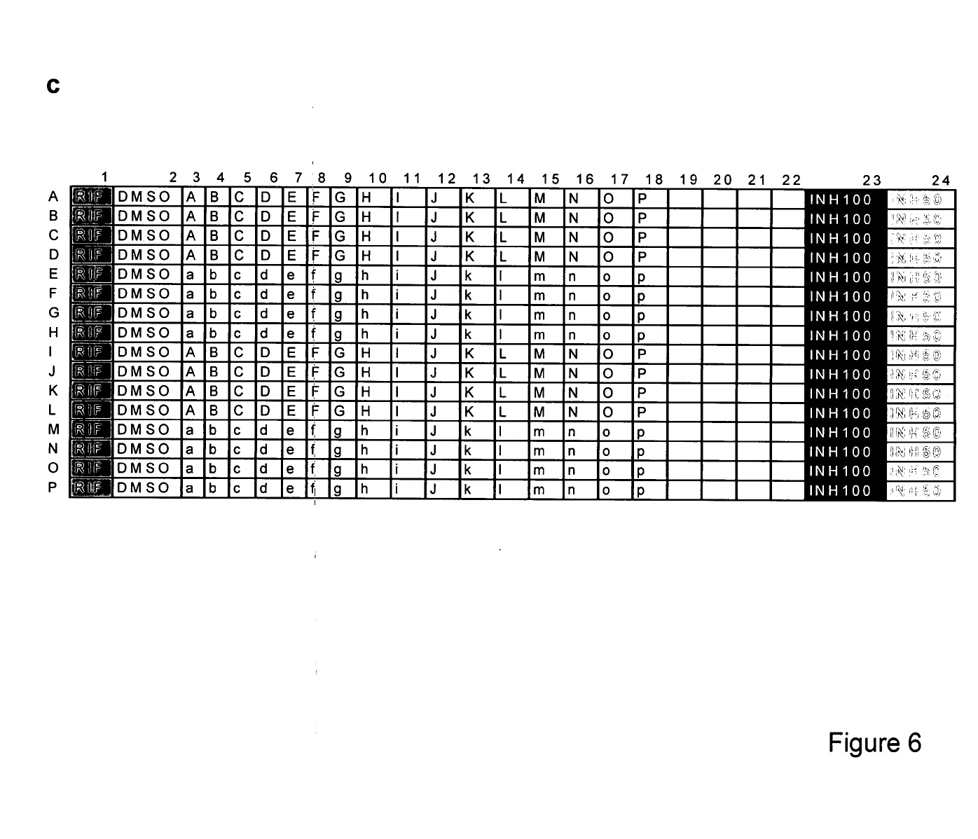

Positive control antibiotics (Isoniazid (Sigma, 13377-50G) and Rifampin (Euromedex, 1059-8, 5 g)) as well as negative controls (DMSO) were added manually in each plate in columns 1-2 and 23-24 (see FIG. 6 b for plate description).

A total of 26500 compounds were tested. These compounds came from commercial libraries from Timtec (25000 from the ActiProbe diverse library, 1000 from the Kinase inhibitors ActiTargK library and 500 from the Protease inhibitors ActitargP library). The screened compounds were selected based on high diversity and drug-like properties (using Lipinski rule-of-five (Lipinski et al., 2001)). They were first screened at one concentration (primary screen, concentration=20 μM). The “positive” compounds selected from the primary screen were then confirmed by testing at 3 concentrations (20, 2 and 0.2 μM) to identify the most active and/or by ten 3-fold ten dilutions (from 20 μM to 0.5 nM).

### Macrophage Invasion Assay Set-Up

Cells were first seeded in 50 μl at a density of 20,000 cells per well of a 384-well plate (Evotec technologies #781058) for 16 hours and then infected with bacterial suspensions at a multiplicity of infection (MOI) varying from 10:1 to 1:1 (bacteria:host cells). After 2 hours, cells were washed three times with phosphate buffered saline (PBS) and the compounds diluted in fresh culture medium were added. Cells were incubated at 37° C., 5% CO₂ for up to seven days.

### Macrophage Batch Infection Assay Scale-Up

Cells (1.5×10⁸ cells) were infected with H37Rv-GFP suspension at a MOI of 1:1 in 300 mL for 2 hours at 37° C. with shaking (100 rpm). After two washes by centrifugation at 1100 rpm (Beckman SX4250, 165 g) for 5 min., the remaining extracellular bacilli from the infected cells suspension were killed by a 1 hour amykacin (20 μM, Sigma, A2324-5G) treatment. After a final centrifugation step, cells were dispensed with the Wellmate (Matrix) into 384-well Evotec plates (#781058) preplated with 10 μl of the respective compound diluted in cell medium. Infected cells were then incubated in the presence of the compound for 5 days at 37° C., 5% CO₂. After five days, macrophages were stained with SYTO 60 (Invitrogen, S11342) followed by plate sealing and image acquisition. During screening, staining of the live cells was carried out on a set of three plates every two hours to limit cell death due to prolonged incubation with cell chemical stain.

### Image Acquisition and Data Analysis

Confocal images were recorded on an automated fluorescent confocal microscope Opera™ (Evotec Technologies) using a 20×-water objective (NA 0.70), 488-nm and 635-nm lasers and a 488/635 primary dichroic mirror. Each image was then processed using dedicated in-house image analysis software (IM). Parameters determined were the total cell number and the number of infected cells. Briefly, the algorithm first segments the cells on the red channel using a sequence of processing steps as described elsewhere (Fenistein et al., 2008). It is generally based on a succession of 1) thresholding the histogram of the original image (3 classes K-means) 2) gaussian filtering the original image with a standard deviation that is set equal to the cells' average radius, 3) searching for the local maxima of the filtered image that provides cell centers as seeds for 4) region growing that defines each cell's own surface and finally 5) removing extremely small cells as potential artifacts or noise. This step provides the total number of cells in the red channel. Infected cells are then defined as those having at least a given number of pixels (usually 3) whose intensity in the green channel is above a given intensity threshold. The ratio of infected cells to the total number of cells is the measure of interest (named infection ratio). For each well, 4 pictures were recorded and for each parameter, the mean of the four images was used.

Data obtained from either the intracellular assay image analysis or from the conventional antibacterial assay (see below) were then processed using ActivityBase (IDBS) to calculate the statistical data (% of inhibition, Z score for each compound, Z′, CV etc. for the control plates) and to store the data in an Oracle database. Additional analyses with regards to both quality control of the screens and hit identification were performed with various software packages including Spotfire (Tibco) and Pipelinepilot (Accelrys).

### In Vitro Aerobic Bacterial Growth Assay

A frozen aliquot of M. tuberculosis H37Rv-GFP was diluted at 1.5×10⁶ CFU/mL in Middlebrook 7H9-ADC medium supplemented with 0.05% Tween 80. Greiner μclear-black 384-well plates (Greiner, #781091) were first preplated with 0.5 μl of compound dispensed by EVOBird (Evotec) in 10 μl of Middlebrook 7H9-ADC medium supplemented with 0.05% Tween 80. 40 μl of the diluted H37Rv-GFP bacterial suspension was then added on top of the diluted compound resulting in a final volume of 50 μl containing 1% DMSO. Plates were incubated at 37° C., 5% CO₂ for 10 days after which GFP-fluorescence was recorded using a Victor 3 reader (Perkin-Elmer Life Sciences).

### Macrophage Infection Assay and Image Analysis

Raw 264.7 (ATCC #TIB-71) (1.5*10⁸ cells) were infected with H37Rv-GFP (Abadie et al., 2005, Cremer et al., 2002) in suspension at a MOI of 1:1 for 2 hours at 37° C. with shaking. After two washes by centrifugation, the remaining extracellular bacilli from the infected cell suspension were killed by a 1 hour Amikacin (20 μM, Sigma, A2324) treatment. After a final centrifugation step, cells were dispensed into 384-well Evotec plates (#781058) preplated with compounds and controls. Infected cells were then incubated for 5 days at 37° C., 5% CO₂. Murine Bone Marrow-Derived Macrophages (BMDM) were produced as described previously (Brodin et al., 2006). Briefly, cells were extracted from the femurs and tibia of 6 weeks old female mice (C57BL/6, Orientbio) and cultivated in RPMI 1640 media containing 10% heat-inactivated fetal calf serum (FCS) (both from Gibco® at Invitrogen, Carlsbad, Calif.) and 10% L-929 cell conditioned medium. Peripheral Blood Mononuclear Cells (PBMC) were isolated from Buffy coat from healthy volunteers. Buffy coat diluted in PBS supplemented with 1% FCS was treated with 15 ml of Ficoll-Paque Plus (Amersham Biosciences, Sweden) and centrifuged at 2500×g for 20 min. PBMC were obtained by CD14⁺ bead separation (Miltenyi Biotec, Germany), washed 3-times with PBS (1% FCS) and transferred to 75 cm² culture flasks containing RPMI 1640 media, 10% FCS and 50 ng/ml of recombinant-human macrophage colony stimulating factor (R & D systems, Minneapolis). Six day old adherent murine BMDM and PBMC derived human macrophages were infected with H37Rv-GFP (Abadie et al., 2005) in suspension at a MOI of 1:1 for 2 hours at 37° C. and then extensively washed and finally incubated with compounds or controls. After several days, macrophages were stained with SYTO 60 (Invitrogen, S11342) and image acquisition was performed on an EVOscreen-MarkIII fully automated platform (PerkinElmer) integrated with an Opera™ (20×-water objective, NA 0.70) and located in a BSL-3 safety laboratory. Mycobacteria-GFP were detected using a 488-nm laser coupled with a 535/50 nm detection filter and cells labeled with a 635-nm laser coupled with a 690/40 nm detection filter. Four fields were recorded for each plate well and each image was then processed using dedicated in-house image analysis software (IM) as described elsewhere (Fenistein et al., 2008).

### Mycobacterial Strains and In Vitro Bacterial Growth Assay

Mycobacterium tuberculosis H37Rv, H37Ra and BCG Pasteur were used as reference strains. All strains were diluted at 1.5×10⁶ CFU/mL in Middlebrook 7H9-ADC medium supplemented with 0.05% Tween 80. 384-well plates (Greiner, #781091) were first preplated with 0.5 μl of compound dispensed by EVOBird (Evotec) in 10 μl of Middlebrook 7H9-ADC medium supplemented with 0.05% Tween 80. Forty microliters of the diluted H37Rv-GFP bacterial suspension was then added to the diluted compound resulting in a final volume of 50 μl containing 1% DMSO. Plates were incubated at 37° C., 5% CO₂ for 10 days. Mycobacterial growth was determined by measuring GFP-fluorescence using a Victor 3 reader (Perkin-Elmer Life Sciences) for H37Rv-GFP or with resazurin method. Isoniazid at 0.05 μg/mL and 1 μg/mL (Sigma, I3377), Rifampin at 1 μg/mL (Euromedex) and DMSO were used as controls.

### Cytotoxicity Assay

In order to address compound toxicity, seven cell lines originating from different body tissues were cultivated in the presence of 3-fold dilutions of compounds starting from 100 μM. After 5 days of culture, cell viability was assessed by the resazurin test. Briefly, cells were incubated with 10 μg/mL of resazurin (Sigma-Aldrich St. Louis, Mo.) for 4 h at 37° C. under 5% CO2. Resofurin fluorescence (RFU) was measured as indicated above. Percentage of toxicity on cells was calculated as follows: Cytotoxicity (%)=(RFU_{DMSO}−RFU_{Blank})−(RFU_{compound}−RFU_{blank})/(RFU_{DMSO}−RFU_{Blank})×100. Percentage of cytotoxicity was plotted against compound concentration and the minimal toxic concentration (MTC₅₀) was determined by non-linear regression analysis as the lowest compound concentration where fifty percent toxicity was observed on the corresponding cell line.

### Frequency of Spontaneous Resistance

The frequency of spontaneous mutations was determined on 7H10 plates containing increasing concentrations of dintirobenzamide (0.2, 0.8, 1.6 and 3.2 μg/ml) or pyridopyrimidinone (0.4, 0.8, 1.6 and 3.2 μg/ml) compounds. 10⁶, 10⁷ and 10⁸ CFU containing bacterial suspensions were spread on compound containing agar plates. After 5-6 weeks at 37° C., colonies were counted and frequency of mutation was evaluated as the ratio of colonies relative to the original inoculum. DMSO and INH were used as negative and positive controls, respectively.

### Example 1

### Phenotypic Macrophage-Based Assay Set-Up and Automated Image Quantification

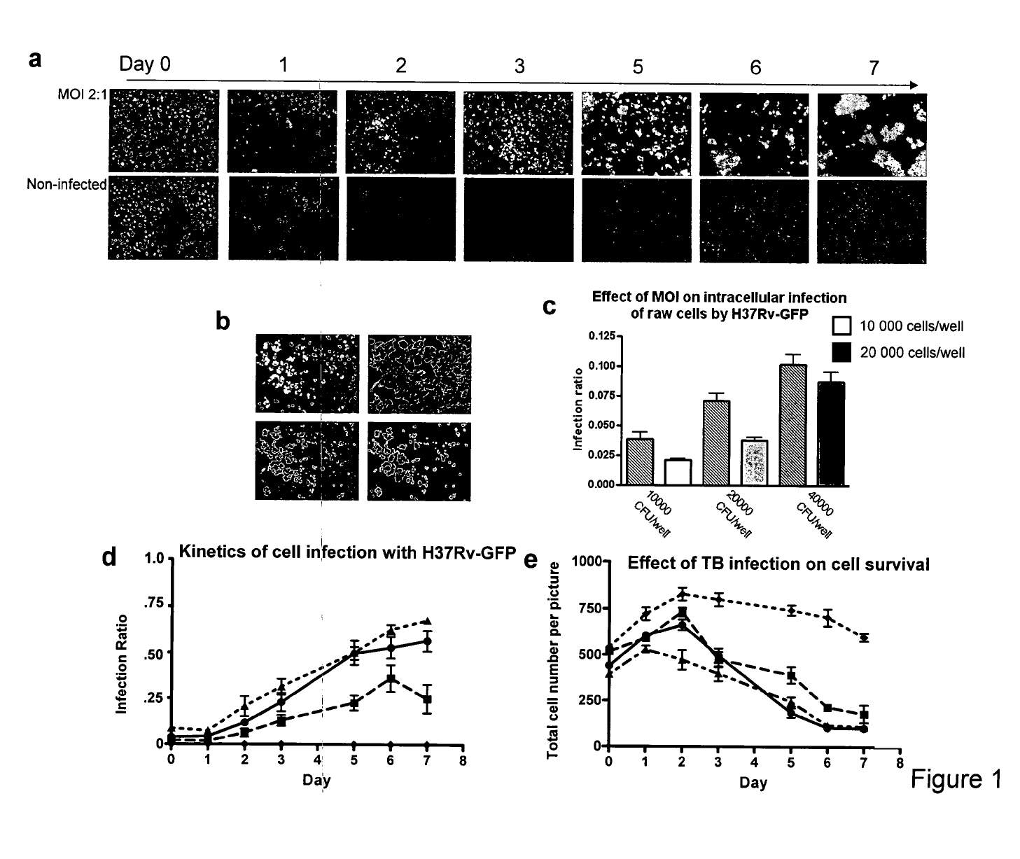

To set-up the optimal conditions of M. tuberculosis infection, Raw264.7 macrophages were first infected with mycobacteria that constitutively express green fluorescent protein (GFP) at different multiplicities of infection (MOI) followed by kinetic analysis. Up to 7 days post bacillus infection, the host live cells were daily labeled with the red chemical fluorescent dye Syto60, and confocal images of live samples were acquired using an automated confocal microscope. Typical images are displayed in FIG. 1a. During the first 24 hours, a few discrete weakly fluorescent bacteria localized within the cells. By day 2, the average number of cells had increased and mycobacteria had started to spread into neighboring cells leading to zones of strongly fluorescent bacteria. The localization of the green signal is always within a distance of 5 μm to that of the red cell signal and in most cases actually overlaps with the cell signal. This confirms the intracellular nature of the mycobacteria growth. By day 4, the cell number has significantly diminished and the bacteria have formed large, highly fluorescent aggregates, which cover almost the entire image from day 5 onwards. As a control, non-infected cells grew up to confluence at day 2 and remained alive until day 7.

In order to automatically quantify the intracellular bacterial load, an in-house image analysis script was developed. This script enables the automated quantification of the number of cells and the percentage of infected cells, whereby an infected cell is a cell containing at least three green pixels with an intensity above a defined threshold (FIG. 1b). 2 hours after infection, between 2 and 10% of Raw264.7 cells were found to harbor a low number of bacilli (FIG. 1c). The percentage of infected cells, hereafter named infection ratio, continued to increase from 72 hours post-infection reaching up to 70% at seven days post infection. This increase in infection ratio correlated with an increase in cell mortality (FIG. 1d/e).

### Example 2

### Comparative Minimal Inhibitory Concentration of Known Anti-Tubercular Drugs

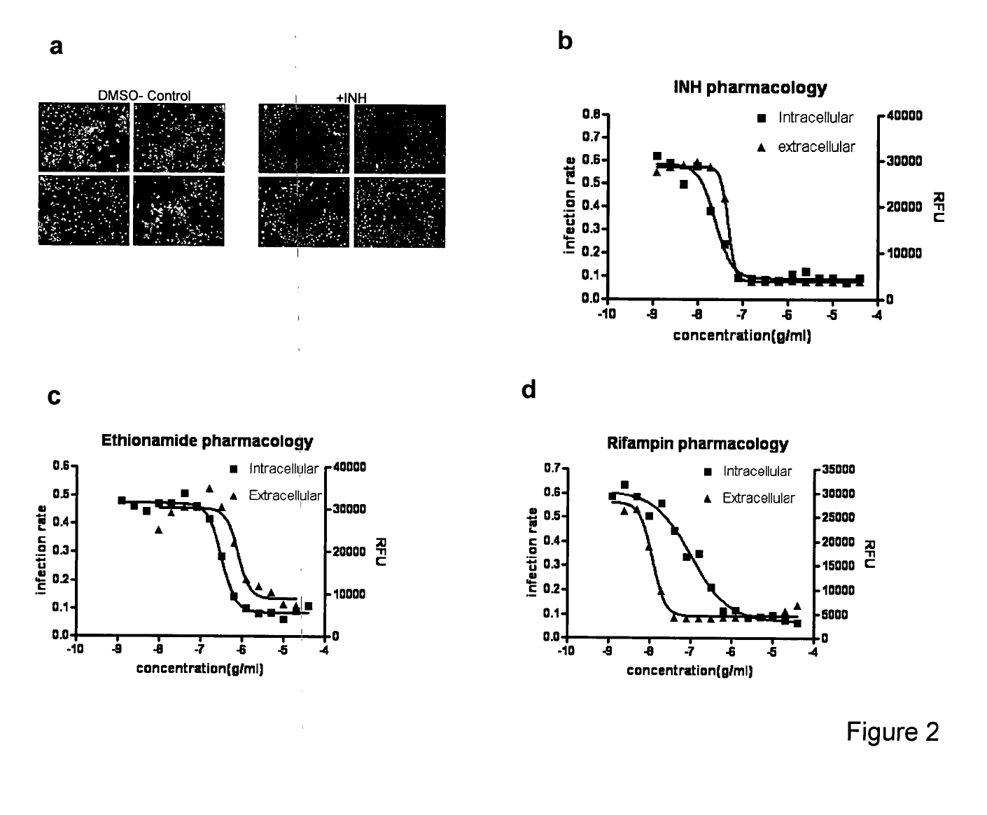

To validate the assay set-up, the effect of current anti-tuberculosis drugs on M. tuberculosis intracellular growth was investigated. 2-fold serial dilutions of isoniazid (INH), rifampin and ethionamide were performed, followed by testing on macrophages that had previously been infected with M. tuberculosis H37Rv-GFP. After 5 days of incubation, macrophages were stained, and images acquired on an automated confocal microscope as described above. A larger number of cells and a fewer number of bacteria are clearly seen on pictures corresponding to samples that were incubated with INH compared to the DMSO negative control. This shows that INH prevents both intracellular M. tuberculosis growth and bacillus mediated cytotoxicity (FIG. 2a). A clear inhibition dose-response curve was obtained by image-extracted analysis (FIG. 2b). In parallel, inhibition of M. tuberculosis H37Rv-GFP in vitro growth by INH was monitored by recording green fluorescence intensity under the same conditions. In both experiments, the minimal inhibitory concentration (MIC) for INH was 0.1 μg/mL, which is in accordance with the MIC reported in the literature for extracellular M. tuberculosis growth (Andries et al., 2005). Similar results were obtained with the standard anti-tuberculosis drugs ethionamide (FIG. 2c) and ethambutol (data not shown), whereas for rifampin, there was a log-fold decrease in the MIC in the cell-based assay compared to the in vitro assay (FIG. 2d). The diminished efficacy of rifampin in the cell-based assay is likely due to impaired cell penetration and further demonstrates that it is the intracellular antibacterial activity that is being monitored in this assay. Thus, adaptation of both the intracellular and the in vitro M. tuberculosis growth assay for high throughput screening (HTS) was performed.

### Example 3

### Assay Scale-Up and Validation

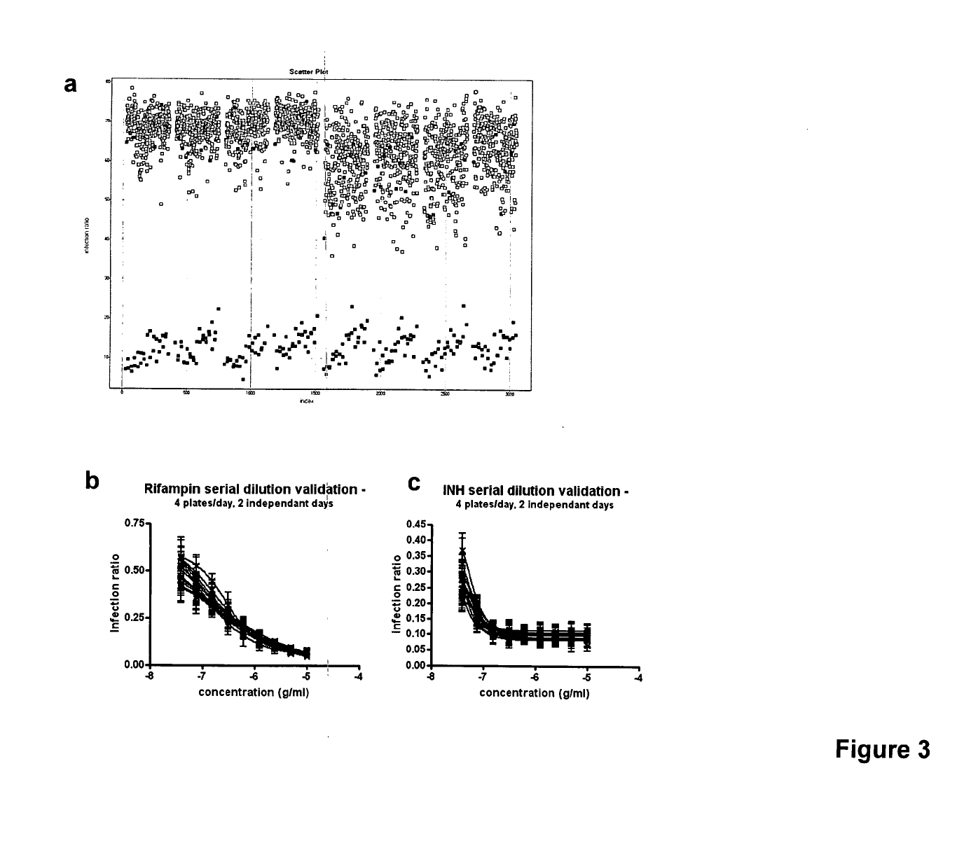

To simplify the protocol for HTS purposes, macrophages were infected in batch with M. tuberculosis before being dispensed onto the compounds. The batch infection was carried out with macrophages in suspension at 37° C. under mild shaking. Free unbound mycobacteria were removed by washing three times with PBS and differential centrifugation, as well as by an additional one-hour incubation step with amykacin, an antibiotic known to selectively kill extracellular microbes (FIG. 6a). M. tuberculosis infected macrophages were then seeded in plates that had been previously dispensed with the compounds, DMSO or antibiotic controls. The day-to-day as well as plate-to plate reproducibility was first tested. To this end, either serial dilutions of INH or rifampin were dispensed into 8 plates along with the regular DMSO and positive control (INH at 1 μg/mL (MIC100) and at 0.05 μg/mL (MIC90) and rifampin at 1 μg/mL) wells that were subsequently seeded with infected cells. The same experiment was repeated over 2 consecutive days. After incubation for 5 days and macrophage staining, pictures from each plate were acquired. The mean infection ratio determined for the DMSO negative controls in each plate for the 2 days of experiments was between 50% and 70%, whereas for the INH and rifampin samples, the mean infection ratio fell to below 20% (FIG. 3a). Despite some variation in the mean infection ratio between the two experiments, the difference between the INH-positive and DMSO-negative controls was above five-fold for both days. P values calculated for each plate using a paired t-student test also confirmed a significant difference between the positive and negative controls (p<0.000001, data not shown). In addition, the inventors performed an experiment to determine if inhibitors of M. tuberculosis intracellular growth infection dispensed in any well on the plate could be detected by performing double-blind controls (spike of INH and rifampin at 3 different concentrations). Indeed, one hundred percent of the spikes were identified (data not shown). Taken together, these results prove that the assay is sensitive enough to be able to identify inhibitors under HTS conditions. Finally, the robustness of the assay was checked by monitoring the dose-response of reference compounds. Almost identical MICs for the antibiotic positive controls were determined independent of the plate or the day of the experiment (FIG. 3b/c). Calculated MICs from the image based quantification of the infection ratio were 0.16+/−0.05 μg/mL and 2.4+/−1.3 μg/mL for INH and rifampin, respectively, and were confirmed by CFU plating (data not shown). In parallel, the extracellular growth assay was validated with a similar approach (data not shown).

### Example 4

### Primary Screening of a Large Library of Small Synthetic Compounds Using the Phenotypic Cell-Based Assay

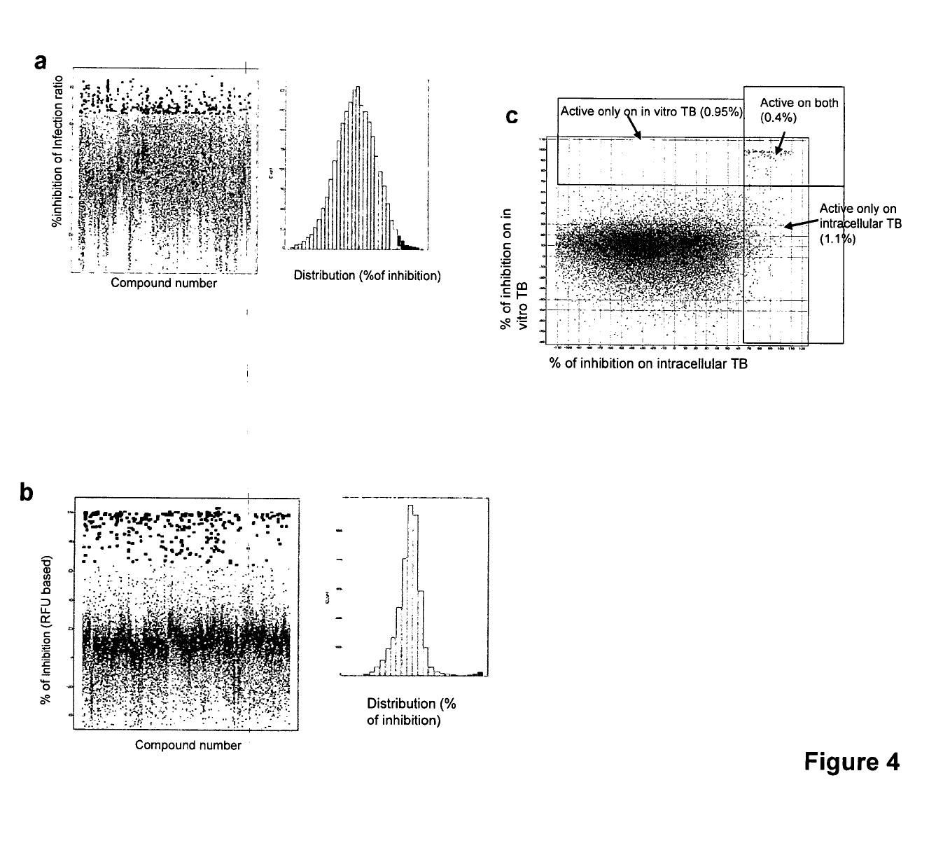

A 26500 small molecule compound library, that was selected for its high chemical diversity and drug-like properties according to the Lipinski rules (Lipinski et al., 2001), was chosen as the first library to be screened using the validated phenotypic cell-based assay. The primary screen was carried out with compounds at 20 μM in singleton. The throughput was set to about 6000 compounds per working day encompassing 25 plates. The screening was performed with Raw264.7 cells that had been expanded from frozen stocks for ten days before infection with M. tuberculosis H37Rv-GFP. To accept the screening results, the MICs obtained from 2 serial dilutions of INH and Rifampin processed at the beginning and at the end of the screening day should show similar results compared to the values obtained during the validation (see above). Each screened plate is then accepted by the quality control procedure if the window between DMSO and INH (1 μg/ml) is higher than 3 and the CV calculated for the 320 compounds present in each plate is lower than 25. Such quality control criteria allow the identification of hits with an activity higher than 75%. Subsequently, the percent inhibition for each compound was determined relative to the corresponding mean infection ratio between 1 μg/mL INH (100%) and DMSO (0%) in the same 384-well plate. The percent inhibition distribution is centered around −20% of inhibition (FIG. 4a). It was decided to select compounds that have an inhibitory effect greater than 65% which corresponds to a little less than 1.5% of the total compounds.

In parallel, the same compounds were only tested for their inhibitory activity on the M. tuberculosis H37Rv-GFP bacterial growth. The results from this assay, which are based on classical fluorescence intensity, showed a higher degree of reproducibility and the criteria for plate validation was set to a Z′ value (DMSO/INH) greater than 0.35. The throughput for this fluorescence based assay was approximately 20,000 compounds per day. Compounds that prevented M. tuberculosis growth in vitro with an inhibitory effect above 65% were then selected as hits (1.4%) as they belong to a clear independent population compared to the inactive population centered to 0% (FIG. 4b).

The results gathered from the two different screenings were then compiled and compared (FIG. 4c). Four different populations could be identified: compounds that are i) active only on extracellular bacteria, ii) active only on intracellular bacteria, iii) active in both settings or iv) not active. 657 compounds (2.5%) belonged to one of the first three categories and, thus, were selected for further investigation.

An important parameter that can be measured during image analysis is the total cell number, also referred to as cell cytotoxicity. A low cell number can be the result of two independent phenomena, the compound toxicity and M. tuberculosis growth mediated cell toxicity. Indeed, at day 5 after infection with M. tuberculosis, the cell number decreased to less than 100 cells per image compared to more than 500 cells per image for uninfected cells (FIG. 1e). In contrast, a high cell number is obtained only when the compound is not toxic and prevents mycobacterial growth. This turns out to be a second relevant measurement of a compound's anti-mycobacterial activity. However, this criterion was not applied for the selection of hits from the primary screen as a low cell number was found for only a few compounds and the inventors wanted to avoid failing to select highly active compounds that would later on prove to be active at much lower concentrations despite a cell toxicity at 20 μM. An additional validation criterion of a Z′ (DMSO/INH) value of the total cell number greater than 0.2 was added for the following screening steps.

### Example 5

### Confirmation of Screening Results, Dose-Response Analysis and Hit Classification

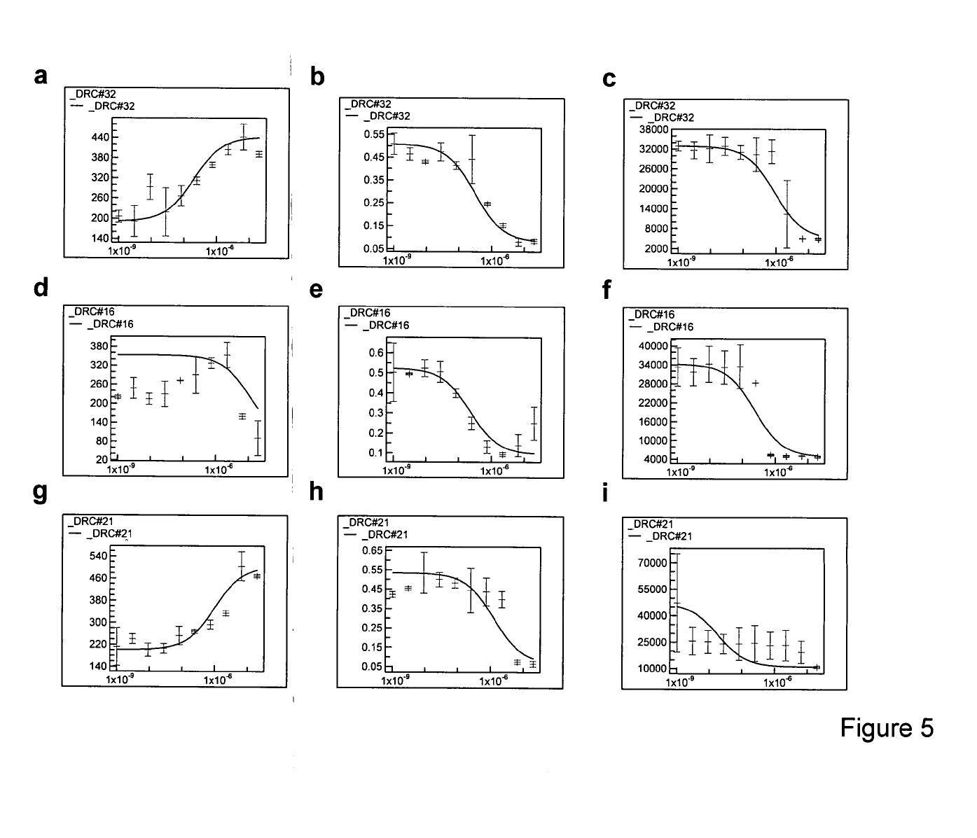

The 657 selected hits were first confirmed at 3 different concentrations, 20 μM, 2 μM and 0.2 μM. For 340 hits the activity was confirmed either at 20 μM or 2 μM, on the intracellular or the in vitro assay. From this latter list, 121 compounds demonstrated an inhibitory activity above 65% at 2 μM without any apparent cell toxicity at 20 μM and consequently were selected for further confirmation by ten 3-fold serial dilutions. All 121 compounds were confirmed by serial dilution with a MIC ranging between 250 nM and 20 μM. The results shown in FIG. 5 are representative of the three types of behavior observed: most of the compounds exhibited a clear dose response curve when activity was measured as infection ratio (FIG. 5b/e/h). Compounds active on the bacilli level present a similar activity in the extracellular assay (FIG. 5c/f) even if the MIC is different from one assay to the other. A few compounds don't present clear activity on the in vitro bacilli (FIG. 5i) and may represent drugs acting through a cellular target or on a bacilli target involved only during the infection process. Furthermore, toxic compounds can be identified thanks to a dramatic decrease in the cell number when the compound concentration increases (FIG. 5d) and activity of non-toxic compounds are validated by a dose response protective effect on the cell number (FIG. 5a). Consequently cell number detection represents an independent secondary assay in the same well as the primary assay.

The 121 confirmed hits can be clustered as various independent/general scaffolds. The number of compounds for each scaffold varied, ranging from 1 to 69 molecules. The molecules from the 69-compound scaffold share a common structure which is similar to INH thereby validating the screening results. The pyridopyrimidinone general scaffold is the focus of the present invention.

### Example 6

### Derivatization of the Pyridopyrimidinone Compounds

The pyridopyrimidinone compounds (scaffolds I and II) underwent derivatization according to the methods outlined below. (Schemes 1-6). Resulting derivatives were examined for inhibitory activity using the assay described above and the results are summarized in Table 1.

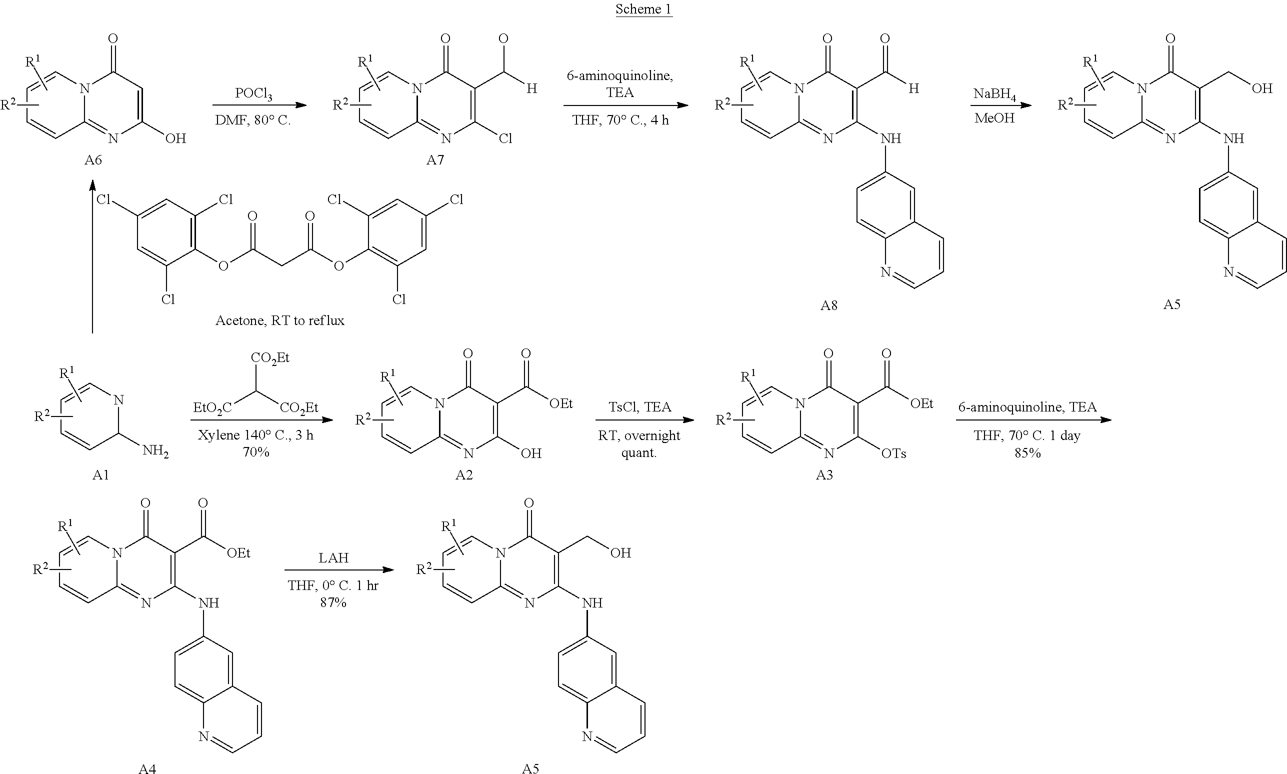

### Method A.

### General Procedure for the Synthesis of A2

To a stirred solution of A1 (12.0 mmol) in Xylene (20 mL) was added diethyl ethoxymethylenemalonate (36.0 mmol). The mixture was stirred at 140° C. for overnight. After cooling, the dark residue was triturated with EtOAc (50 mL). The residual pale solid was collected by filtration and washed with EtOAc to give A2.

### General Procedure for the Synthesis of A3

To a stirred solution of A2 (10.0 mmol) in THF (20 mL) was added triethylamine (12.0 mmol) and p-toluenesulfonyl chloride (11.0 mmol) at 0° C. The reaction mixture was refluxed overnight. t The organic solvent was then evaporated, diluted with CH₂Cl₂ (100 mL) and washed with brine (100 mL). The organic layer was dried over anhydrous MgSO₄ and concentrated in vacuo. The crude product was purified by flash column chromatography to give A3.

### General Procedure for the Synthesis of A4

To a stirred solution of A3 (1.0 mmol) in THF (5.0 mL) was added triethylamine (1.5 mmol) and 6-aminoquinoline (1.1 mmol). The reaction mixture was stirred at 70° C. overnight. The reaction mixture was then concentrated, diluted with CH₂Cl₂ (50 mL) and washed with 1N HCl (50 mL) and saturated NaHCO₃ solution (50 mL). The organic layer was dried over anhydrous MgSO₄ and concentrated in vacuo. The crude product was purified by flash column chromatography to give A4.

### General Procedure for the Synthesis of A5

To a stirred solution of A4 (0.06 mmol) in THF (2.0 mL) was added LiAlH₄ (0.10 mmol) at 0° C. The reaction mixture was stirred at room temperature. After 1 hr, H₂O (0.1 mL) was added dropwise. The reaction mixture was filtered off and concentrated in vacuo. The crude product was purified by flash column chromatography to give A5.

### Method B.

### General Procedure for the Synthesis of A6

A solution of aminopyridine (0.034 mol) and bis-(2,4,6-trichlorophenyl) malonate (0.034 mol) in acetone (150 mL) was stirred at room temperature. After 30 min, triethylamine (0.068 mol) was added and the reaction mixture was further stirred for 30 min. The resulting solid was filtered, washed with ethyl acetate and dried in vacuo to give A6.

### General Procedure for the Synthesis of A7

To a DMF (2.0 mL) was added POCl₃ (3.0 mmol) at 0° C. After the mixture was stirred at 0° C. for 40 min, a solution of A6 (1.0 mmol) in DMF (2.0 mL) was added and stirred at 80° C. for 1 h. The mixture was poured to the ice and then the resulting solid was filter, washed with water and dried in vacuo to give A7.

### General Procedure for the Synthesis of A8

To a stirred solution of A7 (1.0 mmol) in THF (5.0 mL) was added triethylamine (1.5 mmol) and 6-aminoquinoline (1.1 mmol). The reaction mixture was stirred at 70° C. After 4 h, the organic solvent was removed under reduced pressure. The resulting solid was washed with MeOH and dried. The crude residue was further purified by column chromatography to give A8.

General Procedure for the Synthesis of A5 from A8

To a stirred solution of A8 (0.57 mmol) in MeOH (5.0 mL) was added NaBH₄ (0.87 mmol) at 0° C. and the reaction mixture was stirred at room temperature. After 1 h, the reaction mixture was quenched with water (1.0 mL) and concentrated under reduced pressure. The resulting crude residue was purified by column chromatography to give A5.

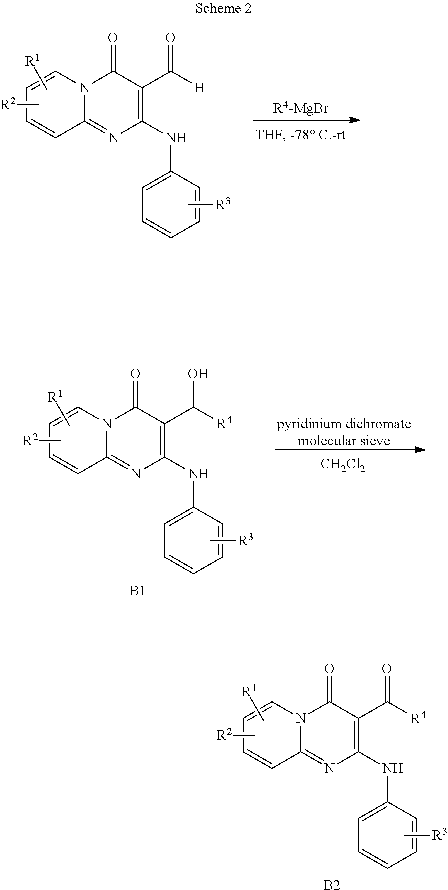

### General Procedure for the Synthesis of B1

To s suspension of aldehyde (0.060 mmol) in THF (500 uL) was added alkyl or phenylmagnesium bromide (3.0 M solution in ether, 0.070 mmol) at −78° C. After 20 min, the reaction temperature was raised to room temperature and the resulting mixture was further stirred for 10 min. The reaction mixture was quenched with water (3.0 mL) and extracted with MC (5 mL×2). The organic phase was washed with brine (10.0 mL), dried over anhydrous MgSO₄ and concentrated in vacuo. The crude residue was purified by a flash column chromatography (n-hexane:ethylacetate) to give B1.

### General Procedure for the Synthesis of B2

To a solution of B1 (0.14 mmol) in THF (5.0 mL) was added pyridinium dichromate (0.20 mmol) and molecular sieve (200 mg). The mixture was stirred at room temperature h. After 6 h, the reaction mixture was filtered off and the filtrate was concentrated under reduced pressure. The crude residue was purified by flash column chromatography to give B2.

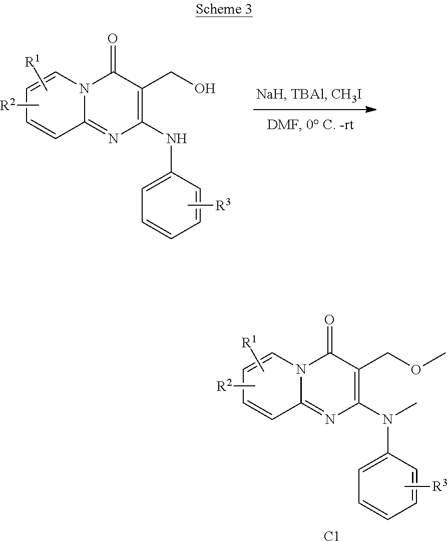

### General Procedure for the Synthesis of C1

To a solution of alcohol (0.085 mmol) in DMF (500.0 uL) was added NaH (60% dispersion in mineral oil, 0.13 mmol) at 0° C. After 10 min, TBAI (0.0086 mmol) and CH₃I (0.13 mmol) was added and the resulting mixture was stirred at room temperature. After 4 h, the reaction mixture was quenched with water and concentrated under reduced pressure. The resulting crude residue was purified by column chromatography (methylene chloride:methanol) to give C1.

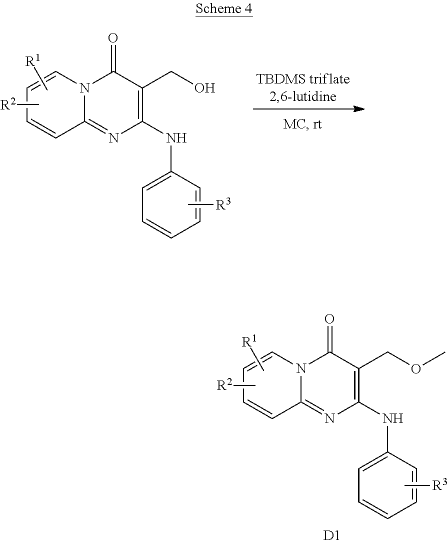

### General Procedure for the Synthesis of D1

To a stirred solution of alcohol (0.57 mmol) and 2,6-lutidine (1.15 mmol) in MC (5.0 mL) was added tert-butyldimethylsilyl trifluoromethanesulfonate (0.86 mmol) under ice bath. After addition of reagent, the ice bath was removed and the reaction mixture was further stirred at room temperature for 1 h. The reaction mixture was diluted with MC (10.0 mL) and washed with water (10.0 mL) and brine (10.0 mL). The organic layer was dried over anhydrous MgSO₄ and concentrated in vacuo. The resulting crude residue was purified by column chromatography (methylene chloride:methanol) to give D1.

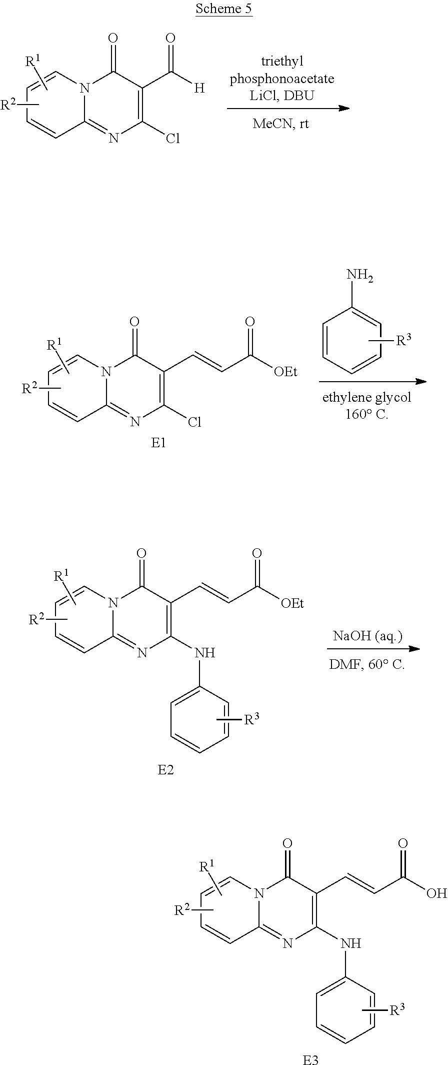

### General Procedure for the Synthesis of E1

To a stirred suspension of LiCl (0.50 mmol) in dry MeCN (2.0 mL) was added triethyl phosphonoacetate (0.50 mmol), DBU (0.42 mmol) and finally aldehyde (0.42 mmol) and the resulting solution was stirred at room temperature. After 3 h, the reaction mixture was concentrated under reduced pressure and the resulting crude residue was purified by column chromatography (n-hexane:ethylacetate) to give E1.

### General Procedure for the Synthesis of E2

A mixture of starting chloride (0.16 mmol) and aniline (1.62 mmol) in ethylene glycol (1 mL) was heated at 160° C. with stirring. After 2 h, the reaction mixture was cooled to room temperature, poured to the ice and extracted with MC (5.0 mL×3). The organic layer was washed with brine (15.0 mL), dried over anhydrous MgSO₄ and concentrated in vacuo. The crude residue was dissolved with MC and the resulting insoluble precipitate was filtered to give E2.

### General Procedure for the Synthesis of E3

To a suspension of E2 (0.074 mmol) in DMF (500.0 uL) was added aqueous NaOH (0.22 mmol). The resulting mixture was stirred at 60° C. After 5 h, the reaction mixture was neutralized with 1 M HCl (aq.) and resulting precipitate was filtered, washed with water and dried in vacuo to give E3.

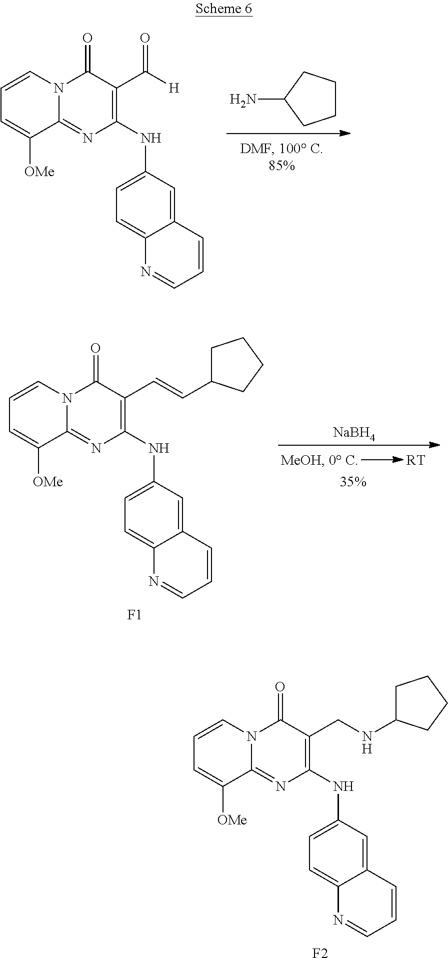

### General Procedure for the Synthesis of F1

To a solution of 3-(Hydroxymethyl)-9-methoxy-2-(quinolin-6-ylamino)-4H-pyrido[1,2-a]pyrimidin-4-one (0.14 mmol) in DMF (1.0 mL) was added cyclopentylamine (0.28 mmol).

The mixture was stirred at 100° C. After 3 h, the reaction mixture was concentrated in vacuo. The crude product was purified by flash column chromatography to give F1.

### General Procedure for the Synthesis of F2

To a solution of F1 (0.10) in methanol (7.0 mL) was added sodium borohydride (0.15 mmol) at 0° C. The mixture was stirred at 0° C. After 1 h, the reaction temperature was raised to room temperature and the resulting mixture was further stirred for overnight. After reaction was completed, the reaction mixture was quenched with water and concentrated in vacuo. The crude product was purified by flash column chromatography to give F2.

### 3-(Hydroxymethyl)-9-methoxy-2-(6-methoxypyridin-3-ylamino)-4H-pyrido[12-a]-pyrimidin-4-one (1)

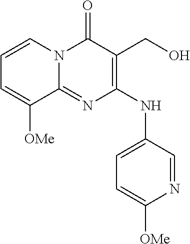 [SMILES: COc1ccc(Nc2nc3c(OC)cccn3c(=O)c2CO)cn1]

White solid; mp=over 250° C. (decomp.); ¹H NMR (400 MHz, CDCl₃+CD₃OD) δ 3.89 (s, 3H), 3.94 (s, 3H), 4.82 (s, 2H), 6.72 (d, J=8.8 Hz, 1H), 6.86 (dd, J=7.2, 7.6 Hz, 1H), 6.91 (dd, J=1.6, 7.6 Hz, 1H), 8.03 (dd, J=2.8, 8.8 Hz, 1H), 8.44 (d, J=2.8 Hz, 1H), 8.51 (dd, J=1.6, 7.2 Hz, 1H); ¹³C NMR (100 MHz, DMSO-d₆) δ 53.8, 56.3, 56.8, 95.1, 110.4, 111.8, 113.0, 119.5, 130.6, 133.0, 138.8, 144.3, 151.2, 157.3, 157.6, 160.3; LC-MS (ESL m/z): 329[M+H]⁺.

### N-(5-(3-(Hydroxymethyl)-9-methoxy-4-oxo-4H-pyrido[1,2-a]pyrimidin-2-ylamino)-pyridin-2-yl)acetamide (2)

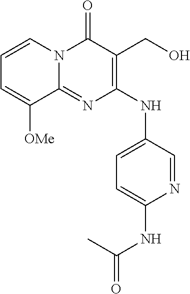 [SMILES: COc1cccn2c(=O)c(CO)c(Nc3ccc(NC(C)=O)nc3)nc12]

Yellow solid; mp=over 390° C. (decomp.); ¹H NMR (400 MHz, DMSO-d₆) δ 2.07 (s, 3H), 3.93 (s, 3H), 4.70 (s, 2H), 5.15 (brs, 1H), 7.08 (dd, J=7.2, 7.2 Hz, 1H), 7.29 (dd, J=1.2, 7.2 Hz, 1H), 8.01 (d, J=9.2 Hz, 1H), 8.18 (dd, J=2.8, 9.2 Hz, 1H), 8.47 (dd, J=1.2, 7.2 Hz, 1H), 8.71 (brs, 1H), 8.85 (d, J=2.8 Hz, 1H), 10.41 (brs, 1H); ¹³C NMR (100 MHz, CDCl₃) δ 23.7, 54.1, 56.7, 94.9, 112.8, 112.9, 113.2, 118.8, 129.7, 132.8, 139.8, 143.3, 146.7, 150.5, 155.7, 156.4, 168.6; LC-MS (ESI, m/z): 356[M+H]⁺.

### 3-(Hydroxymethyl)-2-(6-hydroxypyridin-3-ylamino)-9-methoxy-4H-pyrido[1,2-a]-pyrimidin-4-one (3)

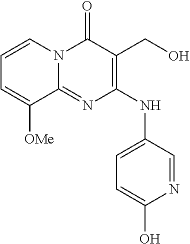 [SMILES: COc1cccn2c(=O)c(CO)c(Nc3ccc(O)nc3)nc12]

Green solid; mp=over 320° C. (decomp.); ¹H NMR (400 MHz, DMSO-d₆) δ 3.89 (s, 3H), 4.61 (s, 2H), 6.30 (d, J=10.0 Hz, 1H), 7.01 (dd, J=7.2, 7.6 Hz, 1H), 7.22 (d, J=7.6 Hz, 1H), 7.63 (dd, J=2.8, 10.0 Hz, 1H), 8.12 (d, J=2.8 Hz, 1H), 8.41 (d, J=7.2 Hz, 1H); LC-MS (ESI, m/z): 315[M+H]⁺.

### 2-(2-Chloropyridin-4-ylamino)-3-(hydroxymethyl)-9-methoxy-4H-pyrido[1,2-a]-pyrimidin-4-one (4)

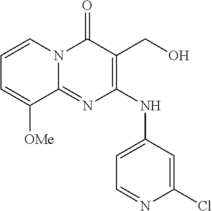 [SMILES: COc1cccn2c(=O)c(CO)c(Nc3ccnc(Cl)c3)nc12]

Pale gray solid; mp=over 240° C. (decomp.); ¹H NMR (400 MHz, DMSO-d₆) δ 3.99 (s, 3H), 4.70 (s, 2H), 7.15 (dd, J=7.2, 7.6 Hz, 1H), 7.35 (d, J=7.6 Hz, 1H), 7.68 (dd, J=1.6, 5.6 Hz, 1H), 8.14 (d, J=5.6 Hz, 1H), 8.48-8.50 (m, 2H); LC-MS (ESI, m/z): 333, 335[M+H]⁺, Cl isotope pattern.

### 2-(5-Bromopyridin-3-ylamino)-3-(hydroxymethyl)-9-methoxy-4H-pyrido[1,2-a]-pyrimidin-4-one (5)

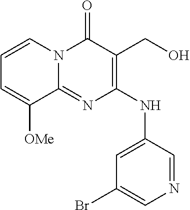 [SMILES: COc1cccn2c(=O)c(CO)c(Nc3cncc(Br)c3)nc12]

Pale yellow solid; mp=over 280° C. (decomp.); ¹H NMR (400 MHz, DMSO-d₆) δ 3.98 (s, 3H), 4.71 (s, 2H), 7.14 (dd, J=7.2, 7.6 Hz, 1H), 7.34 (d, J=7.6 Hz, 1H), 8.29 (d, J=2.0 Hz, 1H), 8.49 (dd, J=1.2, 7.2 Hz, 1H), 8.87 (d, J=2.0 Hz, 1H), 9.10 (dd, J=2.0, 2.0 Hz, 1H); LC-MS (ESI, m/z): 377, 379[M+H]⁺, Br isotope pattern.

### 3-(Hydroxymethyl)-9-methoxy-2-(6-methylpyridin-3-ylamino)-4H-pyrido[1,2-a]pyrimidin-4-one (6)

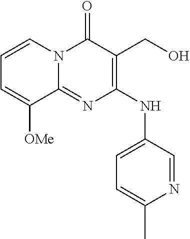 [SMILES: COc1cccn2c(=O)c(CO)c(Nc3ccc(C)nc3)nc12]

Pale yellow solid; mp=over 240° C. (decomp.); ¹H NMR (400 MHz, DMSO-d₆) δ 2.42 (s, 3H), 3.93 (s, 3H), 4.70 (s, 2H), 7.07 (dd, J=7.6, 7.6 Hz, 1H), 7.18 (d, J=8.4 Hz, 1H), 7.28 (d, J=7.6 Hz, 1H), 8.17 (dd, J=2.8, 8.4 Hz, 1H), 8.47 (dd, J=1.2, 7.6 Hz, 1H), 8.85 (d, J=2.8 Hz, 1H); LC-MS (ESI, m/z): 313[M+H]⁺.

### 5-(3-(Hydroxymethyl)-9-methoxy-4-oxo-4H-pyrido[1,2-a]pyrimidin-2-ylamino)-picolinonitrile (7)

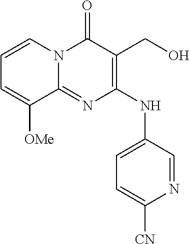

White solid; mp=over 350° C. (decomp.); ¹H NMR (400 MHz, CDCl₃+CD₃OD) δ 4.00 (s, 3H), 4.85 (s, 2H), 6.98-7.03 (m, 2H), 7.63 (d, J=8.4 Hz, 1H), 8.49 (dd, J=2.4, 8.4 Hz, 1H), 8.55 (dd, J=2.4, 8.4 Hz, 1H), 8.98 (d, J=2.4 Hz, 1H).

### 3-(Hydroxymethyl)-9-methoxy-2-(6-(trifluoromethyl)pyridin-3-ylamino)-4H-pyrido[1,2-a]-pyrimidin-4-one (8)

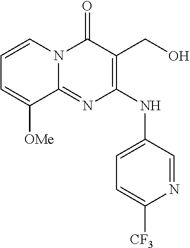 [SMILES: COc1cccn2c(=O)c(CO)c(Nc3ccc(C(F)(F)F)nc3)nc12]

White solid; mp=over 400° C. (decomp.); ¹H NMR (400 MHz, CDCl₃+CD₃OD) δ 3.99 (s, 3H), 4.85 (s, 2H), 6.94-7.00 (m, 2H), 7.61 (d, J=8.4 Hz, 1H), 8.44 (dd, J=2.4, 8.4 Hz, 1H), 8.53-8.55 (m, 1H), 9.02 (d, J=2.4 Hz, 1H); LC-MS (ESI, m/z): 367[M+H]⁺.

### Methyl 6-(3-(hydroxymethyl)-9-methoxy-4-oxo-4H-pyrido[1,2-a]pyrimidin-2-ylamino)nicotinate (9)

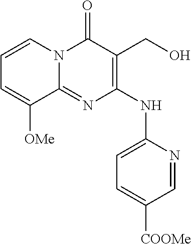 [SMILES: COC(=O)c1ccc(Nc2nc3c(OC)cccn3c(=O)c2CO)nc1]

Yellow solid; mp=231-232° C. (decomp.); ¹H NMR (400 MHz, DMSO-d₆) δ 3.71 (s, 3H), 3.92 (s, 3H), 4.59 (d, J=4.8 Hz, 2H), 5.21 (t, J=4.8 Hz, 1H), 6.92 (d, J=7.6 Hz, 1H), 7.02 (d, J=3.2 Hz, 1H), 7.24 (d, J=7.8 Hz, 1H), 7.62 (s, 1H), 7.87 (dd, J=3.2 Hz, 7.6 Hz, 1H), 8.32 (d, J=6.8 Hz, 1H), 9.10 (s, 1H); ¹³C NMR (100 MHz, DMSO-d₆) δ 52.1, 55.2, 56.4, 94.8, 113.1, 113.2, 114.6, 118.5, 122.4 128.9, 132.2, 142.4, 142.8, 150.1, 155.2, 156.9, 160.2.

### 3-(Hydroxymethyl)-9-methoxy-2-(5-methylpyridin-2-ylamino)-4H-pyrido[1,2-a]pyrimidin-4-one (10)

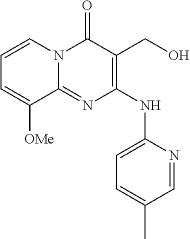 [SMILES: COc1cccn2c(=O)c(CO)c(Nc3ccc(C)cn3)nc12]

Yellow solid; mp=282° C. (decomp.); ¹H NMR (400 MHz, DMSO-d₆) δ 1.91 (s, 3H), 3.88 (s, 3H), 4.62 (s, 2H), 5.19 (brs, 1H), 6.91 (d, J=7.6 Hz, 1H), 7.12 (d, J=2.8 Hz, 1H), 7.22 (d, J=7.6 Hz, 1H), 7.68 (s, 1H), 7.72 (dd, J=2.8 Hz, 7.6 Hz, 1H), 8.32 (s, 1H), 8.92 (s, 1H); ¹³C NMR (100 MHz, DMSO-d₆) δ 18.1, 54.2, 55.2, 95.6, 113.2, 113.4, 115.2, 117.2, 123.2 128.8, 132.2, 140.4, 142.0, 146.9, 154.2, 158.9.

### 2-(6-Chloropyridin-3-ylamino)-3-(hydroxymethyl)-9-methoxy-4H-pyrido[1,2-a]pyrimidin-4-one (11)

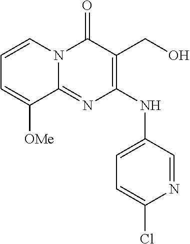 [SMILES: COc1cccn2c(=O)c(CO)c(Nc3ccc(Cl)nc3)nc12]

White solid; mp=245° C. (decomp.); ¹H NMR (400 MHz, DMSO-d₆) δ 3.93 (s, 3H), 4.67-4.68 (m, 2H), 5.15 (brs, 1H), 7.10 (d, J=7.2 Hz, 1H), 7.30 (d, J=7.2 Hz, 1H), 7.42 (d, J=8.8 Hz, 1H), 8.36 (dd, J=2.8 Hz, 8.8 Hz, 1H), 8.46 (d, J=7.2 Hz, 1H), 8.86 (s, 1H), 8.92 (d, J=2.8 Hz, 1H).

### 2-(5-(Dimethylamino)pyrimidin-2-ylamino)-3-(hydroxymethyl)-9-methoxy-4H-pyrido[1,2-a]pyrimidin-4-one (12)

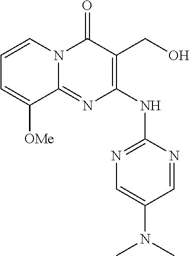 [SMILES: COc1cccn2c(=O)c(CO)c(Nc3ncc(N(C)C)cn3)nc12]

¹H NMR (400 MHz, DMSO-d₆) δ 3.11 (s, 6H), 3.87 (s, 3H), 4.64 (s, 2H), 4.96 (brs, 1H), 7.04 (dd, J=7.2, 7.2 Hz, 1H), 7.24 (d, J=7.2 Hz, 1H), 8.42 (s, 1H), 8.44 (d, J=7.2 Hz, 1H), 8.64 (s, 2H); ¹³C NMR (100 MHz, DMSO-d₆) δ 159.3, 157.1, 156.9, 152.3, 151.1, 144.2, 125.3, 119.5, 113.5, 95.1, 57.4, 54.7, 37.6; LC-MS (ESI, m/z): 339[M+H]⁺.

### 2-(1H-Indol-5-ylamino)-3-(hydroxymethyl)-9-methoxy-4H-pyrido[1,2-a]pyrimidin-4-one (13)

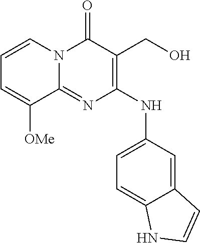 [SMILES: COc1cccn2c(=O)c(CO)c(Nc3ccc4[nH]ccc4c3)nc12]

Pale yellow solid; mp=162° C. (decomp.); ¹H NMR (400 MHz, DMSO-d₆) δ 3.96 (s, 3H), 4.77 (d, J=5.2 Hz, 2H), 5.27 (t, J=5.4 Hz, 1H), 6.43-6.44 (m, 1H), 7.08 (dd, J=7.2, 7.6 Hz, 1H), 7.28 (d, J=6.8 Hz, 1H), 7.35-7.36 (m, 3H), 8.14 (s, 1H), 8.52 (dd, J=0.8, 7.2 Hz, 1H), 8.59 (s, 1H), 11.01 (s, 1H).

### 3-(Hydroxymethyl)-9-methoxy-2-(1-methyl-1H-indol-5-ylamino)-4H-pyrido[1,2-a]pyrimidin-4-one (14)

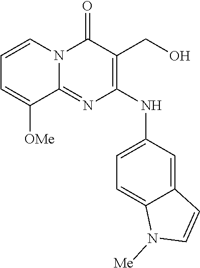 [SMILES: COc1cccn2c(=O)c(CO)c(Nc3ccc4c(ccn4C)c3)nc12]

Pale yellow solid; mp=195-197° C.; ¹H NMR (400 MHz, DMSO-d₆) δ 3.82 (s, 3H), 3.97 (s, 3H), 4.77 (d, J=5.2 Hz, 2H), 5.28 (t, J=5.2 Hz, 1H), 6.42 (d, J=3.0 Hz, 1H), 7.09 (dd, J=7.2, 7.6 Hz, 1H), 7.28-7.30 (m, 1H), 7.33 (d, J=3.0 Hz, 1H), 7.41 (d, J=8.8 Hz, 1H), 7.46 (dd, J=2.0, 8.8 Hz, 1H), 8.18 (d, J=2.0 Hz, 1H), 8.52 (dd, J=1.2, 6.8 Hz, 1H), 8.62 (br s, 1H).

### 2-(1H-Indol-6-ylamino)-3-(hydroxymethyl)-9-methoxy-4H-pyrido[1,2-a]pyrimidin-4-one (15)

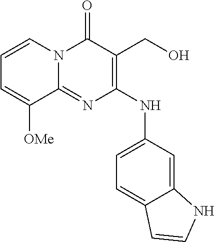 [SMILES: COc1cccn2c(=O)c(CO)c(Nc3ccc4cc[nH]c4c3)nc12]

Orange solid; mp=over 300° C. (decomp.); ¹H NMR (400 MHz, CDCl₃+CD₃OD), δ 3.94 (s, 3H), 4.82 (s, 2H), 6.43 (dd, J=0.8, 3.6 Hz, 1H), 6.82 (dd, J=7.2, 7.6 Hz, 1H), 6.90 (dd, J=1.2, 7.6 Hz, 1H), 7.11 (d, J=2.8 Hz, 1H), 7.18 (dd, J=2.0, 8.4 Hz, 1H), 7.49 (d, J=8.4 Hz, 1H), 8.04-8.05 (m, 1H), 8.50 (dd, J=1.6, 7.2 Hz, 1H); ¹³C NMR (400 MHz, CDCl₃+CD₃OD) δ 56.5, 56.9, 94.9, 102.0, 103.3, 112.0, 112.6, 114.6, 119.7, 120.5, 124.1, 124.4, 134.2, 136.3, 144.4, 151.2, 157.4, 157.6; LC-MS (ESI, m/z): 337[M+H]⁺.

### 2-(3-Chloro-1H-indol-5-ylamino-3-(hydroxymethyl)-9-methoxy-4H-pyrido[1,2-a]pyrimidin-4-one (16)

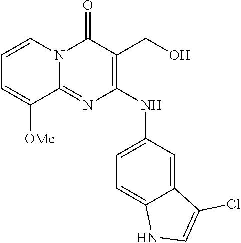 [SMILES: COc1cccn2c(=O)c(CO)c(Nc3ccc4[nH]cc(Cl)c4c3)nc12]

Yellow solid; mp=230° C. (decomp.); ¹H NMR (400 MHz, CDCl₃+CD₃OD) δ 3.57 (s, 3H), 3.93 (s, 2H), 6.84 (dd, J=7.2, 7.2 Hz, 1H), 7.01 (d, J=7.2 Hz, 1H), 7.07 (s, 1H), 7.15 (dd, J=2.0, 8.4 Hz, 1H), 7.21 (d, J=8.8 Hz, 1H), 8.39 (d, J=7.2 Hz, 1H), 8.64 (s, 1H), 10.16 (s, 1H), 11.57 (br s, 1H).

### 2-(2-Methyl-1H-indol-5-ylamino)-3-(hydroxymethyl)-9-methoxy-4H-pyrido[1,2-a]pyrimidin-4-one (17)

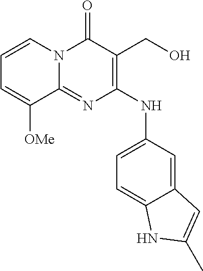 [SMILES: COc1cccn2c(=O)c(CO)c(Nc3ccc4[nH]c(C)cc4c3)nc12]

Pale yellow solid; mp=186° C. (decomp); ¹H NMR (400 MHz, DMSO d-6) δ 2.36 (s, 3H), 3.89 (s, 3H), 4.71 (d, J=5.6 Hz, 2H), 5.22 (t, J=5.6 Hz, 1H), 6.07 (s, 1H), 6.99 (dd, J=7.6 Hz, 7.6 Hz, 1H), 7.16-7.23 (m, 3H), 7.93 (s, 1H), 8.45 (d, J=6.8 Hz, 1H), 8.50 (s, 1H), 10.76 (s, 1H); ¹³C NMR (100 MHz, DMSO) δ 14.1, 55.4, 57.3, 94.2, 99.9, 110.7, 111.9, 113.1, 113.3, 116.0, 119.5, 129.3, 132.4, 133.3, 136.7, 144.3, 151.1, 156.8, 157.3.

### 2-(1-Acetyl-1H-indol-5-ylamino)-3-(hydroxymethyl)-9-methoxy-4H-pyrido[1,2-a]pyrimidin-4-one (18)

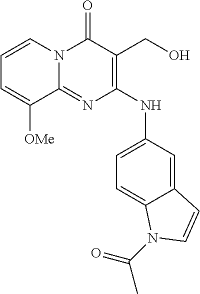 [SMILES: COc1cccn2c(=O)c(CO)c(Nc3ccc4c(ccn4C(C)=O)c3)nc12]

Pale yellow solid; mp=175° C. (decomp); ¹H NMR (400 MHz, DMSO d-6) δ 2.62 (s, 3H), 3.95 (s, 3H), 4.72 (s, 2H), 5.31 (brs, 1H), 6.71 (d, J=3.6 Hz, 1H), 7.07 (dd, J=7.6 Hz, 7.6 Hz, 1H), 7.27 (d, J=7.6 Hz, 1H), 5.58 (dd, J=1.6 Hz, 8.8 Hz, 1H), 7.81 (d, J=4.0 Hz, 1H), 8.21 (d, J=8.8 Hz, 1H), 8.33 (d, J=1.6 Hz, 1H), 8.47 (d, J=6.8 Hz, 1H), 8.72 (brs, 1H); ¹³C NMR (100 MHz, DMSO) δ 24.2. 55.2. 57.4. 95.1. 109.1. 112.6. 113.5. 113.7. 116.3. 118.8. 119.5. 128.3. 131.3. 131.4. 136.8. 144.1. 151.3. 156.8. 156.9. 169.8.

### Ethyl 5-(3-(hydroxymethyl)-9-methoxy-4-oxo-4H-pyrido[1,2-a]pyrimidin-2-ylamino)-1H-indole-2-carboxylate (19)

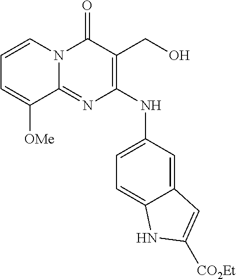 [SMILES: CCOC(=O)c1cc2cc(Nc3nc4c(OC)cccn4c(=O)c3CO)ccc2[nH]1]

Pale yellow solid; mp=222-223° C. (decomp.); ¹H NMR (400 MHz, DMSO-d₆) δ 1.39 (t, J=7.2 Hz, 3H), 3.99 (s, 3H), 4.04 (s, 3H), 4.39 (q, J=7.2 Hz, 2H), 4.79 (d, J=5.2 Hz, 2H), 5.40 (t, J=5.4 Hz, 1H), 7.10 (dd, J=7.2, 7.2 Hz, 1H), 7.15 (d, J=1.2 Hz, 1H), 7.31 (dd, J=1.2, 7.6 Hz, 1H), 7.55 (dd, J=2.0, 9.2 Hz, 1H), 8.33 (d, J=1.6 Hz, 1H), 8.52 (dd, J=1.2, 7.2 Hz, 1H), 8.68 (br s, 1H), 11.84 (br s, 1H); LC-MS (ESI, m/z); [M+H]⁺ 409.23

### 5-(3-(Hydroxymethyl)-9-methoxy-4-oxo-4H-pyrido[1,2-a]pyrimidin-2-ylamino)-1H-indole-2-carboxylic acid (20)

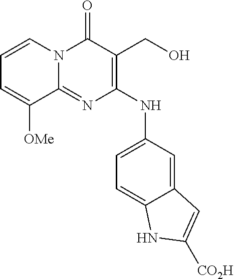 [SMILES: COc1cccn2c(=O)c(CO)c(Nc3ccc4[nH]c(C(=O)O)cc4c3)nc12]

Pale yellow solid; mp=213° C. (decomp.); ¹H NMR (400 MHz, DMSO-d₆) δ 3.96 (s, 3H), 4.70 (s, 2H), 5.79 (s, 1H), 7.08-7.12 (m, 2H), 7.31 (d, J=7.6 Hz, 1H), 7.41 (d, J=8.8 Hz, 1H), 7.55 (dd, J=2.0, 8.8 Hz, 1H), 8.24 (s, 1H), 8.36 (s, 1H), 8.51 (dd, J=1.2, 7.2 Hz, 1H), 11.71 (br s, 1H).

### 9-Fluoro-3-(hydroxymethyl)-2-(1-methyl-1H-indol-5-ylamino)-4H-pyrido[1,2-a]pyrimidin-4-one (21)

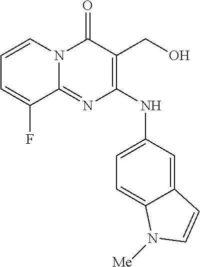 [SMILES: Cn1ccc2cc(Nc3nc4c(F)cccn4c(=O)c3CO)ccc21]

Yellow solid; mp=253-254° C.; ¹H NMR (400 MHz, CDCl₃) δ 3.78 (s, 3H), 4.85 (d, J=0.2 Hz, 2H), 5.10 (t, J=5.4 Hz, 1H), 6.37 (d, J=2.8 Hz, 1H), 6.85-6.90 (m, 1H), 7.06 (d, J=2.8 Hz, 1H), 7.26 (d, J=8.8 Hz, 1H), 7.35-7.40 (m, 2H), 7.87-7.88 (m, 1H), 8.68 (s, 1H), 8.70 (s, 1H).

### 2-(1-Acetyl-1H-indol-5-ylamino)-9-fluoro-3-(hydroxymethyl)-4H-pyrido[1,2-a]pyrimidin-4-one (22)

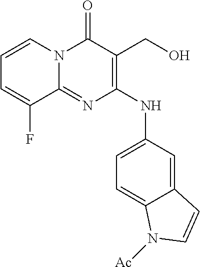 [SMILES: CC(=O)n1ccc2cc(Nc3nc4c(F)cccn4c(=O)c3CO)ccc21]

Pale yellow solid; ¹H NMR (400 MHz, DMSO-d₆) δ 2.69 (s, 2H), 4.78 (d, J=3.6 Hz, 2H), 5.33 (s, 1H), 6.76 (d, J=3.2 Hz, 1H), 7.13-7.18 (m, 1H), 5.57 (dd, J=2.0, 8.8 Hz, 1H), 7.82 (dd, J=8.0, 9.2 Hz, 1H), 7.89 (d, J=3.2 Hz, 1H), 8.09 (s, 1H), 8.30 (d, J=8.8 Hz, 1H), 8.72 (d, J=7.2 Hz, 1H), 8.90 (s, 1H).

### 2-(1H-indol-5-ylamino)-3-(hydroxymethyl)-7-methyl-4H-pyrido[1,2-a]pyrimidin-4-one (23)

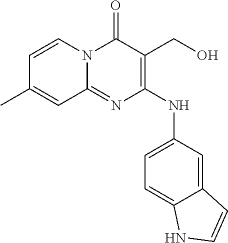 [SMILES: Cc1ccn2c(=O)c(CO)c(Nc3ccc4[nH]ccc4c3)nc2c1]

Dark-green solid, mp=195° C. (decomp); ¹H NMR (400 MHz, MeOH-d4) δ 2.39 (s, 3H), 4.57 (s, 1H), 4.87 (s, 2H), 6.44 (d, J=2.8 Hz, 1H), 6.95 (dd, J=7.6 Hz, 1H), 7.15 (s, 1H), 7.25 (t, J=8.0 Hz, 2H), 7.37 (d, J=8.8 Hz, 1H), 7.76 (d, J=1.6 Hz, 1H), 8.78 (d, J=7.2 Hz, 1H).

### 3-(Hydroxymethyl)-7-methyl-2-(1-methyl-1H-indol-5-ylamino)-4H-pyrido[1,2-a]pyrimidin-4-one (24)

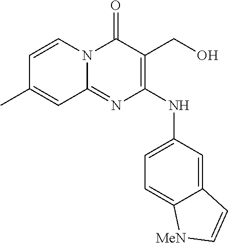 [SMILES: Cc1ccn2c(=O)c(CO)c(Nc3ccc4c(ccn4C)c3)nc2c1]

Pale-green solid, mp=195° C. (decomp); ¹H NMR (400 MHz, MeOH-d₄) δ 2.40 (s, 3H), 3.81 (s, 3H), 4.51 (s, 1H), 4.87 (s, 2H), 6.42 (d, J=2.8 Hz, 1H), 6.95 (dd, J=7.6 Hz, 1H), 7.14 (d, J=2.8 Hz, 1H), 7.30 (dd, J=2.0 Hz, 2H), 7.35 (d, J=8.4 Hz, 1H), 7.76 (d, J=1.6 Hz, 1H), 7.79 (s, 1H), 8.79 (d, J=7.2 Hz, 1H).

### 2-(1-Acetyl-1H-indol-5-ylamino)-3-(hydroxymethyl)-7-methyl-4H-pyrido[1,2-a]pyrimidin-4-one (25)

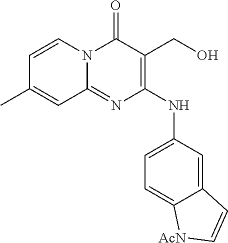

Pale-green solid, mp=200° C. (decomp); ¹H NMR (400 MHz, CDCl₃) δ 2.22 (s, 3H), 2.65 (s, 3H), 3.92 (s, 1H), 4.68 (s, 2H), 6.30 (d, J=2.8 Hz, 1H), 6.67 (d, J=7.2 Hz, 1H), 6.99 (s, 1H), 7.04-7.08 (m, 2H), 7.19 (s, 1H), 7.61 (d, J=1.6 Hz, 1H), 8.61 (d, J=7.2 Hz, 1H).

### 2-(1-Acetylindolin-5-ylamino)-3-(hydroxymethyl)-7-methyl-4H-pyrido[1,2-a]pyrimidin-4-one (26)

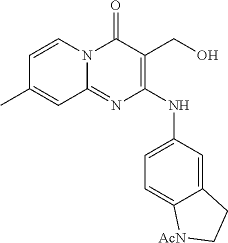

White solid, mp=200° C. (decomp); ¹H NMR (400 MHz, MeOH-d₆) δ 2.24 (s, 3H), 2.45 (s, 3H), 4.15 (t, J=8.8 Hz, 2H), 4.43 (s, 2H), 4.65 (s, 1H), 4.85 (s, 2H), 6.97 (dd, J=7.2 Hz, 1H), 7.23 (s, 1H), 7.35 (dd, J=8.8 Hz, 1H), 7.56 (s, 1H), 8.07 (d, J=8.8 Hz, 1H), 8.80 (d, J=7.2 Hz, 1H).

### Ethyl 2-(1H-indol-5-ylamino)-9-methoxy-4-oxo-4H-pyrido[1,2-a]pyrimidine-3-carboxylate (27)

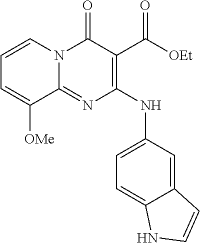 [SMILES: CCOC(=O)c1c(Nc2ccc3[nH]ccc3c2)nc2c(OC)cccn2c1=O]

Pale yellow solid; ¹H NMR (400 MHz, DMSO d-6) 1.30 (t, J=7.2 Hz, 3H), δ 3.92 (s, 3H), 4.29 (q, J=7.2 Hz, 2H), 6.49 (s, 1H), 7.05 (dd, J=7.2 Hz, 7.2 Hz, 1H), 7.29-7.36 (m, 4H), 8.27 (s, 1H), 8.44 (d, J=6.8 Hz, 1H), 11.11, (s, 1H), 11.2 (s, 1H).

### Ethyl 2-(1H-indol-5-ylamino)-9-fluoro-4-oxo-4H-pyrido[1,2-a]pyrimidine-3-carboxylate (28)

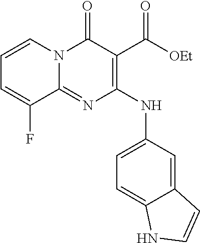 [SMILES: CCOC(=O)c1c(Nc2ccc3[nH]ccc3c2)nc2c(F)cccn2c1=O]

Yellow solid; mp=222-223° C.; ¹H NMR (400 MHz, CDCl₃) δ 1.48 (t, J=7.0 Hz, 3H), 4.47 (q, J=7.2 Hz, 2H), 6.55-5.56 (m, 1H), 6.79-6.84 (m, 1H), 7.21-7.23 (m, 1H), 7.38 (d, J=8.4 Hz, 2H), 7.48 (dd, J=2.0, 8.8 Hz, 1H), 8.09 (d, J=1.6 Hz, 1H), 8.17 (s, 1H), 8.78 (d, J=7.2 Hz, 1H), 11.53 (s, 1H).

### Ethyl 2-(1H-indol-5-ylamino)-7-methyl-4-oxo-4H-pyrido[1,2-a]pyrimidine-3-carboxylate (29)

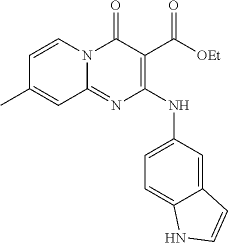 [SMILES: CCOC(=O)c1c(Nc2ccc3[nH]ccc3c2)nc2cc(C)ccn2c1=O]

Colorless solid, mp=235° C.; ¹H NMR (400 MHz, CDCl₃) δ 1.47 (s, 3H), 2.36 (s, 3H), 4.46 (q, J=7.2 Hz, 2H), 6.54 (t, J=2.8 Hz, 1H), 6.72 (dd, J=7.2 Hz, 1H), 7.04 (s, 1H), 7.21 (t, J=2.8 Hz, 1H), 7.35 (s, 2H), 7.92 (s, 1H), 8.19 (s, 1H), 8.85 (d, J=7.2 Hz, 1H), 11.28 (s, 1H).

### Ethyl 2-(1-acetyl-1H-indol-5-ylamino)-7-methyl-4-oxo-4H-pyrido[1,2-a]pyrimidine-3-carboxylate (30)

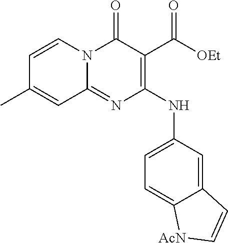

Colorless solid, mp=225° C.; ¹H NMR (400 MHz, CDCl₃) δ 1.44 (s, 3H), 2.45 (s, 3H), 2.66 (s, 3H), 4.45 (q, J=7.2 Hz, 2H), 6.69 (d, J=4.0 Hz, 1H), 6.97 (dd, J=7.2 Hz, 1H), 7.18 (s, 1H), 7.51 (dd, J=8.8 Hz, 1H), 7.63 (d, J=3.6 Hz, 1H), 7.98 (d, J=2.0 Hz, 1H), 8.36 (d, J=8.8 Hz, 1H), 8.85 (d, J=7.2 Hz, 1H).

### Ethyl 2-(1-acetylindolin-5-ylamino)-7-methyl-4-oxo-4H-pyrido[1,2-a]pyrimidine-3-carboxylate (31)

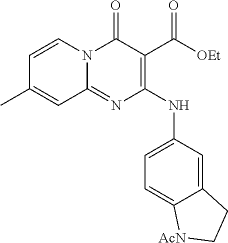

Colorless solid, mp=225° C.; ¹H NMR (400 MHz, CDCl₃) δ 1.43 (s, 3H), 2.24 (s, 3H), 2.44 (s, 3H), 3.27 (t, J=8.4 Hz, 2H), 4.16 (t, J=8.4 Hz, 2H), 4.43 (q, J=7.2 Hz, 2H), 6.95 (dd, J=7.2 Hz, 1H), 7.15 (s, 1H), 7.42 (dd, J=8.8 Hz, 1H), 7.60 (s, 1H), 8.08 (d, J=8.8 Hz, 1H), 8.82 (d, J=7.2 Hz, 1H).

### 2-(1H-indol-7-ylamino)-3-(hydroxymethyl)-9-methoxy-4H-pyrido[1,2-a]pyrimidin-4-one (32)

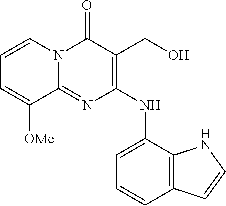 [SMILES: COc1cccn2c(=O)c(CO)c(Nc3cccc4cc[nH]c34)nc12]

Orange solid; mp=over 380° C. (decomp.); ¹H NMR (400 MHz, CDCl₃+CD₃OD) δ 3.93 (s, 3H), 4.86 (s, 2H), 6.53 (d, J=2.8 Hz, 1H), 6.70-6.78 (m, 2H), 6.82 (d, J=7.6 Hz, 1H), 6.96 (dd, J=7.6, 7.6 Hz, 1H), 7.20 (d, J=2.8 Hz, 1H), 7.37 (d, J=8.0 Hz, 1H), 8.47 (d, J=6.8 Hz, 1H); LC-MS (ESI, m/z): 337[M+H]⁺.

### 2-(1H-indol-3-ylamino)-3-(hydroxymethyl)-9-methoxy-4H-pyrido[1,2-a]pyrimidin-4-one (33)

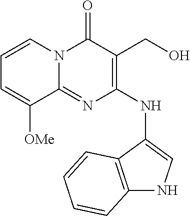 [SMILES: COc1cccn2c(=O)c(CO)c(Nc3c[nH]c4ccccc34)nc12]

Brown solid; ¹H NMR (400 MHz, DMSO-d₆) δ 3.99 (s, 3H), 4.82 (s, 2H), 5.61 (brs, 1H), 7.02-7.14 (m, 3H), 7.30 (d, J=7.6 Hz, 1H), 7.36 (d, J=8.4 Hz, 1H), 7.53 (d, J=8.0 Hz, 1H), 8.17 (s, 1H), 8.49 (d, J=7.2 Hz, 1H), 8.90 (s, 1H), 10.82 (s, 1H); LC-MS (ESI, m/z): 337[M+H]⁺.

### 2-(1H-indazol-5-ylamino)-3-(hydroxymethyl)-9-methoxy-4H-pyrido[1,2-a]pyrimidin-4-one (34)

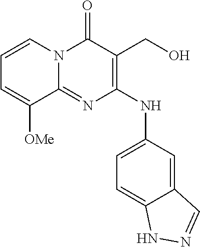 [SMILES: COc1cccn2c(=O)c(CO)c(Nc3ccc4[nH]ncc4c3)nc12]

Pale yellow solid; mp=165° C. (decomp); ¹H NMR (400 MHz, DMSO d-6) δ 3.92 (s, 3H), 4.72 (s, 2H), 5.22 (s, 1H), 7.05 (d, J=6.8 Hz, 1H), 7.23 (d, J=6.8 Hz, 1H), 7.47 (d, J=7.6 Hz, 1H), 7.55 (d, J=8.4 Hz, 1H), 8.01 (s, 1H), 8.39 (s, 1H), 8.46 (d, J=6.8 Hz, 1H), 8.67 (s, 1H), 12.93, (s, 1H); ¹³C NMR (100 MHz, DMSO) δ 55.2, 57.4, 94.9, 110.4, 111.4, 113.4, 113.5, 119.5, 122.9, 123.7, 133.8, 134.0, 137.3, 144.2, 151.2, 156.9, 157.0.

### 3-(Hydroxymethyl)-9-methoxy-2-(1-methyl-1H-indazol-5-ylamino)-4H-pyrido[1,2-a]pyrimidin-4-one (35)

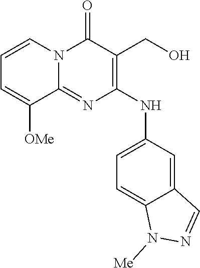 [SMILES: COc1cccn2c(=O)c(CO)c(Nc3ccc4c(cnn4C)c3)nc12]

White solid; mp=205° C. (decomp.); ¹H NMR (400 MHz, DMSO-d₆) δ 3.40 (s, 3H), 4.08 (s, 3H), 4.78 (d, J=4.8 Hz, 2H), 5.28 (t, J=5.0 Hz, 1H), 7.12 (dd, J=7.2, 7.6 Hz, 1H), 7.32 (dd, J=1.2, 7.6 Hz, 1H), 7.62 (d, J=9.0 Hz, 1H), 7.68 (dd, J=2.0, 9.0 Hz, 1H), 8.04 (m, 1H), 8.07 (d, J=1.2 Hz, 1H), 8.53 (dd, J=1.2, 6.8 Hz, 1H), 8.75 (br s, 1H).

### 3-(Hydroxymethyl)-9-methoxy-2-(2-methyl-2H-indazol-5-ylamino)-4H-pyrido[1,2-a]pyrimidin-4-one (36)

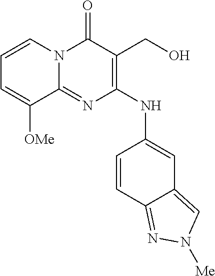 [SMILES: COc1cccn2c(=O)c(CO)c(Nc3ccc4nn(C)cc4c3)nc12]

White solid; mp=209-210° C.; ¹H NMR (400 MHz, DMSO-d₆) δ 4.00 (s, 3H), 4.19 (s, 3H), 4.77 (d, J=5.2 Hz, 2H), 5.30 (t, J=5.2 Hz, 1H), 7.122 (dd, J=7.2, 7.6 Hz, 1H), 7.32 (d, J=8.0 Hz, 1H), 7.44 (dd, J=2.0, 9.2 Hz, 1H), 7.57 (d, J=9.2 Hz, 1H), 8.30 (s, 1H), 8.45 (d, J=1.2 Hz, 1H), 8.52-8.54 (m, 1H), 8.69 (s, 1H).

### 2-(1H-Indazol-5-ylamino)-9-fluoro-3-(hydroxymethyl)-4H-pyrido[1,2-a]pyrimidin-4-one (37)

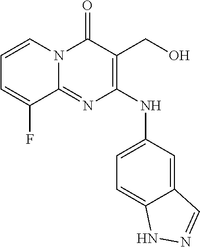 [SMILES: O=c1c(CO)c(Nc2ccc3[nH]ncc3c2)nc2c(F)cccn12]

Pale yellow solid; mp=220° C. (decomp.); ¹H NMR (400 MHz, DMSO-d₆) δ 4.78 (d, J=5.2 Hz, 2H), 5.26 (t, J=5.2 Hz, 1H), 7.11-7.16 (m, 1H), 7.53-7.59 (m, 2H), 8.06 (s, 1H), 8.13 (s, 1H), 8.71 (d, J=7.6 Hz, 1H), 8.85 (s, 1H), 13.03 (s, 1H).

### 9-Fluoro-3-(hydroxymethyl)-2-(1-methyl-1H-indazol-5-ylamino)-4H-pyrido[1,2-a]pyrimidin-4-one (38)

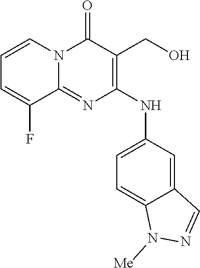 [SMILES: Cn1ncc2cc(Nc3nc4c(F)cccn4c(=O)c3CO)ccc21]

Pale yellow solid; mp=216° C. (decomp.); ¹H NMR (400 MHz, DMSO-d₆) δ 4.08 (s, 3H), 4.78 (s, 2H), 5.29 (s, 1H), 7.11-7.16 (m, 1H), 7.65 (s, 2H), 7.80 (dd, J=8.4, 9.2 Hz, 1H), 8.03 (s, 1H), 8.12 (s, 1H), 8.71 (d, J=6.8 Hz, 1H), 8.88 (s, 1H).

### 9-Fluoro-3-(hydroxymethyl)-2-(2-methyl-2H-indazol-5-ylamino)-4H-pyrido[1,2-a]pyrimidin-4-one (39)

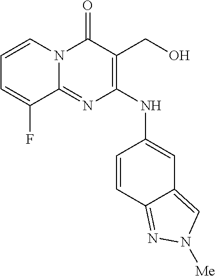 [SMILES: Cn1cc2cc(Nc3nc4c(F)cccn4c(=O)c3CO)ccc2n1]

Yellow solid; mp=226-227° C.; ¹H NMR (400 MHz, DMSO-d₆) δ 4.18 (s, 3H), 4.77 (m, 2H), 5.30 (m, 1H), 7.12-7.17 (m, 1H), 7.42 (dd, J=2.0, 9.2 Hz, 1H), 7.59 (d, J=9.2 Hz, 1H), 7.78-7.83 (m, 1H), 8.12 (s, 1H), 8.31 (s, 1H), 8.72 (d, J=7.2 Hz, 1H), 8.81 (s, 1H).

### Ethyl 2-(1H-indazol-5-ylamino)-9-fluoro-4-oxo-4H-pyrido[1,2-a]pyrimidine-3-carboxylate (40)

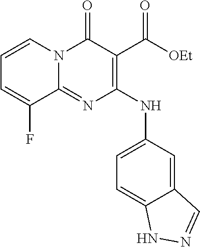 [SMILES: CCOC(=O)c1c(Nc2ccc3[nH]ncc3c2)nc2c(F)cccn2c1=O]

Yellow solid; mp=284-286° C.; ¹H NMR (400 MHz, CDCl₃+DMSO-d₆) δ 1.40 (t, J=7.0 Hz, 3H), 4.39 (q, J=7.2 Hz, 1H), 7.01-7.06 (m, 1H), 7.49-7.51 (m, 2H), 7.65-7.70 (m, 1H), 7.96 (s, 1H), 8.21 (s, 1H), 8.72 (d, J=7.6 Hz, 1H), 11.44 (s, 1H), 12.84 (s, 1H).

### 2-(1H-indazol-5-ylamino)-3-(hydroxymethyl)-7-methyl-4H-pyrido[1,2-a]pyrimidin-4-one (41)

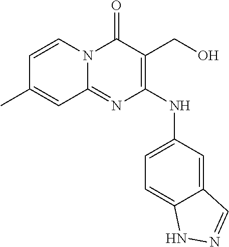 [SMILES: Cc1ccn2c(=O)c(CO)c(Nc3ccc4[nH]ncc4c3)nc2c1]

Pale-yellow solid, mp=197° C. (decomp); ¹H NMR (400 MHz, MeOH-d₄) δ 2.43 (s, 3H), 3.65 (s, 1H), 4.57 (s, 1H), 4.89 (s, 2H), 7.01 (dd, J=7.2 Hz, 1H), 7.22 (s, 1H), 7.53 (s, 2H), 8.02 (s, 1H), 8.07 (s, 1H), 8.82 (d, J=7.6 Hz, 1H).

### 3-(Hydroxymethyl)-7-methyl-2-(1-methyl-1H-indazol-5-ylamino)-4H-pyrido[1,2-a]pyrimidin-4-one (42)

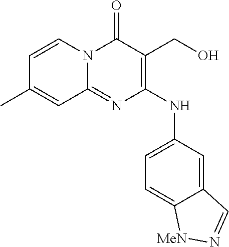 [SMILES: Cc1ccn2c(=O)c(CO)c(Nc3ccc4c(cnn4C)c3)nc2c1]

Pale-yellow solid, mp=197° C. (decomp); ¹H NMR (400 MHz, CDCl₃) δ 2.37 (s, 3H), 4.07 (s, 3H), 4.97 (d, J=5.2 Hz, 2H), 6.77 (dd, J=7.2 Hz, 1H), 7.15 (s, 1H), 7.36 (d, J=8.8 Hz, 1H), 7.48 (dd, J=8.8 Hz, 1H), 7.93 (s, 2H), 8.0 (s, 1H), 8.84 (d, J=7.2 Hz, 1H).

### 3-(Hydroxymethyl)-7-methyl-2-(2-methyl-2H-indazol-5-ylamino)-4H-pyrido[1,2-a]pyrimidin-4-One (43)

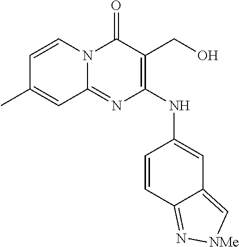 [SMILES: Cc1ccn2c(=O)c(CO)c(Nc3ccc4nn(C)cc4c3)nc2c1]

Pale-yellow solid, mp=200° C. (decomp); ¹H NMR (400 MHz, CDCl₃) δ 2.33 (s, 3H), 4.20 (s, 3H), 4.93 (s, 2H), 6.70 (d, J=7.2 Hz, 1H), 7.12 (s, 1H), 7.37 (dd, J=9.2 Hz, 1H), 7.65 (d, J=9.2 Hz, 1H), 7.83 (s, 1H), 7.92 (s, 1H), 8.02 (s, 1H), 8.79 (d, J=7.2 Hz, 1H).

### 2-(1-Acetyl-1H-indazol-5-ylamino)-3-(hydroxymethyl)-7-methyl-4H-pyrido[1,2-a]pyrimidin-4-one (44)

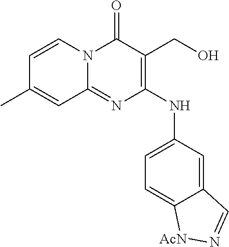

Pale-yellow solid, mp=200° C. (decomp); ¹H NMR (400 MHz, CDCl₃) δ 1.95 (s, 3H), 2.35 (s, 3H), 4.72 (s, 2H), 6.79 (d, J=7.2 Hz, 1H), 7.08 (s, 1H), 7.43 (d, J=9.2 Hz, 1H), 7.71 (dd, J=2.0 Hz, 1H), 7.93 (s, 1H), 8.19 (s, 1H), 8.87 (d, J=7.2 Hz, 1H).

### Ethyl 2-(1H-indazol-5-ylamino)-7-methyl-4-oxo-4H-pyrido[1,2-a]pyrimidine-3-carboxylate (45)

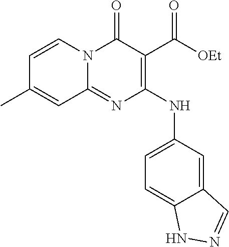 [SMILES: CCOC(=O)c1c(Nc2ccc3[nH]ncc3c2)nc2cc(C)ccn2c1=O]

Colorless solid, mp=225° C.; ¹H NMR (400 MHz, CDCl₃) δ 1.44 (s, 3H), 2.41 (s, 3H), 4.44 (q, J=7.2 Hz, 2H), 6.87 (dd, J=7.6 Hz, 1H), 7.11 (s, 1H), 7.49 (s, 2H), 8.00 (s, 1H), 8.10 (d, J=6.0 Hz, 1H), 8.83 (d, J=7.6 Hz, 1H), 11.27 (s, 1H).

### Ethyl 7-methyl-2-(2-methyl-2H-indazol-5-ylamino)-4-oxo-4H-pyrido[1,2-a]pyrimidine-3-carboxylate (46)

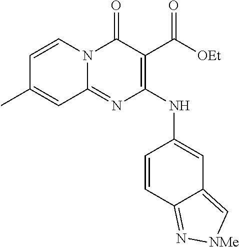 [SMILES: CCOC(=O)c1c(Nc2ccc3nn(C)cc3c2)nc2cc(C)ccn2c1=O]

Pale-yellow solid, mp=230° C.; ¹H NMR (400 MHz, CDCl₃) δ 1.47 (t, J=7.2 Hz, 3H), 2.39 (s, 3H), 4.20 (s, 3H), 4.46 (q, J=7.2 Hz, 2H), 6.76 (dd, J=1.6, 1.6 Hz, 1H), 7.09 (s, 1H), 7.43 (dd, J=2.0, 2.0 Hz, 1H), 7.66 (d, J=8.8 Hz, 1H), 7.85 (s, 1H), 8.00 (s, 1H), 8.87 (d, J=7.2 Hz, 1H), 11.35 (s, 1H).

### Ethyl 2-(1-acetyl-1H-indazol-5-ylamino)-7-methyl-4-oxo-4H-pyrido[1,2-a]pyrimidine-3-carboxylate (47)

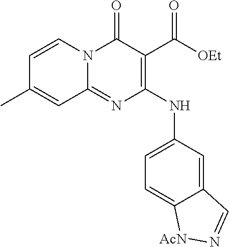

Colorless solid, mp=227° C.; ¹H NMR (400 MHz, CDCl₃) δ 1.48 (t, J=7.2 Hz, 3H), 2.43 (s, 3H), 2.78 (s, 3H), 4.47 (q, J=7.2 Hz, 2H), 6.82 (dd, J=1.6 Hz, 1H), 7.14 (s, 1H), 7.71 (dd, J=2.0, 2.4 Hz, 1H), 8.11 (s, 1H), 8.24 (s, 1H), 8.40 (d, J=8.8 Hz, 1H), 8.90 (d, J=7.2 Hz, 1H), 11.56 (s, 1H).

### 2-(1H-Benzo[d]imidazol-5-ylamino)-3-(hydroxymethyl)-9-methoxy-4H-pyrido[1,2-a]pyrimidin-4-one (48)

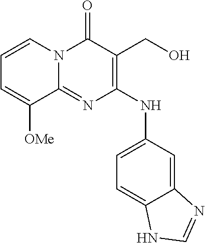 [SMILES: COc1cccn2c(=O)c(CO)c(Nc3ccc4[nH]cnc4c3)nc12]

Yellow solid; mp=205° C. (decomp.); ¹H NMR (400 MHz, CDCl₃+CD₃OD) δ 4.01 (s, 3H), 4.89 (s, 2H), 6.98 (dd, J=7.2, 7.2 Hz, 1H), 7.07 (d, J=7.6 Hz, 1H), 7.36-7.58 (m, 2H), 7.98 (s, 1H), 8.37 (s, 1H), 8.54 (dd, J=1.2, 7.2 Hz, 1H), 12.45 (s, 1H).

### 3-(Hydroxymethyl)-9-methoxy-2-(1-methyl-1H-benzo[d]imidazol-5-ylamino)-4H-pyrido[1,2-a]pyrimidin-4-one (49)

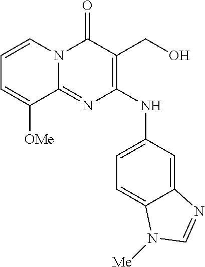 [SMILES: COc1cccn2c(=O)c(CO)c(Nc3ccc4c(c3)ncn4C)nc12]

Pale yellow solid; mp=186° C. (decomp.); ¹H NMR (400 MHz, DMSO-d₆) δ 3.87 (s, 3H), 3.98 (s, 3H), 4.79 (d, J=5.6 Hz, 2H), 5.31 (t, J=5.6 Hz, 1H), 7.08 (dd, J=7.2, 7.2 Hz, 1H), 7.28 (dd, J=0.8, 7.6 Hz, 1H), 7.50 (d, J=8.8 Hz, 1H), 7.56 (dd, J=2.0, 8.8 Hz, 1H), 8.13 (s, 1H), 8.34 (d, J=1.6 Hz, 1H), 8.53 (dd, J=0.8, 7.2 Hz, 1H), 8.73 (br s, 1H). mp=186° C. (decomp=.)

### 3-(Hydroxymethyl)-9-methoxy-2-(1-methyl-1H-benzo[d]imidazol-6-ylamino)-4H-pyrido[1,2-a]pyrimidin-4-one (50)

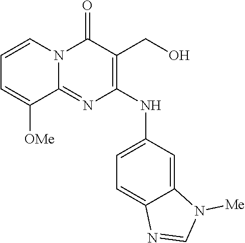 [SMILES: COc1cccn2c(=O)c(CO)c(Nc3ccc4ncn(C)c4c3)nc12]

Pale yellow solid; mp=237° C. (decomp.); ¹H NMR (400 MHz, CD₃OD) δ 3.94 (s, 3H), 4.07 (s, 3H), 7.10 (dd, J=7.2, 7.6 Hz, 1H), 7.27-7.31 (m, 2H), 7.59 (d, J=8.4 Hz, 1H), 8.09 (s, 1H), 8.56 (dd, J=1.2, 7.2 Hz, 1H), 8.91 (d, J=2.0 Hz, 1H).

### 2-(1-Isopropyl-1H-benzo[d]imidazol-5-ylamino)-3-(hydroxymethyl)-9-methoxy-4H-pyrido[1,2-a]pyrimidin-4-one (51)

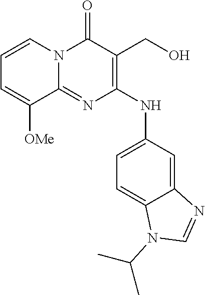 [SMILES: COc1cccn2c(=O)c(CO)c(Nc3ccc4c(c3)ncn4C(C)C)nc12]

Pale yellow solid; mp=181° C. (decomp); ¹H NMR (400 MHz, DMSO d-6) δ 1.52 (d, J=6.8 Hz, 6H), 3.90 (s, 3H), 4.67-4.73 (m, 1H), 4.72 (d, J=5.2 Hz, 2H), 5.25 (t, J=5.2 Hz, 1H), 7.04 (dd, J=7.6 Hz, 7.6 Hz, 1H), 7.25 (d, J=7.6 Hz, 1H), 7.48 (dd, J=4.0 Hz, 8.8 Hz, 1H), 7.54 (d, J=9.2 Hz, 1H), 8.24 (s, 1H), 8.26 (d, J=2.0 Hz, 1H), 8.46 (d, J=7.2 Hz, 1H), 8.65 (s, 1H); ¹³C NMR (100 MHz, DMSO) δ 22.9, 47.7, 55.3, 57.4, 94.8, 110.8, 112.0, 113.4, 113.5, 117.7, 119.5, 129.9, 135.2, 142.4, 144.2, 144.5, 151.2, 156.9, 157.0.

### 2-(1-Isopropyl-1H-benzo[d]imidazol-6-ylamino)-3-(hydroxymethyl)-9-methoxy-4H-pyrido[1,2-a]pyrimidin-4-one (52)

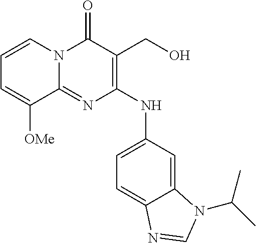 [SMILES: COc1cccn2c(=O)c(CO)c(Nc3ccc4ncn(C(C)C)c4c3)nc12]

Pale yellow solid; mp=191° C. (decomp); ¹H NMR (400 MHz, DMSO d-6) δ 1.61 (d, J=6.8 Hz, 6H), 3.94 (s, 3H), 4.66-4.73 (m, 1H), 4.73 (d, J=5.2 Hz, 2H), 5.33 (t, J=5.2 Hz, 1H), 7.08 (dd, J=7.2 Hz, 7.2 Hz, 1H), 7.13 (dd, J=2.0 Hz, 8.4 Hz, 1H), 7.30 (d, J=7.6 Hz, 1H), 7.53 (d, J=8.8 Hz, 1H), 8.19 (s, 1H), 8.49 (dd, J=1.2 Hz, 7.2 Hz, 1H), 8.78 (s, 1H), 8.80 (d, J=1.6 Hz, 1H); ¹³C NMR (100 MHz, DMSO) δ 22.5, 48.0, 55.3, 57.2, 95.1, 102.6, 113.4, 113.7, 116.2, 119.5, 119.8, 133.8, 135.9, 140.0, 142.0, 144.0, 151.2, 156.8, 159.9.

### 2-(1H-benzo[d]imidazol-5-ylamino)-3-(hydroxymethyl)-7-methyl-4H-pyrido[1,2-a]pyrimidin-4-one (53)

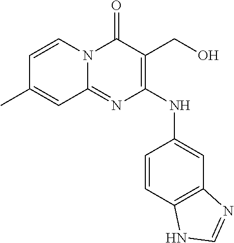 [SMILES: Cc1ccn2c(=O)c(CO)c(Nc3ccc4[nH]cnc4c3)nc2c1]

White solid, mp=200° C. (decomp); ¹H NMR (400 MHz, MeOH-d₄) δ 2.44 (s, 3H), 3.60 (s, 1H), 4.57 (s, 2H), 7.03 (dd, J=7.2 Hz, 1H), 7.24 (s, 1H), 7.37 (d, J=8.4 Hz, 1H), 7.58 (brs, 1H), 8.11-8.16 (m, 2H), 8.83 (d, J=7.2 Hz, 1H).

### 3-(Hydroxymethyl)-7-methyl-2-(1-methyl-1H-benzo[d]imidazol-5-ylamino)-4H-pyrido[1,2-a]pyrimidin-4-one (54)

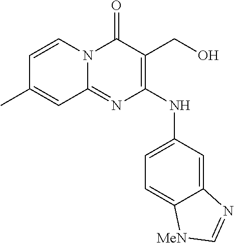 [SMILES: Cc1ccn2c(=O)c(CO)c(Nc3ccc4c(c3)ncn4C)nc2c1]

White solid, decompose at 205° C.; ¹H NMR (400 MHz, MeOH-d₄) δ 2.42 (s, 3H), 3.89 (s, 3H), 4.30 (s, 1H), 4.88 (s, 2H), 6.93 (d, J=7.2 Hz, 1H), 7.20 (s, 1H), 7.43 (s, 1H), 7.61 (s, 1H), 7.99 (s, 1H), 8.09 (s, 1H), 8.80 (d, J=7.2 Hz, 1H).

### 3-(Hydroxymethyl)-7-methyl-2-(1-methyl-1H-benzo[d]imidazol-6-ylamino)-4H-pyrido[1,2-a]pyrimidin-4-one (55)

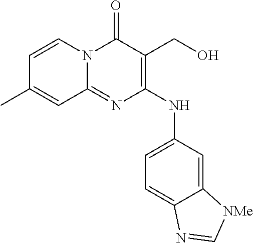 [SMILES: Cc1ccn2c(=O)c(CO)c(Nc3ccc4ncn(C)c4c3)nc2c1]

White solid, decompose at 207° C.; ¹H NMR (400 MHz, MeOH-d₄) δ 2.44 (s, 3H), 3.57 (s, 1H), 3.89 (s, 3H), 4.45 (s, 1H), 4.89 (s, 2H), 6.97 (dd, J=7.6 Hz, 1H), 7.38 (dd, J=8.8 Hz, 1H), 7.62 (d, J=8.8 Hz, 1H), 7.73 (s, 1H), 8.00 (d, J=8.4 Hz, 2H), 8.81 (d, J=7.2 Hz, 1H).

### Ethyl 2-(1H-benzo[d]imidazol-5-ylamino)-9-methoxy-4-oxo-4H-pyrido[1,2-a]pyramidine-3-carboxylate (56)

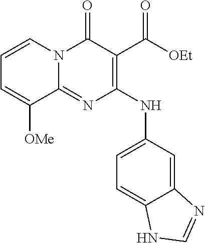 [SMILES: CCOC(=O)c1c(Nc2ccc3[nH]cnc3c2)nc2c(OC)cccn2c1=O]

Yellow solid; mp=227-229° C.; ¹H NMR (400 MHz, CDCl₃+DMSO-d₆) δ 1.42 (t, J=7.0 Hz, 3H), 2.82 (s, 3H), 4.41 (q, J=7.2 Hz, 2H), 6.90 (dd, J=7.2, 7.6 Hz, 1H), 7.04-7.06 (m, 1H), 7.38-7.66 (m, 2H), 7.96 (s, 1H), 8.46 (s, 1H), 8.57 (d, J=6.8 Hz, 1H), 11.52 (s, 1H), 11.95 (s, 1H).

### 2-(1H-Benzo[d]imidazol-5-ylamino)-9-fluoro-3-(hydroxymethyl)-4H-pyrido[1,2-a]pyrimidin-4-one (57)

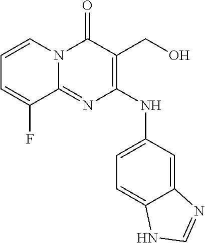 [SMILES: O=c1c(CO)c(Nc2ccc3[nH]cnc3c2)nc2c(F)cccn12]

Yellow solid; mp=184-185° C.; ¹H NMR (400 MHz, DMSO-d₆) δ 4.79 (s, 2H), 5.33 (brs, 1H), 7.12-7.17 (m, 1H), 7.30-7.41 (m, 1H), 7.46-7.68 (m, 1H), 7.82 (dd, J=8.4 Hz, 8.8 Hz, 1H), 8.19 (s, 1H), 8.72 (d, J=7.2 Hz, 1H), 8.87 (br s, 1H), 12.45 (br s, 1H).

### 9-Fluoro-3-(hydroxymethyl)-2-(1-methyl-1H-benzo[d]imidazol-5-ylamino)-4H-pyrido[1,2-a]pyrimidin-4-one (58)

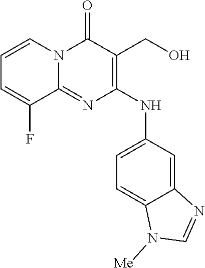 [SMILES: Cn1cnc2cc(Nc3nc4c(F)cccn4c(=O)c3CO)ccc21]

Yellow solid; mp=240° C. (decomp.); ¹H NMR (400 MHz, DMSO-d₆) δ 3.88 (s, 3H), 4.79 (d, J=5.2 Hz, 2H), 5.29 (t, J=5.2 Hz, 1H), 7.11-7.16 (m, 1H), 7.47 (dd, J=1.6, 8.4 Hz, 1H), 7.56 (d, J=8.4 Hz, 1H), 7.78-7.83 (m, 1H), 8.14-8.15 (m, 1H), 8.18 (s, 1H), 8.72 (d, J=7.2 Hz, 1H), 8.84 (s, 1H).

### 9-Fluoro-3-(hydroxymethyl)-2-(1-methyl-1H-benzo[d]imidazol-6-ylamino)-4H-pyrido[1,2-a]pyrimidin-4-one (59)

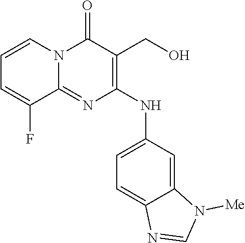 [SMILES: Cn1cnc2ccc(Nc3nc4c(F)cccn4c(=O)c3CO)cc21]

Yellow solid; mp=209° C. (decomp.); ¹H NMR (400 MHz, DMSO-d₆) δ 3.84 (s, 3H), 4.80 (s, 2H), 5.37 (s, 1H), 7.14-7.18 (m, 1H), 7.39 (dd, J=2.0, 8.4 Hz, 1H), 7.62 (d, J=8.8 Hz, 1H), 8.16 (s, 1H), 8.25-8.26 (m, 1H), 8.72 (d, J=7.2 Hz, 1H), 8.95 (s, 1H).

### Ethyl 2-(1H-benzo[d]imidazol-5-ylamino)-7-methyl-4-oxo-4H-pyrido[1,2-a]pyrimidine-3-carboxylate (60)

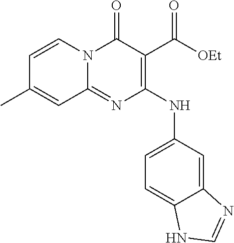 [SMILES: CCOC(=O)c1c(Nc2ccc3[nH]cnc3c2)nc2cc(C)ccn2c1=O]

Pale-yellow solid, mp=227° C.; ¹H NMR (400 MHz, CDCl₃) δ 1.44 (t, J=6.8 Hz, 3H), 2.51 (s, 3H), 4.45 (q, J=7.2 Hz, 2H), 7.13 (d, J=6.8 Hz, 1H), 7.30 (s, 1H), 7.72-7.83 (m, 3H), 8.70 (s, 1H), 8.94 (d, J=7.2 Hz, 1H), 9.34 (s, 1H).

### 2-(Benzo[d]oxazol-6-ylamino)-3-(hydroxymethyl)-9-methoxy-4H-pyrido[1,2-a]-pyrimidin-4-one (61)

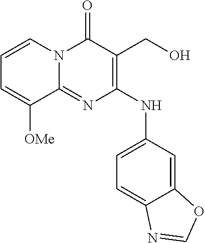 [SMILES: COc1cccn2c(=O)c(CO)c(Nc3ccc4ncoc4c3)nc12]

Pale yellow solid; mp=over 360° C. (decomp.); ¹H NMR (400 MHz, CDCl₃+CD₃OD) δ 3.96 (s, 3H), 4.82 (s, 2H), 6.81 (dd, J=7.2, 7.6 Hz, 1H), 6.88 (dd, J=1.2, 7.6 Hz, 1H), 7.26 (dd, J=2.0, 8.8 Hz, 1H), 7.57 (d, J=8.8 Hz, 1H), 8.03 (s, 1H), 8.45 (dd, J=1.2, 7.2 Hz, 1H), 8.59 (d, J=2.0 Hz, 1H); ¹³C NMR (100 MHz, CDCl₃) δ 56.3, 56.8, 95.5, 102.8, 111.8, 113.1, 118.0, 119.3, 119.7, 134.8, 138.2, 144.0, 150.6, 151.2, 152.4, 156.9, 157.4; LC-MS (ESI, m/z): 339[M+H]⁺.

### 3-(Hydroxymethyl)-9-methoxy-2-(2-methylbenzo[d]oxazol-6-ylamino)-4H-pyrido[1,2-a]-pyrimidin-4-one (62)

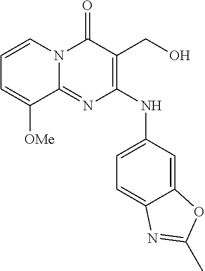 [SMILES: COc1cccn2c(=O)c(CO)c(Nc3ccc4nc(C)oc4c3)nc12]

Pale yellow solid; mp=over 380° C. (decomp.); ¹H NMR (400 MHz, CDCl₃+CD₃OD) δ 2.60 (s, 3H), 3.98 (s, 3H), 4.84 (s, 2H), 6.77 (dd, J=7.2, 7.6 Hz, 1H), 6.85 (dd, J=1.2, 7.6 Hz, 1H), 7.24 (dd, J=2.0, 8.4 Hz, 1H), 7.48 (d, J=8.4 Hz, 1H), 8.43-8.44 (m, 1H), 8.46 (dd, J=1.2, 7.2 Hz, 1H), 8.57 (brs, 1H); ¹³C NMR (100 MHz, CDCl₃+CD₃OD) δ 14.6, 56.5, 56.9, 95.3, 102.5, 111.7, 113.0, 117.3, 118.8, 119.5, 136.5, 137.1, 144.0, 151.2, 151.5, 156.8, 157.4, 163.7; LC-MS (ESI, m/z): 353[M+H]⁺.

### 2-(Benzo[d]oxazol-5-ylamino)-3-(hydroxymethyl)-9-methoxy-4H-pyrido[1,2-a]pyrimidin-4-one (63)

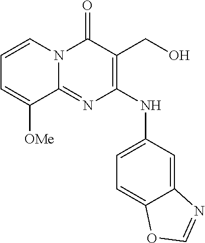 [SMILES: COc1cccn2c(=O)c(CO)c(Nc3ccc4ocnc4c3)nc12]

¹H NMR (400 MHz, DMSO-d₆) δ 3.86 (s, 3H), 4.65 (s, 2H), 5.18 (brs, 1H), 7.02 (dd, J=7.2, 7.2 Hz, 1H), 7.22 (d, J=7.2 Hz, 1H), 7.54-7.61 (m, 2H), 8.41 (d, J=7.2 Hz, 1H), 8.49 (s, 1H), 8.60 (s, 1H), 8.73 (s, 1H); LC-MS (ESI, m/z): 343[M+H]⁺.

### 3-(Hydroxymethyl)-9-methoxy-2-(2-methylbenzo[d]oxazol-5-ylamino)-4H-pyrido[1,2-a]-pyrimidin-4-one (64)

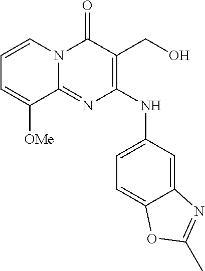 [SMILES: COc1cccn2c(=O)c(CO)c(Nc3ccc4oc(C)nc4c3)nc12]

White solid; mp=over 350° C. (decomp.); ¹H NMR (400 MHz, DMSO-d₆) δ 2.59 (s, 3H), 3.97 (s, 3H), 4.73 (s, 2H), 5.34 (brs, 1H), 7.09 (dd, J=7.2, 7.6 Hz, 1H), 7.31 (dd, J=1.2, 7.6 Hz, 1H), 7.42 (dd, J=2.0, 8.4 Hz, 1H), 7.53 (d, J=8.4 Hz, 1H), 8.48 (dd, J=1.2, 7.2 Hz, 1H), 8.65 (d, J=2.0 Hz, 1H), 8.86 (brs, 1H); LC-MS (ESI, m/z): 353[M+H]⁺.

### 2-(Benzo[d]oxazol-6-ylamino)-9-methoxy-4-oxo-4H-pyrido[1,2-a]pyrimidine-3-carbaldehyde (65)

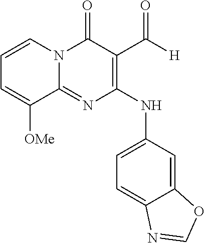 [SMILES: COc1cccn2c(=O)c(C=O)c(Nc3ccc4ncoc4c3)nc12]

Dark yellow solid; mp=291.0-292.2° C.; ¹H NMR (400 MHz, DMSO-d₆) δ 4.00 (s, 3H), 7.21 (dd, J=7.6, 7.6 Hz, 1H), 7.48 (d, J=8.8 Hz, 1H), 7.55 (d, J=7.6 Hz, 1H), 7.74 (d, J=8.8 Hz, 1H), 8.52 (d, J=7.6 Hz, 1H), 8.70 (s, 1H), 8.93 (s, 1H), 10.13 (s, 1H), 11.77 (s, 1H); LC-MS (ESI, m/z): 337[M+H]⁺.

### (2-(Benzo[d]oxazol-6-ylamino)-9-methoxy-4-oxo-4H-pyrido[1,2-a]pyrimidin-3-yl)methyl isobutyrate (66)

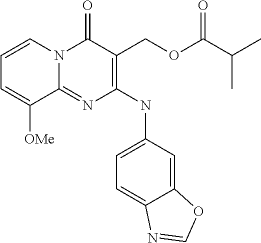 [SMILES: COc1cccn2c(=O)c(COC(=O)C(C)C)c(Nc3ccc4ncoc4c3)nc12]

Bright yellow solid; mp=137-139° C.; ¹H NMR (400 MHz, CDCl₃) δ 1.18 (d, J=6.4 Hz, 6H), 2.64-2.67 (m, 1H), 4.04 (s, 3H), 5.44 (s, 2H), 6.92 (dd, J=7.2, 7.2 Hz, 1H), 6.99 (d, J=7.2 Hz, 1H), 7.47 (dd, J=2.0, 8.8 Hz, 1H), 7.69-7.71 (d, J=8.8 Hz, 1H), 8.04 (s, 1H), 8.64-8.67 (m, 2H), 9.29 (brs, 1H); LC-MS (ESI, m/z): 409[M+H]⁺.

### 2-(Benzo[d]oxazol-6-ylamino)-9-(difluoromethoxy)-3-(hydroxymethyl)-4H-pyrido[1,2-a]pyrimidin-4-one (67)

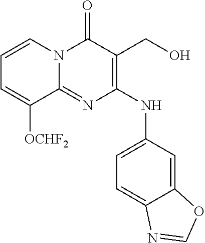 [SMILES: O=c1c(CO)c(Nc2ccc3ncoc3c2)nc2c(OC(F)F)cccn12]

Pale yellow solid; ¹H NMR (400 MHz, CDCl₃+CD₃OD) δ 4.77 (s, 2H), 6.78 (t. J=74 Hz, 1H due to F₂), 6.92 (dd, J=7.2, 7.2 Hz, 1H), 7.28 (dd, J=2.0, 8.8 Hz, 1H), 7.46 (d, J=7.6 Hz, 1H), 7.54 (d, J=8.4 Hz, 1H), 8.07 (s, 1H), 8.26 (d, J=1.6 Hz, 1H), 8.70 (dd, J=1.2, 7.2 Hz, 1H).

### 2-(Benzo[d]oxazol-6-ylamino)-9-fluoro-3-(hydroxymethyl)-4H-pyrido[1,2-a]pyrimidin-4-one (68)

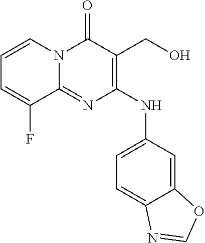 [SMILES: O=c1c(CO)c(Nc2ccc3ncoc3c2)nc2c(F)cccn12]

Pale yellow solid; mp=236-237° C. (decomp.); ¹H NMR (400 MHz, CDCl₃+CD₃OD) δ 4.79 (s, 2H), 5.38 (br s, 1H), 7.17-7.22 (m, 1H), 7.55 (dd, J=2.4, 8.4 Hz, 1H), 7.77 (d, J=8.8 Hz, 1H), 7.84-7.89 (m, 1H), 8.51 (d, J=2.0 Hz, 1H), 8.69 (s, 1H), 8.74 (d, J=6.8 Hz, 1H), 9.08 (br s, 1H); LC-MS (ESI, m/z): 327.26 [M+H]⁺.

### 3-(Hydroxymethyl)-9-methoxy-2-(2-methylbenzo[d]thiazol-6-ylamino)-4H-pyrido[1,2-a]-pyrimidin-4-one (69)

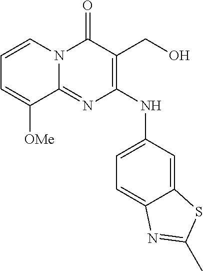 [SMILES: COc1cccn2c(=O)c(CO)c(Nc3ccc4nc(C)sc4c3)nc12]

White solid; mp=217-219° C. (decomp.); ¹H NMR (400 MHz, DMSO-d₆) δ 2.76 (s, 3H), 3.96 (s, 3H), 4.73 (s, 2H), 5.33 (brs, 1H), 7.09 (dd, J=7.2, 7.6 Hz, 1H), 7.30 (d, J=7.6 Hz, 1H), 7.70 (dd, J=2.4, 8.8 Hz, 1H), 7.80 (d, J=8.8 Hz, 1H), 8.48 (dd, J=1.2, 7.2 Hz, 1H), 8.80 (d, J=2.4 Hz, 1H), 8.87 (brs, 1H); LC-MS (ESI, m/z): 369[M+H]⁺.

### 3-(Hydroxymethyl)-9-methoxy-2 (2-(trifluoromethyl)-1H-benzo[d]imidazol-5-ylamino)-4H-pyrido[1,2-a]pyrimidin-4-one (70)

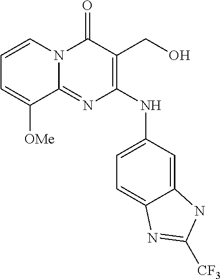 [SMILES: COc1cccn2c(=O)c(CO)c(Nc3ccc4nc(C(F)(F)F)[nH]c4c3)nc12]

White solid; mp=197-198° C. (decomp.); ¹H NMR (400 MHz, CD₃OD) δ 4.07 (s, 3H), 4.58 (s, 2H), 7.08 (dd, J=7.2, 7.6 Hz, 1H), 7.25 (d, J=8.0 Hz, 1H), 7.47 (dd, J=1.6, 8.8 Hz, 1H), 7.64 (d, J=8.8 Hz, 1H), 8.55 (d, J=7.2 Hz, 1H), 8.69 (d, J=1.6 Hz, 1H); LC-MS (ESI, m/z); [M+H]⁺ 406.17

### 3-(Hydroxymethyl)-9-methoxy-2-(quinolin-6-ylamino)-4H-pyrido[1,2-a]pyrimidin-4-one (71)

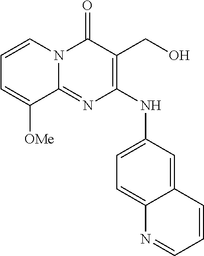 [SMILES: COc1cccn2c(=O)c(CO)c(Nc3ccc4ncccc4c3)nc12]

Pale yellow solid; mp=207° C. (decomp); ¹H NMR (400 MHz, DMSO d-6) δ 4.01 (s, 3H), 4.76 (d, J=5.2 Hz, 2H), 5.35 (t, J=5.2 Hz, 1H), 7.11 (dd, J=7.2 Hz, 7.2 Hz, 1H), 7.31 (d, J=6.8 Hz, 1H), 7.46 (dd, J=4.0 Hz, 8.4 Hz, 1H), 7.93 (d, J=9.2 Hz, 1H), 8.03 (dd, J=2.4 Hz, 9.2 Hz, 1H), 8.18 (d, J=8.4 Hz, 1H), 8.48 (d, J=7.2 Hz, 1H), 8.73 (d, J=2.4 Hz, 1H), 8.78 (d, J=2.4 Hz, 1H), 9.00 (s, 1H); ¹³C NMR (100 MHz, DMSO) δ 59.9, 62.3, 100.8, 118.3, 118.9, 120.7, 124.3, 127.1, 130.3, 134.0, 134.4, 140.7, 143.7, 148.7, 153.9, 156.1, 161.3, 161.9, 207.7.

### 3-(Hydroxymethyl)-9-methoxy-2-(2-methylquinolin-6-ylamino)-4H-pyrido[1,2-a]-pyrimidin-4-one (72)

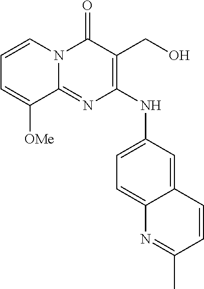 [SMILES: COc1cccn2c(=O)c(CO)c(Nc3ccc4nc(C)ccc4c3)nc12]

Pale yellow solid; mp=over 270° C. (decomp.); ¹H NMR (400 MHz, DMSO-d₆) δ 2.61 (s, 3H), 4.00 (s, 3H), 4.75 (s, 2H), 5.34 (brs, 1H), 7.10 (dd, J=7.2, 7.6 Hz, 1H), 7.31 (dd, J=1.2, 7.6 Hz, 1H), 7.34 (d, J=8.4 Hz, 1H), 7.81 (d, J=8.8 Hz, 1H), 7.96 (dd, J=2.4, 8.8 Hz, 1H), 8.07 (d, J=8.4 Hz, 1H), 8.48 (dd, J=1.2, 7.2 Hz, 1H), 8.70 (d, J=2.4 Hz, 1H), 8.94 (brs, 1H).

### 9-Fluoro-3-(hydroxymethyl)-2-(quinolin-6-ylamino)-4H-pyrido[1,2-a]pyrimidin-4-one (73)

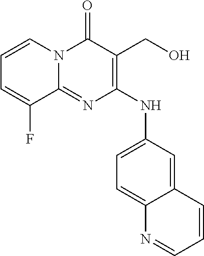 [SMILES: O=c1c(CO)c(Nc2ccc3ncccc3c2)nc2c(F)cccn12]

Pale yellow solid; mp=243-244° C. (decomp.); ¹H NMR (400 MHz, CDCl₃+CD₃OD) δ 4.82 (s, 2H), 5.43 (br s, 1H), 7.18-7.23 (m, 1H), 7.51-7.55 (m, 1H), 7.86-7.90 (m, 1H), 8.11 (dd, J=2.4, 8.8 Hz, 1H), 8.26 (d, J=7.6 Hz, 1H), 8.44 (d, J=2.4 Hz, 1H), 8.75 (d, J=7.2 Hz, 1H), 8.81-8.83 (m, 1H), 9.19 (br s, 1H); LC-MS (ESI, m/z); [M+H]⁺ 337.19

### 2-(3-Chloroquinolin-6-ylamino)-3-(hydroxymethyl)-9-methoxy-4H-pyrido[1,2-a]pyrimidin-4-one (74)

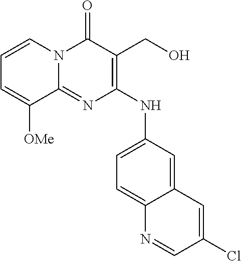 [SMILES: COc1cccn2c(=O)c(CO)c(Nc3ccc4ncc(Cl)cc4c3)nc12]

¹H NMR (400 MHz, DMSO-d₆) δ 4.03 (s, 3H), 4.78 (s, 2H), 5.40 (brs, 1H), 7.16 (dd, J=7.2, 7.2 Hz, 1H), 7.36 (d, J=7.2 Hz, 1H), 7.98 (d, J=8.8 Hz, 1H), 8.19 (dd, J=2.4, 8.8 Hz, 1H), 8.33 (d, J=2.4 Hz, 1H), 8.51 (d, J=7.2 Hz, 1H), 8.63 (d, J=2.4 Hz, 1H), 8.72 (d, J=2.4 Hz, 1H), 9.12 (s, 1H); LC-MS (ESI, m/z): 383[M+H]⁺.

### 2-(3,8-Dichloroquinolin-6-ylamino)-3-(hydroxymethyl)-9-methoxy-4H-pyrido[1,2-a]pyrimidin-4-one (75)

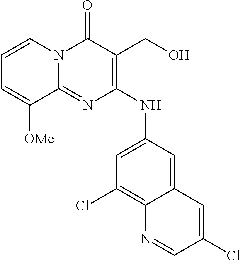 [SMILES: COc1cccn2c(=O)c(CO)c(Nc3cc(Cl)c4ncc(Cl)cc4c3)nc12]

¹H NMR (400 MHz, DMSO-d₆) δ 4.01 (s, 3H), 4.66 (s, 2H), 7.14 (dd, J=7.2, 7.2 Hz, 1H), 7.34 (d, J=7.2 Hz, 1H), 8.34 (d, J=2.4 Hz, 1H), 8.43 (d, J=2.0 Hz, 1H), 8.46 (d, J=7.2 Hz, 1H), 8.77 (d, J=2.0 Hz, 1H), 8.78 (d, J=2.4 Hz, 1H), 8.79 (s, 1H); LC-MS (ESI, m/z): 417 [M+H]⁺.

### 3-(1-Hydroxyethyl)-9-methoxy-2-(quinolin-6-ylamino)-4H-pyrido[1,2-a]pyrimidin-4-one (76)

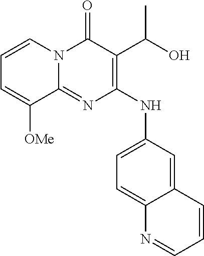 [SMILES: COc1cccn2c(=O)c(C(C)O)c(Nc3ccc4ncccc4c3)nc12]

White solid (racemic); ¹H NMR (400 MHz, CDCl₃) δ 1.49 (d, J=6.4 Hz, 2H), 3.97 (s, 3H), 4.50 (brs, 1H), 5.58 (q, J=6.4 Hz, 1H), 6.54 (dd, J=7.2 Hz, 1H), 6.64 (d, J=7.6 Hz, 1H), 7.37 (dd, J=4.0 Hz, 8.0 Hz, 1H), 7.82 (dd, J=2.4 Hz, 9.2 Hz, 1H), 8.03 (d, J=9.2 Hz, 1H), 8.05 (d, J=8.0 Hz, 1H), 8.40 (dd, J=1.2 Hz, 7.2 Hz, 1H), 8.53 (d, J=2.0 Hz, 1H), 8.79 (dd, J=1.6 Hz, 4.0 Hz, 1H), 9.47 (s, 1H).

### 3-Acetyl-9-methoxy-2-(quinolin-6-ylamino)-4H-pyrido[1,2-a]pyrimidin-4-one (77)

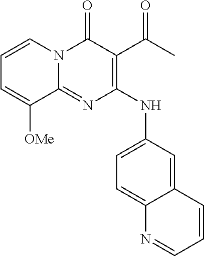 [SMILES: COc1cccn2c(=O)c(C(C)=O)c(Nc3ccc4ncccc4c3)nc12]

White solid; ¹H NMR (400 MHz, CDCl₃) δ 2.80 (s, 3H), 4.03 (s, 3H), 6.92 (d, J=6.8 Hz, 1H), 7.05 (d, J=7.2 Hz, 1H), 7.36-7.38 (m, 1H), 7.96 (d, J=8.8 Hz, 1H), 8.04 (d, J=9.2 Hz, 1H), 8.08 (d, J=7.6 Hz, 1H), 8.60 (d, J=7.2 Hz, 1H), 8.75 (s, 1H), 8.81 (s, 1H), 12.9 (s, 1H); ¹³C NMR (100 MHz, CDCl₃) δ 33.6, 57.1, 95.5, 113.1, 114.5, 118.3, 120.0, 121.6, 125.8, 129.1, 130.0, 136.0, 137.1, 145.8, 146.1, 149.4, 151.3, 158.4, 158.7, 201.1.

### 9-Methoxy-4-oxo-2-(quinolin-6-ylamino)-4H-pyrido[1,2-a]pyrimidine-3-carbaldehyde (78)

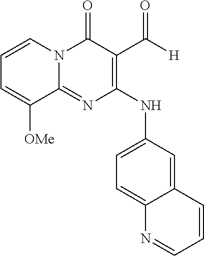 [SMILES: COc1cccn2c(=O)c(C=O)c(Nc3ccc4ncccc4c3)nc12]

Yellow solid; mp=238-240° C.; ¹H NMR (400 MHz, DMSO-d₆) δ 3.99 (s, 3H), 7.16 (dd, J=7.2, 7.6 Hz, 1H), 7.46-7.50 (m, 2H), 7.92-7.99 (m, 2H), 8.17 (d, J=8.0 Hz, 1H), 8.45 (d, J=7.2 Hz, 1H), 8.77 (dd, J=2.0, 4.4 Hz, 1H), 8.84 (d, J=2.0 Hz, 1H), 10.07 (s, 1H), 11.71 (s, 1H); LC-MS (ESI, m/z): 347[M+H]⁺.

### (9-Methoxy-4-oxo-2-(quinolin-6-ylamino)-4H-pyrido[1,2-a]pyrimidin-3-yl)methyl isobutyrate (79)

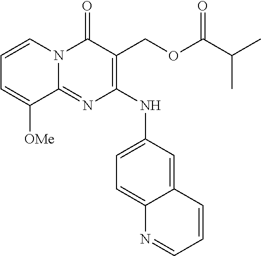 [SMILES: COc1cccn2c(=O)c(COC(=O)C(C)C)c(Nc3ccc4ncccc4c3)nc12]

Bright yellow solid; mp=153.5-157.2° C.; ¹H NMR (400 MHz, CDCl₃) δ 1.16 (d, J=6.8 Hz, 6H), 2.61-2.67 (m, 1H), 3.99 (s, 3H), 5.42 (s, 2H), 6.87 (dd, J=1.2, 7.6 Hz, 1H), 6.94 (dd, J=1.2, 7.6 Hz, 1H), 7.31 (dd, J=4.4, 8.4 Hz, 1H), 7.94 (dd, J=2.4, 9.2 Hz, 1H), 8.03 (d, J=9.2 Hz, 1H), 8.06 (d, J=8.4 Hz, 1H), 8.59-8.62 (m, 2H), 8.76 (dd, J=1.2, 4.4 Hz, 1H), 9.31 (brs, 1H); ¹³C NMR (100 MHz, CDCl₃) δ 19.1, 19.3, 34.2, 56.9, 58.6, 92.6, 112.4, 112.9, 115.4, 119.8, 121.4, 124.7, 129.2, 129.6, 135.8, 138.2, 144.9, 145.0, 148.5, 151.3, 156.6, 159.0, 180.5; LC-MS (ESI, m/z): 419[M+H]⁺.

### (9-Methoxy-4-oxo-2-(quinolin-6-ylamino)-4H-pyrido[1,2-a]pyrimidin-3-yl)methyl 2-amino-3-methylbutanoate (80)

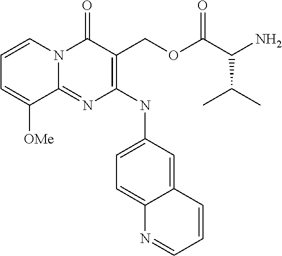

Yellow solid; ¹H NMR (400 MHz, CDCl₃) δ 1.01-1.03 (m, 6H), 2.01-2.13 (m, 1H), 3.26 (d, J=5.2 Hz, 1H), 3.81 (s, 3H), 3.82 (d, J=13.2 Hz, 1H), 4.07 (s, 2H), 4.19 (d, J=13.2 Hz, 1H), 6.90 (dd, J=7.2, 7.6 Hz, 1H), 6.96 (dd, J=1.2, 7.6 Hz, 1H), 7.34 (dd, J=4.0, 8.0 Hz, 1H), 7.91 (dd, J=2.4, 9.2 Hz, 1H), 8.04 (d, J=9.2 Hz, 1H), 8.09 (d, J=8.0 Hz, 1H), 8.63 (dd, J=1.2, 7.2 Hz, 1H), 8.73 (d, J=2.4 Hz, 1H), 8.77 (dd, J=1.2, 4.0 Hz, 1H), 10.25 (brs, 1H).

### 9-Methoxy-4-oxo-2-(quinolin-6-ylamino)-4H-pyrido[1,2-a]pyrimidine-3-carboxylic acid (81)

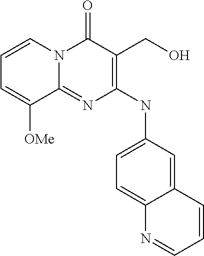 [SMILES: COc1cccn2c(=O)c(CO)c(Nc3ccc4ncccc4c3)nc12]

Bright yellow solid; mp=243.4-245.2° C.; ¹H NMR (400 MHz, DMSO-d₆) δ 4.08 (s, 3H), 7.29 (dd, J=7.2, 7.6 Hz, 1H), 7.52 (dd, J=4.0, 8.4 Hz, 1H), 7.56 (d, J=7.6 Hz, 1H), 7.99 (d, J=8.4 Hz, 1H), 8.06 (d, J=9.2 Hz, 1H), 8.26 (d, J=8.4 Hz, 1H), 8.50 (d, J=7.2 Hz, 1H), 8.81-8.82 (m, 1H), 8.91 (s, 1H), 11.82 (s, 1H); LC-MS (ESI, m/z): 363[M+H]⁺.

### Ethyl 9-methoxy-4-oxo-2-(quinolin-6-ylamino)-4H-pyrido[1,2-a]pyrimidine-3-carboxylate (82)

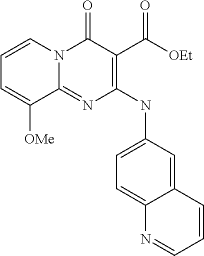 [SMILES: CCOC(=O)c1c(Nc2ccc3ncccc3c2)nc2c(OC)cccn2c1=O]

Yellow solid; mp=199.0-200.2° C.; ¹H NMR (400 MHz, DMSO-d₆) δ 1.31 (t, J=6.8 Hz, 3H), 4.04 (s, 3H), 4.30 (q, J=6.8 Hz, 2H), 7.17 (dd, J=7.2, 7.6 Hz, 1H), 7.47-7.53 (m, 2H), 7.95-8.01 (m, 2H), 8.22 (d, J=8.0 Hz, 1H), 8.50 (d, J=7.2 Hz, 1H), 8.79 (s, 1H), 8.94 (s, 1H), 11.50 (s, 1H); LC-MS (ESI, m/z): 391[M+H]⁺.

### 9-Methoxy-3-(methoxymethyl)-2-(quinolin-6-ylamino)-4H-pyrido[1,2-a]pyrimidin-4-one (83)

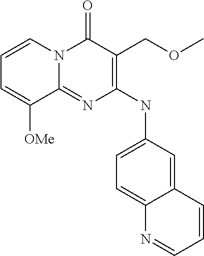 [SMILES: COCc1c(Nc2ccc3ncccc3c2)nc2c(OC)cccn2c1=O]

Yellow solid; mp=over 230° C. (decomp.); ¹H NMR (400 MHz, DMSO-d₆) δ 3.34 (s, 3H), 4.05 (s, 3H), 4.72 (s, 2H), 7.18 (dd, J=7.2, 7.6 Hz, 1H), 7.39 (d, J=8.0 Hz, 1H), 7.88 (dd, J=4.8, 8.0 Hz, 1H), 8.12 (d, J=9.2 Hz, 1H), 8.39 (dd, J=2.4, 9.2 Hz, 1H), 8.52 (d, J=7.6 Hz, 1H), 8.79 (d, J=7.6 Hz, 1H), 8.96 (brs, 1H), 9.05 (d, J=4.8 Hz, 1H), 9.11 (s, 1H); LC-MS (ESI, m/z): 363[M+H]⁺.

### 9-Methoxy-3-(methoxymethyl)-2-(methyl(quinolin-6-yl)amino)-4H-pyrido[1,2-a]-pyrimidin-4-one (84)

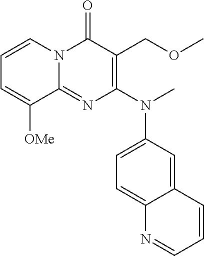 [SMILES: COCc1c(N(C)c2ccc3ncccc3c2)nc2c(OC)cccn2c1=O]

Yellow solid; ¹H NMR (400 MHz, DMSO-d₆) δ 2.84 (s, 3H), 3.66 (s, 3H), 4.01 (s, 3H), 4.06 (s, 2H), 7.15 (dd, J=7.6, 7.6 Hz, 1H), 7.30 (d, J=7.6 Hz, 1H), 7.51 (dd, J=4.4, 8.0 Hz, 1H), 7.60-7.65 (m, 2H), 7.95 (d, J=9.2 Hz, 1H), 8.31 (d, J=8.4 Hz, 1H), 8.57 (d, J=7.6 Hz, 1H), 8.74 (s, 1H); LC-MS (ESI, m/z): 377[M+H]⁺.

### 3-(9-Methoxy-4-oxo-2-(quinolin-6-ylamino)-4H-pyrido[1,2-a]pyrimidin-3-yl)acrylic acid (85)

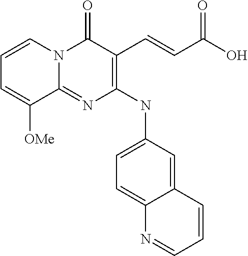 [SMILES: COc1cccn2c(=O)c(/C=C/C(=O)O)c(Nc3ccc4ncccc4c3)nc12]

Bright yellow solid; mp=over 240° C. (decomp.); ¹H NMR (400 MHz, DMSO-d₆) δ 3.97 (s, 3H), 7.18 (dd, J=7.2, 7.6 Hz, 1H), 7.25 (d, J=15.2 Hz, 1H), 7.40 (d, J=7.6 Hz, 1H), 7.81-7.84 (m, 1H), 7.93 (d, J=15.2 Hz, 1H), 8.13 (d, J=9.2 Hz, 1H), 8.36 (d, J=9.2 Hz, 1H), 8.56 (dd, J=1.2, 7.2 Hz, 1H), 8.70 (d, J=7.6 Hz, 1H), 8.82 (s, 1H), 8.99 (d, J=4.8 Hz, 1H), 9.98 (s, 1H); LC-MS (ESI, m/z): 389[M+H]⁺.

### Ethyl 3-(9-methoxy-4-oxo-2-(quinolin-6-ylamino)-4H-pyrido[1,2-a]pyrimidin-3-yl)-acrylate (86)

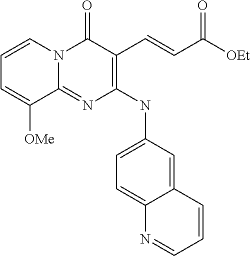 [SMILES: CCOC(=O)/C=C/c1c(Nc2ccc3ncccc3c2)nc2c(OC)cccn2c1=O]

Yellow solid; mp=239.0-241.0° C.; ¹H NMR (400 MHz, DMSO-d₆) δ 1.24 (t, J=6.8 Hz, 3H), 3.95 (s, 3H), 4.15 (q, J=6.8 Hz, 2H), 7.15 (dd, J=7.2, 7.6 Hz, 1H), 7.30 (d, J=15.2 Hz, 1H), 7.37 (d, J=7.6 Hz, 1H), 7.48 (dd, J=4.0, 8.4 Hz, 1H), 7.94 (d, J=8.4 Hz, 1H), 8.00 (d, J=15.2 Hz, 1H), 8.12 (dd, J=2.4 Hz, 9.2 Hz, 1H), 8.21 (d, J=8.4 Hz, 1H), 8.55-8.57 (m, 2H), 8.78 (dd, J=1.6, 4.0 Hz, 1H), 9.83 (brs, 1H); LC-MS (ESI, m/z): 417[M+H]⁺.

### 3-Bromo-9-methoxy-2-(quinolin-6-ylamino)-4H-pyrido[1,2-a]pyrimidin-4-one (87)

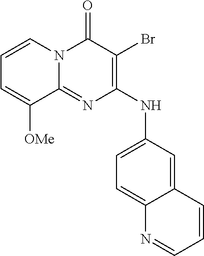 [SMILES: COc1cccn2c(=O)c(Br)c(Nc3ccc4ncccc4c3)nc12]

Pale yellow solid; mp=248-249° C.; ¹H NMR (400 MHz, DMSO-d₆) δ 3.96 (s, 3H), 7.14 (dd, J=7.2, 7.6 Hz, 1H), 7.36 (d, J=7.2 Hz, 1H), 7.47 (dd, J=4.0, 8.0 Hz, 1H), 7.92 (d, J=9.2 Hz, 1H), 8.13 (dd, J=2.4, 9.2 Hz, 1H), 8.20 (d, J=7.6 Hz, 1H), 8.47 (dd, J=1.2, 7.2 Hz, 1H), 8.68 (d, J=4.0 Hz, 1H), 8.77 (dd, J=1.2, 4.0 Hz, 1H), 8.85 (brs, 1H); LC-MS (ESI, m/z): 397, 399[M+H]⁺, Br isotope pattern.

### 3-((Cyclopentylamino)methyl)-9-methoxy-2-(quinolin-6-ylamino)-4H-pyrido[1,2-a]pyrimidin-4-one (88)

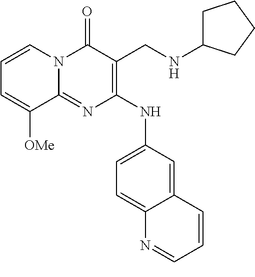 [SMILES: COc1cccn2c(=O)c(CNC3CCCC3)c(Nc3ccc4ncccc4c3)nc12]

White solid; ¹H NMR (400 MHz, DMSO-d₆) δ 1.48 (m, 4H), 1.67-1.76 (m, 2H), 1.79-1.81 (m, 2H), 3.05-3.08 (m, 1H), 3.93 (s, 2H), 4.02 (s, 3H), 7.10 (dd, J=7.6 Hz, 1H), 7.29 (d, J=7.2 Hz, 1H), 7.46 (d, J=7.2 Hz, 1H), 7.77 (dd, J=2.0 Hz, 8.8 Hz, 1H), 7.94 (d, J=8.8 Hz, 1H), 8.17 (d, J=8.4 Hz, 1H), 8.48 (d, J=7.2 Hz, 1H), 8.72 (dd, J=2.0 Hz, 8.0 Hz, 1H), 8.75 (d, J=2.0 Hz, 1H).

### 3-((Benzylamino)methyl)-9-methoxy-2-(quinolin-6-ylamino)-4H-pyrido[1,2-a]pyrimidin-4-one (89)

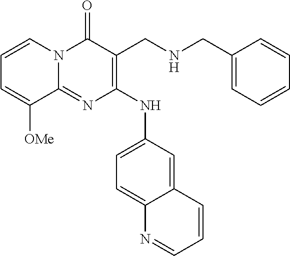 [SMILES: COc1cccn2c(=O)c(CNCc3ccccc3)c(Nc3ccc4ncccc4c3)nc12]

White solid; ¹H NMR (400 MHz, CDCl₃) δ 3.89 (s, 2H), 4.05 (s, 3H), 4.21 (s, 2H), 6.89-6.96 (m, 2H), 7.29-7.40 (m, 5H), 7.79 (dd, J=2.4 Hz, 9.2 Hz, 1H), 8.02 (d, J=9.2 Hz, 1H), 8.07 (d, J=8.4 Hz, 1H), 8.59 (d, J=2.4 Hz, 1H), 8.65 (dd, J=2.0 Hz, 6.8 Hz, 1H), 8.77 (dd, J=2.0 Hz, 4.4 Hz, 1H), 10.88 (s, 1H); ¹³C NMR (100 MHz, CDCl₃) δ 44.6, 53.3, 57.0, 92.7, 111.4, 112.7, 114.8, 117.2, 119.8, 121.5, 124.6, 127.6, 128.7, 128.9, 129.5, 129.9, 135.6, 138.6, 139.7, 145.1, 148.5, 151.4, 157.5, 157.9; LC-MS (ESI, m/z); [M+H]⁺ 438.34

### 3-(Hydroxymethyl)-9-methoxy-2-(quinazolin-6-ylamino)-4H-pyrido[1,2-a]pyrimidin-4-one (90)

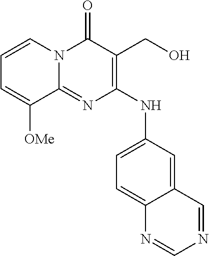 [SMILES: COc1cccn2c(=O)c(CO)c(Nc3ccc4ncncc4c3)nc12]

White solid; ¹H NMR (400 MHz, DMSO-d₆) δ 4.06 (s, 3H), 4.78 (s, 2H), 7.15 (dd, J=7.2, 7.6 Hz, 1H), 7.36 (d, J=7.6 Hz, 1H), 7.95 (d, J=8.8 Hz, 1H), 8.28 (dd, J=2.0, 8.8 Hz, 1H), 8.51 (d, J=7.2 Hz, 1H), 9.02 (d, J=2.0 Hz, 1H), 9.16 (s, 1H), 9.44 (s, 1H).

### 3-(Hydroxymethyl)-9-methoxy-2-(quinoxalin-6-ylamino)-4H-pyrido[1,2-a]pyrimidin-4-one (91)

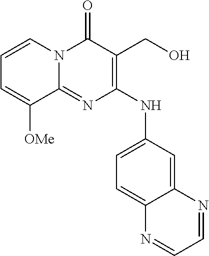 [SMILES: COc1cccn2c(=O)c(CO)c(Nc3ccc4nccnc4c3)nc12]

Pale yellow solid; ¹H NMR (400 MHz, CDCl₃+CD₃OD) δ 4.06 (s, 3H), 4.97 (s, 2H), 6.95-7.02 (m, 2H), 8.00 (d, J=9.2 Hz, 1H), 8.06 (dd, J=9.2, 2.4 Hz, 1H), 8.59-8.70 (m, 3H), 8.75 (s, 1H).

### 3-(Hydroxymethyl)-9-methoxy-2-(1,2,3,4-tetrahydroquinazolin-6-ylamino)-4H-pyrido-[1,2-a]pyrimidin-4-one (92)

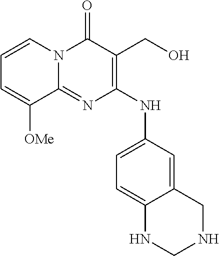 [SMILES: COc1cccn2c(=O)c(CO)c(Nc3ccc4c(c3)CNCN4)nc12]

Yellow solid; mp=147.5-149.3° C.; ¹H NMR (400 MHz, CDCl₃) δ 3.96 (s, 3H), 4.05 (s, 2H), 4.24 (s, 2H), 4.88 (s, 2H), 6.52 (d, J=8.8 Hz, 1H), 6.77 (dd, J=7.2, 7.6 Hz, 1H), 6.86 (dd, J=1.2, 7.6 Hz, 1H), 7.19 (dd, J=2.4, 8.8 Hz, 1H), 7.48 (d, J=2.4 Hz, 1H), 7.85 (brs, 1H), 8.50 (dd, J=1.2, 7.2 Hz, 1H); LC-MS (ESI, m/z): 354[M+H]⁺.

### 2-(3,4-Difluorophenylamino)-3-(hydroxy(phenyl)methyl)-9-methoxy-4H-pyrido[1,2-a]pyrimidin-4-one (93)

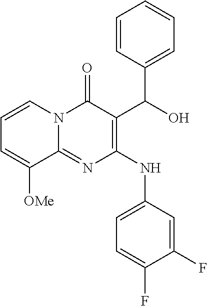 [SMILES: COc1cccn2c(=O)c(C(O)c3ccccc3)c(Nc3ccc(F)c(F)c3)nc12]

Pale yellow solid; mp=226-227° C.; ¹H NMR (400 MHz, CDCl₃+CD₃OD) δ 3.98 (s, 3H), 6.53 (s, 1H), 6.84-6.88 (m, 1H), 6.91 (d, J=6.8 Hz, 1H), 6.98-7.04 (m, 2H), 7.16-7.20 (m, 1H), 7.24-7.27 (m, 2H), 7.48 (d, J=7.2 Hz, 2H), 8.02-8.08 (m, 1H), 8.54 (dd, J=0.8, 6.8 Hz, 1H); LC-MS (ESI, m/z): 410[M+H]⁺.

### 2-(3,4-Difluorophenylamino)-3-(1-hydroxyethyl)-9-methoxy-4H-pyrido[1,2-a]pyrimidin-4-one (94)

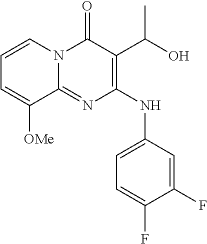 [SMILES: COc1cccn2c(=O)c(C(C)O)c(Nc3ccc(F)c(F)c3)nc12]

Pale yellow solid; mp=211.0-212.8° C.; ¹H NMR (400 MHz, CDCl₃+CD₃OD) δ 1.42 (d, J=6.8 Hz, 3H), 3.96 (s, 3H), 5.41 (q, J=6.8 Hz, 1H), 6.84 (dd, J=7.2, 7.6 Hz, 1H), 6.90 (dd, J=1.6, 7.6 Hz, 1H), 6.98-7.05 (m, 1H), 7.08-7.12 (m, 1H), 8.07-8.13 (m, 1H), 8.45 (dd, J=1.6, 7.2 Hz, 1H); LC-MS (ESI, m/z): 348[M+H]⁺.

### 2-(4-(1H-Imidazol-1-yl)phenylamino)-3-(hydroxymethyl)-9-methoxy-4H-pyrido[1,2-a]pyrimidin-4-one (95)

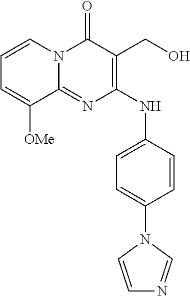 [SMILES: COc1cccn2c(=O)c(CO)c(Nc3ccc(-n4ccnc4)cc3)nc12]

Yellow solid; mp=240-243° C. (decomp.); ¹H NMR (400 MHz, CDCl₃) δ 3.21 (s, 3H), 4.99 (s, 2H), 6.88 (dd, J=7.2, 7.2 Hz, 1H), 6.95 (d, J=7.2 Hz, 1H), 7.20 (s, 1H), 7.35 (d, J=8.8 Hz, 2H), 7.83 (s, 1H), 7.87 (d, J=8.8 Hz, 2H), 8.37 (s, 1H), 8.59 (d, J=7.2 Hz, 1H); LC-MS (ESI, m/z): 364[M+H]⁺.

### 2-(3-Chloro-4-methoxyphenylamino)-3-(hydroxymethyl)-9-methoxy-4H-pyrido[1,2-a]pyrimidin-4-one (96)

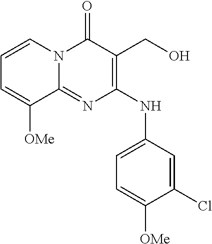 [SMILES: COc1ccc(Nc2nc3c(OC)cccn3c(=O)c2CO)cc1Cl]

Pale yellow solid; mp=318-320° C.; ¹H NMR (400 MHz, DMSO-d₆) δ 3.81 (s, 3H), 3.93 (s, 3H), 4.67 (s, 2H), 5.13 (brs, 1H), 7.04-7.09 (m, 2H), 7.26 (d, J=7.6 Hz, 1H), 7.57 (dd, J=2.8, 9.2 Hz, 1H), 8.36 (d, J=2.8 Hz, 1H), 8.44-8.46 (m, 1H), 8.59 (brs, 1H); ¹³C NMR (100 MHz, DMSO-d₆) δ 54.2, 56.1, 56.7, 94.6, 112.6, 112.8, 113.1, 118.7, 119.8, 120.4, 122.0, 134.1, 143.2, 149.7, 150.5, 155.7, 156.4; LC-MS (ESI, m/z): 362, 364[M+H]⁺, Cl isotope pattern.

### 2-(4-Chloro-3-fluorophenylamino)-3-(hydroxymethyl)-9-methoxy-4H-pyrido[1,2-a]pyrimidin-4-one (97)

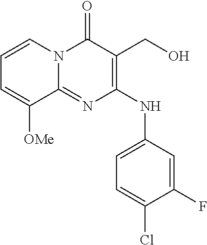 [SMILES: COc1cccn2c(=O)c(CO)c(Nc3ccc(Cl)c(F)c3)nc12]

White solid; mp=238-239° C. (decomp.); ¹H NMR (400 MHz, CD₃OD) δ 4.01 (s, 3H), 4.75 (s, 2H), 5.29 (br s, 1H), 7.18 (dd, J=7.2, 7.6 Hz, 1H), 7.38 (d, J=8.0 Hz, 1H), 7.50 (dd, J=8.4, 8.4 Hz, 1H), 7.56 (dd, J=2.0, 9.2 Hz, 1H), 8.48 (dd, J=2.0, 13.2 Hz, 1H), 8.53 (dd, J=1.2, 6.8 Hz, 1H), 8.94 (d, J=1.6 Hz, 1H); LC-MS (ESI, m/z); [M+H]⁺ 350.12

### 2-(3-Hydroxy-4-methylphenylamino)-3-(hydroxymethyl)-9-methoxy-4H-pyrido[1,2-a]pyrimidin-4-one (98)

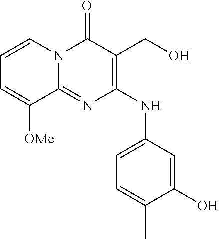 [SMILES: COc1cccn2c(=O)c(CO)c(Nc3ccc(C)c(O)c3)nc12]

White solid; mp=201-202° C.; ¹H NMR (400 MHz, DMSO-d₆) δ 1.96 (s, 3H), 3.91 (s, 3H), 4.67 (d, J=4.8 Hz, 2H), 5.29 (brs, 1H), 6.95 (d, J=8.4 Hz, 1H), 7.03 (s, 1H), 7.05 (d, J=8.0 Hz, 1H), 7.24 (d, J=8.0 Hz, 1H), 7.28 (s, 1H), 8.45 (d, J=6.8 Hz, 1H), 8.50 (s, 1H), 9.16 (s, 1H).

### 2-(4-Hydroxy-3-methylphenylamino)-3-(hydroxymethyl)-9-methoxy-4H-pyrido[1,2-a]pyrimidin-4-one (99)

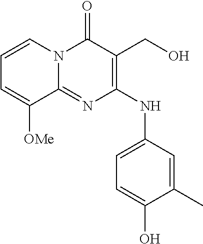 [SMILES: COc1cccn2c(=O)c(CO)c(Nc3ccc(O)c(C)c3)nc12]

White solid; mp=196-198° C.; ¹H NMR (400 MHz, DMSO-d₆) δ 2.13 (s, 3H), 3.81 (s, 3H), 4.68 (s, 2H), 5.25 (brs, 1H), 6.56 (dd, J=2.8 Hz, 8.4 Hz, 1H), 6.92 (d, J=2.8 Hz, 1H), 6.98 (dd, J=7.6 Hz, 7.6 Hz, 1H), 7.16 (d, J=7.6 Hz, 1H), 7.57 (d, J=8.4 Hz, 1H), 8.24 (s, 1H), 8.43 (d, J=7.2 Hz, 1H), 9.12 (s, 1H); ¹³C NMR (100 MHz, DMSO-d₆) δ 18.8, 55.7, 57.3, 94.1, 113.1, 113.2, 113.4, 117.2, 119.6, 126.0, 130.2, 132.6, 144.4, 151.0, 154.4, 156.7, 157.8.

### 2-(4-Hydroxy-2-methylphenylamino)-3-(hydroxymethyl)-9-methoxy-4H-pyrido[1,2-a]pyrimidin-4-one (100)

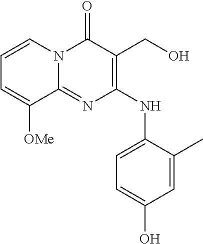 [SMILES: COc1cccn2c(=O)c(CO)c(Nc3ccc(O)cc3C)nc12]

White solid; mp=187-188° C.; ¹H NMR (400 MHz, DMSO-d₆) δ 2.13 (s, 3H), 3.92 (s, 3H), 4.68 (d, J=6.4 Hz, 2H), 5.18 (brs, 1H), 6.70 (d, J=8.4 Hz, 1H), 7.03 (dd, J=7.6 Hz, 7.6 Hz, 1H), 7.23 (d, J=7.6 Hz, 1H), 7.36 (dd, J=2.8 Hz, 8.4 Hz, 1H), 7.60 (d, J=2.8 Hz, 1H), 8.38 (s, 1H), 8.45 (d, J=6.8 Hz, 1H), 9.00 (s, 1H); ¹³C NMR (100 MHz, DMSO-d₆) δ 16.9, 55.2, 57.4, 94.3, 113.3, 113.4, 115.0, 119.5, 119.9, 124.2, 132.2, 133.4, 144.1, 151.1, 151.5, 156.8, 156.9; LC-MS (ESL m/z); [M+H]⁺ 328.21

### 3-(Hydroxymethyl)-9-methoxy-2-(4-methoxyphenylamino)-4H-pyrido[1,2-a]pyrimidin-4-one (101)

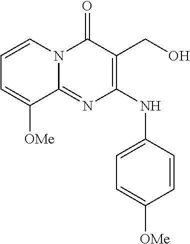 [SMILES: COc1ccc(Nc2nc3c(OC)cccn3c(=O)c2CO)cc1]

Pale yellow solid; mp=267-269° C. (decomp.); ¹H NMR (400 MHz, DMSO-d₆) δ 3.72 (s, 3H), 3.90 (s, 3H), 4.67 (d, J=5.2 Hz, 2H), 5.18 (t, J=5.2 Hz, 1H), 6.88 (d, J=9.2 Hz, 2H), 7.02 (dd, J=7.2 Hz, 7.2 Hz, 1H), 7.22 (d, J=7.2 Hz, 1H), 7.70 (d, J=9.2 Hz, 2H), 8.44 (d, J=1.2 Hz, 6.8 Hz, 1H), 8.50 (s, 1H); ¹³C NMR (100 MHz, DMSO-d₆) δ 55.1, 55.8, 57.4, 94.7, 113.3, 113.4, 114.3, 119.5, 122.6, 133.9, 144.1, 151.2, 155.3, 156.8, 156.9; LC-MS (ESI, m/z); [M+H]⁺ 328.21

### 2-(4-Fluoro-3-methylphenylamino)-3-(hydroxymethyl)-9-methoxy-4H-pyrido[1,2-a]pyrimidin-4-one (102)

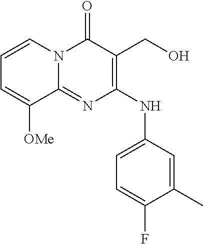 [SMILES: COc1cccn2c(=O)c(CO)c(Nc3ccc(F)c(C)c3)nc12]

Pale yellow solid; mp=281° C. (decomp.); ¹H NMR (400 MHz, DMSO-d₆) δ 2.48 (s, 3H), 3.91 (s, 3H), 4.68 (d, J=4.8 Hz, 2H), 5.22 (t, J=5.2 Hz, 1H), 7.01 (d, J=8.0 Hz, 1H), 7.05 (d, J=2.8 Hz, 1H), 7.22 (d, J=7.6 Hz, 1H), 7.58-7.62 (m, 1H), 7.87 (dd, J=2.4 Hz, 7.2 Hz, 1H), 8.42 (d, J=6.8 Hz, 1H), 8.58 (s, 1H).

### 3-(3-(Hydroxymethyl)-9-methoxy-4-oxo-4H-pyrido[1,2-a]pyrimidin-2-ylamino)benzonitrile (103)

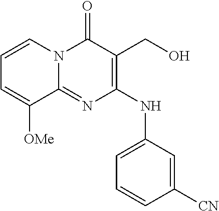 [SMILES: COc1cccn2c(=O)c(CO)c(Nc3cccc(C#N)c3)nc12]

White solid; mp=305-307° C. (decomp.); ¹H NMR (400 MHz, DMSO-d₆) δ 3.94 (s, 3H), 4.69 (d, J=4.4 Hz, 2H), 5.21 (brs, 1H), 7.09 (dd, J=7.2 Hz, 7.2 Hz, 1H), 7.29 (d, J=7.6 Hz, 1H), 7.39 (d, J=7.6 Hz, 1H), 7.46 (dd, J=8.0 Hz, 8.0 Hz, 1H), 7.91 (dd, J=1.2 Hz, 8.0 Hz, 1H), 8.45 (d, J=6.8 Hz, 1H), 8.78 (s, 1H), 8.89 (s, 1H); ¹³C NMR (100 MHz, DMSO-d₆) δ 59.5, 62.2, 101.1, 116.8, 118.3, 119.0, 124.1, 124.5, 128.5, 129.8, 130.4, 135.1, 146.6, 148.4, 156.0, 160.8, 162.0; LC-MS (ESI, m/z); [M+H]⁺ 323.25

### 3′-(3-(Hydroxymethyl)-9-methoxy-4-oxo-4H-pyrido[1,2-a]pyrimidin-2-ylamino)-biphenyl-4-carbonitrile (104)

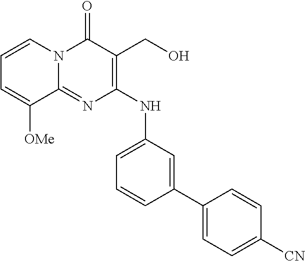 [SMILES: COc1cccn2c(=O)c(CO)c(Nc3cccc(-c4ccc(C#N)cc4)c3)nc12]

Pale yellow solid; mp=over 370° C. (decomp.); ¹H NMR (400 MHz, DMSO-d₆) δ 3.98 (s, 3H), 4.74 (s, 2H), 5.30 (brs, 1H), 7.08 (dd, J=7.2, 7.6 Hz, 1H), 7.32 (d, J=7.6 Hz, 1H), 7.38-7.44 (m, 2H), 7.50-7.52 (m, 1H), 7.91-7.99 (m, 4H), 8.48 (d, J=6.8 Hz, 1H), 8.83 (brs, 1H), 8.88 (s, 1H); ¹³C NMR (100 MHz, DMSO-d₆) δ 54.4, 56.7, 95.0, 110.0, 112.7, 113.2, 118.5, 118.7, 118.8, 120.1, 120.4, 127.2, 129.2, 132.8, 138.3, 140.9, 134.1, 144.9, 150.5, 155.8, 156.4; LC-MS (ESI, m/z): 399[M+H]⁺.

### 4-(3-(Hydroxymethyl)-9-methoxy-4-oxo-4H-pyrido[1,2-a]pyrimidin-2-ylamino)-benzonitrile (105)

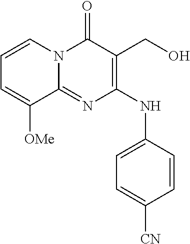 [SMILES: COc1cccn2c(=O)c(CO)c(Nc3ccc(C#N)cc3)nc12]

Pale yellow solid; mp=over 300° C. (decomp.); ¹H NMR (400 MHz, DMSO-d₆) δ 3.96 (s, 3H), 4.71 (s, 2H), 5.32 (brs, 1H), 7.12 (dd, J=7.2 Hz, 7.6 Hz, 1H), 7.32 (d, J=7.6 Hz, 1H), 7.71 (d, J=8.8 Hz, 2H), 8.08 (d, J=8.8 Hz, 2H), 8.48 (dd, J=1.2, 7.2 Hz, 1H), 7.07 (brs, 1H); ¹³C NMR (100 MHz, DMSO-d₆) δ 54.2, 56.8, 96.5, 103.0, 113.0, 113.8, 118.7, 119.4, 119.7, 132.9, 143.0, 144.6, 150.7, 155.2, 156.6; LC-MS (ESI, m/z): 323[M+H]⁺.

### 2-(2-Hydroxy-4-methylphenylamino)-3-(hydroxymethyl)-9-methoxy-4H-pyrido[1,2-a]-pyrimidin-4-one (106)

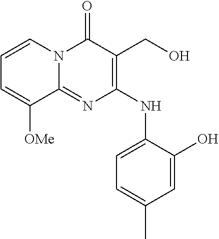 [SMILES: COc1cccn2c(=O)c(CO)c(Nc3ccc(C)cc3O)nc12]

Pale yellow solid; mp=343-345° C. (decomp.); ¹H NMR (400 MHz, DMSO-d₆) δ 2.19 (s, 3H), 3.94 (s, 3H), 4.67 (d, J=4.4 Hz, 2H), 5.32 (brt, J=4.4 Hz, 1H), 6.58 (dd, J=1.2, 8.4 Hz, 1H), 6.67 (s, 1H), 7.05 (dd, J=7.2, 7.2 Hz, 1H), 7.26 (dd, J=7.2, 1.2 Hz, 1H), 8.45 (d, J=8.4 Hz, 1H), 8.46 (dd, J=7.2, 1.2 Hz, 1H), 8.88 (s, 1H), 9.87 (s, 1H); ¹³C NMR (100 MHz, DMSO-d₆) δ 20.6, 55.2, 56.7, 94.1, 112.8, 112.9, 115.3, 118.8, 119.5, 119.9, 126.1, 131.0, 143.4, 146.3, 150.5, 155.8, 156.2; LC-MS (ESI, m/z): 328[M+H]⁺

### 2-(Biphenyl-2-ylamino)-3-(hydroxymethyl)-9-methoxy-4H-pyrido[1,2-a]pyrimidin-4-one (107)

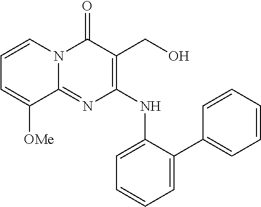 [SMILES: COc1cccn2c(=O)c(CO)c(Nc3ccccc3-c3ccccc3)nc12]

Pale yellow solid; mp=191.0-192.8° C.; ¹H NMR (400 MHz, CDCl₃) δ 1.89 (t, J=5.6 Hz, 1H), 4.58 (d, J=5.6 Hz, 2H), 6.83 (dd, J=7.2, 7.6 Hz, 1H), 6.91 (dd, J=1.2, 7.6 Hz, 1H), 7.11 (ddd, J=1.2, 7.6, 7.6 Hz, 1H), 7.25-7.27 (m, 1H), 7.36-7.47 (m, 6H), 7.93 (brs, 1H), 8.52 (dd, J=1.2, 7.2 Hz, 1H), 8.58 (d, J=8.4 Hz, 1H); ¹³C NMR (100 MHz, CDCl₃) δ 56.9, 57.0, 95.3, 111.7, 112.6, 119.8, 121.3, 123.1, 127.9, 128.3, 129.8, 130.3, 133.1, 137.0, 138.8, 144.4, 151.3, 156.8, 157.5; LC-MS (ESI, m/z): 374[M+H]⁺.

### Methyl 5-(4-(3-(hydroxymethyl)-9-methoxy-4-oxo-4H-pyrido[1,2-a]pyrimidin-2-ylamino)-phenyl)furan-2-carboxylate (108)

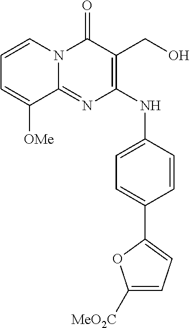 [SMILES: COC(=O)c1ccc(-c2ccc(Nc3nc4c(OC)cccn4c(=O)c3CO)cc2)o1]

Pale yellow solid; mp=230-231° C. (decomp.); ¹H NMR (400 MHz, DMSO-d₆+CDCl₃) δ 3.88 (s, 3H), 4.04 (s, 3H), 4.79 (s, 2H), 5.40 (br s, 1H), 7.06 (d, J=3.6 Hz, 1H), 7.14 (dd, J=7.2, 7.6 Hz, 1H), 7.40 (d, J=3.4 Hz, 1H), 7.80 (d, J=8.4 Hz, 1H), 8.06 (d, J=8.8 Hz, 1H), 8.54 (d, J=7.2 Hz, 1H), 8.96 (br s, 1H); LC-MS (ESI, m/z); [M+H]⁺ 422.22

### 2-(3-Chloro-4-(4-chlorophenoxy)phenylamino)-3-(hydroxymethyl)-9-methoxy-4H-pyrido[1,2-a]pyrimidin-4-one (109)

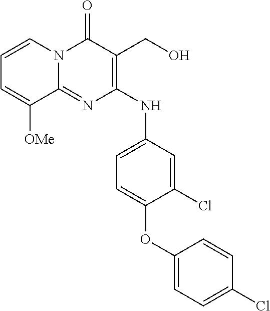 [SMILES: COc1cccn2c(=O)c(CO)c(Nc3ccc(Oc4ccc(Cl)cc4)c(Cl)c3)nc12]

White solid; mp=211-212° C.; ¹H NMR (400 MHz, DMSO-d₆) δ 4.01 (s, 3H), 4.76 (s, 2H), 5.26 (s, 1H), 6.98 (d, J=9.2 Hz, 2H), 7.18 (dd, J=7.2, 7.2 Hz, 1H), 7.22 (d, J=9.2 Hz, 1H), 7.49 (d, J=8.8 Hz, 2H), 7.76 (dd, J=2.4, 8.8 Hz, 1H), 8.54 (dd, J=1.2, 7.2 Hz, 1H), 8.72 (d, J=2.4 Hz, 2H), 8.87 (br s, 1H); LC-MS (ESI, m/z); [M+H]⁺ 458.20

### 4-(3-(Hydroxymethyl)-9-methoxy-4-oxo-4H-pyrido[1,2-a]pyrimidin-2-ylamino)-N-phenylbenzamide (110)

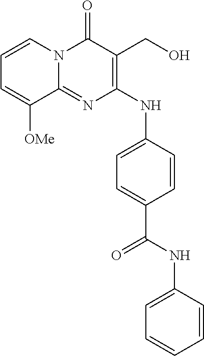 [SMILES: COc1cccn2c(=O)c(CO)c(Nc3ccc(C(=O)Nc4ccccc4)cc3)nc12]

Pale yellow solid; mp=227-228° C. (decomp.); ¹H NMR (400 MHz, DMSO-d₆) δ 4.04 (s, 3H), 4.80 (s, 2H), 5.43 (s, 1H), 7.13 (dd, J=7.2, 7.2 Hz, 1H), 7.19 (dd, J=7.2, 7.2 Hz, 1H), 7.37-7.41 (m, 3H), 7.83 (dd, J=0.8, 8.4 Hz, 2H), 8.01 (d, J=8.8 Hz, 2H), 8.11 (d, J=8.8 Hz, 2H), 8.56 (dd, J=0.8, 8.4 Hz, 1H), 9.03 (br s, 1H), 10.15 (br s, 1H); LC-MS (ESI, m/z); [M+H]⁺ 417.25

### 3-(Hydroxymethyl)-9-methoxy-2-(4-oxo-2-phenyl-4H-chromen-6-ylamino)-4H-pyrido[1,2-a]pyrimidin-4-one (111)

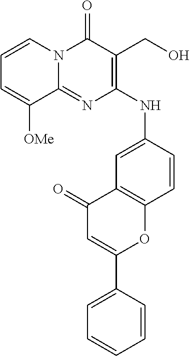 [SMILES: COc1cccn2c(=O)c(CO)c(Nc3ccc4oc(-c5ccccc5)cc(=O)c4c3)nc12]

Yellow solid; mp=247° C. (decomp.); ¹H NMR (400 MHz, DMSO-d₆) δ 3.40 (s, 3H), 4.79 (s, 2H), 7.02 (s, 1H), 7.15 (dd, J=7.2, 7.6 Hz, 1H), 7.34 (d, J=7.6 Hz, 1H), 7.61-7.64 (m, 3H), 7.98 (d, J=9.2 Hz, 1H), 8.12-8.15 (m, 2H), 8.38 (dd, J=2.8, 8.8 Hz, 1H), 8.54 (d, J=7.6 Hz, 1H), 8.79 (d, J=2.8 Hz, 1H); LC-MS (ESI, m/z); [M+H]⁺ 442.22

### 3-(Hydroxymethyl)-9-methoxy-2-(4-oxo-2-phenyl-4H-chromen-7-ylamino)-4H-pyrido-[1,2-a]pyrimidin-4-one (112)

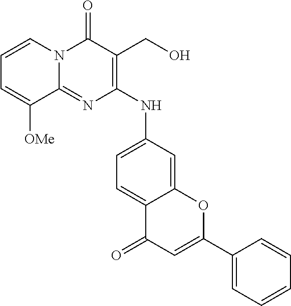 [SMILES: COc1cccn2c(=O)c(CO)c(Nc3ccc4c(=O)cc(-c5ccccc5)oc4c3)nc12]

Yellow solid; mp=251° C. (decomp.); ¹H NMR (400 MHz, DMSO-d₆) δ 4.13 (s, 3H), 4.82 (s, 2H), 7.01 (s, 1H), 7.24 (dd, J=7.2, 7.2 Hz, 1H), 7.45 (d, J=6.8 Hz, 1H), 7.66-7.70 (m, 3H), 7.98 (d, J=8.8 Hz, 1H), 8.12-8.15 (m, 2H), 8.57 (dd, J=0.8, 6.8 Hz, 1H), 9.09 (d, J=2.0 Hz, 1H); LC-MS (ESI, m/z); [M+H]⁺ 442.22

### 1-(4-(3-(Hydroxymethyl)-9-methoxy-4-oxo-4H-pyrido[1,2-a]pyrimidin-2-ylamino)-phenyl)-N-methylmethanesulfonamide (113)

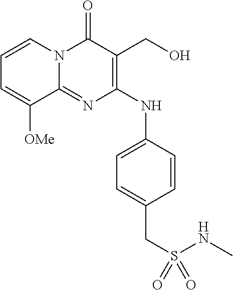 [SMILES: CNS(=O)(=O)Cc1ccc(Nc2nc3c(OC)cccn3c(=O)c2CO)cc1]

White solid; mp=223-224° C.; ¹H NMR (400 MHz, DMSO-d₆) δ 2.60 (s, 3H), 4.01 (s, 3H), 4.32 (s, 2H), 4.77 (s, 2H), 5.36 (br s, 1H), 6.93 (d, J=3.2 Hz, 1H), 7.15 (dd, J=7.2, 7.2 Hz, 1H), 7.35 (d, J=8.4 Hz, 1H), 7.93 (d, J=8.4 Hz, 2H), 8.53 (dd, J=1.2, 7.2 Hz, 1H), 8.79 (br s, 1H); LC-MS (ESI, m/z); [M+H]⁺ 405.21

### Diethyl 4-(3-(hydroxymethyl)-9-methoxy-4-oxo-4H-pyrido[1,2-a]pyrimidin-2-ylamino)-benzylphosphonate (114)

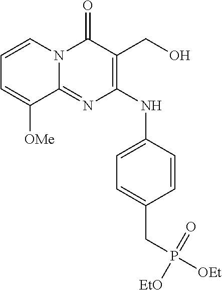 [SMILES: CCOP(=O)(Cc1ccc(Nc2nc3c(OC)cccn3c(=O)c2CO)cc1)OCC]

Pale yellow solid; mp=172-173° C.; ¹H NMR (400 MHz, DMSO-d₆) δ 1.23 (t, J=7.0 Hz, 6H), 3.19 (s, 1H), 3.24 (s, 1H), 3.96-4.03 (m, 7H), 4.76 (d, J=5.2 Hz, 2H), 5.32 (t, J=5.2 Hz, 1H), 7.13 (dd, J=7.2, 7.6 Hz, 1H), 7.26 (dd, J=2.4, 8.8 Hz, 2H), 7.33 (dd, J=0.8, 7.6 Hz, 1H), 7.83 (d, J=8.4 Hz, 2H), 8.53 (dd, J=1.2, 7.2 Hz, 1H), 8.71 (br s, 1H); LC-MS (ESI, m/z); [M+H]⁺ 448.27

### 3-(Hydroxymethyl)-9-methoxy-2-(4-(piperazin-1-yl)phenylamino)-4H-pyrido[1,2a]-pyrimidin-4-one (115)

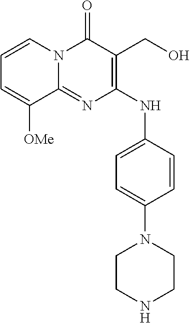 [SMILES: COc1cccn2c(=O)c(CO)c(Nc3ccc(N4CCNCC4)cc3)nc12]

Pale yellow solid; mp=over 195° C. (decomp.); ¹H NMR (400 MHz, DMSO-d₆) δ 3.01-3.03 (m, 4H), 3.81-3.83 (m, 4H), 3.92 (s, 3H), 4.44 (d, J=4.8 Hz, 2H), 4.58 (br, 1H), 4.90 (t, J=4.8 Hz, 1H), 6.50 (d, J=8.8 Hz, 2H), 6.73 (d, J=8.8 Hz, 2H), 7.00 (dd, J=7.2, 7.6 Hz, 1H), 7.21 (dd, J=0.8, 7.6 Hz, 1H), 8.40 (dd, J=0.8, 7.2 Hz, 1H).

### (2-(3-Chloro-4-fluorophenylamino)-9-fluoro-4-oxo-4H-pyrido[1,2-a]pyrimidin-3-yl)methyl isobutyrate (116)

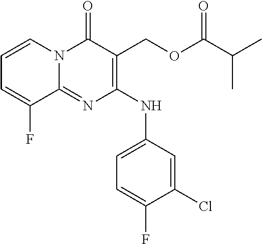 [SMILES: CC(C)C(=O)OCc1c(Nc2ccc(F)c(Cl)c2)nc2c(F)cccn2c1=O]

Pale yellow solid; mp=156-157° C.; ¹H NMR (400 MHz, CDCl₃) δ 1.19 (s, 3H), 1.21 (s, 3H), 2.61-2.71 (m, 1H), 5.38 (s, 2H), 6.91-6.96 (m, 1H), 7.12 (dd, J=8.8, 9.2 Hz, 1H), 7.41-7.45 (m, 1H), 7.53-7.57 (m, 1H), 8.00 (dd, J=2.8, 6.4 Hz, 1H), 8.80 (d, J=7.2 Hz, 1H), 9.22 (s, 1H).

### (2-(3-Chloro-4-fluorophenylamino)-9-methoxy-4-oxo-4H-pyrido[1,2-a]pyrimidin-3-yl)methyl isobutyrate (117)

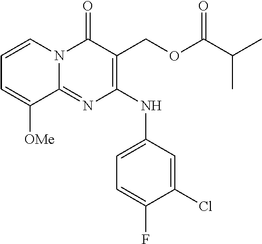 [SMILES: COc1cccn2c(=O)c(COC(=O)C(C)C)c(Nc3ccc(F)c(Cl)c3)nc12]

White solid; mp=152-154° C.; ¹H NMR (400 MHz, CDCl₃) δ 1.17 (s, 3H), 1.19 (s, 3H), 2.61-2.68 (m, 1H), 4.03 (s, 3H), 5.40 (s, 2H), 6.93 (dd, J=7.2, 7.2 Hz, 1H), 6.99 (dd, J=1.2, 7.6 Hz, 1H), 7.44-7.47 (m, 1H), 8.36 (dd, J=2.8, 6.8 Hz, 1H), 8.63 (dd, J=1.2, 7.2 Hz, 1H), 9.11 (s, 1H).

### 2-(3-Chloro-4-fluorophenylamino)-3-((isopropylamino)methyl)-9-methoxy-4H-pyrido[1,2-a]pyrimidin-4-one (118)

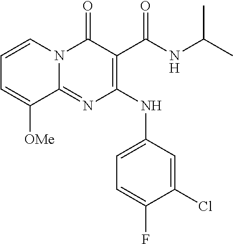 [SMILES: COc1cccn2c(=O)c(C(=O)NC(C)C)c(Nc3ccc(F)c(Cl)c3)nc12]

Pale yellow solid; mp=196° C. (decomp.); solid; ¹H NMR (400 MHz, CDCl₃) δ 1.30 (d, J=6.8 Hz, 6H), 4.02 (s, 3H), 4.20-4.28 (m, 1H), 6.92 (dd, J=7.2 Hz, 7.2 Hz, 1H), 7.00 (d, J=6.8 Hz, 1H), 7.08 (dd, J=8.8 Hz, 8.8 Hz, 1H), 7.45-7.49 (m, 1H), 8.48 (dd, J=2.4 Hz, 6.8 Hz, 1H), 8.54 (dd, J=1.2 Hz, 6.8 Hz, 1H); 9.86 (d, J=7.2 Hz, 1H), 13.09 (s, 1H); ¹³C NMR (100 MHz, CDCl₃) δ 23.0, 29.9, 41.3, 57.0, 113.2, 116.3, 116.5, 119.4, 120.8 (d, J=6.7 Hz, due to F), 123.4, 136.2, 144.6, 151.2, 153.1, 155.6, 158.2, 158.7, 167.5.

### Ethyl 2-(3-chloro-4-fluorophenylamino)-9-methoxy-4-oxo-4H-pyrido[1,2-a]pyrimidine-3-carboxylate (119)

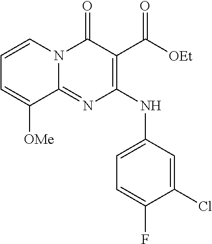 [SMILES: CCOC(=O)c1c(Nc2ccc(F)c(Cl)c2)nc2c(OC)cccn2c1=O]

Pale yellow solid; ¹H NMR (400 MHz, CDCl₃) 1.46 (t, J=7.2 Hz, 3H), δ 4.02 (s, 3H), 4.45 (q, J=7.2 Hz, 2H), 6.92 (dd, J=7.2 Hz, 7.2 Hz, 1H), 7.05 (dd, J=1.2 Hz, 7.6 Hz, 1H), 7.09 (dd, J=8.8 Hz, 8.8 Hz, 1H), 7.42-7.46 (m, 1H), 7.49 (dd, J=2.8 Hz, 6.8 Hz, 1H), 8.64 (dd, J=0.8, 6.8 Hz, 1H), 11.51 (s, 1H).

### 2-(3-Chloro-4-fluorophenylamino)-9-methoxy-4H-pyrido[1,2-a]pyrimidin-4-one (120)

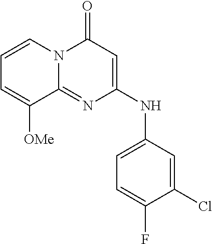 [SMILES: COc1cccn2c(=O)cc(Nc3ccc(F)c(Cl)c3)nc12]

Pale yellow solid; ¹H NMR (400 MHz, DMSO d-6) δ 3.91 (s, 3H), 4.87 (s, 1H), 7.10 (dd, J=7.6 Hz, 7.6 Hz, 1H), 7.27 (dd, J=9.2 Hz, 9.2 Hz, 1H), 7.37 (d, J=7.6 Hz, 1H), 7.41-7.44 (m, 1H), 7.39 (d, J=7.2 Hz, 1H), 7.68 (dd, J=2.4 Hz, 7.2 Hz, 1H), 10.86 (s, 1H).

### Example 7

### Derivatization of the Pyridopyrimidinone Compounds

The pyridopyrimidinone compounds (scaffold VIII and VIIIa) underwent derivatization according to the methods outlined below (Schemes 7-24). Resulting derivatives were examined for inhibitory activity using the assay described above and the results are summarized in Table 2.

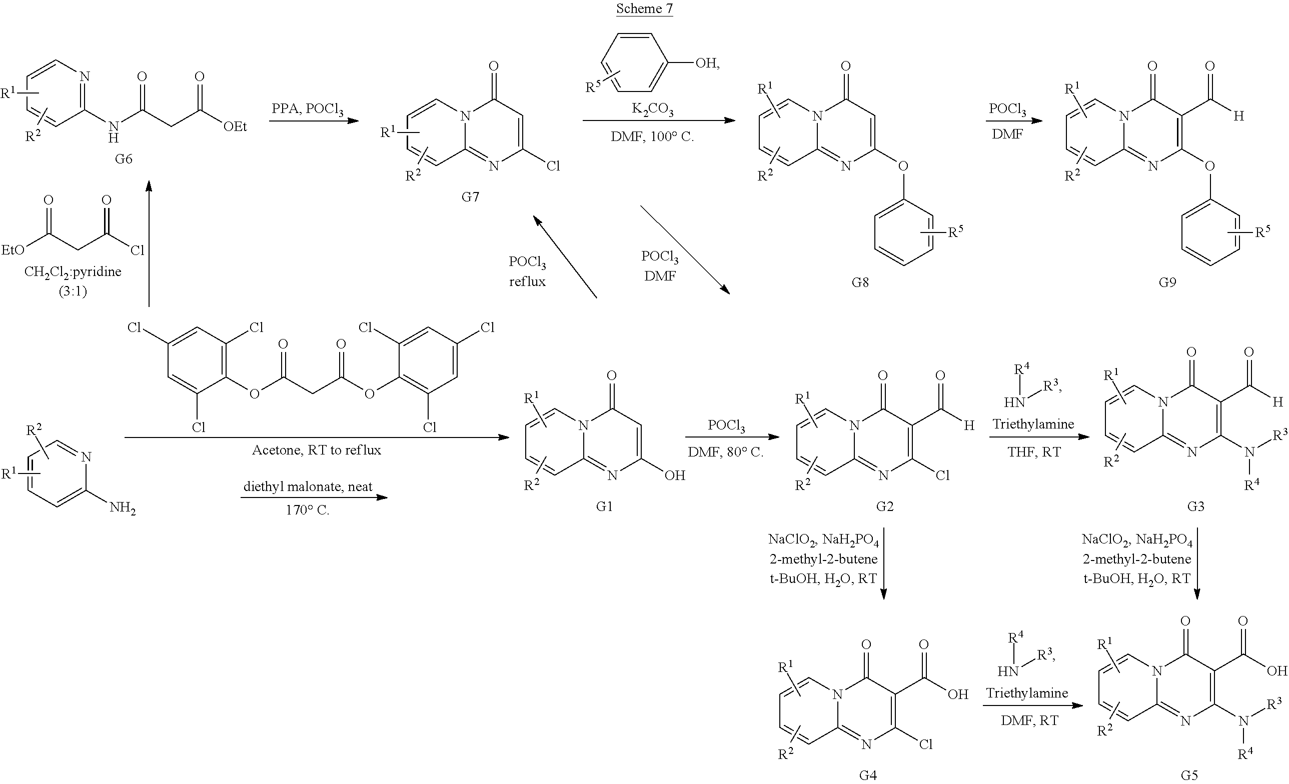

### General Procedure for the Synthesis of G1

2-Amino-3-picoline (1.0 mmol) was dissolved in diethyl malonate (1.0 mmol). The solution was heated to 170° C. for 12 h. After cooling, the dark residue was triturated with CH₂Cl₂ (10 mL). The residual pale solid was collected by filtration and washed with CH₂Cl₂ to give G1.

### General Procedure for the Synthesis of G2

To a DMF (2.0 mL) was added POCl₃ (3.0 mmol) at 0° C. After the mixture was stirred at 0° C. for 40 min, a solution of G1 (1.0 mmol) in DMF (2.0 mL) was added and stirred at 80° C. for 1 h. The mixture was cooled and concentrated in vacuo. The residue was diluted with water and extracted with CH₂Cl₂ (10 mL×3). The combined organic layers were washed with brine, dried over MgSO₄ and concentrated. The residue was purified by flash column chromatography to give G2.

### General Procedure for the Synthesis of G3

To a stirred solution of G2 (1.0 mmol) in THF (2.0 mL) was added Et₃N (2.0 mmol). The mixture was cooled to 0° C. After 5 min, an amine (1.0 mmol) was added dropwise and the mixture was stirred at room temperature overnight. The reaction mixture was diluted with CH₂Cl₂ (10 mL) and washed with brine (10 mL). The organic layer was dried over anhydrous MgSO₄ and concentrated in vacuo. The crude product was purified by flash column chromatography to give G3.

### General Procedure for the Synthesis of G4

G2 (0.5 mmol) was dissolved in 10.4 mL of tert-butyl alcohol and 2.5 mL of 2-methyl-2-butene. A solution of sodium chlorite (4.59 mmol) and sodium dihydrogenphosphate (3.46 mmol) in 4.2 mL of water was added dropwise. The reaction mixture was stirred at room temperature overnight. Volatile components were then removed under vacuum, and the residue was dissolved in 10 ml of water and extracted with two 10 ml portions of hexane. The aqueous layer was acidified to pH=3 with HCl(aq) and extracted with 10 mL portions of methylene chloride. The combined organic layers were washed with 20 mL of cold water, dried and concentrated to give G4.

General Procedure for the Synthesis of G5 from G3

G3 (36.6 μmol) was dissolved in 760 μl of tert-butyl alcohol and 180 μl of 2-methyl-2-butene. A solution of sodium chlorite (335 μmol) and sodium dihydrogenphosphate (253 μmol) in 300 μl of water was added dropwise. The reaction mixture was stirred at room temperature overnight. Volatile components were then removed under vacuum and the residue was dissolved in 10 ml of water and extracted with two 10 ml portions of hexane. The aqueous layer was acidified to pH=3 with HCl(aq) and extracted with 10 ml portions of methylene chloride. The combined organic layers were washed with 20 ml of cold water, dried and concentrated to give G5.

General Procedure for the Synthesis of G5 from G4

To a stirred solution of G4 (1.0 mmol) in DMF (2.0 mL) was added Et₃N (2.0 mmol) and amine (1.5 mmol) and the mixture was stirred at 60° C. overnight. The reaction mixture was diluted with CH₂Cl₂ (10 mL) and washed with brine (10 ml). The organic layer was dried over anhydrous MgSO₄ and concentrated in vacuo. The crude product was purified by recrystallization from a mixture of hexanes and methylene chloride to give G5.

### General Procedure for the Synthesis of G6

The solution of 2-amino-3-picoline (4.0 mmol) in a solution of CH₂Cl₂ (3 mL) and dried pyridine (1 mL) was added dropwise at room temperature to a stirred solution of ethyl 3-chloro-3-oxo-propionate (5.3 mmol) in CH₂Cl₂ (3 mL) (an exothermic reaction with emission of white fume occurred during the addition). The resulting warm mixture was stirred at room temperature for 30 min and then poured into 30 mL of cold water; an excess of sodium carbonate was carefully added with stirring and the mixture was further stirred at room temperature for 1 h. The organic layer was then collected and the aqueous phase was extracted several times with CH₂Cl₂. The combined organic layers were washed with water, dried over anhydrous Na₂SO₄, and concentrated in vacuo. The crude product was purified by flash column chromatography to give G6.

### General Procedure for the Synthesis of G7

A mixture of G6 (1.83 mmol), POCl₃ (0.5 mL) and polyphosphoric acid (137 mg) was heated with stirring at 130° C. for 3 h. After cooling, anhydrous ethanol was added and the mixture was refluxed for 30 min, then allowed to cool. The mixture was treated with aqueous sodium carbonate and exhaustively extracted with CH₂Cl₂ (10 mL×3). The combined layers were washed with water (10 mL), brine (10 mL), dried over MgSO₄, filtered and concentrated in vacuo. The crude product was purified by flash column chromatography to give G7.

### General Procedure for the Synthesis of G8

To a solution of G6 (1 mmol) in DMF (0.96 mL) was added potassium carbonate (5.0 mmol) followed by phenol (1.94 mmol). After 12 h at 100° C., the solution was allowed to cool to 23° C. The reaction mixture was washed with H₂O (50 mL), and the aqueous layer was extracted with CH₂Cl₂ (20 mL×3). The combined organic layers were washed with 1 N HCl (20 mL×2), filtered, and concentrated in vacuo. The crude product was purified by flash column chromatography to give G8.

### General Procedure for the Synthesis of G9

To DMF (2.0 mL) was added POCl₃ (3.0 mmol) at 0° C. After the mixture was stirred at 0° C. for 40 min, a solution of G8 (1.0 mmol) in DMF (2.0 mL) was added and stirred at 80° C. for 1 h. The mixture was cooled and concentrated in vacuo. The residue was diluted with water and extracted with CH₂Cl₂ (10 mL×3). The combined organic layers were washed with brine, dried over MgSO₄ and concentrated. The residue was purified by flash column chromatography to give G9.

### Ethyl 3-(3-methylpyridin-2-ylamino)-3-oxopropanoate (124)

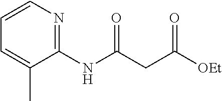 [SMILES: CCOC(=O)CC(=O)Nc1ncccc1C]

¹H NMR (400 MHz, CDCl₃) δ 1.25 (t, J=7.0 Hz, 3H), 2.25 (s, 3H), 3.45 (s, 2H), 4.20 (q, J=7.2 Hz, 2H), 7.47 (d, J=8.4 Hz, 1H), 8.03 (d, J=8.4 Hz, 1H), 8.07 (s, 1H), 9.67 (brs, 1H); ¹³C NMR (100 MHz, CDCl₃) δ 13.9, 17.7, 42.6, 61.7, 113.8, 129.3, 138.8, 147.6, 148.8, 163.5, 168.4.

### 2-Hydroxy-9-methyl-4H-pyrido[1,2-a]pyrimidin-4-one (125)

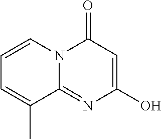

¹H NMR (400 MHz, DMSO-d₆) δ 2.48 (s, 3H), 5.44 (s, 1H), 7.20 (t, J=7.0 Hz, 1H), 7.87 (d, J=6.8 Hz, 1H), 8.84 (d, J=6.8 Hz, 1H), 11.52 (brs, 1H).

### 2-Hydroxy-8-methyl-4H-pyrido[1,2-a]pyrimidin-4-one (126)

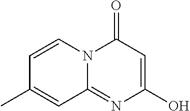

¹H NMR (400 MHz, DMSO-d₆) δ 2.50 (s, 3H), 4.88 (s, 1H), 7.20-7.24 (m, 2H), 8.85 (d, J=6.8 Hz, 1H), 11.98 (br s, 1H); ¹³C NMR (100 MHz, DMSO-d₆) δ 20.6, 80.3, 114.4, 117.1, 127.7, 146.7, 153.5, 155.3, 162.3.

### 2-Chloro-9-methyl-4H-pyrido[1,2-a]pyrimidin-4-one (127)

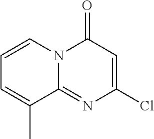

¹H NMR (400 MHz, CDCl₃) δ 2.57 (s, 3H), 6.45 (s, 1H), 7.12 (t, J=7.0 Hz, 1H), 7.68 (d, J=6.8 Hz, 1H), 8.93 (d, J=6.8 Hz, 1H); ¹³C NMR (100 MHz, CDCl₃) δ 18.0, 102.3, 115.8, 125.7, 134.7, 136.9, 150.0, 157.6, 157.9.

### 2-Chloro-9-methyl-4-oxo-4H-pyrido[1,2-a]pyrimidine-3-carbaldehyde (128)

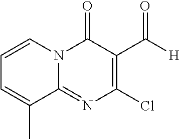

¹H NMR (400 MHz, CDCl₃) δ 2.64 (s, 3H), 7.30 (t, J=7.0 Hz, 1H), 7.92 (d, J=7.2 Hz, 1H), 9.10 (d, J=6.4 Hz, 1H), 10.42 (s, 1H); ¹³C NMR (100 MHz, CDCl₃) δ 17.7, 107.3, 117.7, 127.0, 135.6, 140.6, 150.0, 156.4, 160.2, 187.1.

### 2-Chloro-8-methyl-4-oxo-4H-pyrido[1,2-a]pyrimidine-3-carbaldehyde (129)

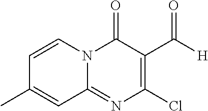

¹H NMR (400 MHz, CDCl₃) δ 2.59 (s, 3H), 7.24 (d, J=7.2 Hz, 1H), 7.52 (s, 1H), 9.09 (d, J=7.2 Hz, 1H), 10.40 (s, 1H).

### 2-Chloro-7-methyl-4-oxo-4H-pyrido[1,2-a]pyrimidine-3-carbaldehyde (130)

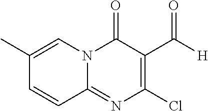

¹H NMR (400 MHz, DMSO-d₆) δ 2.32 (s, 3H), 7.49 (d, J=8.8 Hz, 1H), 7.78 (d, J=8.8 Hz, 1H), 8.79 (s, 1H), 10.16 (s, 1H).

### 2-Chloro-6-methyl-4-oxo-4H-pyrido[1,2-a]pyrimidine-3-carbaldehyde (131)

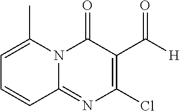

¹H NMR (400 MHz, CDCl₃) δ 3.11 (s, 3H), 6.98 (d, J=7.2 Hz, 1H), 7.51 (d, J=8.8 Hz, 1H), 7.79 (t, J=8.0 Hz, 1H), 10.29 (s, 1H).

### 9-Methyl-4-oxo-2-(phenylamino)-4H-pyrido[1,2-a]pyrimidine-3-carbaldehyde (132)

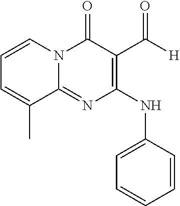 [SMILES: Cc1cccn2c(=O)c(C=O)c(Nc3ccccc3)nc12]

¹H NMR (400 MHz, CDCl₃) δ 2.44 (s, 3H), 6.89 (t, J=6.8 Hz, 1H), 7.11 (t, J=7.2 Hz, 1H), 7.34 (t, J=7.6 Hz, 2H), 7.62 (d, J=6.4 Hz, 1H), 7.76 (d, J=8.0 Hz, 2H), 8.80 (d, J=6.8 Hz, 1H), 10.27 (s, 1H), 11.67 (brs, 1H); ¹³C NMR (100 MHz, CDCl₃) δ 18.1, 94.6, 113.6, 121.8, 124.2, 125.9, 128.7, 133.6, 138.1, 138.9, 152.5, 153.8, 160.2, 190.2.

### 2-(3-Chlorophenylamino)-9-methyl-4-oxo-4H-pyrido[1,2-a]pyrimidine-3-carbaldehyde (133)

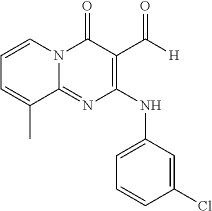 [SMILES: Cc1cccn2c(=O)c(C=O)c(Nc3cccc(Cl)c3)nc12]

¹H NMR (400 MHz, CDCl₃) δ 2.50 (s, 3H), 6.97 (t, J=6.8 Hz, 1H), 7.08 (d, J=8.0 Hz, 1H), 7.25 (t, J=8.0 Hz, 1H), 7.42 (d, J=8.0H, 1H), 7.69 (d, J=6.8 Hz, 1H), 8.18 (s, 1H), 8.84 (d, J=6.8 Hz, 1H), 10.27 (s, 1H), 11.72 (brs, 1H).

### 9-Methyl-4-oxo-2-(3-(trifluoromethoxy)phen ylamino)-4H-pyrido[1,2-a]pyrimidine-3-carbaldehyde (134)

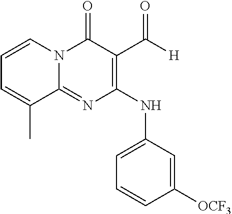 [SMILES: Cc1cccn2c(=O)c(C=O)c(Nc3cccc(OC(F)(F)F)c3)nc12]

¹H NMR (400 MHz, CDCl₃) δ 2.50 (s, 3H), 6.99 (t, J=7.0 Hz, 1H), 7.36 (t, J=8.0 Hz, 1H), 7.42 (d, J=8.0 Hz, 1H), 7.70 (d, J=6.8 Hz, 1H), 8.16 (s, 1H), 8.88 (d, J=8.0 Hz, 1H), 10.32 (s, 1H), 11.86 (brs, 1H); ¹³C NMR (100 MHz, CDCl₃) δ 18.0, 94.7, 114.2, 114.7, 116.5, 119.7, 126.1, 129.7, 133.8, 139.4, 139.7, 149.4, 152.6, 157.0, 160.1, 190.4.

### 9-Methyl-4-oxo-2-(3-(trifluoromethyl)phenylamino)-4H-pyrido[1,2-a]pyrimidine-3-carbaldehyde (135)

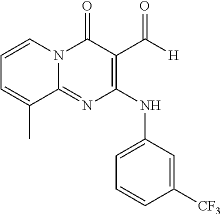 [SMILES: Cc1cccn2c(=O)c(C=O)c(Nc3cccc(C(F)(F)F)c3)nc12]

¹H NMR (400 MHz, CDCl₃) δ 2.49 (s, 1H), 6.98 (t, J=6.8 Hz, 1H), 7.37 (d, J=7.6 Hz, 1H), 7.45 (d, J=7.6 Hz, 1H), 7.61 (d, J=8.0 Hz, 1H), 7.70 (d, J=6.0 Hz, 1H), 8.61 (s, 1H), 8.87 (d, J=6.8 Hz, 1H), 10.30 (s, 1H), 11.85 (brs, 1H).

### 2-(4-tert-Butylphenylamino)-9-methyl-4-oxo-4H-pyrido[1,2-a]pyrimidine-3-carbaldehyde (136)

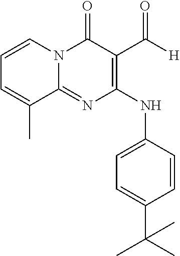 [SMILES: Cc1cccn2c(=O)c(C=O)c(Nc3ccc(C(C)(C)C)cc3)nc12]

¹H NMR (400 MHz, CDCl₃) δ 1.32 (s, 9H), 2.48 (s, 3H), 6.89 (t, J=7.0 Hz, 1H), 7.37 (d, J=8.4 Hz, 1H), 7.62 (d, J=6.8 Hz, 1H), 7.73 (d, J=8.8 Hz, 1H), 8.81 (d, J=7.2 Hz, 1H), 10.30 (s, 1H), 11.68 (br s, 1H); ¹³C NMR (100 MHz, CDCl₃) δ 18.2, 31.3, 34.3, 94.6, 113.5, 121.4, 125.6, 125.9, 133.6, 135.6, 138.8, 147.2, 152.6, 156.7, 160.4, 190.2.

### 2-(3-Chlorobenzylamino)-9-methyl-4-oxo-4H-pyrido[1,2-a]pyrimidine-3-carbaldehyde (137)

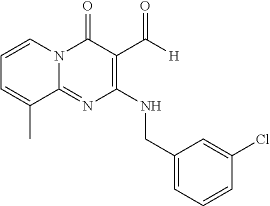 [SMILES: Cc1cccn2c(=O)c(C=O)c(NCc3cccc(Cl)c3)nc12]

¹H NMR (400 MHz, CDCl₃) δ 2.40 (s, 3H), 4.80 (d, J=6.0 Hz, 2H), 6.87 (t, J=7.0 Hz, 1H), 7.24-7.26 (m, 3H), 7.37 (s, 1H), 7.59 (d, J=6.8 Hz, 1H), 8.79 (d, J=7.2 Hz, 1H), 10.34 (brs, 1H), 10.30 (s, 1H).

### 9-Methyl-2-morpholino-4-oxo-4H-pyrido[1,2-a]pyrimidine-3-carbaldehyde (138)

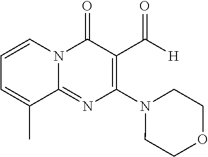 [SMILES: Cc1cccn2c(=O)c(C=O)c(N3CCOCC3)nc12]

¹H NMR (400 MHz, CDCl₃) δ 2.30 (s, 3H), 3.65 (d, J=2.4 Hz, 4H), 3.72 (d, J=3.2 Hz, 4H), 6.74-6.77 (m, 1H), 7.49 (d, J=6.8 Hz, 1H), 8.62 (d, J=7.2 Hz, 1H), 10.01 (s, 1H); ¹³C NMR (100 MHz, CDCl₃) δ 17.6, 49.5, 67.0, 95.9, 112.9, 125.7, 133.0, 138.1, 150.5, 158.4, 162.3, 186.2

### 2-(4-(2-Chlorophenyl)piperazin-1-yl)-9-methyl-4-oxo-4H-pyrido[1,2-a]pyrimidine-3-carbaldehyde (139)

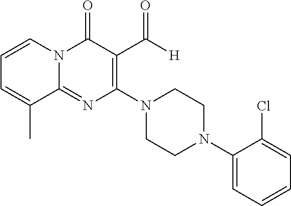 [SMILES: Cc1cccn2c(=O)c(C=O)c(N3CCN(c4ccccc4Cl)CC3)nc12]

¹H NMR (400 MHz, CDCl₃) δ 2.41 (s, 3H), 3.19 (t, J=4.8 Hz, 4H), 3.92 (t, J=4.6 Hz, 4H), 6.82 (t, J=7.0 Hz, 1H), 6.98 (t, J=7.6 Hz, 1H), 7.04 (d, J=7.2 Hz, 1H), 7.21 (t, J=7.6 Hz, 1H), 7.36 (d, J=7.6 Hz, 1H), 7.55 (d, J=6.4 Hz, 1H), 8.73 (d, J=6.8 Hz, 1H), 10.15 (s, 1H); ¹³C NMR (100 MHz, CDCl₃) δ 17.6, 49.3, 51.4, 96.1, 112.7, 120.5, 124.0, 125.8, 127.6, 128.8, 130.6, 133.0, 137.8, 148.7, 150.5, 158.6, 162.5, 186.4.

### 2-(3,4-Dihydroisoquinolin-2 (1H)-yl)-9-methyl-4-oxo-4H-pyrido[1,2-a]pyrimidine-3-carbaldehyde (140)

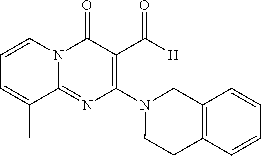 [SMILES: Cc1cccn2c(=O)c(C=O)c(N3CCc4ccccc4C3)nc12]

¹H NMR (400 MHz, CDCl₃) δ 2.43 (s, 3H), 3.05 (t, J=5.8 Hz, 2H), 4.03 (t, J=5.8 Hz, 2H), 4.73 (s, 2H), 6.78 (t, J=7.0 Hz, 1H), 7.06-7.17 (m, 4H), 7.52 (d, J=6.8 Hz, 1H), 8.70 (d, J=7.6 Hz, 1H), 10.21 (s, 1H); ¹³C NMR (100 MHz, CDCl₃) δ 17.6, 28.7, 46.3, 52.0, 96.1, 112.5, 125.8, 126.2, 126.6, 128.4, 133.0, 133.9, 134.6, 137.5, 150.3, 158.6, 162.3, 186.7.

### 2-(Isobutylamino)-9-methyl-4-oxo-4H-pyrido[1,2-a]pyrimidine-3-carbaldehyde (141)

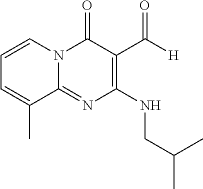 [SMILES: Cc1cccn2c(=O)c(C=O)c(NCC(C)C)nc12]

¹H NMR (400 MHz, CDCl₃) δ 0.95 (d, J=4 Hz, 6H), 1.90 (m, 1H), 2.37 (s, 3H), 3.41 (t, J=6.8 Hz, 2H), 6.76 (t, J=6.8 Hz, 1H), 7.24-7.52 (m, 1H), 8.69 (dd, J=0.8, 7.2 Hz, 1H), 9.67 (brs, 1H), 10.22 (s, 1H); ¹³C NMR (100 MHz, CDCl₃) δ 17.9, 20.4, 28.7, 48.1, 94.4, 112.5, 125.9, 133.2, 138.1, 152.8, 159.5, 160.7, 190.2.

### 2-(Diethylamino)-9-methyl-4-oxo-4H-pyrido[1,2-a]pyrimidine-3-carbaldehyde (142)

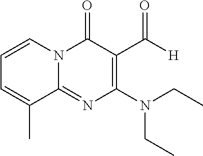 [SMILES: CCN(CC)c1nc2c(C)cccn2c(=O)c1C=O]

¹H NMR (400 MHz, CDCl₃) δ 1.25 (t, J=6.8 Hz, 6H), 2.36 (s, 3H), 3.65 (q, J=6.8 Hz, 4H), 6.72 (t, J=6.8 Hz, 1H), 7.47 (d, J=6.8 Hz, 1H), 8.65 (d, J=6.4 Hz, 1H), 10.12 (s, 1H); ¹³C NMR (100 MHz, CDCl₃) δ 13.2, 17.7, 45.3, 96.2, 112.2, 125.8, 133.0, 137.3, 150.2, 158.5, 162.6, 186.9.

### 2-(Cyclohexylmethylamino)-9-methyl-4-oxo-4H-pyrido[1,2-a]pyrimidine-3-carbaldehyde (143)

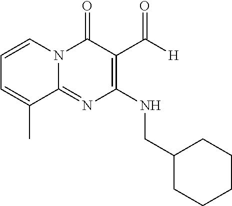 [SMILES: Cc1cccn2c(=O)c(C=O)c(NCC3CCCCC3)nc12]

¹H NMR (400 MHz, CDCl₃) δ 0.93-1.02 (m, 2H), 1.11-1.25 (m, 3H), 1.57-1.77 (m, 6H), 2.36 (s, 3H), 3.43 (t, J=6.0 Hz, 2H), 6.75 (t, J=7.2 Hz, 1H), 7.50 (d, J=7.2 Hz, 1H), 8.67 (d, J=6.8 Hz, 1H), 9.65 (brs, 1H), 10.21 (s, 1H); ¹³C NMR (100 MHz, CDCl₃) δ 17.9, 26.0, 26.5, 31.1, 38.2, 47.0, 94.4, 112.5, 125.8, 133.2, 138.0, 152.8, 159.4, 160.6, 190.2

### 2-Chloro-9-methyl-4-oxo-4H-pyrido[1,2-a]pyrimidine-3-carboxylic acid (144)

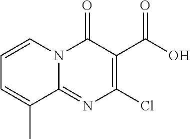 [SMILES: Cc1cccn2c(=O)c(C(=O)O)c(Cl)nc12]

¹H NMR (400 MHz, DMSO-d₆) δ 2.58 (s, 3H), 7.53 (t, J=7.0 Hz, 1H), 8.14 (d, J=7.2 Hz, 1H), 8.97 (d. J=6.8 Hz, 1H), 13.53 (brs, 1H); ¹³C NMR (100 MHz, DMSO-d₆) δ 16.7, 108.1, 117.1, 125.6, 133.3, 138.7, 148.2, 152.0, 154.6, 163.9.

### 2-Chloro-7-methyl-4-oxo-4H-pyrido[1,2-a]pyrimidine-3-carboxylic acid (145)

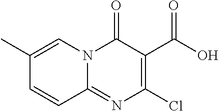 [SMILES: Cc1ccc2nc(Cl)c(C(=O)O)c(=O)n2c1]

¹H NMR (400 MHz, DMSO-d₆) δ 2.49 (s, 3H), 7.76 (d, J=8.8 Hz, 1H), 8.11 (d, J=8.8 Hz, 1H), 8.89 (s, 1H), 13.46 (br s, 1H).

### 2-Chloro-6-methyl-4-oxo-4H-pyrido[1,2-a]pyrimidine-3-carboxylic acid (146)

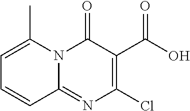 [SMILES: Cc1cccc2nc(Cl)c(C(=O)O)c(=O)n12]

¹H NMR (400 MHz, DMSO-d₆) δ 3.00 (s, 3H), 7.19 (d, J=7.6 Hz, 1H), 7.52 (d, J=8.0 Hz, 1H), 7.92 (t, J=8.0 Hz, 1H), 13.35 (br s, 1H).

### 9-Methyl-4-oxo-2-(phenylamino)-4H-pyrido[1,2-a]pyrimidine-3-carboxylic acid (147)

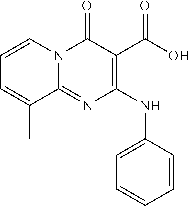 [SMILES: Cc1cccn2c(=O)c(C(=O)O)c(Nc3ccccc3)nc12]

¹H NMR (400 MHz, CDCl₃) δ 2.50 (s, 3H), 6.70 (dd, J=6.8, 7.2 Hz, 1H), 7.15 (dd, J=7.2, 7.2 Hz, 1H), 7.37 (dd, J=7.2, 7.6 Hz, 2H), 7.65 (d, J=6.8 Hz, 1H), 7.76 (d, J=8.4 Hz, 2H), 8.76 (d, J=7.2 Hz, 1H), 11.70 (brs, 1H), 14.31 (s, 1H).

### 2-(3-Chlorophenylamino)-9-methyl-4-oxo-4H-pyrido[1,2-a]pyrimidine-3-carboxylic acid (148)

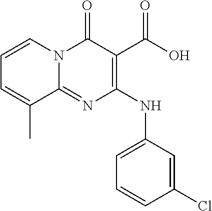 [SMILES: Cc1cccn2c(=O)c(C(=O)O)c(Nc3cccc(Cl)c3)nc12]

¹H NMR (400 MHz, DMSO-d₆) δ 2.55 (s, 3H), 7.04 (t, J=7.0 Hz, 1H), 7.12 (d, J=8.0 Hz, 1H), 7.28 (J=8.0 Hz, 1H), 7.71 (d, J=8.0 Hz, 1H), 8.17 (s, 1H), 8.79 (d, J=7.6 Hz, 1H), 11.78 (brs, 1H).

### 2-(3-Chlorophenylamino)-8-methyl-4-oxo-4H-pyrido[1,2-a]pyrimidine-3-carboxylic acid (149)

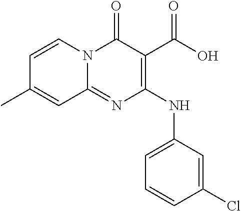 [SMILES: Cc1ccn2c(=O)c(C(=O)O)c(Nc3cccc(Cl)c3)nc2c1]

¹H NMR (400 MHz, CDCl₃) δ 2.49 (s, 3H), 6.93 (d, J=7.6 Hz, 1H), 7.12 (d, J=7.6 Hz, 1H), 7.25-7.29 (m, 2H), 7.46 (d, J=7.2 Hz, 1H), 7.96 (s, 1H), 8.76 (d, J=7.2 Hz, 1H), 11.72 (br s, 1H), 14.19 (s, 1H).

### 2-(3-Chlorophenylamino)-7-methyl-4-oxo-4H-pyrido[1,2-a]pyrimidine-3-carboxylic acid (150)

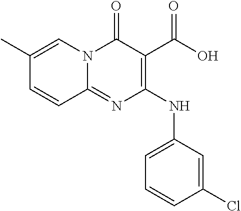 [SMILES: Cc1ccc2nc(Nc3cccc(Cl)c3)c(C(=O)O)c(=O)n2c1]

¹H NMR (400 MHz, CDCl₃) δ 2.41 (s, 3H), 7.12 (d, J=8.0 Hz, 1H), 7.27 (t, J=8.6 Hz, 1H), 7.41 (d, J=8.8 Hz, 1H), 7.47 (d, J=7.6 Hz, 1H), 7.96 (s, 1H), 8.68 (s, 1H), 11.70 (br s, 1H), 14.28 (s, 1H).

### 2-(3-Chlorophenylamino)-6-methyl-4-oxo-4H-pyrido[1,2-a]pyrimidine-3-carboxylic acid (151)

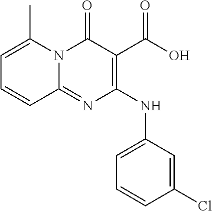 [SMILES: Cc1cccc2nc(Nc3cccc(Cl)c3)c(C(=O)O)c(=O)n12]

¹H NMR (400 MHz, CDCl₃) δ 3.03 (s, 3H), 6.70 (d, J=6.8 Hz, 1H), 7.10 (d, J=8.0 Hz, 1H), 7.23-7.27 (m, 2H), 7.44 (d, J=8.0 Hz, 1H), 7.56 (t, J=8.0 Hz, 1H), 7.91 (s, 1H), 11.76 (br s, 1H), 14.37 (s, 1H).

### 2-(3-Fluorophenylamino)-9-methyl-4-oxo-4H-pyrido[1,2-a]pyrimidine-3-carboxylic acid (152)

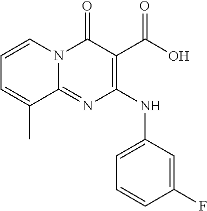 [SMILES: Cc1cccn2c(=O)c(C(=O)O)c(Nc3cccc(F)c3)nc12]

¹H NMR (400 MHz, CDCl₃) δ 2.54 (s, 3H), 6.81-6.87 (m, 1H), 7.03 (t, J=7.2 Hz, 1H), 7.28-7.31 (m, 2H), 7.71 (d, J=6.8 Hz, 1H), 7.89 (d, J=10.4 Hz, 1H), 8.79 (d, J=7.2 Hz 1H), 11.83 (b s, 1H), 14.26 (br s, 1H).

### 9-Methyl-4-oxo-2-(3-(trifluoromethyl)phenylamino)-4H-pyrido[1,2-a]pyrimidine-3-carboxylic acid (153)

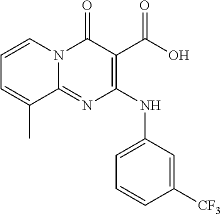 [SMILES: Cc1cccn2c(=O)c(C(=O)O)c(Nc3cccc(C(F)(F)F)c3)nc12]

¹H NMR (400 MHz, CDCl₃) δ 2.54 (s, 3H), 7.05 (t, J=7.0 Hz, 1H), 7.40 (d, J=7.6 Hz, 1H), 7.47 (t, J=8.0 Hz, 1H), 7.61 (d, J=8.0 Hz, 1H), 7.73 (d, J=6.8 Hz, 1H), 8.58 (s 1H), 8.81 (d, J=6.8 Hz, 1H), 11.91 (br s, 1H).

### 9-Methyl-4-oxo-2-(3-(trifluoromethoxy)phenylamino)-4H-pyrido[1,2-a]pyrimidine-3-carboxylic acid (154)

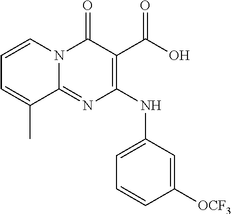 [SMILES: Cc1cccn2c(=O)c(C(=O)O)c(Nc3cccc(OC(F)(F)F)c3)nc12]

¹H NMR (400 MHz, CDCl₃) δ 2.58 (s, 3H), 7.00 (d, J=8.0 Hz, 1H), 7.05 (t, J=7.0 Hz, 1H), 7.36 (t, J=8.0 Hz, 1H), 7.42 (d, J=8.0 Hz, 1H), 7.72 (d, J=6.8 Hz, 1H), 8.09 (s, 1H), 8.81 (d, J=7.2 Hz, 1H), 11.89 (br s, 1H), 14.26 (br s, 1H).

### 9-Methyl-2-(3-nitrophenylamino)-4-oxo-4H-pyrido[1,2-a]pyrimidine-3-carboxylic acid (155)

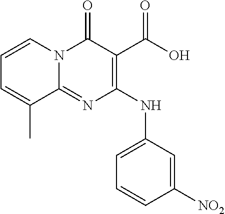 [SMILES: Cc1cccn2c(=O)c(C(=O)O)c(Nc3cccc([N+](=O)[O-])c3)nc12]

¹H NMR (400 MHz, DMSO-d₆) δ 2.60 (s, 3H), 7.40 (t, J=7.0 Hz, 1H), 7.73 (t, J=8.2 Hz, 1H), 7.96 (d, J=7.6 Hz, 1H), 8.02 (d, J=7.6 Hz, 1H), 8.13 (d, J=6.8 Hz, 1H), 8.90 (d, J=7.2 Hz, 1H), 9.33 (s, 1H), 11.84 (br s, 1H), 14.43 (br s, 1H).

### 2-(3-(Methoxycarbonyl)phenylamino)-9-methyl-4-oxo-4H-pyrido[1,2-a]pyrimidine-3-carboxylic acid (156)

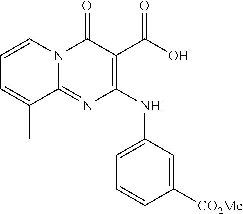 [SMILES: COC(=O)c1cccc(Nc2nc3c(C)cccn3c(=O)c2C(=O)O)c1]

¹H NMR (400 MHz, CDCl₃) δ 2.57 (s, 3H), 3.92 (s, 3H), 7.052 (t, J=6.8 Hz, 1H), 7.43 (t, J=8.0 Hz, 1H), 7.71 (t, J=7.0 Hz, 2H), 7.82 (d, J=8.0 Hz, 1H), 8.79 (d, J=6.8 Hz, 1H), 8.83 (s, 1H), 11.83 (br s, 1H), 14.28 (br s, 1H).

### 2-(3-Hydroxyphenylamino)-9-methyl-4-oxo-4H-pyrido[1,2-a]pyrimidine-3-carboxylic acid (157)

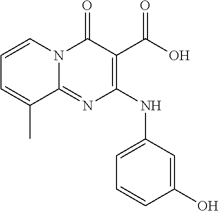 [SMILES: Cc1cccn2c(=O)c(C(=O)O)c(Nc3cccc(O)c3)nc12]

¹H NMR (400 MHz, CD₃OD) δ 2.55 (s, 3H), 6.61 (d, J=8.0 Hz, 1H), 7.15-7.24 (m, 3H), 7.34 (s, 1H), 7.88 (d, J=6.8 Hz, 1H), 8.82 (d, J=7.2 Hz, 1H).

### 2-(4-Hydroxyphenylamino)-9-methyl-4-oxo-4H-pyrido[1,2-a]pyrimidine-3-carboxylic acid (158)

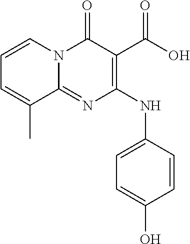 [SMILES: Cc1cccn2c(=O)c(C(=O)O)c(Nc3ccc(O)cc3)nc12]

¹H NMR (400 MHz, CD₃OD) δ 2.45 (s, 3H), 6.81 (d, J=8.8 Hz, 2H), 7.10 (t, J=7.0 Hz, 1H), 7.57 (d, J=8.8 Hz, 1H), 7.81 (d, J=6.8 Hz, 1H), 8.78 (d, J=7.2 Hz, 1H), 11.26 (br s, 1H).

### 2-(4-tert-Butylphenylamino)-9-methyl-4-oxo-4H-pyrido[1,2-a]pyrimidine-3-carboxylic acid (159)

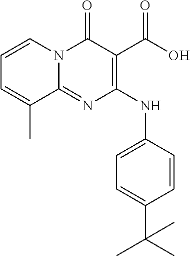 [SMILES: Cc1cccn2c(=O)c(C(=O)O)c(Nc3ccc(C(C)(C)C)cc3)nc12]

¹H NMR (400 MHz, CDCl₃) δ 1.33 (s, 9H), 2.49 (s, 3H), 6.95 (t, J=7.0 Hz, 1H), 7.37 (d, J=7.2 Hz, 2H), 7.63 (d, J=5.6 Hz, 1H), 7.69 (d, J=6.8 Hz, 2H), 8.71 (d, J=6.8 Hz, 1H), 11.64 (br s, 1H) 14.31 (br s, 1H); ¹³C NMR (100 MHz, CDCl₃) δ 18.2, 31.3, 34.4, 85.3, 114.1, 121.3, 125.5, 125.7, 133.6, 135.4, 138.2, 147.4, 150.2, 157.0, 161.8, 169.7.

### 2-(3-Chlorobenzylamino)-9-methyl-4-oxo-4H-pyrido[1,2-a]pyrimidine-3-carboxylic acid (160)

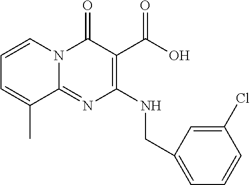 [SMILES: Cc1cccn2c(=O)c(C(=O)O)c(NCc3cccc(Cl)c3)nc12]

¹H NMR (400 MHz, CDCl₃) δ 2.38 (s, 3H), 4.83 (d, J=6.0 Hz, 2H), 7.17 (t, J=7.0 Hz, 1H), 7.32-7.40 (m, 3H), 7.50 (s, 1H), 7.89 (d, J=6.8 Hz, 1H), 8.68 (d, J=7.2 Hz, 1H), 9.82 (d, J=6.2 Hz, 1H), 14.25 (br s, 1H).

### 2-(Diethylamino)-9-methyl-4-oxo-4H-pyrido[1,2-a]pyrimidine-3-carboxylic acid (161)

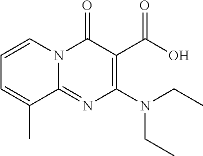 [SMILES: CCN(CC)c1nc2c(C)cccn2c(=O)c1C(=O)O]

¹H NMR (400 MHz, CDCl₃) δ 1.32 (t, J=6.8 Hz, 6H), 2.41 (s, 3H), 3.68 (q, J=6.8 Hz, 4H), 6.67 (t, J=7.2 Hz, 1H), 7.38 (d, J=6.8 Hz, 1H), 8.71 (d, J=7.2 Hz, 1H), 14.08 (s, 1H); ¹³C NMR (100 MHz, CDCl₃) δ 13.8, 17.8, 45.4, 96.2, 112.2, 125.8, 133.0, 137.3, 150.2, 158.5, 162.6, 171.6.

### 2-(Isobutylamino)-9-methyl-4-oxo-4H-pyrido[1,2-a]pyrimidine-3-carboxylic acid (162)

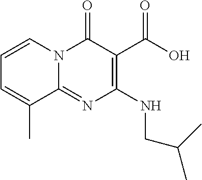 [SMILES: Cc1cccn2c(=O)c(C(=O)O)c(NCC(C)C)nc12]

¹H NMR (400 MHz, CDCl₃) δ 0.97 (d, J=6.8 Hz, 6H), 1.93-1.99 (m, 1H), 2.40 (s, 3H), 3.43 (t, J=6.4 Hz, 2H), 6.84 (t, J=7.2 Hz, 1H), 7.53 (d, J=6.4 Hz, 1H), 8.62 (d, J=7.6 Hz, 1H), 9.52 (brs, 1H), 14.12 (s, 1H); ¹³C NMR (100 MHz, CDCl₃) δ 17.9, 20.4, 28.7, 48.6, 84.8, 113.2, 125.7, 133.2, 137.5, 150.5, 159.7, 162.0, 169.9.

### 2-(Cyclohexylmethylamino)-9-methyl-4-oxo-4H-pyrido[1,2-a]pyrimidine-3-carboxylic acid (163)

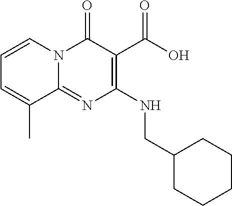 [SMILES: Cc1cccn2c(=O)c(C(=O)O)c(NCC3CCCCC3)nc12]

¹H NMR (400 MHz, CDCl₃) δ 0.98-1.05 (m, 2H), 1.13-1.24 (m, 3H), 1.60-1.79 (m, 6H), 2.42 (s, 3H), 3.45 (t, J=6.4 Hz, 2H), 6.83 (t, J=7.2 Hz, 1H), 7.54 (d, J=6.8 Hz, 1H), 8.62 (d, J=7.2 Hz, 1H), 9.57 (brs, 1H), 14.13 (s, 1H); ¹³C NMR (100 MHz, CDCl₃) δ 18.0, 26.0, 26.2, 31.2, 38.2, 47.4, 84.8, 113.2, 125.7, 133.2, 137.5, 150.5, 159.6, 162.0, 170.0.

### 2-(Cyclohexylamino)-9-methyl-4-oxo-4H-pyrido[1,2-a]pyrimidine-3-carboxylic acid (164)

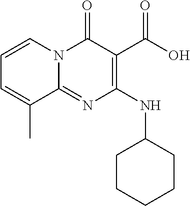 [SMILES: Cc1cccn2c(=O)c(C(=O)O)c(NC3CCCCC3)nc12]

¹H NMR (400 MHz, CDCl₃) δ 1.19-1.42 (m, 5H), 1.56-1.60 (m, 2H), 1.70-1.76 (m, 2H), 1.94-1.98 (m, 2H), 2.38 (s, 3H), 6.79 (t, J=6.8 Hz, 1H), 7.51 (d, J=6.8 Hz, 1H), 8.56 (d, J=6.8 Hz, 1H), 9.42 (d, J=6.8 Hz, 1H), 14.14 (s, 1H); ¹³C NMR (100 MHz, CDCl₃) δ 17.8, 24.7, 25.7, 32.6, 50.0, 84.7, 113.1, 125.6, 133.1, 137.4, 150.5, 158.5, 162.0, 169.9.

### 2-(Cyclopentylamino)-9-methyl-4-oxo-4H-pyrido[1,2-a]pyrimidine-3-carboxylic acid (165)

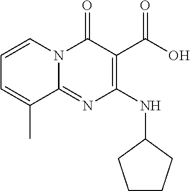 [SMILES: Cc1cccn2c(=O)c(C(=O)O)c(NC3CCCC3)nc12]

¹H NMR (400 MHz, CDCl₃) δ 1.54-1.67 (m, 4H), 1.73-1.78 (m, 2H), 2.04-2.10 (m, 2H), 2.42 (s, 3H), 4.51 (q, J=6.8 Hz, 1H), 6.83 (t, J=6.8 Hz, 1H), 7.53 (d, J=6.8 Hz, 1H), 8.59 (d, J=6.8 Hz, 1H), 9.47 (d, J=6.8 Hz, 1H), 14.15 (s, 1H); ¹³C NMR (100 MHz, CDCl₃) δ 18.0, 24.1, 33.3, 53.0, 84.8, 113.3, 125.7, 133.3, 137.5, 150.5, 158.9, 162.0, 169.9.

### 2-(Cycloheptylamino)-9-methyl-4-oxo-4H-pyrido[1,2-a]pyrimidine-3-carboxylic acid (166)

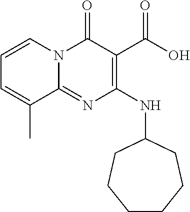 [SMILES: Cc1cccn2c(=O)c(C(=O)O)c(NC3CCCCCC3)nc12]

¹H NMR (400 MHz, CDCl₃) δ 1.23-1.57 (m, 4H), 1.59-1.68 (m, 4H), 1.69-1.74 (m, 2H), 1.98-2.04 (m, 2H), 2.43 (s, 3H), 4.30-4.36 (m, 1H), 6.83 (t, J=6.8 Hz, 1H), 7.53 (d, J=6.8 Hz, 1H), 8.64 (d, J=6.8 Hz, 1H), 9.53 (d, J=6.8 Hz, 1H), 14.19 (s, 1H); ¹³C NMR (100 MHz, CDCl₃) δ 18.0, 24.6, 28.1, 34.7, 52.3, 84.8, 113.1, 125.8, 133.2, 137.4, 150.4, 158.3, 162.1, 170.0.

### 2-(1-(tert-Butoxycarbonyl)piperidin-4-ylamino)-9-methyl-4-oxo-4H-pyrido[1,2-a]pyrimidine-3-carboxylic acid (167)

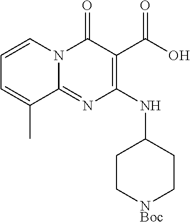 [SMILES: Cc1cccn2c(=O)c(C(=O)O)c(NC3CCN(C(=O)OC(C)(C)C)CC3)nc12]

¹H NMR (400 MHz, CDCl₃) δ 1.51 (s, 9H), 1.61-1.65 (m, 2H), 2.01-2.03 (m, 2H), 2.42 (s, 3H), 2.99-3.05 (m, 2H), 3.98-4.00 (m, 2H), 4.26-4.33 (m, 1H), 6.88 (t, J=7.2 Hz, 1H), 7.58 (d, J=6.8 Hz, 1H), 8.67 (d, J=7.2 Hz, 1H), 9.56 (d, J=6.8 Hz), 14.12 (s, 1H); ¹³C NMR (100 MHz, CDCl₃) δ 17.9, 28.6, 31.6, 48.5, 66.4, 79.9, 85.0, 113.5, 125.9, 133.2, 137.8, 150.6, 154.9, 158.9, 162.0, 169.9.

### 2-(2-(4-Fluorophenoxy)ethylamino)-9-methyl-4-oxo-4H-pyrido[1,2-a]pyrimidine-3-carboxylic acid (168)

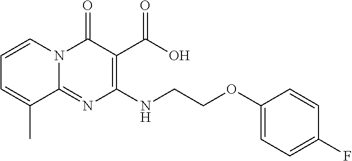 [SMILES: Cc1cccn2c(=O)c(C(=O)O)c(NCCOc3ccc(F)cc3)nc12]

¹H NMR (400 MHz, CDCl₃) δ 2.44 (s, 3H), 4.01 (t, J=5.6 Hz, 2H), 4.15 (t, J=5.6 Hz, 2H), 6.83-6.96 (m, 5H), 7.59 (d, J=6.8 Hz, 1H), 8.68 (d, J=7.2 Hz, 1H), 9.81 (brs, 1H), 14.01 (s, 1H); ¹³C NMR (100 MHz, CDCl₃) δ 18.0, 40.5, 67.1, 85.3, 113.6, 115.8, 115.9, 116.0, 116.1, 125.9, 133.2, 137.9, 150.6, 154.8, 159.8, 161.9, 169.7.

### 9-Methyl-4-oxo-2-(2-(4-(trifluoromethoxy)phenoxy)ethylamino)-4H-pyrido[1,2-a]pyrimidine-3-carboxylic acid (169)

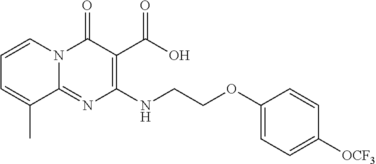 [SMILES: Cc1cccn2c(=O)c(C(=O)O)c(NCCOc3ccc(OC(F)(F)F)cc3)nc12]

¹H NMR (400 MHz, CDCl₃) δ 2.44 (s, 3H), 4.03 (t, J=5.6 Hz, 2H), 4.18 (t, J=5.6 Hz, 2H), 6.90 (d, J=9.2 Hz, 2H), 6.91 (t, J=6.8 Hz, 1H), 7.11 (d, J=9.2 Hz, 2H), 7.60 (d, J=6.8 Hz, 1H), 9.70 (d, J=7.2 Hz, 1H), 9.82 (brs, 1H), 14.08 (s, 1H); ¹³C NMR (100 MHz, CDCl₃) δ 18.0, 40.5, 66.9, 77.4, 85.4, 113.7, 115.7, 122.6, 126.0, 133.2, 138.0, 155.8, 157.6, 159.9, 162.0, 169.0, 170.4.

### 9-Methyl-2-morpholino-4-oxo-4H-pyrido[1,2-a]pyrimidine-3-carboxylic acid (170)

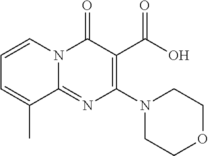 [SMILES: Cc1cccn2c(=O)c(C(=O)O)c(N3CCOCC3)nc12]

¹H NMR (400 MHz, CDCl₃) δ 2.42 (s, 3H), 3.65 (t, J=4.8 Hz, 4H), 3.74 (t, J=4.8 Hz, 4H), 6.86 (t, J=6.8 Hz, 1H), 7.51 (d, J=6.8 Hz, 1H), 8.67 (d, J=6.8 Hz, 1H), 13.98 (s, 1H); ¹³C NMR (100 MHz, CDCl₃) δ 18.1, 58.4, 64.8, 97.5, 113.6, 124.6, 132.6, 136.0, 148.1, 160.5, 161.7, 171.3.

### 2-(3,4-Dihydroisoquinolin-2 (1H)-yl)-9-methyl-4-oxo-4H-pyrido[1,2-a]pyrimidine-3-carboxylic acid (171)

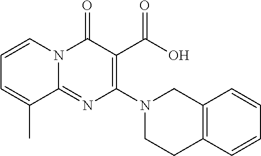 [SMILES: Cc1cccn2c(=O)c(C(=O)O)c(N3CCc4ccccc4C3)nc12]

¹H NMR (400 MHz, CDCl₃) δ 2.45 (s, 3H), 3.03 (t, J=5.8 Hz, 2H), 4.08 (m, 2H), 4.73 (m, 2H), 6.83 (t, J=7.0 Hz, 1H), 7.06-7.18 (m, 4H), 7.52 (d, J=6.8 Hz, 1H), 8.60 (d, J=7.2 Hz, 1H), 13.73 (br s, 1H); ¹³C NMR (100 MHz, CDCl₃) δ 17.6, 28.5, 46.1, 52.4, 86.4, 113.0, 125.5, 126.1, 126.2, 126.6, 128.4, 132.9, 133.7, 134.4, 136.8, 148.1, 159.9, 163.2, 165.3.

### 2-(4-(2-Chlorophenyl)piperazin-1-yl)-9-methyl-4-oxo-4H-pyrido[1,2-a]pyrimidine-3-carboxylic acid (172)

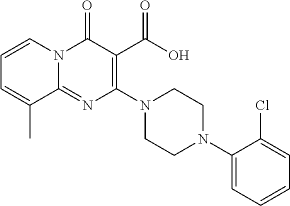 [SMILES: Cc1cccn2c(=O)c(C(=O)O)c(N3CCN(c4ccccc4Cl)CC3)nc12]

¹H NMR (400 MHz, CDCl₃) δ 2.44 (s, 3H), 3.19 (t, J=4.8 Hz, 4H), 3.96 (m, 4H), 6.87 (t, J=7.0 Hz, 1H), 6.98 (t, J=7.6 Hz, 1H), 7.02 (d, J=8.4 Hz, 1H), 7.20 (t, J=7.8 Hz, 1H), 7.36 (d, J=8.0 Hz, 1H), 7.55 (d, J=6.8 Hz, 1H), 8.66 (d, J=7.2 Hz, 1H), 13.74 (br s, 1H).

### 2-(3-Chlorophenylamino)-8-(4-methylpiperazin-1-yl)-4-oxo-4H-pyrido[1,2-a]pyrimidine-3-carboxylic acid (173)

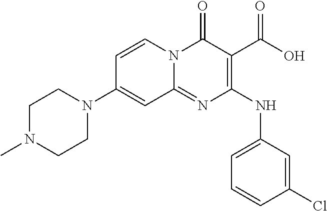 [SMILES: CN1CCN(c2ccn3c(=O)c(C(=O)O)c(Nc4cccc(Cl)c4)nc3c2)CC1]

¹H NMR (400 MHz, CDCl₃) δ 2.34 (s, 3H), 2.53 (t, J=4.8 Hz, 4H), 3.54 (t, J=4.8 Hz, 4H), 6.34 (d, J=2.8 Hz, 1H), 6.55 (dd, J=2.8, 8.4 Hz, 1H), 7.04 (d, J=7.2 Hz, 1H), 7.22 (t, J=8.0 Hz, 1H), 7.49 (dd, J=1.6, 8.0 Hz, 1H), 7.86 (t, J=2.0 Hz, 1H), 8.53 (d, J=8.4 Hz, 1H), 11.5 (s, 1H), 14.18 (s, 1H); ¹³C NMR (100 MHz, CDCl₃) δ 46.1, 46.4, 54.4, 83.6, 98.8, 105.1, 120.0, 121.9, 124.0, 128.8, 129.9, 134.4, 139.9, 151.4, 155.6, 158.2, 161.8, 170.2.

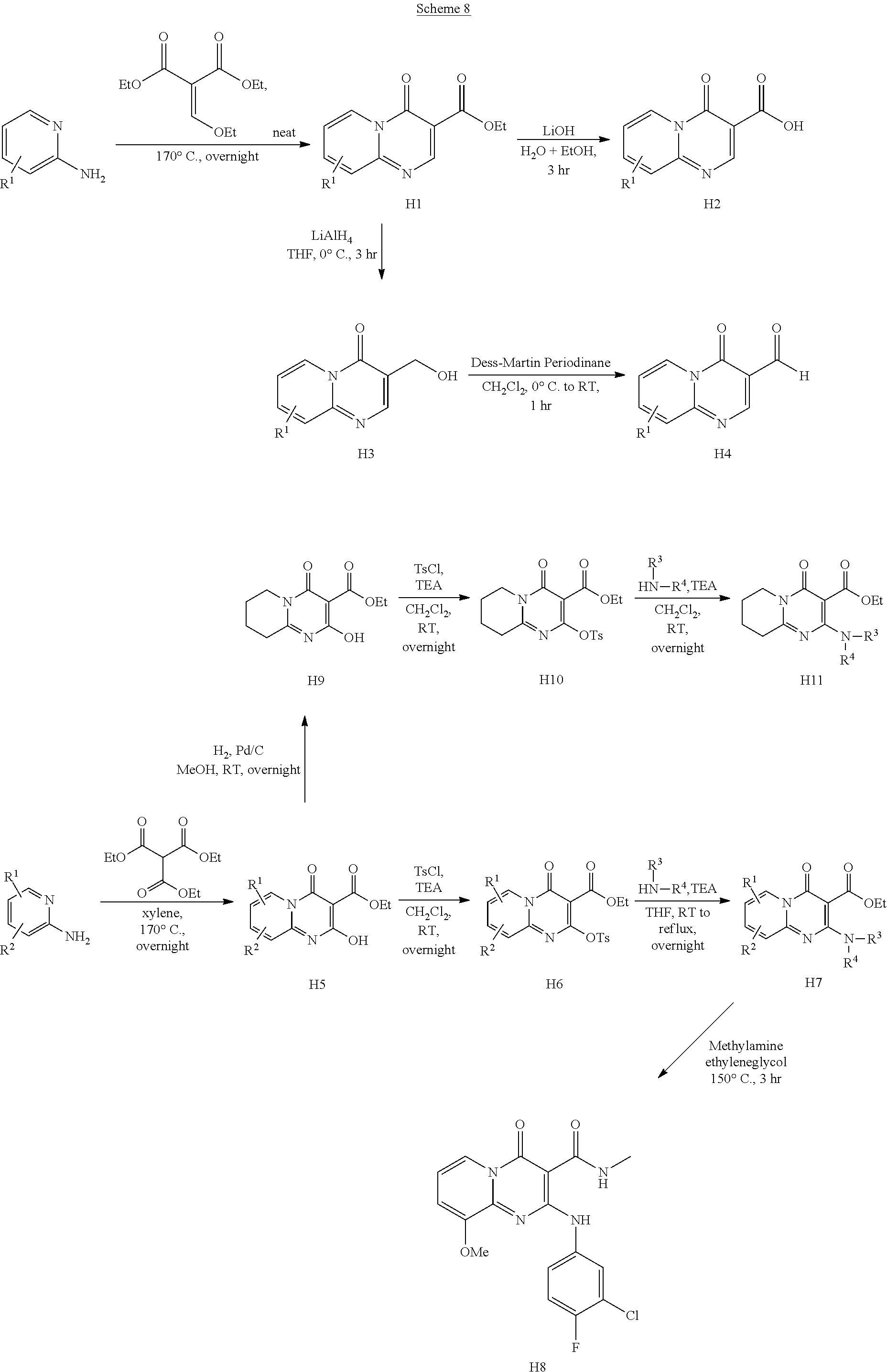

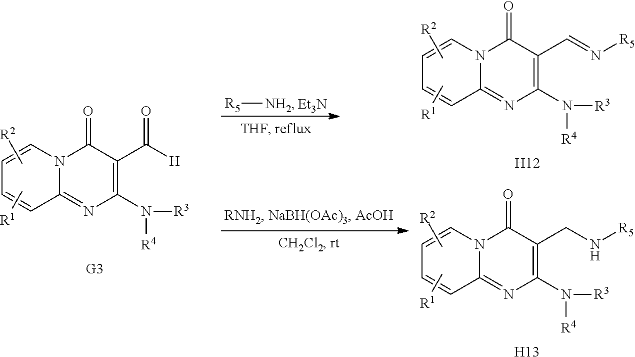

### General Procedure for the Synthesis of H1

2-Amino-3-picoline (1.0 mmol) was dissolved in diethyl ethoxymethylenemalonate (1.0 mmol). The solution was heated to 170° C. for 12 h. After cooling, the dark residue was triturated with EtOAc (10 mL). The residual pale solid was collected by filtration and washed with EtOAc to give H1.

### General Procedure for the Synthesis of H2

To a stirred solution of H1 (0.43 mmol) in H₂O (3.0 mL) and EtOH (1.0 mL) was added LiOH (0.86 mmol). The mixture was stirred at room temperature for 3 h. The reaction mixture was diluted with CH₂Cl₂ (10 mL) and washed with 1 N HCl (10 ml). The organic layer was dried over anhydrous MgSO₄ and concentrated in vacuo. The crude product was purified by flash column chromatography to give H2.

### General Procedure for the Synthesis of H3

To a stirred solution of H1 (0.38 mmol) in THF (2.0 mL) was added LiAlH₄ (0.57 mmol) at 0° C. The reaction mixture was stirred at 0° C. for 3 h. After reaction was completed, 1N NaOH (2 mL) was added dropwise. The mixture was diluted with CH₂Cl₂ (10 mL) and washed with H₂O (10 ml). The organic layer was dried over anhydrous MgSO₄ and concentrated in vacuo. The crude product was purified by flash column chromatography to give H3.

### General Procedure for the Synthesis of H4

To a stirred solution of 113 (95 μmol) in CH₂Cl₂ (1.0 mL) was added NaHCO₃ (285 μmol) and Dess-Martin Periodinane (114 μmol) at 0° C. The mixture was stirred at 0° C. for 1 h. The reaction mixture was filtered off and concentrated in vacuo. The crude product was purified by flash column chromatography to give H4.

### General Procedure for the Synthesis of H5

To a stirred solution of 2-Amino-pyridine (10.6 mmol) in xylene (10.0 mL) was added diethyl ethoxymethylenemalonate (21.2 mmol). The mixture was stirred at 140° C. for 3 hr. After reaction was completed, the residual pale solid was collected by filtration and washed with diethyl ether to give H5.

### General Procedure for the Synthesis of H6

To a stirred solution of H5 (0.42 mmol) in THF (5.0 mL) was added triethylamine (0.63 mmol) and p-toluenesulfonylchloride (0.46 mmol) at 0° C. The reaction mixture was stirred at room temperature for overnight. After reaction was completed, the mixture was diluted with CH₂Cl₂ (40 mL) and washed with 1N HCl (50 ml), saturated NaHCO₃ (50 ml) and brine (50 ml). The organic layer was dried over anhydrous MgSO₄ and concentrated in vacuo. The crude product was purified by flash column chromatography to give H6.

### General Procedure for the Synthesis of H7

To a stirred solution of H6 (0.25 mmol) in THF (1.2 mL) was added triethylamine (0.5 mmol) and an amine (0.26 mmol) at 0° C. The reaction mixture was stirred at room temperature for overnight. After reaction was completed, the mixture was diluted with CH₂Cl₂ (10 mL) and washed with 1N HCl (10 ml), saturated NaHCO₃ (10 ml) and brine (10 ml). The organic layer was dried over anhydrous MgSO₄ and concentrated in vacuo. The crude product was purified by flash column chromatography to give H7.

### General Procedure for the Synthesis of H8

To a stirred solution of H7 (0.27 mmol) in ethylene glycol (3.0 mL) was added methylamine (2 N solution in THF 1.3 mL). The mixture was stirred at 150° C. for 3 hr. The reaction mixture was added with ethylacetate (10 mL) and the residual pale solid was collected by filtration and washed with EtOAc. The crude product was purified by flash column chromatography to give H8.

### General Procedure for the Synthesis of H9

To a stirred solution of H5 (2.13 mmol) in MeOH (8.0 mL) was added Pd/C (113 mg). The mixture was stirred at room temperature under H₂ for 3 h. After reaction was completed, the reaction mixture was filtered off and concentrated in vacuo. The crude product was recrystallized with EtOAc and hexane (1:4) to give H9.

### General Procedure for the Synthesis of H10

To a stirred solution of H9 (0.42 mmol) in CH₂Cl₂ (5.0 mL) was added triethylamine (0.63 mmol) and p-toluenesulfonylchloride (0.46 mmol) at 0° C. The reaction mixture was stirred at room temperature for overnight. After reaction was completed, the mixture was diluted with CH₂Cl₂ (40 mL) and washed with 1N HCl (50 ml), saturated NaHCO₃ (50 ml) and brine (50 ml). The organic layer was dried over anhydrous MgSO₄ and concentrated in vacuo. The crude product was purified by flash column chromatography (Hexane:EtOAc=1:2) to give H10.

### General Procedure for the Synthesis of H11

To a stirred solution of H10 (0.25 mmol) in THF (2.0 mL) was added triethylamine (0.5 mmol) and an amine (0.37 mmol) at 0° C. The reaction mixture was stirred at room temperature for overnight. After reaction was completed, the mixture was diluted with CH₂Cl₂ (10 mL) and washed with 1N HCl (10 ml), saturated NaHCO₃ (10 ml) and brine (10 ml). The organic layer was dried over anhydrous MgSO₄ and concentrated in vacuo. The crude product was purified by flash column chromatography (Hexane:EtOAc=1:1) to give H11.

### General Procedure for the Synthesis of H12

A solution of G3 (1.0 mmol), an amine (1.1 mmol) and triethylamine (2.0 mmol) in THF (2 mL) was refluxed for 1 h and cooled to room temperature. The solvent was evaporated to dryness, which was extracted with CH₂Cl₂ (20 mL×3).

The reaction mixture was washed with 5% sodium bicarbonate. The organic layer was dried (MgSO₄), filtered, and concentrated in vacuo. The crude product was purified by flash column chromatography to give H12.

### General Procedure for the Synthesis of H13

To a solution of G3 (1.1 mmol), an amine (1.0 mmol) in CH₂Cl₂ (5 mL) were added sodium triacetoxyborohydride (2.0 mmol) and glacial acetic acid (2.0 mmol) at room temperature for 20 h. The reaction mixture was added saturated ammonium chloride solution and stirred for 10 min. The reaction mixture was extracted with CH₂Cl₂ (20 mL). The organic layer was dried (MgSO₄), filtered, and concentrated in vacuo. The crude product was purified by flash column chromatography to give H13.

### Ethyl 9-methyl-4-oxo-4H-pyrido[1,2-a]pyrimidine-3-carboxylate (174)

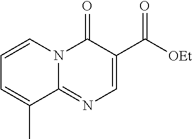 [SMILES: CCOC(=O)c1cnc2c(C)cccn2c1=O]

¹H NMR (400 MHz, CDCl₃) δ 1.39 (t, J=7.2 Hz, 3H), 2.62 (s, 3H), 4.39 (q, J=7.2 Hz, 2H), 7.20 (t, J=7.2 Hz, 1H), 7.77 (d, J=7.2 Hz, 1H), 9.05 (s, 1H), 9.16 (d, J=7.2 Hz, 1H); ¹³C NMR (100 MHz, CDCl₃) 14.6, 18.2, 61.2, 105.3, 116.8, 127.0, 135.9, 138.2, 155.3, 158.4, 165.0, 189.1.

### 9-Methyl-4-oxo-4H-pyrido[1,2-a]pyrimidine-3-carboxylic acid (175)

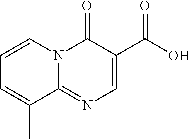

¹H NMR (400 MHz, CDCl₃) δ 2.56 (s, 3H), 7.12 (t, J=6.8 Hz, 1H), 7.79 (d, J=6.8 Hz, 1H), 8.87 (s, 1H), 9.21 (d, J=7.2 Hz), 14.13 (s, 1H); ¹³C NMR (100 MHz, CDCl₃). 818.3, 110.9, 117.1, 128.1, 137.6, 141.1, 155.0, 157.1, 158.3, 171.3.

### 3-(Hydroxymethyl)-9-methyl-4H-pyrido[1,2-a]pyrimidin-4-one (176)

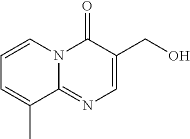

¹H NMR (400 MHz, CDCl₃) δ 2.51 (s, 3H), 3.27 (brs, 1H), 4.66 (s, 2H), 7.01 (t, J=6.8 Hz, 1H), 7.51 (d, J=6.8 Hz, 1H), 8.32 (s, 1H), 8.87 (s, 1H); ¹³C NMR (100 MHz, CDCl₃) 18.2, 44.1, 111.2, 117.9, 127.1, 135.7, 139.8, 153.9, 155.6, 158.2.

### 9-Methyl-4-oxo-4H-pyrido[1,2-a]pyrimidine-3-carbaldehyde (177)

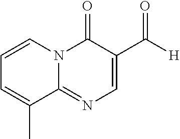

¹H NMR (400 MHz, CDCl₃) δ 2.63 (s, 3H), 7.29 (t, J=7.2 Hz, 1H), 7.86 (d, J=7.2 Hz, 1H), 8.85 (s, 1H), 9.14 (d, J=7.2 Hz, 1H), 10.33 (s, 1H); ¹³C NMR (100 MHz, CDCl₃). δ 18.2, 110.9, 117.5, 126.7, 136.5, 139.5, 153.1, 155.6, 158.1, 188.5.

### Ethyl 2-hydroxy-4-oxo-4H-pyrido[1,2-a]pyrimidin-2-carboxylate (178)

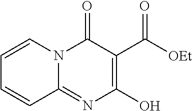 [SMILES: CCOC(=O)c1c(O)nc2ccccn2c1=O]

¹H NMR (400 MHz, CDCl₃) δ 1.42 (t, J=7.2 Hz 3H), 4.45 (q, J=7.2 Hz, 2H), 7.13 (ddd, J=1.2, 6.8, 7.2 Hz, 1H), 7.49 (d, J=8.8 Hz, 1H), 7.82-7.86 (m, 1H), 9.00 (d, J=7.2 Hz, 1H), 13.64 (brs, 1H, NH); ¹³C NMR (100 MHz, CDCl₃) δ 14.2, 62.3, 87.1, 115.3, 125.1, 128.7, 140.3, 148.4, 152.6, 155.5, 171.7.

### 2-Hydroxy-4-oxo-4H-pyrido[1,2-a]pyrimidine-3-carboxylic acid (179)

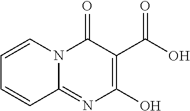 [SMILES: O=C(O)c1c(O)nc2ccccn2c1=O]

¹H NMR (400 MHz, CDCl₃) δ 2.50 (s, 3H), 6.70 (d4, J=6.8, 7.2 Hz, 1H), 7.15 (dd, J=7.2, 7.2 Hz, 1H), 7.37, (dd, J=7.2, 7.6 Hz, 1H), 7.65 (d, J=6.8 Hz, 1H), 7.76 (d, J=8.4 Hz, 1H), 8.76 (d, J=7.2 Hz, 1H), 11.70 (brs, 1H), 14.31 (s, 1H).

### Ethyl 4-oxo-2-(phenylamino)-4H-pyrido[1,2-a]pyrimidine-3-carboxylate (180)

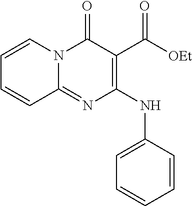 [SMILES: CCOC(=O)c1c(Nc2ccccc2)nc2ccccn2c1=O]

¹H NMR (400 MHz, CDCl₃) δ 1.45 (t, J=7.2 Hz, 3H), 4.44 (q, J=7.2 Hz, 2H), 6.93 (dd, J=6.8, 6.8 Hz, 1H), 7.29-7.36 (m, 3H), 7.65-7.68 (m, 3H), 8.97 (d, J=7.2 Hz, 1H), 11.39 (brs, 1H); ¹³C NMR (100 MHz, CDCl₃) δ 14.4, 61.0, 85.5, 113.6, 122.5, 124.2, 124.5, 128.4, 128.6, 138.4, 139.0, 151.6, 155.9, 159.5, 169.6.

### Ethyl 2-(3-hydroxyphenylamino)-4-oxo-4H-pyrido[1,2-a]pyrimidine-3-carboxylate (181)

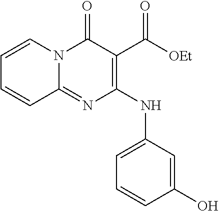 [SMILES: CCOC(=O)c1c(Nc2cccc(O)c2)nc2ccccn2c1=O]

¹H NMR (400 MHz, CDCl₃+CD₃OD) δ 1.38 (t, J=7.0 Hz, 3H), 4.37 (q, J=7.2 Hz, 2H), 6.56-6.58 (m, 1H), 6.92 (dd, J=6.8, 7.2 Hz, 1H0, 7.05 (d, J=8.4 Hz, 1H0, 7.12 (dd, J=8.0, 8.0 Hz, 1H), 7.26 (m, 1H), 7.31 (d, J=8.8 Hz, 1H), 7.66 (dd, J=7.2, 7.6 Hz, 1H), 8.90 (d, J=7.2 Hz, 1H), 11.22 (brs, 1H).

### Ethyl 2-(2-hydroxyphenylamino)-4-oxo-4H-pyrido[1,2-a]pyrimidine-3-carboxylate (182)

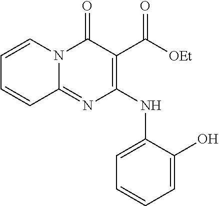 [SMILES: CCOC(=O)c1c(Nc2ccccc2O)nc2ccccn2c1=O]

¹H NMR (400 MHz, CDCl₃) δ 1.45 (t, J=7.2 Hz, 3H), 4.45 (q, J=6.8 Hz, 2H), 6.90 (dd, J=7.2, 8.0 Hz, 1H), 7.05-7.08 (m, 2H), 7.13 (dd, J=7.6, 8.4 Hz, 2H), 7.37 (d, J=8.4 Hz, 1H), 7.81 (dd, J=7.6, 8.0 Hz, 1H), 9.03 (d, J=6.8 Hz, 1H), 11.52 (brs, 1H); ¹³C NMR (100 MHz, CDCl₃) 14.4, 61.3, 114.7, 120.1, 120.5, 122.9, 124.4, 127.0, 127.1, 129.0, 140.8, 149.3, 151.1, 158.6, 169.5.

### Ethyl 2-(3-nitrophenylamino)-4-oxo-4H-pyrido[1,2-a]pyrimidine-3-carboxylate (183)

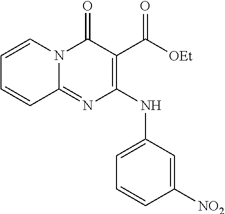 [SMILES: CCOC(=O)c1c(Nc2cccc([N+](=O)[O-])c2)nc2ccccn2c1=O]

¹H NMR (400 MHz, CDCl₃) δ 1.46 (t, J=6.4 Hz, 3H), 4.45 (q, J=7.2 Hz, 2H), 7.05 (ddd, J=1.2, 6.8, 6.8 Hz, 1H), 7.43 (d, J=8.8 Hz, 1H), 7.47 (dd, J=8.0, 8.4 Hz, 2H), 7.77-7.82 (m, 2H), 7.93-7.96 (m, 1H), 8.97-8.98 (m, 1H), 9.04 (dd, J=0.8, 7.2 Hz, 1H), 11.74 (brs, 1H); ¹³C NMR (100 MHz, CDCl₃) 14.4, 61.3, 86.1, 114.5, 116.9, 118.4, 124.7, 127.4, 128.6, 129.2, 139.8, 148.5, 151.5, 155.7, 159.5, 169.6.

### Ethyl 4-oxo-2-phenoxy-4H-pyrido[1,2-a]pyrimidine-3-carboxylate (184)

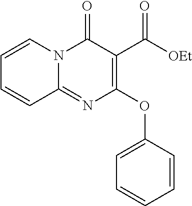 [SMILES: CCOC(=O)c1c(Oc2ccccc2)nc2ccccn2c1=O]

¹H NMR (400 MHz, CDCl₃) δ 1.38 (t, J=7.2 Hz, 3H), 4.42 (q, J=7.2 Hz, 2H), 7.15-7.17 (m, 3H), 7.24 (d, J=6.4 Hz, 1H), 7.36-7.41 (m, 3H), 7.77 (ddd, J=1.6, 6.8, 6.8 Hz, 1H), 9.10 (dd, J=0.8, 6.8 Hz, 1H)); ¹³C NMR (100 MHz, CDCl₃) δ 14.2, 61.3, 115.7, 121.8, 125.3, 128.5, 129.2, 128.7, 150.3, 152.5, 156.7, 164.1, 165.0.

### Ethyl 2-(3-fluorophenoxy)-4-oxo-4H-pyrido[1,2-a]pyrimidine-3-carboxylate (185)

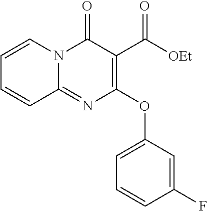 [SMILES: CCOC(=O)c1c(Oc2cccc(F)c2)nc2ccccn2c1=O]

¹H NMR (400 MHz, CDCl₃) δ 1.37 (t, J=7.0 Hz, 3H), 4.40 (q, J=6.8 Hz, 2H), 6.91-6.98 m, 3H), 7.19 (ddd, J=1.2, 7.2, 7.2 Hz, 1H), 7.32-7.36 (m, 1H), 7.39 (d, J=8.8 Hz, 1H), 7.78-7.82 (m, 1H), 9.10 (d, J=6.8 Hz, 1H); ¹³C NMR (100 MHz, CDCl₃) δ 14.2, 61.4, 94.6, 109.8, 110.0, 112.2, 112.4, 115.9, 117.5, 117.6, 125.3, 128.5, 129.8, 129.9, 139.9, 150.3, 153.3, 156.6, 161.6, 163.8, 164.0, 164.5.

### Ethyl 4-oxo-2-(3-(trifluoromethyl)phenoxy)-4H-pyrido[1,2-a]pyrimidine-3-carboxylate (186)

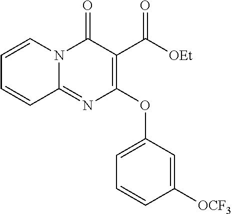 [SMILES: CCOC(=O)c1c(Oc2cccc(OC(F)(F)F)c2)nc2ccccn2c1=O]

¹H NMR (400 MHz, CDCl₃) δ 1.39 (t, J=7.2 Hz, 3H), 4.43 (q, J=7.0 Hz 2H), 7.21 (dd, J=6.8, 6.8 Hz, 1H), 7.38 (d, J=8.0 Hz, 2H), 7.47-7.52 (m, 2H), 7.81 (dd, J=7.2, 8.4 Hz, 1H), 9.12 (d, J=6.8 Hz, 1H).

### Methyl 2-chloro-9-methyl-4-oxo-4H-pyrido[1,2-a]pyrimidine-3-carboxylate (187)

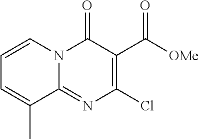 [SMILES: COC(=O)c1c(Cl)nc2c(C)cccn2c1=O]

¹H NMR (400 MHz, CDCl₃) δ 2.56 (s, 3H), 3.93 (s, 3H), 7.19 (t, J=7.2 Hz, 1H), 7.75 (d, J=6.8 Hz, 1H), 8.91 (d, J=7.2 Hz, 1H); ¹³C NMR (100 MHz, CDCl₃) δ 17.1, 52.8, 108.0, 116.7, 126.1, 134.9, 138.3, 149.1, 155.1, 155.2, 164.2.

### Methyl 2-(3-chlorophenylamino)-9-methyl-4-oxo-4H-pyrido[1,2-a]pyrimidine-3-carboxylate (188)

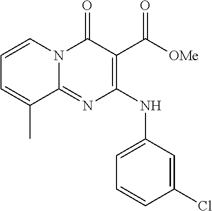 [SMILES: COC(=O)c1c(Nc2cccc(Cl)c2)nc2c(C)cccn2c1=O]

¹H NMR (400 MHz, CDCl₃) δ 2.51 (s, 3H), 3.99 (s, 3H), 6.94 (t, J=7.0 Hz, 1H), 7.09 (d, J=7.6 Hz, 1H), 7.27 (d, J=8.4 Hz, 1H), 7.41 (d, J=8.0 Hz, 1H), 7.64 (d, J=6.8 Hz, 1H), 8.18 (s, 1H), 8.91 (d, J=7.2 Hz, 1H), 11.52 (br s, 1H); ¹³C NMR (100 MHz, CDCl₃) δ 18.0, 52.1, 85.3, 113.7, 119.6, 121.9, 123.5, 126.4, 129.4, 133.2, 134.1, 138.4, 139.9, 151.0, 156.2, 158.6, 170.1.

### Methyl 2-(3-chlorobenzylamino)-9-methyl-4-oxo-4H-pyrido[1,2-a]pyrimidine-3-carboxylate (189)

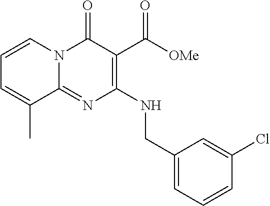 [SMILES: COC(=O)c1c(NCc2cccc(Cl)c2)nc2c(C)cccn2c1=O]

¹H NMR (400 MHz, CDCl₃) δ 2.35 (s, 3H), 3.92 (s, 3H), 4.77 (d, J=6.0 Hz, 2H), 6.80 (t, J=6.8 Hz, 1H), 7.20-7.24 (m, 3H), 7.34 (s, 3H), 7.50 (d, J=6.8 Hz, 1H), 8.82 (d, J=7.2 Hz, 1H), 9.69 (br s, 1H); ¹³C NMR (100 MHz, CDCl₃). δ 17.8, 44.4, 51.8, 84.6, 112.6, 125.5, 126.4, 127.2, 127.7, 129.7, 132.7, 134.3, 137.6, 141.1, 151.3, 156.4, 160.8, 170.1.

### Ethyl 2-hydroxy-4-oxo-6,7,8,9-tetrahydro-4H-pyrido[1,2-a]pyrimidine-3-carboxylate (190)

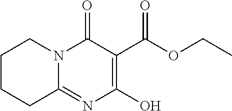 [SMILES: CCOC(=O)c1c(O)nc2n(c1=O)CCCC2]

¹H NMR (400 MHz, CDCl₃) δ 1.36 (t, J=7.2 Hz, 3H), 1.82-1.93 (m, 4H), 2.86 (t, J=6.8 Hz, 2H), 3.84 (t, J=6.0 Hz, 2H), 4.39 (q, J=7.2 Hz, 2H); ¹³C NMR (100 MHz, CDCl₃) δ 14.4, 18.9, 21.9, 32.2, 43.0, 62.4, 90.9, 159.8, 165.1., 171.7, 173.5.

### Ethyl 4-oxo-2-(tosyloxy)-6,7,8,9-tetrahydro-4H-pyrido[1,2-a]pyrimidine-3-carboxylate (191)

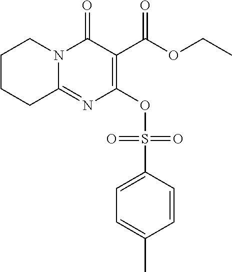 [SMILES: CCOC(=O)c1c(OS(=O)(=O)c2ccc(C)cc2)nc2n(c1=O)CCCC2]

¹H NMR (400 MHz, CDCl₃) δ 1.25 (t, J=7.2 Hz, 3H), 1.79-1.91 (m, 4H), 2.41 (s, 3H), 2.79 (t, J=6.4 Hz, 2H), 3.84 (t, J=6.4 Hz, 2H), 4.25 (q, J=7.2 Hz, 2H), 7.31 (d, J=8.0 Hz, 2H), 7.89 (d, J=8.0 Hz, 2H); ¹³C NMR (100 MHz, CDCl₃) δ 14.2, 18.8, 21.6, 21.9, 31.8, 43.6, 61.9, 104.2, 129.1, 129.7, 134.2, 145.8, 159.4, 160.8, 162.0, 162.2.

### Ethyl 4-oxo-2-(phenylamino)-6,7,8,9-tetrahydro-4H-pyrido[1,2-a]pyrimidine-3-carboxylate (192)

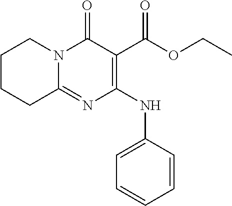 [SMILES: CCOC(=O)c1c(Nc2ccccc2)nc2n(c1=O)CCCC2]

¹H NMR (400 MHz, CDCl₃) δ 1.40 (t, J=7.2 Hz, 3H), 1.80-1.92 (m, 4H), 2.80 (t, J=6.8 Hz, 2H), 3.87 (t, J=6.0 Hz, 2H), 4.36 (q, J=7.2 Hz, 2H), 7.08 (t, J=7.2 Hz, 1H), 7.29 (t, J=7.2 Hz, 2H), 7.53 (d, J=7.6 Hz, 2H), 11.2 (s, 1H); ¹³C NMR (100 MHz, CDCl₃) δ 14.6, 19.2, 22.2, 32.2, 42.4, 61.0, 88.4, 122.9, 124.4, 128.8, 138.4, 160.5, 160.8, 162.2, 169.8.

### Ethyl 2-(3-chlorophenylamino)-4-oxo-6,7,8,9-tetrahydro-4H-pyrido[1,2-a]pyrimidine-3-carboxylate (193)

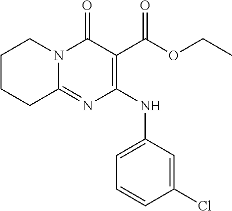 [SMILES: CCOC(=O)c1c(Nc2cccc(Cl)c2)nc2n(c1=O)CCCC2]

¹H NMR (400 MHz, CDCl₃) δ 1.32 (t, J=7.2 Hz, 3H), 1.76-1.88 (m, 4H), 2.76 (t, J=6.8 Hz, 2H), 3.78 (t, J=6.0 Hz, 2H), 4.29 (q, J=7.06 (d4, J=7.2 Hz, 2H), J=1.2, 8.0 Hz, 1H), 7.27 (t, J=8.0 Hz, 1H), 7.51 (dd, J=1.2, 8.0 Hz, 1H), 7.58 (d, J=2.0 Hz, 1H); ¹³C NMR (100 MHz, CDCl₃) δ 14.3, 18.6, 22.1, 32.1, 42.6, 61.1, 81.4, 111.2, 111.7, 113.0, 128.4, 140.4, 149.6, 158.7, 161.12, 163.2, 170.4

### Ethyl 4-oxo-2-(3-(trifluoromethyl)phenylamino)-6,7,8,9-tetrahydro-4H-pyrido[1,2-a]pyrimidine-3-carboxylate (194)

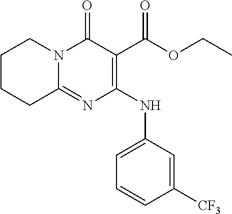 [SMILES: CCOC(=O)c1c(Nc2cccc(C(F)(F)F)c2)nc2n(c1=O)CCCC2]

¹H NMR (400 MHz, CDCl₃) δ 1.45 (t, J=7.2 Hz, 3H), 1.88-1.97 (m, 4H), 2.87 (t, J=6.4 Hz, 2H), 3.93 (t, J=5.6 Hz, 2H), 4.41 (q, J=7.2 Hz, 2H), 7.35 (t, J=7.2 Hz, 1H), 7.35 (d, J=7.6 Hz, 1H), 7.67 (d, J=7.6 Hz, 1H), 8.05 (s, 1H), 11.2 (s, 1H);

### Ethyl 2-(2-hydroxyphenylamino)-4-oxo-6,7,8,9-tetrahydro-4H-pyrido[1,2-a]pyrimidine-3-carboxylate (195)

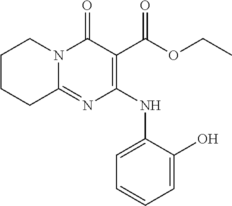 [SMILES: CCOC(=O)c1c(Nc2ccccc2O)nc2n(c1=O)CCCC2]

¹H NMR (400 MHz, CDCl₃) δ 1.40 (t, J=7.2 Hz, 3H), 1.81-1.94 (m, 4H), 2.65 (t, J=6.8 Hz, 2H), 3.65 (t, J=6.0 Hz, 2H), 4.18 (q, J=6.8 Hz, 2H), 6.85 (t, J=7.2 Hz, 1H), 7.00 (d, J=7.2 Hz, 1H), 7.06-7.12 (m, 2H), 9.98 (s, 1H), 11.3 (s, 1H); ¹³C NMR (100 MHz, CDCl₃) δ 14.6, 18.8, 21.9, 31.6, 42.6, 61.3, 88.4, 120.2, 120.7, 124.5, 127.1, 127.2, 149.1, 159.4, 159.5, 163.0, 169.6.

### Ethyl 2-(3-hydroxyphenylamino)-4-oxo-6,7,8,9-tetrahydro-4H-pyrido[1,2-a]pyrimidine-3-carboxylate (196)

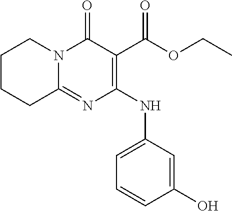 [SMILES: CCOC(=O)c1c(Nc2cccc(O)c2)nc2n(c1=O)CCCC2]

¹H NMR (400 MHz, CDCl₃+MeOD−d₄) δ 1.26 (t, J=7.2 Hz, 3H), 1.71-1.81 (m, 4H), 2.72 (t, J=6.4 Hz, 2H), 3.74 (t, J=6.4 Hz, 2H), 4.23 (q, J=7.2 Hz, 2H), 6.47 (d, J=7.6 Hz, 1H), 6.88 (d, J=8.0 Hz, 1H), 6.99 (d, J=8.0 Hz, 1H), 7.02 (t, J=2.0 Hz, 1H); ¹³C NMR (100 MHz, CDCl₃+MeOD−d₄) δ 14.2, 18.8, 21.9, 31.8, 42.4, 60.9, 79.8, 109.8, 111.6, 114.0, 129.4, 139.4, 149.7, 159.3, 160.2, 163.1, 169.6

### Ethyl 2 (4-hydroxyphenylamino)-4-oxo-6,7,8,9-tetrahydro-4H-pyrido[1,2-a]pyrimidine-3-carboxylate (197)

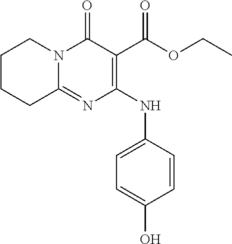 [SMILES: CCOC(=O)c1c(Nc2ccc(O)cc2)nc2n(c1=O)CCCC2]

¹H NMR (400 MHz, DMSO-d₆) δ 1.21 (t, J=7.2 Hz, 3H), 1.67-1.80 (m, 4H), 2.65 (t, J=6.8 Hz, 2H), 3.65 (t, J=6.0 Hz, 2H), 4.18 (q, J=7.2 Hz, 2H), 6.68 (d, J=8.8 Hz, 2H), 7.25 (d, J=8.8 Hz, 2H), 9.29 (s, 1H), 10.7 (s, 1H); ¹³C NMR (100 MHz, CDCl₃) δ 14.9, 18. 9, 21.9, 32.1, 42.3, 60.4, 87.2, 115.7, 125.0, 130.1, 154.9, 159.4, 160.6, 163.3, 169.6.

### 2-(3-Chloro-4-fluorophenylamino)-9-methoxy-N-methyl-4-oxo-4H-pyrido[1,2-a]pyrimidine-3-carboxamide (198)

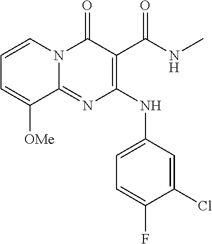 [SMILES: CNC(=O)c1c(Nc2ccc(F)c(Cl)c2)nc2c(OC)cccn2c1=O]

mp=218° C. (decomp.); ¹H NMR (400 MHz, CDCl₃) δ 2.97 (d, J=4.8 Hz, 3H), 4.41 (s, 3H), 6.89 (dd, J=7.2 Hz, 7.2 Hz, 1H), 6.97 (dd, J=1.2 Hz, 8.0 Hz, 1H), 7.05 (dd, J=8.8 Hz, 8.8 Hz, 1H), 7.40-7.44 (m, 1H), 8.46-8.51 (m, 2H), 8.82 (d, J=2.0 Hz, 1H), 12.98 (s, 1H);

### (E)-2-(3-Chlorophenylamino)-3-((cyclohexylimino)methyl)-4H-pyrido[1,2-a]pyrimidin-4-one (199)

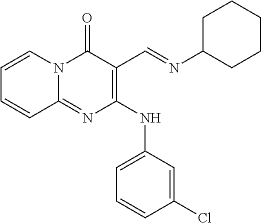 [SMILES: O=c1c(/C=N/C2CCCCC2)c(Nc2cccc(Cl)c2)nc2ccccn12]

¹H NMR (400 MHz, CDCl₃) δ 1.23-1.37 (m, 3H), 1.41-1.50 (m, 2H), 1.56-1.59 (m, 1H), 1.73-1.76 (m, 4H), 3.16-3.22 (m, 1H), 6.85 (ddd, J=1.2, 6.8, 6.8 Hz, 1H), 6.94 (ddd, J=0.8, 1.2, 8.0 Hz, 1H), 7.14 (dd, J=8.0, 8.0 Hz, 1H), 7.38 (ddd, J=0.8, 1.2, 8.0 Hz, 1H), 7.54-7.58 (m, 1H), 7.90-7.91 (m, 1H), 8.83 (s, 1H), 8.85 (dd, J=0.8, 1.2 Hz, 1H), 13.40 (brs, 1H); ¹³C NMR (100 MHz, CDCl₃) δ 24.4, 25.6, 34.9, 68.4, 91.6, 113.4, 119.2, 121.2, 123.0, 124.7, 127.6, 129.5, 134.2, 137.6, 140.8, 150.6, 156.3, 157.0, 158.3.

### (E)-2-(3-Chlorophenylamino)-3-((3-chlorophenylimino)methyl)-4H-pyrido[1,2-a]pyrimidin-4-one (200)

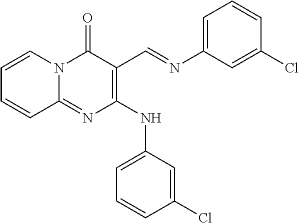 [SMILES: O=c1c(/C=N/c2cccc(Cl)c2)c(Nc2cccc(Cl)c2)nc2ccccn12]

¹H NMR (400 MHz, CDCl₃) δ 7.01 (dd, J=0.8, 1.2, 8.0 Hz, 1H), 7.28 (d, J=8.4 Hz, 1H), 7.29 (dd, J=2.0, 4.0 Hz, 1H), 7.33 (d, J=8.0 Hz, 1H), 7.44 (d, J=8.8 Hz, 1H), 7.52 (ddd, J=0.8, 1.2, 8.0 Hz, 1H), 7.17-7.76 (m, 1H), 8.02-8.04 (m, 1H), 8.98 (dd, J=0.8, 6.8 Hz, 1H), 9.17 (s, 1H), 12.94 (brs, 1H); ¹³C NMR (100 MHz, CDCl₃) δ 92.6, 114.0, 119.5, 119.8, 121.8, 123.9, 125.0, 125.7, 128.0, 129.7, 130.2, 134.4, 134.8, 138.7, 140.1, 151.3, 151.8, 157.0, 158.0, 158.9.

### 2-(3-Chlorophenylamino)-3-((cyclopentylamino)methyl)-4H-pyrido[1,2-a]pyrimidin-4-one (201)

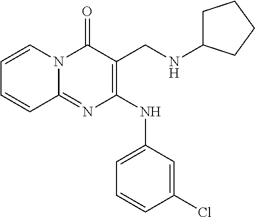 [SMILES: O=c1c(CNC2CCCC2)c(Nc2cccc(Cl)c2)nc2ccccn12]

¹H NMR (400 MHz, CDCl₃) δ 1.54-1.57 (m, 2H), 1.74-1.83 (m, 4H), 2.05-2.08 (m, 2H), 3.23-3.24 (m, 1H), 4.19 (s, 2H), 6.93-6.98 (m, 2H), 7.11-7.15 (m, 1H), 7.32 (d, J=8.4 Hz, 1H), 7.51 (dd, J=2.0, 8.4 Hz, 1H), 7.61-7.65 (m, 1H), 7.74-7.75 (m, 1H), 8.73 (d, J=7.2 Hz, 1H).

### 2-(3-Chlorophenylamino)-3-((cyclohexylamino)methyl)-4H-pyrido[1,2-a]pyrimidin-4-one (202)

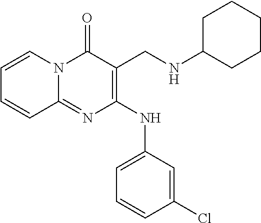 [SMILES: O=c1c(CNC2CCCCC2)c(Nc2cccc(Cl)c2)nc2ccccn12]

¹H NMR (400 MHz, CDCl₃) δ 1.20-1.35 (m, 4H), 1.66-1.72 (m, 2H), 1.86-1.89 (m, 2H), 2.23-2.39 (m, 2H), 3.12-3.18 (m, 1H), 6.93 (ddd, J=1.2, 6.8, 7.2 Hz, 1H), 6.99 (ddd, J=0.8, 1.2, 7.6 Hz, 1H), 7.20 (dd, J=8.0, 8.0 Hz, 1H), 7.25 (d, J=8.8 Hz, 1H), 7.52-7.57 (m, 1H), 7.61 (dd, J=1.2, 8.0 Hz, 1H), 7.84-7.85 (m, 1H), 8.76 (d, J=6.4 Hz, 1H), 9.77 (brs, 1H); ¹³C NMR (100 MHz, CDCl₃) δ 24.6, 25.0, 41.2, 57.9, 88.9, 114.6, 119.2, 121.1, 122.8, 124.6, 127.3, 129.4, 133.7, 137.3, 140.8, 149.6, 157.2, 158.8.

### 2-(3-Chlorophenylamino)-3-((cycloheptylamino)methyl)-4H-pyrido[1,2-a]pyrimidin-4-one (203)

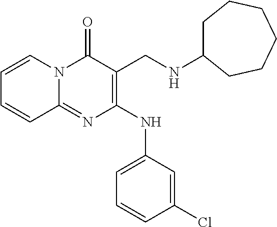 [SMILES: O=c1c(CNC2CCCCCC2)c(Nc2cccc(Cl)c2)nc2ccccn12]

¹H NMR (400 MHz, CDCl₃) δ 1.40-1.59 (m, 6H), 1.72-1.81 (m, 4H), 2.18-2.23 (m, 2H), 3.07-3.12 (m, 1H), 4.05 (m, 2H), 6.82 (ddd, J=1.2, 6.8, 6.8 Hz, 1H), 6.91 (dd, J=1.2, 8.0 Hz, 1H), 7.14 (dd, J=8.0, 8.0 Hz, 1H), 7.44-7.49 (m, 2H), 7.78-7.80 (m, 1H), 8.70 (d, J=6.8 Hz, 1H), 10.00 (brs, 1H); ¹³C NMR (100 MHz, CDCl₃) δ 23.8, 32.3, 41.5, 59.7, 89.7, 114.2, 118.7, 120.6, 122.4, 124.4, 127.2, 129.3, 133.7, 136.8, 140.9, 149.4, 157.2, 158.2.

### 2-(3-Chlorophenylamino)-3-((isopropylamino)methyl)-4H-pyrido[1,2-a]pyrimidin-4-one (204)

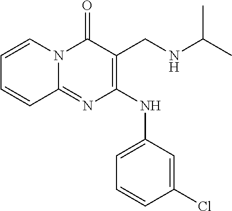 [SMILES: CC(C)NCc1c(Nc2cccc(Cl)c2)nc2ccccn2c1=O]

¹H NMR (400 MHz, CDCl₃) δ 1.25 (s, 3H), 1.26 (s, 3H), 2.30-3.06 (m, 1H), 4.05 (s, 2H), 6.87 (dd, J=6.4, 7.2 Hz, 1H), 6.95 (d, J=7.2 Hz, 1H), 7.17 (dd, J=8.0, 8.0 Hz, 1H), 7.32 (d, J=8.8 Hz, 1H), 7.41 (d, J=8.0 Hz, 1H), 7.54 (dd, J=7.2, 7.2 Hz, 1H), 7.81 (s, 1H), 8.83 (d, J=6.8 Hz, 1H); ¹³C NMR (100 MHz, CDCl₃) δ 22.1, 41.7, 48.9, 91.5, 113.7, 118.2, 120.1, 122.2, 124.6, 127.5, 129.5, 134.1, 136.2, 141.2, 149.5, 157.4, 157.8.

### 2-(3-Chlorophenylamino)-3-((cyclohexylamino)methyl)-8-(4-methylpiperazin-1-yl)-4H-pyrido[1,2-a]pyrimidin-4-one (205)

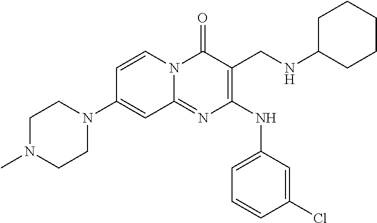 [SMILES: CN1CCN(c2ccn3c(=O)c(CNC4CCCCC4)c(Nc4cccc(Cl)c4)nc3c2)CC1]

¹H NMR (400 MHz, CDCl₃) δ 1.20-1.34 (m, 3H), 1.71-1.91 (m, 3H), 1.92-2.04 (m, 2H), 2.20 (s, 3H), 2.23-2.36 (m, 6H), 3.04-3.10 (m, 5H), 4.01 (s, 2H), 5.87 (s, 1H), 6.55 (s, J=8.0 hz, 1H), 6.90 (d, J=8.0 Hz, 1H), 7.14 (t, J=8.0 Hz, 1H), 7.62 (d, J=7.6 Hz, 1H), 7.84 (s, 1H), 8.46 (d, J=7.6 Hz, 1H), 9.59 (s, 1H); ¹³C NMR (100 MHz, CDCl₃) δ 24.9, 25.3, 30.2, 41.2, 46.1, 46.3, 54.2, 58.4, 86.2, 98.9, 106.5, 119.3, 121.0, 122.3, 128.3, 129.5, 133.9, 141.9, 150.8, 154.8, 157.7, 158.9.

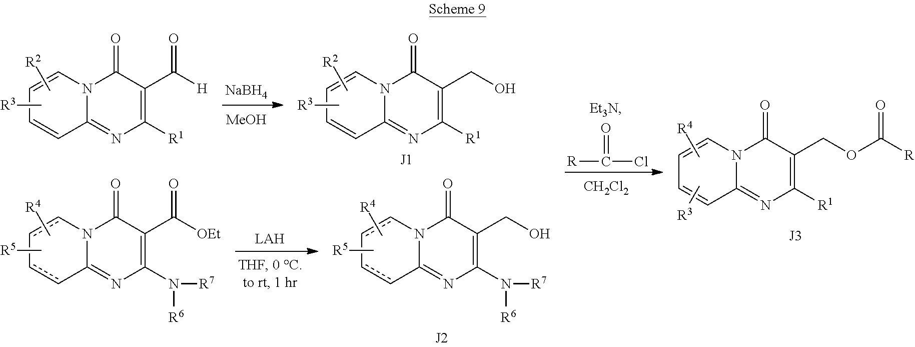

### General Procedure for the Synthesis of J1

To a solution of an aldehyde (0.9 mmol) in methanol (0.5 mL) was added NaBH₄ (1.35 mmol) at room temperature. After stirring 1 h, the reaction mixture was diluted with methylene chloride (10 mL) and washed with brine (10 ml). The organic layer was dried over MgSO₄ and concentrated in vacuo. The crude product was purified by recrystallization from a mixture of hexanes and ethyl acetate to give J1.

### General Procedure for the Synthesis of J2

To a stirred solution of an ester (0.06 mmol) in THF (1.0 mL) was added LiAlH₄ (0.09 mmol). The reaction mixture was stirred at room temperature for 1 hr. After reaction was completed, H₂O (0.1 mL) was added dropwise. The reaction mixture was filtered off and concentrated in vacuo. The crude product was purified by flash column chromatography to give J2.

### General Procedure for the Synthesis of J3

To a stirred solution of J1 or J2 (0.19 mmol) in CH₂Cl₂ (0.6 mL) was added triethylamine (0.38 mmol) and a benzoyl chloride (0.28 mmol) at 0° C. The reaction mixture was stirred at room temperature for 1 h. After reaction was completed, the mixture was diluted with CH₂Cl₂ (10 mL) and washed with brine (10 ml). The organic layer was dried over anhydrous MgSO₄ and concentrated in vacuo. The crude product was purified by flash column chromatography (Hexane:EtOAc=2:1) to give J3.

### 3-(Hydroxymethyl)-2-(phenylamino)-4H-pyrido[1,2-a]pyrimidin-4-one (206)

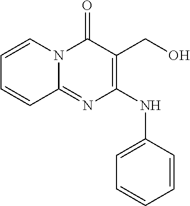 [SMILES: O=c1c(CO)c(Nc2ccccc2)nc2ccccn12]

¹H NMR (400 MHz, CDCl₃+CD₃OD) δ 4.80 (s, 2H), 6.87-6.90 (m, 1H), 8.03 (dd, J=7.2, 7.6 Hz, 1H), 7.27 (dd, J=7.6, 8.0 Hz, 2H), 7.53-7.58 (m, 3H), 8.36 (brs, 1H), 8.82 (d, J=6.8 Hz, 1H); ¹³C NMR (100 MHz, CDCl₃+CD₃OD) δ 56.0, 94.80, 94.85, 113.8, 121.1, 121.2, 123.2, 123.3, 124.5, 127.5, 128.6, 136.4, 138.9, 139.0, 149.7, 157.1, 158.0, 158.1.

### 2-(3-Chlorophenylamino)-3-(hydroxy methyl)-4H-pyrido-[1,2-a]pyrimidin-4-one (207)

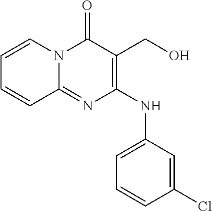 [SMILES: O=c1c(CO)c(Nc2cccc(Cl)c2)nc2ccccn12]

¹H NMR (400 MHz, CDCl₃) δ 4.95 (d, J=6.4 Hz, 2H), 6.93 (t, J=6.8 Hz, 1H), 7.05 (d, J=8.0 Hz, 1H), 7.38 (t, J=4.4 Hz, 2H), 7.42 (s, 1H), 7.63 (t, J=6.8 Hz, 1H), 7.81 (t, J=1.6 Hz, 1H), 8.20 (s, 1H), 8.92 (d, J=7.2 Hz, 1H),

### 2-(3-Fluorophenylamino)-3-(hydroxymethyl)-4H-pyrido[1,2-a]pyrimidine-3-carbaldehyde (208)

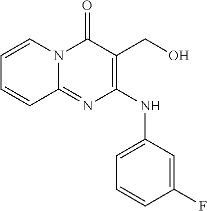 [SMILES: O=c1c(CO)c(Nc2cccc(F)c2)nc2ccccn12]

¹H NMR (400 MHz, CDCl₃) δ 4.94 (s, 2H), 6.94 (t, J=6.0 Hz, 2H), 7.17 (d, J=8.0 Hz, 1H), 7.43 (d, J=8.8 Hz, 2H), 7.63 (t, J=7.2 Hz, 2H), 7.70 (d, J=9.2 Hz, 1H), 8.26 (s, 1H), 8.93 (d, J=7.2 Hz, 1H).

### 3-(Hydroxymethyl)-2-(3-(trifluoromethyl)phenylamino)-4H-pyrido[1,2-a]pyrimidin-4-one (209)

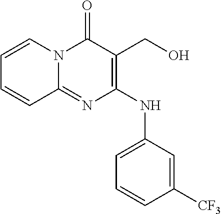 [SMILES: O=c1c(CO)c(Nc2cccc(C(F)(F)F)c2)nc2ccccn12]

¹H NMR (400 MHz, CDCl₃) δ 4.99 (s, 2H), 6.99 (d, J=6.0 Hz, 2H), 7.32 (d, J=8.0 Hz, 1H), 7.43 (d, J=7.6 Hz, 2H), 7.69 (brs, 2H), 8.06 (s, 1H), 8.27 (s, 1H), 8.96 (d, J=7.6 Hz, 1H).

### 3-(Hydroxymethyl)-2-(3-(trifluoromethoxy)phenylamino)-4H-pyrido[1,2-a]pyrimidin-4-one (210)

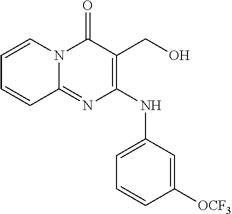 [SMILES: O=c1c(CO)c(Nc2cccc(OC(F)(F)F)c2)nc2ccccn12]

¹H NMR (400 MHz, CDCl₃) δ 4.95 (d, J=6.4 Hz, 2H), 6.84 (t, J=6.8 Hz, 1H), 6.92 (d, J=6.8 Hz, 1H), 7.30-7.34 (m, 3H), 7.59 (t, J=7.2 Hz, 1H), 7.86 (s, 1H), 8.36 (s, 1H), 8.87 (d, J=6.4 Hz, 1H),

### Methyl 3-(3-(hydroxymethyl)-4-oxo-4H-pyrido[1,2-a]pyrimidin-2-ylamino)benzoate (211)

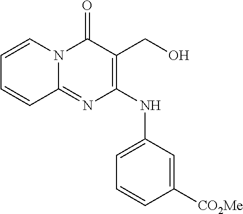 [SMILES: COC(=O)c1cccc(Nc2nc3ccccn3c(=O)c2CO)c1]

¹H NMR (400 MHz, CDCl₃) δ 3.92 (s, 3H), 4.99 (d, J=6.4 Hz, 2H), 6.96 (t, J=7.2 Hz, 1H), 7.38-7.42 (m, 2H), 7.63 (t, J=7.8 Hz, 1H), 7.75 (d, J=7.6 Hz, 1H), 7.88 (d, J=8.0 Hz, 1H), 8.21 (s, 1H), 8.25 (brs, 1H), 8.96 (d, J=7.6 Hz, 1H).

### 3-(3-(hydroxymethyl)-4-oxo-4H-pyrido[1,2-a]pyrimidin-2-ylamino)benzoic acid (212)

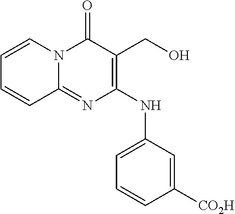 [SMILES: O=C(O)c1cccc(Nc2nc3ccccn3c(=O)c2CO)c1]

¹H NMR (400 MHz, CDCl₃) δ 4.73 (s, 1H), 5.74 (s, 2H), 7.19 (t, J=7.2 Hz, 1H), 7.38-7.42 (m, 2H), 7.45 (d, J=7.6 Hz, 1H), 7.86 (t, J=8.4 Hz, 1H), 8.00 (d, J=8.0 Hz, 1H), 8.19 (s, 1H), 8.82 (s, 1H), 8.89 (d, J=6.8 Hz, 1H).

### 2-(4-Chlorophenylamino)-3-(hydroxymethyl)-4H-pyrido[1,2-a]pyrimidin-4-one (213)

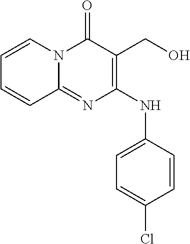 [SMILES: O=c1c(CO)c(Nc2ccc(Cl)cc2)nc2ccccn12]

¹H NMR (400 MHz, DMSO) δ 4.05 (d, J=7.2 Hz, 2H), 7.37 (d, J=8.8 Hz, 2H), 7.44 (d, J=8.8 Hz, 1H), 7.75 (d, J=6.8 Hz, 2H), 7.88 (t, J=8.8 Hz, 1H), 8.81 (s, 1H), 8.88 (d, J=6.4 Hz, 1H).

### 2-(2-Chlorophenylamino)-3-(hydroxymethyl)-4H-pyrido[1,2-a]pyrimidin-4-one (214)

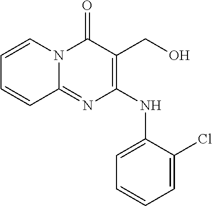 [SMILES: O=c1c(CO)c(Nc2ccccc2Cl)nc2ccccn12]

¹H NMR (400 MHz, CDCl₃) δ 5.01 (d, J=5.6 Hz, 2H), 6.97-7.01 (m, 3H), 7.26-7.29 (m, 1H), 7.42 (t, J=8.8 Hz, 2H), 7.66 (t, J=7.2 Hz, 1H), 8.41 (t, J=5.2 Hz, 1H), 8.53 (s, 1H), 8.99 (d, J=6.8 Hz, 1H).

### 3-(Hydroxymethyl)-2-(3-hydroxyphenylamino)-4H-pyrido[1,2-a]pyrimidin-4-one (215)

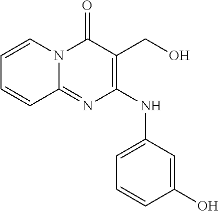 [SMILES: O=c1c(CO)c(Nc2cccc(O)c2)nc2ccccn12]

¹H NMR (400 MHz, CDCl₃+CD₃OD) δ 4.81 (s, 2H), 6.53 (d, J=8.0 Hz, 1H), 6.99 (dd, J=6.8, 6.8 Hz, 1H), 7.04 (d, J=8.0 Hz, 1H), 7.12 (dd, J=6.8, 6.8 Hz, 1H), 7.18 (s, 1H), 7.42 (d, J=9.6 Hz, 1H), 7.64 (dd, J=6.8, 8.8 Hz, 1H), 8.88 (d, J=7.2 Hz, 1H).

### 3-(Hydroxymethyl)-2-(4-hydroxyphenylamino)-4H-pyrido[1,2-a]pyrimidin-4-one (216)

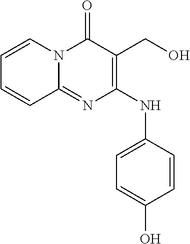 [SMILES: O=c1c(CO)c(Nc2ccc(O)cc2)nc2ccccn12]

¹H NMR (400 MHz, CD₃OD) δ 4.83 (s, 2H), 6.77 (dd, J=2.0, 8.8 Hz, 2H), 7.04 (dd, J=6.8, 6.8 Hz, 1H), 7.32 (d, J=8.8 Hz, 1H), 7.34-7.67 (m, 2H), 7.67-7.73 (m, 1H), 8.84 (d, J=6.8 Hz, 1H).

### 3-(Hydroxymethyl)-2-(2-hydroxyphenylamino)-4H-pyrido[1,2-a]pyrimidin-4-one (217)

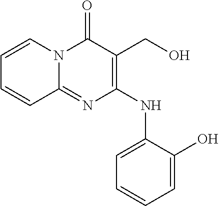 [SMILES: O=c1c(CO)c(Nc2ccccc2O)nc2ccccn12]

¹H NMR (400 MHz, CDCl₃+CD₃OD) δ 3.71 (s, 1H), 4.86 (s, 2H), 6.88 (ddd, J=1.6, 7.6, 8.0 Hz, 1H), 6.93 (dd, J=1.6, 8.0 Hz, 1H), 6.98 (ddd, J=1.6, 7.2, 8.0 Hz, 1H (, 7.05 (ddd, J=1.2, 6.8, 6.8 Hz, 1H), 7.43 (d, J=8.8 Hz, 1H), 7.69-7.73 (m, 2H), 8.91 (dd, J=0.8, 6.8 Hz, 1H).

### 2-(2,6-Dichlorophenylamino)-3-(hydroxymethyl)-4H-pyrido[1,2-a]pyrimidin-4-one (218)

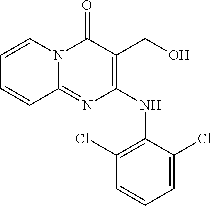 [SMILES: O=c1c(CO)c(Nc2c(Cl)cccc2Cl)nc2ccccn12]

¹H NMR (400 MHz, CDCl₃) δ 5.03 (d, J=6.0 Hz, 2H), 6.96 (t, J=7.2 Hz, 1H), 7.16 (t, J=7.6 Hz, 2H), 7.2 (s, 1H), 7.39 (d, J=8.0 Hz, 2H), 7.56 (t, J=7.6 Hz, 1H), 7.77 (s, 1H), 8.96 (d, J=7.2 Hz, 1H).

### 2-(3,5-Dichlorophenylamino)-3-(hydroxymethyl)-4H-pyrido[1,2-a]pyrimidin-4-one (219)

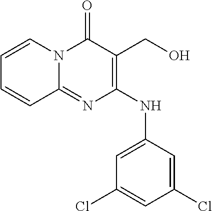 [SMILES: O=c1c(CO)c(Nc2cc(Cl)cc(Cl)c2)nc2ccccn12]

¹H NMR (400 MHz, CDCl₃) δ 4.97 (d, J=6.0 Hz, 2H), 7.01-7.04 (m, 2H), 7.50 (t, J=6.8 Hz, 1H), 7.60 (s, 2H), 7.71 (t, J=8.4 Hz, 2H), 8.24 (s, 1H), 8.98 (d, J=7.2 Hz, 1H).

### 2-(3,5-Difluorophenylamino)-3-(hydroxymethyl)-4H-pyrido[1,2-a]pyrimidin-4-one (220)

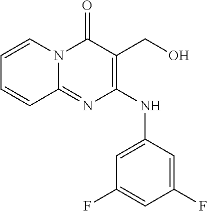 [SMILES: O=c1c(CO)c(Nc2cc(F)cc(F)c2)nc2ccccn12]

¹H NMR (400 MHz, CDCl₃) δ 4.99 (d, J=6.0 Hz, 2H), 6.52 (t, J=8.8 Hz, 1H), 7.05 (t, J=5.6 Hz, 2H), 7.29 (d, J=2.0 Hz, 2H), 7.51 (s, 1H), 7.72 (t, J=7.6 Hz, 1H), 8.30 (s, 1H), 8.99 (d, J=6.4 Hz, 1H).

### 2-(2,6-Dimethylphenylamino)-3-(hydroxymethyl)-4H-pyrido[1,2-a]pyrimidin-4-one (221)

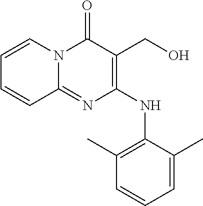 [SMILES: Cc1cccc(C)c1Nc1nc2ccccn2c(=O)c1CO]

¹H NMR (400 MHz, CDCl₃) δ 2.23 (s, 6H), 5.02 (d, J=6.4 Hz, 2H), 6.92 (t, J=6.8 Hz 1H), 7.12 (s, 3H), 7.20 (d, J=8.8 Hz, 1H), 7.33 (s, 1H), 7.53 (t, J=6.8 Hz, 1H), 8.94 (d, J=6.4 Hz, 1H).

### 3-(Hydroxymethyl)-2-phenoxy-4H-pyrido[1,2-a]pyrimidin-4-one (222)

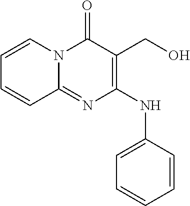 [SMILES: O=c1c(CO)c(Nc2ccccc2)nc2ccccn12]

¹H NMR (400 MHz, CDCl₃) δ 3.31 (brs, 1H), 4.86 (s, 2H), 7.03-7.09 (m, 3H), 7.13-7.18 (m, 1H), 7.28-7.34 (m, 3H), 7.58-7.62 (m, 1H), 8.94-8.96 (m, 1H); ¹³C NMR (100 MHz, CDCl₃) δ 56.0, 99.7, 115.2, 121.7, 125.1, 125.3, 127.4, 129.3, 136.8, 149.2, 152.8, 159.6, 164.0.

### 2-(3-Fluorophenoxy)-3-(hydroxymethyl)-4H-pyrido[1,2-a]pyrimidin-4-one (223)

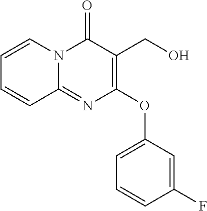 [SMILES: O=c1c(CO)c(Oc2cccc(F)c2)nc2ccccn12]

¹H NMR (400 MHz, CDCl₃) δ 3.62 (brs, 1H), 4.78 (s, 2H), 6.78-6.85 (m, 3H), 7.02 (ddd, J=1.2, 6.8, 7.2 Hz, 1H), 7.18-7.23 (m, 1H), 7.25 (d, J=9.2 Hz, 1H), 7.57-7.62 (m, 1H), 8.89 (d, J=6.8 Hz, 1H); ¹³C NMR (100 MHz, CDCl₃) δ 55.3, 99.7, 109.4, 109.6, 111.7, 111.9, 115.2, 117.2, 117.3, 125.0, 127.3, 129.7, 129.8, 137.0, 149.0, 153.5, 153.6, 159.4, 161.4, 163.6, 163.8.

### 2-(3-Chlorophenoxy)-3-(hydroxymethyl)-4H-pyrido[1,2-a]pyrimidin-4-one (224)

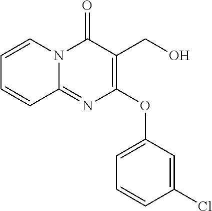 [SMILES: O=c1c(CO)c(Oc2cccc(Cl)c2)nc2ccccn12]

¹H NMR (400 MHz, CDCl₃) δ 3.51 (t, J=6.4 Hz, 1H), 4.79 (d, J=6.4 Hz, 2H), 6.95-6.98 (m, 1H), 7.04 (dd, J=6.8, 7.2 Hz, 1H), 7.08-7.10 (m, 1H), 7.20 (dd, J=8.4, 8.8 Hz, 1H), 7.27 (d, J=8.8 Hz, 1H), 7.59-7.63 (m, 1H), 8.91 9 dd, J=0.4, 7.2 Hz, 1H); ¹³C NMR (100 MHz, CDCl₃) δ 53.3, 55.4, 99.7, 115.3, 120.1, 122.2, 125.1, 127.4, 129.8, 134.3, 137.0, 153.2, 159.2, 163.6.

### 3-(Hydroxymethyl)-2-(phenylamino)-6,7,8,9-tetrahydro-4H-pyrido[1,2-a]pyrimidin-4-one (225)

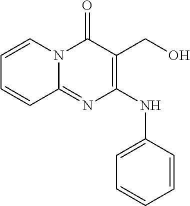 [SMILES: O=c1c(CO)c(Nc2ccccc2)nc2ccccn12]

¹H NMR (400 MHz, CDCl₃) δ 1.85-1.93 (m, 4H), 2.15 (s, 2H), 2.84 (t, J=6.8 Hz, 2H), 3.87 (t, J=6.2 Hz, 2H), 7.06 (t, J=7.0 Hz, 1H), 7.26 (t, J=7.0 Hz, 2H), 7.51 (d, J=7.4 Hz, 2H), 11.2 (s, 1H); ¹³C NMR (100 MHz, CDCl₃) δ 14.6, 19.2, 22.2, 32.2, 42.4, 88.4, 122.9, 124.4, 128.8, 138.4, 160.5, 160.8, 162.2.

### 2-(3-Chlorophenylamino)-3-(hydroxymethyl)-6,7,8,9-tetrahydro-4H-pyrido[1,2-a]pyrimidin-4-one (226)

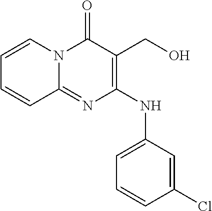 [SMILES: O=c1c(CO)c(Nc2cccc(Cl)c2)nc2ccccn12]

¹H NMR (400 MHz, DMSO-d₆) δ 1.23-1.34 (m, 2H), 1.38-1.51 (m, 4H), 2.35-2.41 (m, 2H), 3.98-4.05 (m, 2H), 4.12 (s, 2H), 7.17-7.22 (m, 2H), 7.31 (t, J=2.0 Hz, 1H), 7.36 (t, J=8.0 Hz, 1H), 7.77 (s, 1H); ¹³C NMR (100 MHz, DMSO-d₆) δ 15.1, 23.1, 31.4, 42.4, 59.2, 61.4, 65.7, 122.8, 123.9, 125.6, 131.6, 134.3, 139.4, 157.9, 164.3

### 3-(Hydroxymethyl)-2-(3-(trifluoromethyl)phenylamino)-6,7,8,9-tetrahydro-4H-pyrido[1,2-a]pyrimidin-4-one (227)

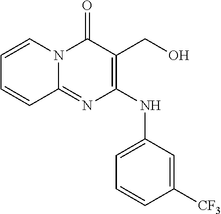 [SMILES: O=c1c(CO)c(Nc2cccc(C(F)(F)F)c2)nc2ccccn12]

¹H NMR (400 MHz, DMSO-d₆) δ 1.19-1.38 (m, 2H), 1.48-1.54 (m, 2H), 1.70-1.73 (m, 2H), 2.38 (t, J=12.8 Hz, 1H), 3.98-4.06 (m, 2H), 4.13 (s, 2H), 7.47 (d, J=7.6 Hz, 1H), 7.52 (d, J=8.8 Hz, 1H), 7.55-7.59 (m, 2H), 7.83 (s, 1H); ¹³C NMR (100 MHz, DMSO-d₆) 814.3, 22.2, 30.5, 41.5, 58.4, 77.9, 119.8, 121.2, 127.0, 129.8, 130.1, (d, J=26.8 due to CF₃), 138.2, 146.1, 157.1, 163.6, 169.1.

### 3-(Hydroxymethyl)-2-(2-hydroxyphenylamino)-6,7,8,9-tetrahydro-4H-pyrido[1,2-a]pyrimidin-4-one (228)

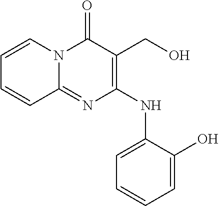 [SMILES: O=c1c(CO)c(Nc2ccccc2O)nc2ccccn12]

¹H NMR (400 MHz, CDCl₃) δ 1.78-1.94 (m, 4H), 2.13-2.23 (m, 2H), 2.61 (t, J=6.0 Hz, 1H), 3.98-4.05 (m, 2H), 4.12 (s, 2H), 6.81 (t, J=7.2 Hz, 1H), 6.89 (d, J=7.2 Hz, 1H), 6.98-7.12 (m, 2H), 10.11 (s, 1H), 11.3 (s, 1H); ¹³C NMR (100 MHz, CDCl₃) δ 14.3, 21.4, 31.3, 42.1, 61.1, 87.7, 121.2, 126.4, 128.3, 128.6, 151.1, 161.3, 162.5, 163.7, 169.4.

### 3-(Hydroxymethyl)-2-(3-hydroxyphenylamino)-6,7,8,9-tetrahydro-4H-pyrido[1,2-a]pyrimidin-4-one (229)

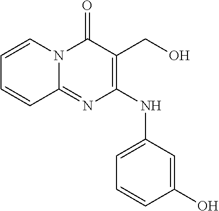 [SMILES: O=c1c(CO)c(Nc2cccc(O)c2)nc2ccccn12]

¹H NMR (400 MHz, CDCl₃) δ 1.41-1.61 (m, 4H), 1.62-1.77 (m, 2H), 2.72 (t, J=10.0 Hz, 1H), 3.78-3.95 (m, 2H), 4.17 (s, 2H), 6.43 (d, J=7.6 Hz, 1H), 6.81 (d, J=8.0 Hz, 1H), 6.87 (d, J=8.0 Hz, 1H), 6.98 (t, J=2.0 Hz, 1H); ¹³C NMR (100 MHz, CDCl₃) δ 14.2, 21.8, 31.9, 42.4, 60.1, 79.8, 109.8, 111.6, 114.0, 129.4, 139.4, 149.7, 159.3, 160.2, 163.1.

### 3-(Hydroxymethyl)-2-(4-hydroxyphenylamino)-6,7,8,9-tetrahydro-4H-pyrido[1,2-a]pyrimidin-4-one (230)

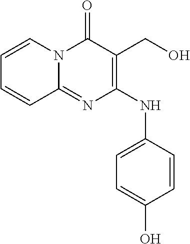 [SMILES: O=c1c(CO)c(Nc2ccc(O)cc2)nc2ccccn12]

¹H NMR (400 MHz, DMSO-d₆) δ 1.21-1.45 (m, 4H), 1.63-1.71 (m, 2H), 2.34 (t, J=12.8 Hz, 1H), 3.98-4.05 (m, 2H), 4.19 (s, 2H), 6.75 (d, J=8.8 Hz, 2H), 7.00 (d, J=8.8 Hz, 2H); ¹³C NMR (100 MHz, DMSO-d₆) δ 14.9, 21.9, 32.1, 42.3, 60.4, 87.2, 115.7, 125.0, 130.1, 154.9, 159.4, 160.6, 163.3.

### 3-(Hydroxymethyl)-9-methyl-2-(phenylamino)-4H-pyrido[1,2-a]pyrimidin-4-one (231)

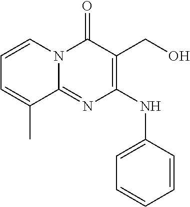 [SMILES: Cc1cccn2c(=O)c(CO)c(Nc3ccccc3)nc12]

¹H NMR (400 MHz, CDCl₃) δ 2.40 (s, 3H), 2.97 (brs, 1H), 4.93 (s, 2H), 6.89 (t, J=6.8 Hz, 1H), 7.11 (t, J=7.2 Hz, 1H), 7.34 (t, J=7.6 Hz, 2H), 7.62 (d, J=6.4 Hz, 1H), 8.02 (d, J=8.0 Hz, 2H), 8.73 (d, J=6.8 Hz, 1H).

### 2-(3-Chlorophenylamino)-3-(hydroxymethyl)-9-methyl-4H-pyrido[1,2-a]pyrimidin-4-one (232)

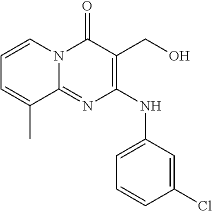 [SMILES: Cc1cccn2c(=O)c(CO)c(Nc3cccc(Cl)c3)nc12]

¹H NMR (400 MHz, CDCl₃) δ 2.43 (s, 3H), 3.06 (t, J=6.4 Hz, 1H), 4.92 (d, J=6.4 Hz, 2H), 6.69 (d, J=7.0 Hz, 1H), 7.03 (d, J=7.6 Hz, 1H), 7.23 (t, J=8.0 Hz, 1H), 7.29 (d, J=8.0 Hz, 1H), 7.44 (d, J=6.8 Hz, 1H), 8.03 (s, 1H), 8.38 (s, 1H), 8.71 (d, J=7.2 Hz, 1H).

### 2-((3-Chlorophenyl)(methyl)amino)-3-(hydroxymethyl)-9-methyl-4H-pyrido[1,2-a]pyrimidin-4-one (233)

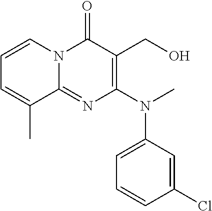 [SMILES: Cc1cccn2c(=O)c(CO)c(N(C)c3cccc(Cl)c3)nc12]

¹H NMR (400 MHz, CDCl₃) δ 2.51 (s, 3H), 4.09 (t, J=6.8 Hz, 1H), 4.12 (d, J=7.2 Hz, 2H), 6.95 (t, J=7.0 Hz, 1H), 7.04-7.06 (m, 2H), 7.20 (t, J=8.4 Hz, 1H), 7.54 (d, J=6.8 Hz, 1H), 8.84 (d, J=7.2 Hz, 1H).

### 2-((3-Chlorophenyl)(methyl)amino)-3-(methoxymethyl)-9-methyl-4H-pyrido[1,2-a]pyrimidin-4-one (234)

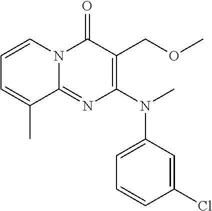 [SMILES: COCc1c(N(C)c2cccc(Cl)c2)nc2c(C)cccn2c1=O]

¹H NMR (400 MHz, CDCl₃) δ 2.49 (s, 3H), 3.01 (s, 3H), 4.04 (s, 3H), 6.91 (t, J=7.0 Hz, 1H), 7.08 (d, J=8.4 Hz, 1H), 7.12 (d, J=7.2 Hz, 1H), 7.20 (s, 1H), 7.26 (t, J=8.0 Hz, 1H), 7.52 (d, J=6.8 Hz, 1H), 8.86 (d, J=7.2 Hz, 1H).

### 3-(Hydroxymethyl)-9-methyl-2-(3-(trifluoromethoxy)phenylamino)-4H-pyrido[1,2-a]pyrimidin-4-one (235)

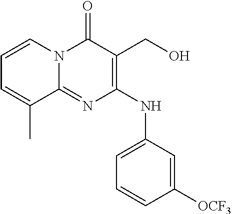 [SMILES: Cc1cccn2c(=O)c(CO)c(Nc3cccc(OC(F)(F)F)c3)nc12]

¹H NMR (400 MHz, CDCl₃) δ 2.40 (s, 3H), 3.15 (t, J=6.2 Hz, 1H), 4.93 (d, J=6.4 Hz, 1H), 6.67 (t, J=7.0 Hz, 1H), 6.91 (d, J=8.0 Hz, 1H), 7.25-7.27 (m, 1H), 7.32 (t, J=8.2 Hz, 1H), 7.43 (d, J=6.8 Hz, 1H), 7.98 (s, 1H), 8.51 (s, 1H), 8.72 (d, J=6.8 Hz, 1H).

### 3-(Hydroxymethyl)-2-(3-hydroxyphenylamino)-9-methyl-4H-pyrido[1,2-a]pyrimidin-4-one (236)

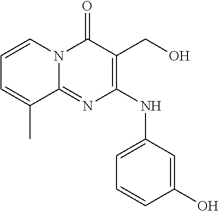 [SMILES: Cc1cccn2c(=O)c(CO)c(Nc3cccc(O)c3)nc12]

¹H NMR (400 MHz, CDCl₃+CD₃OD) δ 2.44 (s, 3H), 4.75 (s, 2H), 6.45 (dd, J=2.4, 8.0 Hz, 1 h), 6.84 (dd, J=6.8, 6.8 Hz, 1H), 7.06 (dd, J=8.0, 8.4 Hz, 1H), 7.11 (dd, J=2.0, 2.4 Hz, 1H), 7.17 (dd, H=2.0, 8.0 Hz, 1H), 7.45 (d, J=6.8 Hzm 1H), 8.72 (d, J=7.2 Hz, 1H).

### 3-(Hydroxymethyl)-2-(4-hydroxyphenylamino)-9-methyl-4H-pyrido[1,2-a]pyrimidin-4-one (237)

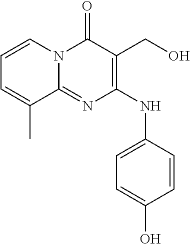 [SMILES: Cc1cccn2c(=O)c(CO)c(Nc3ccc(O)cc3)nc12]

¹H NMR (400 MHz, CDCl₃) δ 2.40 (s, 3H), 4.94 (d, J=4.8 Hz, 1H), 6.81-6.84 (m, 3H), 7.46 (d, J=7.2 Hz, 1H), 7.50 (d, J=8.8 Hz, 2H), 7.84 (s, 1H), 8.82 (d, J=7.2 Hz, 1H).

### 2-(4-tert-Butylphenylamino)-3-(hydroxymethyl)-9-methyl-4H-pyrido[1,2-a]pyrimidin-4-one (238)

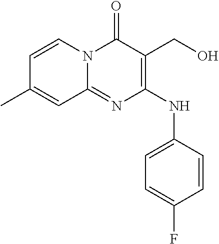 [SMILES: Cc1ccn2c(=O)c(CO)c(Nc3ccc(F)cc3)nc2c1]

¹H NMR (400 MHz, CDCl₃) δ 1.34 (s, 9H), 2.40 (s, 3H), 3.07 (t, J=6.2 Hz, 1H), 4.91 (d, J=6.4 Hz, 2H), 6.61 (t, J=6.8 Hz, 1H), 7.34 (d, J=7.2 Hz, 2H), 7.38 (d, J=6.8 Hz, 1H), 8.21 (br s, 1H), 8.69 (d, J=7.2 Hz, H).

### 2-(3-Chlorobenzylamino)-3-(hydroxymethyl)-9-methyl-4H-pyrido[1,2-a]pyrimidin-4-one (239)

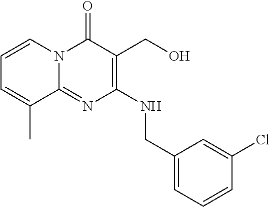 [SMILES: Cc1cccn2c(=O)c(CO)c(NCc3cccc(Cl)c3)nc12]

¹H NMR (400 MHz, CDCl₃+CD₃OD) δ 2.31 (s, 3H), 3.02 (s, 1H), 4.68 (d, J=5.6 Hz, 2H), 4.70 (s, 2H), 6.70 (dd, J=5.6, 6.0 Hz, 1H), 6.74 (dd, J=6.8, 7.2 Hz, 1H), 7.11-7.20 (m, 3H), 7.31 (s, 1H), 7.38 (d, J=6.8 Hz, 1H), 8.66 (d, J=6.8 Hz, 1H); ¹³C NMR (100 MHz, CDCl₃+CD₃OD) δ 17.7, 44.2, 44.3, 55.8, 93.1, 93.2, 112.6, 125.4, 125.5, 126.9, 127.5, 129.5, 132.6, 134.0, 134.9, 141.7, 149.45, 149.47, 157.4, 159.10, 159.16.

### 3-(Hydroxymethyl)-2-(isobutylamino)-9-methyl-4H-pyrido[1,2-a]pyrimidin-4-one (240)

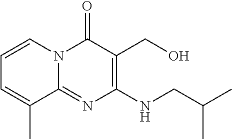 [SMILES: Cc1cccn2c(=O)c(CO)c(NCC(C)C)nc12]

¹H NMR (400 MHz, CDCl₃) δ 0.96 (d, J=6.8 Hz, 6H), 1.88-1.95 (m, 1H), 2.34 (s, 3H), 3.13 (brs, 1H), 3.32 (t, J=6.0 Hz, 2H), 4.78 (d, J=6.0 Hz, 2H), 6.08 (brs, 1H), 6.72 (t, J=6.8 Hz, 1H), 7.37 (d, J=6.8 Hz, 1H), 8.66 (d, J=6.8 Hz, 1H); ¹³C NMR (100 MHz, CDCl₃) δ 17.9, 20.5, 28.9, 48.6, 57.1, 92.5, 112.1, 126.0, 132.5, 134.6, 149.6, 157.1, 159.5.

### 2-(Diethylamino)-3-(hydroxymethyl)-9-methyl-4H-pyrido[1,2-a]pyrimidin-4-one (241)

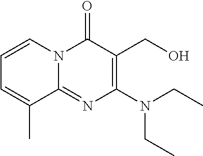 [SMILES: CCN(CC)c1nc2c(C)cccn2c(=O)c1CO]

¹H NMR (400 MHz, CDCl₃) δ 1.22 (t, J=6.8 Hz, 6H), 2.35 (s, 3H), 3.41 (s, 1H), 3.63 (q, J=6.8 Hz, 4H), 4.44 (s, 2H), 6.65 (t, J=7.2 Hz, 1H), 7.31 (d, J=6.8 Hz, 1H), 8.68 (d, J=7.2 Hz, 1H) ¹³C NMR (100 MHz, CDCl₃) δ 13.9, 17.7, 44.0, 67.0, 92.2, 111.7, 125.8, 132.5, 134.4, 148.1, 160.7, 160.8.

### 2-(Cyclohexylmethylamino)-3-(hydroxymethyl)-9-methyl-4H-pyrido[1,2-a]pyrimidin-4-one (242)

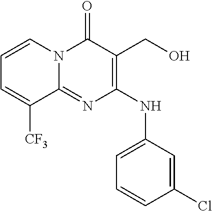 [SMILES: O=c1c(CO)c(Nc2cccc(Cl)c2)nc2c(C(F)(F)F)cccn12]

¹H NMR (400 MHz, CDCl₃) δ 0.95-0.98 (m, 2H), 1.18-1.23 (m, 3H), 1.58-1.79 (m, 6H), 2.42 (s, 3H), 3.27 (t, J=6.4 Hz, 2H), 3.85 (brs, 1H), 4.74 (m, 2H), 6.21 (t, J=7.2 Hz, 1H), 6.68 (d, J=6.8 Hz, 1H), 7.33 (d, J=7.2 Hz, 1H), 8.57 (d, J=7.2 Hz, 1H); ¹³C NMR (100 MHz, CDCl₃) δ 17.9, 26.2, 26.7, 31.3, 38.4, 47.5, 56.9, 92.8, 112.0, 126.0, 132.3, 134.5, 149.4, 156.9, 159.5.

### 3-(Hydroxymethyl)-9-methyl-2-morpholino-4H-pyrido[1,2-a]pyrimidin-4-one (243)

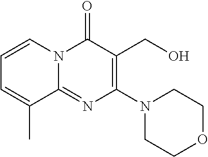 [SMILES: Cc1cccn2c(=O)c(CO)c(N3CCOCC3)nc12]

¹H NMR (400 MHz, CDCl₃) δ 2.01 (brs, 1H), 2.43 (s, 3H), 3.62 (t, J=4.8 Hz, 4H), 3.78 (t, J=4.8 Hz, 4H), 4.62 (s, 2H), 6.85 (t, J=6.8 Hz, 1H), 7.46 (d, J=6.8 Hz, 1H), 8.76 (d, J=6.8 Hz, 1H); ¹³C NMR (100 MHz, CDCl₃) δ 17.9, 49.7, 58.9, 67.1, 95.5, 113.3., 125.2, 133.4, 135.0, 148.2, 160.6, 161.7.

### 3-(Hydroxymethyl)-9-methyl-2-morpholino-4H-pyrido[1,2-a]pyrimidin-4-one hydrochloride (244)

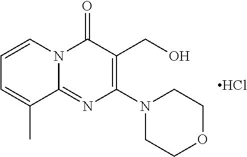 [SMILES: Cc1cccn2c(=O)c(CO)c(N3CCOCC3)nc12 ; Cl]

¹H NMR (400 MHz, CDCl₃) δ 2.43 (s, 3H), 3.42 (s, 1H), 3.62 (t, J=4.8 Hz, 4H), 3.78 (t, J=4.8 Hz, 4H), 4.62 (s, 2H), 6.85 (t, J=6.8 Hz, 1H), 7.46 (d, J=6.8 Hz, 1H), 8.76 (d, J=6.8 Hz, 1H); ¹³C NMR (100 MHz, CDCl₃) δ 17.9, 49.7, 58.9, 67.1, 98.5, 113.3, 125.2, 133.4, 135.0, 148.2, 160.6, 161.7.

### 7-Bromo-2-(3-chlorophenylamino)-3-(hydroxymethyl)-4H-pyrido[1,2-a]pyrimidin-4-one (245)

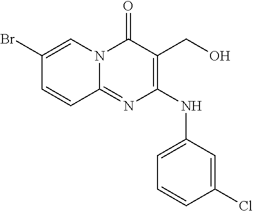 [SMILES: O=c1c(CO)c(Nc2cccc(Cl)c2)nc2ccc(Br)cn12]

¹H NMR (400 MHz, DMSO-d₆) δ 4.78 (s, 2H), 5.37 (s, 1H), 7.12 (dd, J=1.6 Hz, 8.4 Hz, 1H), 7.32 (d, J=8.0 Hz 1H), 7.42 (dd, J=1.6 Hz, 8.4 Hz, 1H), 7.54 (dd, J=0.8 Hz, 8.0 Hz, 1H), 7.64 (d, J=8.0 Hz 1H), 7.91 (d, J=2.0 Hz, 1H), 8.47 (s, 1H), 8.71 (s, 1H);

### 2-(3-Chlorophenylamino)-3-(hydroxymethyl)-7-methoxy-4H-pyrido[1,2-a]pyrimidin-4-one (246)

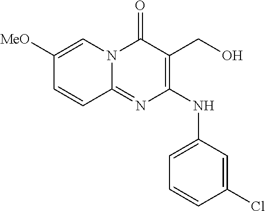 [SMILES: COc1ccc2nc(Nc3cccc(Cl)c3)c(CO)c(=O)n2c1]

¹H NMR (400 MHz, DMSO-d₆) δ 3.86 (s, 3H), 4.70 (s, 2H), 5.22 (s, 1H), 7.02 (dd, J=0.8 Hz, 8.0 Hz, 1H), 7.28-7.32 (m, 1H), 7.41 (dd, J=1.2 Hz, 9.6 Hz, 1H), 7.58 (dd, J=0.8 Hz, 8.0 Hz, 1H), 7.64-7.68 (m, 1H), 7.87 (d, J=2.0 Hz, 1H), 8.36 (s, 1H), 8.69 (s, 1H)

### 2-(3-Chlorophenylamino)-3-(hydroxymethyl)-8-methoxy-4H-pyrido[1,2-a]pyrimidin-4-one (247)

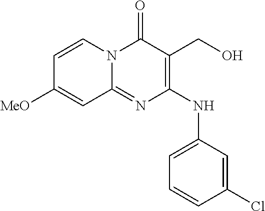 [SMILES: COc1ccn2c(=O)c(CO)c(Nc3cccc(Cl)c3)nc2c1]

¹H NMR (400 MHz, DMSO-d₆) δ 3.92 (s, 3H), 4.62 (s, 2H), 5.07 (s, 1H), 6.71 (d, J=2.8 Hz, 1H), 6.83 (dd, J=2.8 Hz, 8.0 Hz, 1H), 7.01 (d, J=8.0 Hz, 1H), 7.28 (dd, J=8.0 Hz, J=8.0 Hz, 1H), 7.62 (d, J=8.0 Hz, 1H), 7.76 (d, J=2.0 Hz, 1H), 8.62 (s, 1H), 8.71 (d, J=8.0 Hz, 1H); ¹³C NMR (100 MHz, DMSO-d₆) 54.8, 57.3, 93.8, 101.5, 109.3, 120.0, 120.9, 122.5, 129.5, 130.7, 133.4, 142.2, 151.9, 156.9, 157.8, 166.2.

### 8-Chloro-2-(3-chlorophenylamino)-3-(hydroxymethyl)-4H-pyrido[1,2-a]pyrimidin-4-one (248)

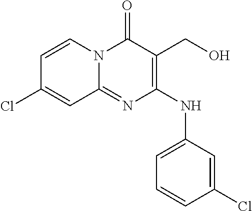 [SMILES: O=c1c(CO)c(Nc2cccc(Cl)c2)nc2cc(Cl)ccn12]

¹H NMR (400 MHz, CDCl₃) δ 4.68 (s, 2H), 5.14 (brs, 1H), 7.03 (dd, J=1.2, 8.0 Hz, 1H), 7.19 (dd, J=2.4, 7.6 Hz, 1H), 7.28 (t, J=8.0 Hz, 1H), 7.54, (d, J=2.0 Hz, 1H), 7.58 (dd, J=1.2, 8.4 Hz, 1H), 7.57 (t, J=2.0 Hz, 1H), 8.78 (d, J=8.0 Hz, 1H).

### 2-(3-Chlorophenylamino)-3-(hydroxymethyl)-8-(methylamino)-4H-pyrido[1,2-a]pyrimidin-4-one (249)

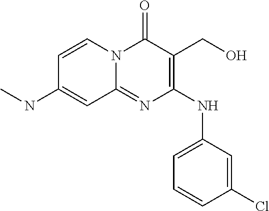 [SMILES: CNc1ccn2c(=O)c(CO)c(Nc3cccc(Cl)c3)nc2c1]

¹H NMR (400 MHz, CDCl₃) δ 2.81 (s, 3H), 3.85 (s, 2H), 6.02 (s, 1H), 6.32 (d, J=7.6 Hz, 1H), 6.93 (d, J=2 Hz, 1H), 7.12 (t, J=8.0 Hz, 1H), 7.38 (d, J=8.0 Hz, 1H), 7.81 (s, 1H), 8.42 (s, 1H), 9.93 (s, 1H).

### 2-(3-Chlorophenylamino)-8-(diethylamino)-3-(hydroxymethyl)-4H-pyrido[1,2-a]pyrimidin-4-one (250)

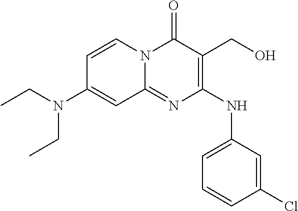 [SMILES: CCN(CC)c1ccn2c(=O)c(CO)c(Nc3cccc(Cl)c3)nc2c1]

¹H NMR (400 MHz, CDCl₃) δ 1.23 (t, J=6.8 Hz, 6H), 3.44 (q, J=6.8 Hz, 4H), 3.99 (s, 2H), 4.82 (t, J=2.1 Hz, 1H), 6.29 (d, J=2.1 Hz, 1H), 6.54 (dd, J=2.4, 8.4 Hz, 1H), 6.92 (d, J=2 Hz, 1H), 7.21 (t, J=8.0 Hz, 1H), 7.81 (d, J=2.4 Hz, 1H), 8.06 (t, J=2.0 Hz, 1H), 8.85 (d, J=8.4 Hz, 1H), 9.71 (s, 1H); ¹³C NMR (100 MHz, CDCl₃) δ 12.7, 20.0, 44.7, 92.8, 97.1, 104.0, 118.9, 120.7, 121.9, 128.5, 129.5, 134.1, 142.8, 150.6, 151.9, 158.3, 159.2.

### 3-(Hydroxymethyl)-8-morpholino-2-(phenylamino)-4H-pyrido[1,2-a]pyrimidin-4-one (251)

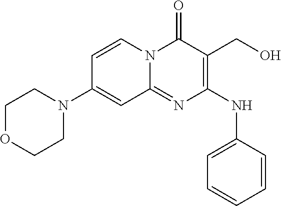 [SMILES: O=c1c(CO)c(Nc2ccccc2)nc2cc(N3CCOCC3)ccn12]

¹H NMR (400 MHz, DMSO-d₆) δ 3.43 (s, 4H), 3.67 (s, 4H), 4.59 (d, J=5.2 Hz, 2H), 5.05, (t, J=4.8 Hz, 1H), 6.41 (d, J=2.0 Hz, 1H), 6.95 (t, J=7.2 Hz, 1H), 7.00 (dd, J=2.8, 8.4 Hz, 1H), 7.25 (t, J=8.0 Hz, 2H), 7.64 (d, J=7.6 Hz, 2H), 8.38 (s, 1H), 8.69 (d, J=8.0 Hz, 1H); ¹³C NMR (100 MHz, DMSO-d₆) δ 46.5, 55.1, 66.3, 91.5, 99.1, 105.4, 121.3, 122.6, 128.5, 129.1, 140.9, 151.4, 155.0, 156.7, 158.5.

### 2-(3-Fluorophenylamino)-3-(hydroxymethyl)-8-morpholino-4H-pyrido[1,2-a]pyrimidin-4-one (252)

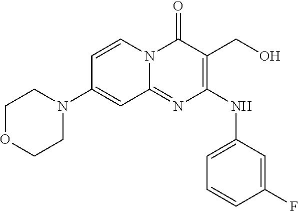 [SMILES: O=c1c(CO)c(Nc2cccc(F)c2)nc2cc(N3CCOCC3)ccn12]

¹H NMR (400 MHz, DMSO-d₆) δ 3.46 (s, 4H), 3.68 (s, 4H), 4.59 (d, J=5.2 Hz, 2H), 5.06, (t, J=5.2 Hz, 1H), 6.47 (d, J=2.4 Hz, 1H), 6.74 (t, J=7.2 Hz, 1H), 7.03 (dd, J=2.8, 8.0 Hz, 1H), 7.26 (t, J=7.2 Hz, 1H), 7.64 (d, J=8.0 Hz, 1H), 7.79 (d, J=12.4 Hz, 1H), 8.52 (s, 1H), 8.60 (d, J=8.0 Hz, 1H); ¹³C NMR (100 MHz, DMSO-d₆) δ 45.8, 54.2, 65.6, 91.3, 98.4, 105.0, 108.0 (d, J=20 Hz, due to F), 116.0, 128.0, 129.8 (d, J=10 Hz, due to F), 142.1 (d, J=11 Hz, due to F), 150.6, 154.4, 156.1, 157.4, 161.0, 163.3.

### 2-(3-Chlorophenylamino)-3-(hydroxymethyl)-8-morpholino-4H-pyrido[1,2-a]pyrimidin-4-one (253)

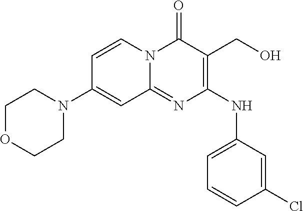 [SMILES: O=c1c(CO)c(Nc2cccc(Cl)c2)nc2cc(N3CCOCC3)ccn12]

¹H NMR (400 MHz, DMSO-d₆) δ 3.45 (t, J=5.6 Hz, 4H), 3.69 (t, J=5.6 Hz, 4H), 4.58 (d, J=5.2 Hz, 2H), 5.01 (t, J=5.2 Hz, 1H), 6.42 (d, J=2.8 Hz, 1H), 6.98 (d, J=8.0 Hz, 1H), 7.05 (dd, J=2.8, 8.0 Hz, 1H), 7.26 (t, J=8.0 Hz, 1H), 7.64 (d, J=8.0 Hz, 1H), 7.80 (t, J=2.0 Hz, 1H), 8.48 (s, 1H), 8.60 (d, J=8.0 Hz, 1H); ¹³C NMR (100 MHz, DMSO-d₆) δ 45.4, 53.6, 65.7, 84.7, 98.6, 105.3, 117.8, 118.7, 119.8, 127.1, 130.2, 129.2, 141.8, 149.7, 153.0, 155.3, 157.4; LC-MS (ESI, m/z): 386 [M+H]⁺.

### 3-(Hydroxymethyl)-8-(4-methylpiperazin-1-yl)-2-(phenylamino)-4H-pyrido[1,2-a]pyrimidin-4-one (254)

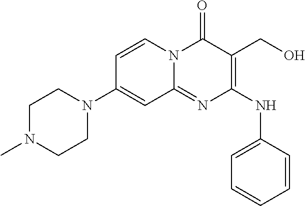 [SMILES: CN1CCN(c2ccn3c(=O)c(CO)c(Nc4ccccc4)nc3c2)CC1]

¹H NMR (400 MHz, CDCl₃) δ 2.34 (s, 3H), 2.52 (t, J=5.2 Hz, 4H), 3.43 (t, J=5.2 Hz, 4H), 4.88 (s, 2H), 5.28 (s, 1H), 6.37 (s, 1H), 6.55 (d, J=8.0 Hz, 1H), 7.05 (t, J=7.2 Hz, 1H), 7.33 (t, J=7.6 Hz, 2H), 7.60 (d, J=7.6 Hz, 2H), 7.91 (s, 1H), 8.64 (d, J=8.0 Hz, 1H).

### 2-(3-Chlorophenylamino)-3-(hydroxymethyl)-8-(4-methylpiperazin-1-yl)-4H-pyrido[1,2-a]pyrimidin-4-one (255)

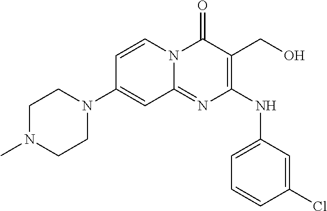 [SMILES: CN1CCN(c2ccn3c(=O)c(CO)c(Nc4cccc(Cl)c4)nc3c2)CC1]

¹H NMR (400 MHz, CDCl₃) δ 2.14 (s, 3H), 2.38 (t, J=4.4 Hz, 4H), 3.45 (t, J=4.4 Hz, 4H), 3.56 (s, 2H), 6.41 (d, J=2.4 Hz, 1H), 6.95 (dd, J=1.6, 8.0 Hz, 1H), 7.01 (dd, J=2.4, 8.0 Hz, 1H), 7.27 (t, J=8.0 Hz, 1H), 7.50 (d, J=1.6 Hz, 1H), 8.0 (d, J=8.0 Hz, 1H), 10.4 (s, 1H), 14.18 (s, 1H); ¹³C NMR (100 MHz, CDCl₃) δ 45.6, 51.6, 54.0, 55.0, 85.3, 98.3, 105.1, 117.7, 118.5, 121.0, 127.9, 130.3, 133.0, 142.1, 150.8, 154.1, 156.4, 157.8; LC-MS (ESI, m/z): 400 [M+H]⁺.

### 2-(3-Fluorophenylamino)-3-(hydroxymethyl)-8-(4-methylpiperazin-1-yl)-4H-pyrido[1,2-a]-pyrimidin-4-one (256)

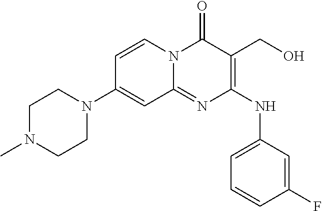 [SMILES: CN1CCN(c2ccn3c(=O)c(CO)c(Nc4cccc(F)c4)nc3c2)CC1]

¹H NMR (400 MHz, CDCl₃) δ 2.35 (s, 3H), 2.54 (t, J=4.4 Hz, 4H), 3.48 (t, J=4.8 Hz, 4H), 4.87 (s, 2H), 5.23 (s, 1H), 6.42 (s, 1H), 6.60 (d, J=8.4 Hz, 1H), 6.73 (t, J=8.4 Hz, 1H), 7.12 (d, J=8.4 Hz, 1H), 7.19 (d, J=8.4 Hz, 1H), 7.71-7.75 (m, 1H), 8.04 (s, 1H), 8.71 (d, J=8.0 Hz, 1H).

### 2-(3-Chlorophenylamino)-3-(hydroxymethyl)-8-methyl-4H-pyrido[1,2-a]pyrimidin-4-one (257)

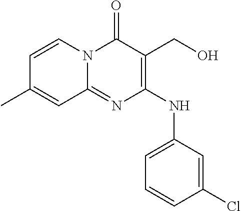 [SMILES: Cc1ccn2c(=O)c(CO)c(Nc3cccc(Cl)c3)nc2c1]

Colorless solid, mp 235° C. (decomp.); ¹H NMR (400 MHz, CDCl₃) δ 2.42 (s, 3H), 4.07 (q, J=7.2 Hz, 2H), 7.03 (d, J=8.8 Hz, 2H), 7.26 (t, J=8.0 Hz, 2H), 7.46 (d, J=8.4 Hz, 1H), 7.84 (t, J=2.0 Hz, 1H), 8.79 (d, J=7.2 Hz, 2H).

### 2-(4-Chlorophenylamino)-3-(hydroxymethyl)-8-methyl-4H-pyrido[1,2-a]pyrimidin-4-one (258)

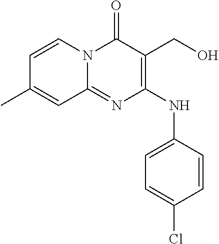 [SMILES: Cc1ccn2c(=O)c(CO)c(Nc3ccc(Cl)cc3)nc2c1]

Colorless solid, mp 227° C. (decomp.); ¹H NMR (400 MHz, CDCl₃) δ 2.42 (s, 3H), 4.10 (s, 2H), 6.85 (d, J=7.2 Hz, 1H), 7.23-7.28 (m, 4H), 7.87 (d, J=6.8 Hz, 2H), 8.94 (d, J=7.6 Hz, 114).

### 2-(4-Fluorophenylamino)-3-(hydroxymethyl)-8-methyl-4H-pyrido[1,2-a]pyrimidin-4-one (259)

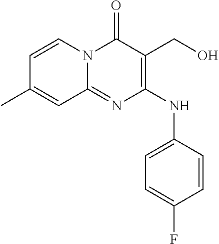 [SMILES: Cc1ccn2c(=O)c(CO)c(Nc3ccc(F)cc3)nc2c1]

Colorless solid, mp 232° C. (decomp.); ¹H NMR (400 MHz, CDCl₃) δ 2.42 (s, 3H), 4.12 (s, 2H), 6.85 (d, J=6.8 Hz, 1H), 7.05 (t, J=8.4 Hz, 2H), 7.21 (s, 1H), 7.31-7.38 (m, 2H), 7.85 (q, J=4.8 Hz, 2H), 8.94 (d, J=7.2 Hz, 1H).

### 2-(3,4-Dichlorophenylamino)-3-(hydroxymethyl)-8-methyl-4H-pyrido[1,2-a]pyrimidin-4-one (260)

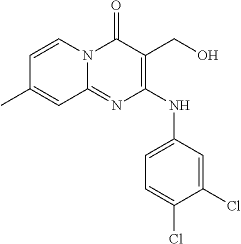 [SMILES: Cc1ccn2c(=O)c(CO)c(Nc3ccc(Cl)c(Cl)c3)nc2c1]

Colorless solid, mp 230° C. (decomp.); ¹H NMR (400 MHz, CDCl₃) δ 2.44 (s, 3H), 4.09 (s, 2H), 6.89 (d, J=7.2 Hz, 1H), 7.26 (s, 1H), 7.36 (d, J=8.8 Hz, 1H), 7.76 (d, J=8.4 Hz, 1H), 8.24 (d, J=2.4 Hz, 1H), 8.95 (d, J=7.2 Hz, 1H), 9.71 (s, 1H).

### 2-(3-Chloro-4-fluorophenylamino)-3-(hydroxymethyl)-8-methyl-4H-pyrido[1,2-a]pyrimidin-4-one (261)

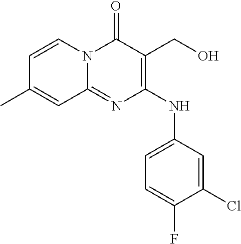 [SMILES: Cc1ccn2c(=O)c(CO)c(Nc3ccc(F)c(Cl)c3)nc2c1]

Colorless solid, mp 225° C. (decomp.); ¹H NMR (400 MHz, CDCl₃) δ 2.43 (s, 3H), 4.09 (s, 2H), 6.88 (d, J=7.2 Hz, 1H), 7.11 (t, J=8.8 Hz, 1H), 7.27 (s, 1H), 7.69-7.73 (m, 1H), 8.12 (d, J=6.8 Hz, 1H), 8.95 (d, J=7.2 Hz, 1H), 9.71 (s, 1H).

### 9-Chloro-2-(3-chlorophenylamino)-3-(hydroxymethyl)-4H-pyrido[1,2-a]pyrimidin-4-one (262)

Colorless solid, mp 230° C. (decomp.); ¹H NMR (400 MHz, CDCl₃) δ 4.95 (d, J=6.0 Hz, 2H), 6.80 (t, J=7.2 Hz, 1H), 7.06 (d, J=8.0 Hz, 1H), 7.27 (d, J=8.4 Hz, 1H), 7.46 (d, J=8.0 Hz, 1H), 7.78 (d, J=7.2 Hz, 1H), 8.18 (t, J=2.4 Hz, 1H), 8.43 (s, 1H), 8.81 (d, J=7.2 Hz, 1H).

### 2-(3-Chlorophenylamino)-3-(hydroxymethyl)-9-(trifluoromethyl)-4H-pyrido[1,2-a]pyrimidin-4-one (263)

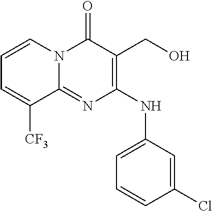 [SMILES: O=c1c(CO)c(Nc2cccc(Cl)c2)nc2c(C(F)(F)F)cccn12]

¹H NMR (400 MHz, DMSO-d₆) δ 4.77 (s, 2H), 7.11-7.13 (m, 1H), 7.32 (dd, J=7.2, 7.2 Hz, 1H), 7.35 (dd, J=8.0, 8.0 Hz, 1H), 7.48-7.50 (m, 1H), 8.13-8.14 (m, 1H), 8.41 (d, J=7.2 Hz, 1H), 9.12 (dd, J=1.2, 7.2 Hz, 1H).

### 2-(3-Chlorophenylamino)-9-fluoro-3-(hydroxymethyl)-4H-pyrido[1,2-a]pyrimidin-4-one (264)

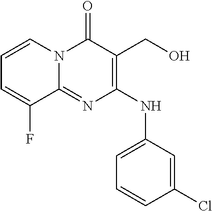 [SMILES: O=c1c(CO)c(Nc2cccc(Cl)c2)nc2c(F)cccn12]

¹H NMR (400 MHz, DMSO-d₆) δ 4.76 (s, 1H), 5.31 (brs, 1H), 7.11-7.13 (m, 1H), 7.18-7.23 (m, 1H), 7.38 (dd, J=8.0, 8.0 Hz, 1H), 7.63-7.65 (m, 1H), 7.86 (dd, J=8.4, 8.8 Hz, 1H), 8.12-8.13 (m, 1H), 8.73 (d, J=7.2 Hz, 1H), 8.96 (brs, 1H).

### 2-(4-Chlorophenylamino)-9-fluoro-3-(hydroxymethyl)-4H-pyrido[1,2-a]pyrimidin-4-one (265)

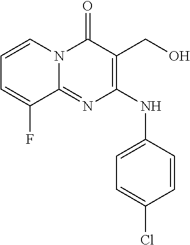 [SMILES: O=c1c(CO)c(Nc2ccc(Cl)cc2)nc2c(F)cccn12]

¹H NMR (400 MHz, DMSO-d₆) δ 4.72 (s, 2H), 5.30 (brs, 1H), 7.15-7.20 (m, 1H), 7.41-7.44 (m, 2H), 7.79-7.82 (m, 2H), 7.84-7.86 (m, 1H), 8.72 (d, J=7.2 Hz, 1H), 8.92 (brs, 1H).

### 9-Fluoro-2-(4-fluorophenylamino)-3-(hydroxymethyl)-4H-pyrido[1,2-a]pyrimidin-4-one (266)

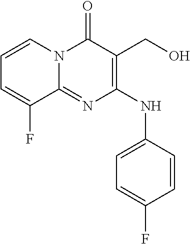 [SMILES: O=c1c(CO)c(Nc2ccc(F)cc2)nc2c(F)cccn12]

¹H NMR (400 MHz, DMSO-d₆) δ 4.75 (s, 2H), 5.25 (brs, 1H), 7.13-7.25 (m, 3H), 7.73-7.77 (m, 2H), 7.80-7.85 (m, 1H), 8.72 (d, J=7.2 Hz, 1H), 8.84 (brs, 1H).

### 2-(3-Chloro-4-fluorophenylamino)-9-fluoro-3-(hydroxymethyl)-4H-pyrido[1,2-a]pyrimidin-4-one (267)

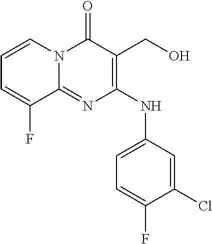 [SMILES: O=c1c(CO)c(Nc2ccc(F)c(Cl)c2)nc2c(F)cccn12]

¹H NMR (400 MHz, DMSO-d₆) δ 4.74 (s, 2H), 5.24 (brs, 1H), 7.18-7.22 (m, 1H), 7.39-7.44 (m, 1H), 7.65-7.69 (m, 1H), 7.83-7.87 (m, 1H), 8.20-8.22 (m, 1H), 8.72 (d, J=7.2 Hz, 1H), 8.91 (brs, 1H).

### 2-(3,4-Difluorophenylamino)-9-fluoro-3-(hydroxymethyl)-4H-pyrido[1,2-d]pyrimidin-4-one (268)

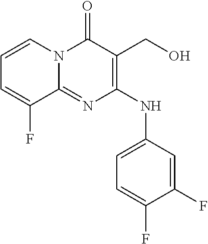 [SMILES: O=c1c(CO)c(Nc2ccc(F)c(F)c2)nc2c(F)cccn12]

¹H NMR (400 MHz, DMSO-d₆) δ 4.75 (s, 2H), 5.26 (brs, 1H), 7.17-7.22 (m, 1H), 7.39-7.49 (m, 1H), 7.84-7.88 (m, 1H), 8.08-8.14 (m, 1H), 8.73 (m, J=7.2 Hz, 1H), 8.93 (brs, 1H).

### 2-(3,4-Dichlorophenylamino)-9-fluoro-3-(hydroxymethyl)-4H-pyrido[1,2-a]pyrimidin-4-one (269)

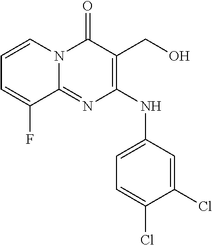 [SMILES: O=c1c(CO)c(Nc2ccc(Cl)c(Cl)c2)nc2c(F)cccn12]

¹H NMR (400 MHz, DMSO-d₆) δ 4.75 (s, 2H), 5.27 (brs, 1H), 7.19-7.23 (m, 1H), 7.60 (d, J=8.8 Hz, 1H), 7.7 (dd, J=2.8, 8.8 Hz, 1H), 7.85-7.89 (m, 1H), 8.83 (d, J=2.8 Hz, 1H), 8.73 (d, J=8.8 Hz, 1H), 9.00 (brs, 1H).

### 2-(1H-Indol-5-ylamino)-9-fluoro-3-(hydroxymethyl)-4H-pyrido[1,2-a]pyrimidin-4-one (270)

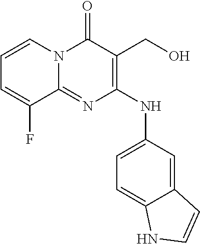 [SMILES: O=c1c(CO)c(Nc2ccc3[nH]ccc3c2)nc2c(F)cccn12]

m.p=184-185° C.; ¹H NMR (400 MHz, DMSO-d₆) δ 4.70 (d, J=5.2 Hz, 2H), 5.18 (t, J=5.2 Hz, 1H), 6.35 (s, 1H), 7.00-7.04 (m, 1H), 7.23 (dd, J=2 Hz, 8.8 Hz, 1H), 7.28-7.32 (m, 2H), 7.68 (dd, J=8 Hz, J=8 Hz, 1H), 7.82 (s, 1H), 8.61 (s, 1H), 8.64 (d, J=6 Hz, 1H), 10.98 (s, 1H); ¹³C NMR (100 MHz, DMSO-d₆) 55.2, 94.6, 101.7 (d, J=5.2 Hz, due to F), 111.6, 112.1 (d, J=7.4 Hz, due to F), 113.7, 118.0, 119.8 (d, J=17.1 Hz, due to F), 124.2 (d, J=4.4 Hz, due to F), 126.5, 128.2, 131.9, 133.5, 151.6, 154.1, 156.3, 157.6.

### 3-(Hydroxymethyl)-9-methoxy-2-(phenylamino)-4H-pyrido[1,2-a]pyrimidin-4-one (271)

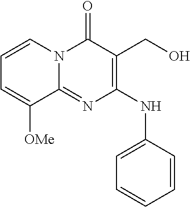 [SMILES: COc1cccn2c(=O)c(CO)c(Nc3ccccc3)nc12]

¹H NMR (400 MHz, DMSO-d₆) δ 3.93 (s, 3H), 4.71 (d, J=5.2 Hz, 2H), 5.29 (t, J=5.2 Hz, 1H), 6.97-7.01 (m, 1H), 7.06-7.10 (m, 1H), 7.27-7.32 (m, 3H), 7.83 (d, J=8.4 Hz, 2H), 8.47 (d, J=7.2 Hz, 1H), 8.68 (s, 1H).

### 3-(Hydroxymethyl)-9-methoxy-2-(phenylamino)-4H-pyrido[1,2-a]pyrimidine-4-thione (272)

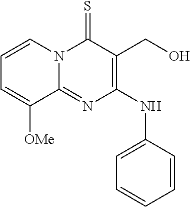 [SMILES: COc1cccn2c(=S)c(CO)c(Nc3ccccc3)nc12]

¹H NMR (400 MHz, CDCl₃) δ 3.98 (s, 3H), 4.11 (d, J=7.2 Hz, 2H), 6.88 (t, J=8.0 Hz, 2H), 7.04 (t, J=7.2 Hz, 1H), 7.31 (t, J=7.2 Hz, 2H), 7.82 (d, J=7.6 Hz, 2H), 7.98 (s, 1H), 8.59 (d, J=5.6 Hz, 1H); ¹³C NMR (100 MHz, CDCl₃) 26.9, 57.1, 94.2, 111.8, 112.7, 119.9, 121.1, 123.3, 128.9, 139.8, 143.7, 151.3, 155.6, 158.6.

### 2-(3-Chlorophenylamino)-3-(hydroxymethyl)-9-methoxy-4H-pyrido[1,2-a]pyrimidin-4-one (273)

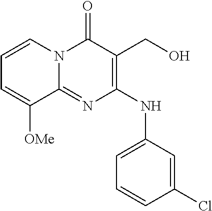 [SMILES: COc1cccn2c(=O)c(CO)c(Nc3cccc(Cl)c3)nc12]

¹H NMR (400 MHz, DMSO-d₆) δ 3.94 (s, 3H), 4.68 (s, 2H), 6.99 (d, J=7.6 Hz, 1H), 7.09 (dd, J=7.2 Hz, J=7.2 Hz, 1H), 7.25-7.29 (m, 2H), 7.56 (d, J=8.0 Hz, 1H), 8.42 (s, 1H), 8.45 (d, J=6.8 Hz, 1H), 8.77 (s, 1H).

### 2-(4-Chlorophenylamino)-3-(hydroxymethyl)-9-methoxy-4H-pyrido[1,2-a]pyrimidin-4-one (274)

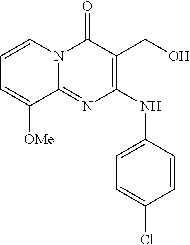 [SMILES: COc1cccn2c(=O)c(CO)c(Nc3ccc(Cl)cc3)nc12]

¹H NMR (400 MHz, DMSO-d₆) δ 3.90 (s, 3H), 4.65 (d, J=5.2 Hz, 2H), 5.19 (t, J=5.2 Hz, 1H), 7.03 (dd, J=7.2 Hz, 7.6 Hz, 1H), 7.23 (d, J=7.6 Hz, 1H), 7.29 (d, J=8.8 Hz, 2H), 7.85 (d, J=9.2 Hz, 2H), 8.42 (d, J=7.2 Hz, 1H), 8.72 (s, 1H).

### 2-(4-Fluorophenylamino)-3-(hydroxymethyl)-9-methoxy-4H-pyrido[1,2-a]pyrimidin-4-one (275)

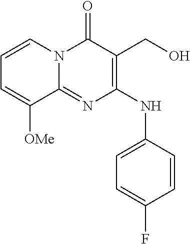 [SMILES: COc1cccn2c(=O)c(CO)c(Nc3ccc(F)cc3)nc12]

¹H NMR (400 MHz, DMSO-d₆) δ 3.91 (s, 3H), 4.69 (d, J=5.2 Hz, 2H), 5.19 (t, J=5.2 Hz, 1H), 7.06 (t, J=6.8 Hz, 1H), 7.13 (t, J=8.8 Hz, 1H), 7.25 (d, J=7.6 Hz, 1H), 7.83-7.86 (m, 1H), 8.45 (dd, J=1.2 Hz, 7.2 Hz, 1H), 8.66 (s, 1H).

### 3-(Hydroxymethyl)-9-methoxy-2-(4-(trifluoromethoxy)phenylamino)-4H-pyrido[1,2-a]pyrimidin-4-one (276)

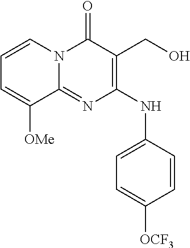 [SMILES: COc1cccn2c(=O)c(CO)c(Nc3ccc(OC(F)(F)F)cc3)nc12]

¹H NMR (400 MHz, DMSO-d₆) δ 3.96 (s, 3H), 4.67 (d, J=4.0 Hz, 2H), 5.20 (s, 1H), 7.07 (dd, J=7.2 Hz, J=7.2 Hz, 1H), 7.23 (s, 1H), 7.27 (d, J=8.0 Hz, 2H), 7.95 (dd, J=8.8 Hz, J=8.8 Hz, 2H), 8.45 (d, J=7.6 Hz, 1H), 8.78 (s, 1H).

### 3-(Hydroxymethyl)-9-methoxy-2-(4-(trifluoromethyl)phenylamino)-4H-pyrido[1,2-a]pyrimidin-4-one (277)

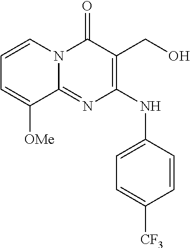 [SMILES: COc1cccn2c(=O)c(CO)c(Nc3ccc(C(F)(F)F)cc3)nc12]

¹H NMR (400 MHz, DMSO-d₆) δ 3.97 (s, 3H), 4.72 (s, 2H), 5.32 (s, 1H), 7.14, (dd, J=7.2 Hz, 7.2 Hz, 1H), 7.33 (d, J=7.6 Hz, 1H), 7.64 (d, J=8.8 Hz, 2H), 8.11 (d, J=8.8 Hz, 2H), 8.49 (d, J=7.2 Hz, 1H), 9.09 (s, 1H).

### 2-(3-Chloro-4-fluorophenylamino)-3-(hydroxymethyl)-9-methoxy-4H-pyrido[1,2-a]pyrimidin-4-one (278)

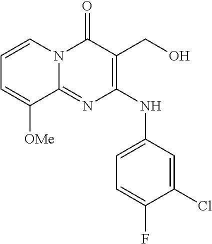 [SMILES: COc1cccn2c(=O)c(CO)c(Nc3ccc(F)c(Cl)c3)nc12]

¹H NMR (400 MHz, DMSO-d₆) δ 3.95 (s, 3H), 4.69 (d, J=4.8 Hz, 2H), 5.16 (t, J=4.8 Hz, 1H), 7.10 (dd, J=7.2 Hz, 7.2 Hz, 1H), 7.30 (dd, J=0.8 Hz, 8.0 Hz, 1H), 7.32 (dd, J=9.2 Hz, 9.2 Hz, 1H), 7.61-7.65 (m, 1H), 8.46 (dd, J=0.8 Hz, 7.2 Hz, 1H), 8.59 (dd, J=2.8 Hz, 7.2 Hz, 1H), 8.76 (s, 1H).

### 2-(3,4-Difluorophenylamino)-3-(hydroxymethyl)-9-methoxy-4H-pyrido[1,2-a]pyrimidin-4-one (279)

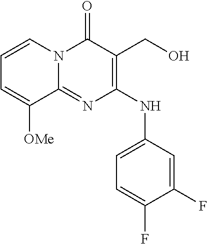 [SMILES: COc1cccn2c(=O)c(CO)c(Nc3ccc(F)c(F)c3)nc12]

m.p=231° C. (decomp.); ¹H NMR (400 MHz, CDCl₃) δ 3.92 (s, 3H), 4.66 (s, 2H), 5.17 (brs, 1H), 7.07 (dd, J=7.2 Hz, 7.2 Hz, 1H), 7.26-7.33 (m, 2H), 7.39-7.41 (m, 1H), 8.34-8.40 (m, 1H), 8.44 (d, J=7.2 Hz, 1H), 8.74 (s, 1H); ¹³C NMR (100 MHz, DMSO) δ 54.1, 56.8, 95.2, 109.1, 113.4, 116.0 (d, J=3.8 Hz, due to F), 116.8, 118.7, 137.5 (d, J=9.7 Hz, due to F), 143.2 (d, J=11.9 Hz, due to F), 145.6, 147.5 (d, J=13.4 Hz, due to F), 149.9 (d, J=13.4 Hz, due to F), 150.6, 155.5.

### 2-(3-Chloro-4-hydroxyphenylamino)-3-(hydroxymethyl)-9-methoxy-4H-pyrido[1,2-a]pyrimidin-4-one (280)

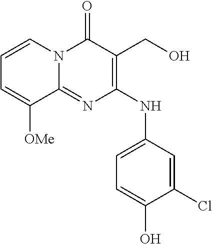 [SMILES: COc1cccn2c(=O)c(CO)c(Nc3ccc(O)c(Cl)c3)nc12]

¹H NMR (400 MHz, DMSO-d₆) δ 3.93 (s, 3H), 4.68 (s, 2H), 5.14 (s, 1H), 6.99 (d, J=8.4 Hz, 1H), 7.06 (dd, J=7.2 Hz, 7.2 Hz, 1H), 7.26 (dd, J=1.2 Hz, 8.0 Hz, 1H), 7.38 (dd, J=1.2 Hz, 8.0 Hz, 1H), 8.25 (d, J=2.8 Hz, 1H), 8.45 (dd, J=1.2 Hz, 7.2 Hz, 1H), 8.52 (s, 1H), 9.79 (s, 1H).

### 2-(3,4-Dichlorophenylamino)-3-(hydroxymethyl)-9-methoxy-4H-pyrido[1,2-a]pyrimidin-4-one (281)

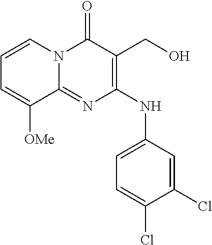 [SMILES: COc1cccn2c(=O)c(CO)c(Nc3ccc(Cl)c(Cl)c3)nc12]

¹H NMR (400 MHz, DMSO-d₆) δ 3.93 (s, 3H), 4.66 (d, J=5.2 Hz, 2H), 5.16 (d, J=5.2 Hz, 1H), 7.09 (t, J=7.2 Hz, 1H), 7.29 (d, J=6.8 Hz, 1H), 7.48 (d, J=8.8 Hz, 1H), 7.64 (dd, J=2.8 Hz, 8.8 Hz, 1H), 8.44 (d, J=7.2 Hz, 1H), 8.67 (d, J=2.8 Hz, 1H), 8.82 (s, 1H).

### 3-(Hydroxymethyl)-9-methoxy-2-(4-methyl-3-(trifluoromethyl)phenylamino)-4H-pyrido[1,2-a]pyrimidin-4-one (282)

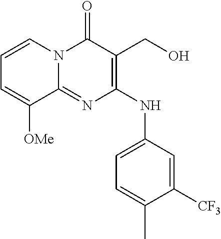 [SMILES: COc1cccn2c(=O)c(CO)c(Nc3ccc(C)c(C(F)(F)F)c3)nc12]

¹H NMR (400 MHz, DMSO-d₆) δ 2.49 (t, J=2.0 Hz, 3H due to CF₃), 3.93 (s, 3H), 4.70 (d, J=4.8 Hz, 2H), 5.19 (t, J=4.8 Hz, 1H), 7.10 (t, J=7.2 Hz, 1H), 7.29 (dd, J=1.2 Hz, 8.0 Hz, 1H), 7.32 (d, J=8.4 Hz, 1H), 7.74 (dd, J=1.6 Hz, 8.0 Hz, 1H), 8.46 (dd, J=1.2 Hz, 6.8 Hz, 1H), 8.81 (s, 1H), 8.85 (d, J=2.0 Hz, 1H).

### 2-(4-Fluoro-3-(trifluoromethyl)phenylamino)-3-(hydroxymethyl)-9-methoxy-4H-pyrido[1,2-a]pyrimidin-4-one (283)

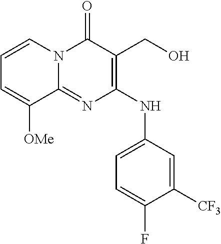 [SMILES: COc1cccn2c(=O)c(CO)c(Nc3ccc(F)c(C(F)(F)F)c3)nc12]

¹H NMR (400 MHz, DMSO-d₆) δ 3.92 (s, 3H), 4.68 (d, J=5.2 Hz, 2H), 5.12 (t, J=5.2 Hz, 1H), 7.07 (dd, J=7.2 Hz, 7.2 Hz, 1H), 7.27 (d, J=7.2 Hz, 1H), 7.37-7.42 (m, 1H), 7.86-7.88 (m, 1H), 8.43 (d, J=7.2 Hz, 1H), 8.87 (s, 1H), 8.99-9.00 (m, 1H).

### 2-(2,3-Dihydro-1H-inden-5-ylamino)-3-(hydroxymethyl)-9-methoxy-4H-pyrido[1,2-a]pyrimidin-4-one (284)

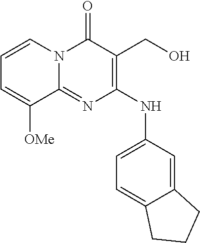 [SMILES: COc1cccn2c(=O)c(CO)c(Nc3ccc4c(c3)CCC4)nc12]

¹H NMR (400 MHz, DMSO-d₆) δ 1.97-2.05 (m, 2H), 2.79 (t, J=7.6 Hz, 2H), 2.85 (t, J=7.6 Hz, 2H), 3.92 (s, 3H), 4.69 (d, J=5.6 Hz, 2H), 5.26 (t, J=5.6 Hz, 1H), 7.04 (dd, J=7.2 Hz, 1H), 7.12 (d, J=8.4 Hz, 1H), 7.24 (dd, J=0.8 Hz, 7.6 Hz, 1H), 7.46 (dd, J=2.0 Hz, 8.0 Hz, 1H), 7.82 (s, 1H), 8.45 (dd, J=1.2 Hz, 7.2 Hz, 1H), 8.59 (s, 1H).

### 2-(Benzo[d][1,3]dioxol-5-ylamino)-3-(hydroxymethyl)-9-methoxy-4H-pyrido[1,2-a]pyrimidin-4-one (285)

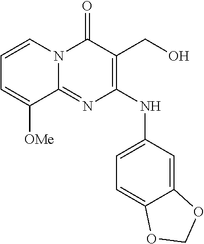 [SMILES: COc1cccn2c(=O)c(CO)c(Nc3ccc4c(c3)OCO4)nc12]

¹H NMR (400 MHz, DMSO-d₆) δ 3.91 (s, 3H), 4.68 (d, J=5.2 Hz, 2H), 5.21 (t, J=5.2 Hz, 1H), 5.98 (s, 2H), 6.84 (d, J=8.4 Hz, 1H), 7.05-7.07 (m, 1H), 7.26 (dd, J=1.2 Hz, 8.0 Hz, 1H), 7.82 (d, J=2.0 Hz, 1H), 8.46 (d, J=2.0 Hz, 1H), 8.45 (dd, J=1.2 Hz, 7.2 Hz, 1H), 8.56 (s, 1H).

### 2-(2,3-Dihydrobenzo[b][1,4]dioxin-6-ylamino)-3-(hydroxymethyl)-9-methoxy-4H-pyrido[1,2-a]pyrimidin-4-one (286)

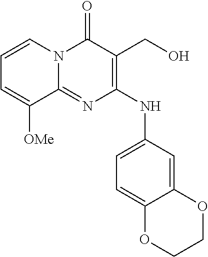 [SMILES: COc1cccn2c(=O)c(CO)c(Nc3ccc4c(c3)OCCO4)nc12]

¹H NMR (400 MHz, DMSO-d₆) δ 3.92 (s, 3H), 4.19-4.24 (m, 4H), 4.67 (d, J=5.2 Hz, 2H), 5.19 (t, J=5.2 Hz, 1H), 6.77 (d, J=8.8 Hz, 1H), 7.05 (dd, J=7.2 Hz, 7.2 Hz, 1H), 7.12 (dd, J=2.4 Hz, 8.4 Hz, 1H), 7.26 (d, J=6.8 Hz, 1H), 7.64 (d, J=2.4 Hz, 1H), 8.46 (dd, J=2.0 Hz, 7.2 Hz, 1H), 8.47 (s, 1H).

### 3-(Hydroxymethyl)-9-methoxy-2-(1-methyl-1H-indol-5-ylamino)-4H-pyrido[1,2-a]pyrimidin-4-one (287)

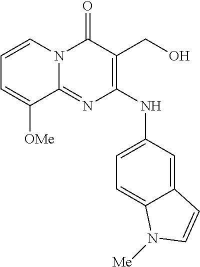 [SMILES: COc1cccn2c(=O)c(CO)c(Nc3ccc4c(ccn4C)c3)nc12]

m.p=195-197° C.; ¹H NMR (400 MHz, DMSO-d₆) δ 3.82 (s, 3H), 3.97 (s, 3H), 4.77 (d, J=5.2 Hz, 2H), 5.28 (t, J=5.2 Hz, 1H), 6.42 (d, J=3.0 Hz, 1H), 7.09 (dd, J=7.2, 7.6 Hz, 1H), 7.28-7.30 (m, 1H), 7.33 (d, J=3.0 Hz, 1H), 7.41 (d, J=8.8 Hz, 1H), 7.46 (dd, J=2.0, 8.8 Hz, 1H), 8.18 (d, J=2.0 Hz, 1H), 8.52 (dd, J=1.2, 6.8 Hz, 1H), 8.62 (br s, 1H).

### 3-(Hydroxymethyl)-9-methoxy-2-(1-methyl-1H-benzo[d]imidazol-5-ylamino)-4H-pyrido[1,2-a]pyrimidin-4-one (288)

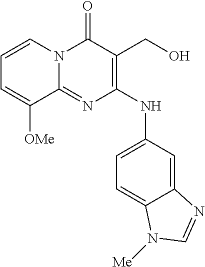 [SMILES: COc1cccn2c(=O)c(CO)c(Nc3ccc4c(c3)ncn4C)nc12]

m.p=186° C. (decomp.); ¹H NMR (400 MHz, DMSO-d₆) δ 3.87 (s, 3H), 3.98 (s, 3H), 4.79 (d, J=5.6 Hz, 2H), 5.31 (t, J=5.6 Hz, 1H), 7.08 (dd, J=7.2, 7.2 Hz, 1H), 7.28 (dd, J=0.8, 7.6 Hz, 1H), 7.50 (d, J=8.8 Hz, 1H), 7.56 (dd, J=2.0, 8.8 Hz, 1H), 8.13 (s, 1H), 8.34 (d, J=1.6 Hz, 1H), 8.53 (dd, J=0.8, 7.2 Hz, 1H), 8.73 (br s, 1H).

### 3-(Hydroxymethyl)-9-methoxy-2-(1-methyl-1H-indazol-5-ylamino)-4H-pyrido[1,2-a]pyrimidin-4-one (289)

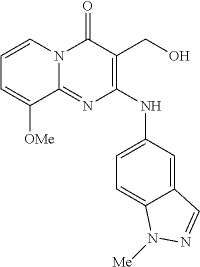 [SMILES: COc1cccn2c(=O)c(CO)c(Nc3ccc4c(cnn4C)c3)nc12]

m.p=205° C. (decomp.); ¹H NMR (400 MHz, DMSO-d₆) δ 3.40 (s, 3H), 4.08 (s, 3H), 4.78 (d, J=4.8 Hz, 2H), 5.28 (t, J=5.0 Hz, 1H), 7.12 (dd, J=7.2, 7.6 Hz, 1H), 7.32 (1H, J=1.2, 7.6 Hz, 1H), 7.62 (d, J=9.0 Hz, 1H), 7.68 (dd, J=2.0, 9.0 Hz, 1H), 8.04 (m, 1H), 8.07 (d, J=1.2 Hz, 1H), 8.53 (dd, J=1.2, 6.8 Hz, 1H), 8.75 (br s, 1H).

### 9-(Difluoromethoxy)-2-(4-fluorophenylamino)-3-(hydroxymethyl)-4H-pyrido[1,2-a]pyrimidin-4-one (290)

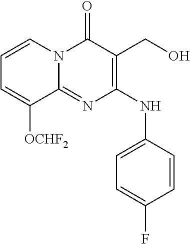 [SMILES: O=c1c(CO)c(Nc2ccc(F)cc2)nc2c(OC(F)F)cccn12]

¹H NMR (400 MHz, DMSO-d₆) δ 4.67 (d, J=5.2 Hz, 2H), 5.14 (t, J=5.2 Hz, 1H), 7.07-7.11 (m, 3H), 7.17 (t, J=74 Hz due to F₂, 1H), 7.63-7.69 (m, 3H), 8.71 (d, J=7.2 Hz, 1H), 8.75 (s, 1H).

### 2-(4-Chlorophenylamino)-9-(difluoromethoxy)-3-(hydroxymethyl)-4H-pyrido[1,2-a]pyrimidin-4-one (291)

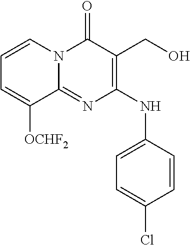 [SMILES: O=c1c(CO)c(Nc2ccc(Cl)cc2)nc2c(OC(F)F)cccn12]

¹H NMR (400 MHz, DMSO-d₆) δ 4.69 (d, J=5.6 Hz, 2H), 5.23 (t, J=5.2 Hz, 1H), 7.13 (dd, J=7.2 Hz, 7.2 Hz, 1H), 7.23 (t, J=74 Hz, 1H, due to F₂), 7.30-7.33 (m, 2H), 7.72-7.75 (m, 3H), 8.75 (dd, J=1.2 Hz, 7.2 Hz, 1H), 8.86 (s, 1H);

### 9-(Difluoromethoxy)-2-(3,4-difluorophenylamino)-3-(hydroxymethyl)-4H-pyrido[1,2-a]pyrimidin-4-one (292)

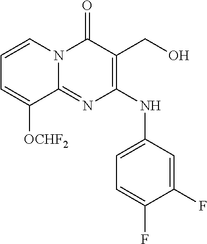 [SMILES: O=c1c(CO)c(Nc2ccc(F)c(F)c2)nc2c(OC(F)F)cccn12]

¹H NMR (400 MHz, DMSO-d₆) δ 4.70 (d, J=5.2 Hz, 2H), 5.22 (s, 1H), 7.16 (dd, J=7.2 Hz, J=7.2 Hz, 1H), 7.26 (t, J=74 Hz, due to F2, 1H), 7.33-7.38 (m, 2H), 7.75 (d, J=7.2 Hz, 1H), 8.12 (dd, J=7.6 Hz, 12.8 Hz, 1H), 8.76 (d, J=6.8 Hz, 1H), 8.90 (s, 1H); LC-MS (ESI, m/z): 370 [M+H]⁺.

### 2-(3,4-Dichlorophenylamino)-9-(difluoromethoxy)-3-(hydroxymethyl)-4H-pyrido[1,2-a]pyrimidin-4-one (293)

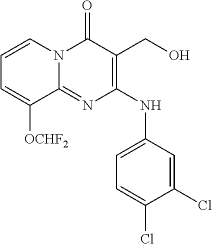 [SMILES: O=c1c(CO)c(Nc2ccc(Cl)c(Cl)c2)nc2c(OC(F)F)cccn12]

¹H NMR (400 MHz, DMSO-d₆) δ 4.68 (s, 2H), 5.19 (s, 1H), 7.15 (t, J=7.2 Hz, 1H), 7.24 (t, J=74 Hz, due to F₂, 1H), 7.47-7.57 (m, 2H), 7.72 (d, J=7.2 Hz, 1H), 8.32 (d, J=2.4 Hz, 1H), 8.73 (dd, J=1.6 Hz, 7.2 Hz, 1H), 8.92 (s, 1H).

### 2-(3-Chloro-4-fluorophenylamino)-9-(difluoromethoxy)-3-(hydroxymethyl)-4H-pyrido[1,2-a]pyrimidin-4-one (294)

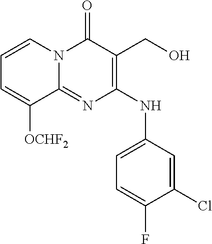 [SMILES: O=c1c(CO)c(Nc2ccc(F)c(Cl)c2)nc2c(OC(F)F)cccn12]

¹H NMR (400 MHz, DMSO-d₆) δ 4.68 (d, J=4.0 Hz, 2H), 5.18 (s, 1H), 7.15 (dd, J=7.2 Hz, 7.2 Hz, 1H), 7.24 (t, J=74 Hz, 1H, due to F₂), 7.32 (dd, J=9.2 Hz, 9.2 Hz, 1H), 7.50-7.54 (m, 1H), 7.73 (d, J=7.6 Hz, 1H), 8.22 (dd, J=2.8 Hz, 6.8 Hz, 1H), 8.74 (dd, J=1.2 Hz, 7.2 Hz, 1H), 8.86 (s, 1H).

### 2-(1H-Indol-5-ylamino)-9-(difluoromethoxy)-3-(hydroxymethyl)-4H-pyrido[1,2-a]pyrimidin-4-one (295)

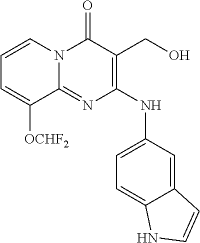 [SMILES: O=c1c(CO)c(Nc2ccc3[nH]ccc3c2)nc2c(OC(F)F)cccn12]

¹H NMR (400 MHz, DMSO-d₆) δ 4.72 (d, J=4.8 Hz, 2H), 5.23 (t, J=4.8 Hz, 1H), 6.34 (s, 1H), 7.05-7.09 (m, 1H), 7.23 (dd, J=8.8 Hz, 8.8 Hz, 1H), 7.25 (t, J=74.4 Hz, 1H due to F₂), 7.31-7.33 (m, 2H), 7.68 (d, J=7.2 Hz, 1H), 7.93 (s, 1H), 8.70 (s, 1H), 8.73 (d, J=1.2 Hz, 1H), 10.99 (s, 1H).

### 2-(3-chlorophenylamino)-3-(hydroxymethyl)-6,8-dimethyl-4H-pyrido[1,2-a]pyrimidin-4-one (296)

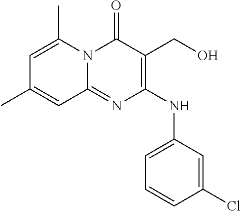 [SMILES: Cc1cc(C)n2c(=O)c(CO)c(Nc3cccc(Cl)c3)nc2c1]

¹H NMR (400 MHz, CDCl₃) δ 2.32 (s, 3H), 2.40 (s, 3H), 3.55 (s, 2H), 6.78 (s, 1H), 7.06 (d, J=2.0 Hz, 1H), 7.21 (dd, J=8.0 Hz, J=8.0 Hz, 1H), 7.39 (d, J=8.4 Hz, 1H), 7.69 (d, J=2.0 Hz, 1H), 7.71 (s, 1H), 9.60 (s, 1H); LC-MS (ESI, m/z): 330 [M+H]⁺.

### 7,9-Dichloro-2-(3-chlorophenylamino)-3-(hydroxymethyl)-4H-pyrido[1,2-a]pyrimidin-4-one (297)

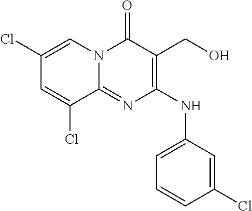 [SMILES: O=c1c(CO)c(Nc2cccc(Cl)c2)nc2c(Cl)cc(Cl)cn12]

¹H NMR (400 MHz, DMSO-d₆) δ 4.65 (s, 2H), 5.70 (d, J=7.6 Hz, 1H), 7.29 (dd, J=8.0 Hz, J=8.0 Hz, 1H), 7.57 (dd, J=8.0 Hz, J=8.0 Hz, 1H), 8.25 (s, 1H), 8.32 (d, J=2.0 Hz, 1H), 8.76 (d, J=2.0 Hz, 1H).

### 2-(3-Chlorophenylamino)-7,9-difluoro-3-(hydroxymethyl)-4H-pyrido[1,2-a]pyrimidin-4-one (298)

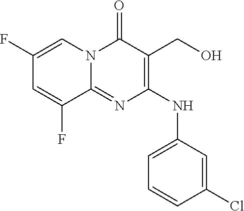 [SMILES: O=c1c(CO)c(Nc2cccc(Cl)c2)nc2c(F)cc(F)cn12]

¹H NMR (400 MHz, CDCl₃) δ 4.69 (d, J=4.8 Hz, 2H), 5.31 (t, J=4.8 Hz, 1H), 7.06 (dd, J=1.2 Hz, 8.0 Hz, 1H), 7.32 (t, J=8.0 Hz, 1H), 7.56 (dd, J=1.2 Hz, 8.0 Hz, 1H), 8.02 (s, 1H), 8.18-8.23 (m, 1H), 8.68 (t, J=2.0 Hz, 1H), 8.90 (s, 1H).

### (4-Oxo-2-(phenylamino)-4H-pyrido[1,2-a]pyrimidin-3-yl)methyl benzoate (299)

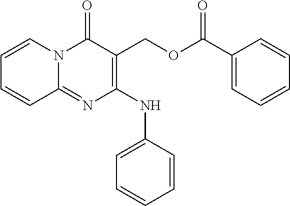 [SMILES: O=C(OCc1c(Nc2ccccc2)nc2ccccn2c1=O)c1ccccc1]

m.p=178-179° C.; ¹H NMR (400 MHz, DMSO-d₆) δ 5.66 (s, 2H), 6.96 (ddd, J=1.2, 1.2, 6.8 Hz, 1H), 7.06-7.10 (m, 1H), 7.33-7.44 (m, 5H), 7.53-7.56 (m, 1H), 7.61-7.65 (m, 1H), 7.72 (m, 2H), 8.12 (dd, J=1.2, 8.4 Hz, 1H), 9.14 (brs, 1H).

### (4-Oxo-2-(phenylamino)-4H-pyrido[1,2-a]pyrimidin-3-yl)methyl acetate (300)

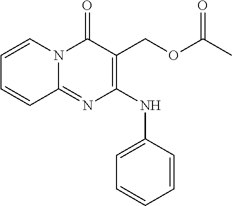 [SMILES: CC(=O)OCc1c(Nc2ccccc2)nc2ccccn2c1=O]

m.p=160-161° C.; ¹H NMR (400 MHz, CDCl₃) δ 2.13 (s, 3H), 6.92 (dd, J=6.8, 7.2 Hz, 1H), 7.04-7.08 (m, 1H), 7.30-7.37 (m, 3H), 7.59-7.66 (m, 3H), 8.91 (brs, 1H), 8.94 (d, J=7.2 Hz, 1H).

### (4-Oxo-2-(phenylamino)-4H-pyrido[1,2-a]pyrimidin-3-yl)methyl isobutyrate (301)

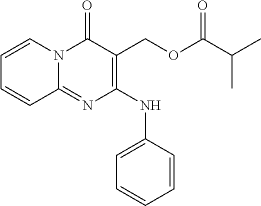 [SMILES: CC(C)C(=O)OCc1c(Nc2ccccc2)nc2ccccn2c1=O]

m.p=161-163° C.; ¹H NMR (400 MHz, CDCl₃) δ 1.17 (d, J=7.2 Hz, 6H), 2.62-2.65 (m, 1H), 6.94 (dd, J=6.8, 7.2 Hz, 1H), 7.04-7.08 (m, 1H), 7.31-7.38 (m, 3H), 7.60-7.67 (m, 3H), 8.95 (brs, 1H), 8.95 (d, J=6.8 Hz, 1H).

### (9-Methoxy-4-oxo-2-(quinolin-6-ylamino)-4H-pyrido[1,2-a]pyrimidin-3-yl)methyl acetate (302)

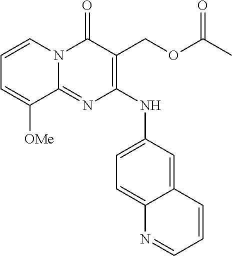 [SMILES: COc1cccn2c(=O)c(COC(C)=O)c(Nc3ccc4ncccc4c3)nc12]

Yellow solid (79%); mp=181.0-183.3° C.; ¹H NMR (400 MHz, CDCl₃) δ 2.16 (s, 3H), 4.02 (s, 3H), 5.41 (s, 2H), 6.90 (dd, J=7.2, 7.6 Hz, 1H), 6.97 (dd, J=1.2, 7.6 Hz, 1H), 7.32 (dd, J=4.4, 8.4 Hz, 1H), 7.97 (dd, J=2.0, 8.4 Hz, 1H), 8.03-8.09 (m, 2H), 8.57 (d, J=2.0 Hz, 1H), 8.61 (dd, J=1.2, 7.2 Hz, 1H), 8.77 (dd, J=1.2, 4.4 Hz, 1H), 9.32 (s, 1H); ¹³C NMR (100 MHz, CDCl₃) δ 21.3, 57.0, 59.0, 92.5, 112.5, 113.0, 115.5, 119.8, 121.4, 124.7, 129.2, 129.9, 135.7, 138.1, 145.0, 145.3, 148.7, 151.4, 156.7, 159.2, 174.6; LCMS (electrospray) m/z 391 (M+H)⁺.

### (9-Methoxy-4-oxo-2-(quinolin-6-ylamino)-4H-pyrido[1,2-a]pyrimidin-3-yl)methyl benzoate (303)

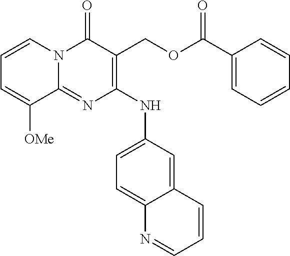 [SMILES: COc1cccn2c(=O)c(COC(=O)c3ccccc3)c(Nc3ccc4ncccc4c3)nc12]

Pale yellow solid (44%); mp=218.8-221.0° C.; ¹H NMR (400 MHz, CDCl₃+CD₃OD) δ 4.00 (s, 3H), 5.64 (s, 2H), 6.91 (dd, J=7.2, 7.6 Hz, 1H), 6.99 (d, J=7.2 Hz, 1H), 7.34-7.42 (m, 3H), 7.50-7.54 (m, 1H), 8.01-8.12 (m, 5H), 8.59 (dd, J=1.2, 7.2 Hz, 1H), 8.63 (d, J=2.0 Hz, 1H), 8.69 (d, J=1.2 Hz, 1H); LCMS (electrospray) m/z 453 (M+H)⁺.

### (9-Methoxy-4-oxo-2-(quinolin-6-ylamino)-4H-pyrido[1,2-a]pyrimidin-3-yl)methyl pivalate (304)

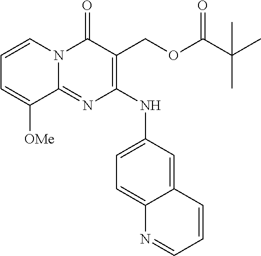 [SMILES: COc1cccn2c(=O)c(COC(=O)C(C)(C)C)c(Nc3ccc4ncccc4c3)nc12]

¹H NMR (400 MHz, CDCl₃) δ 1.22 (s, 9H), 4.06 (s, 3H), 5.48 (s, 2H), 6.93 (dd, J=7.2, 7.6 Hz, 1H), 7.00 (dd, J=1.2, 7.6 Hz, 1H), 7.36 (dd, J=4.4, 8.0 Hz, 1H), 7.98 (dd, J=2.4, 8.8 Hz, 1H), 8.07 (s, 1H), 8.09 (dd, J=8.0, 8.8 Hz, 1H), 8.65-8.67 (m, 2H), 8.79 (d, J=2.4 Hz, 1H), 9.31 (s, 1H); LCMS (electrospray) m/z 433 (M+H)⁺.

### (9-Methoxy-4-oxo-2-(quinolin-6-ylamino)-4H-pyrido[1,2-a]pyrimidin-3-yl)methyl 2-methylbenzoate (305)

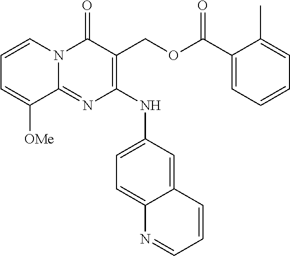 [SMILES: COc1cccn2c(=O)c(COC(=O)c3ccccc3C)c(Nc3ccc4ncccc4c3)nc12]

¹H NMR (400 MHz, CDCl₃) δ 2.65 (s, 3H), 4.05 (s, 3H), 5.69 (s, 2H), 6.92 (dd, J=7.2, 7.6 Hz, 1H), 7.00 (dd, J=1.2, 7.6 Hz, 1H), 7.21-7.27 (m, 2H), 7.36-7.41 (m, 2H), 8.02-8.14 (m, 4H), 8.66-8.69 (m, 2H), 8.80 (brs, 1H), 9.60 (s, 1H); ¹³C NMR (100 MHz, CDCl₃) δ□ 22.1, 57.0, 59.0, 92.8, 112.6, 113.0, 115.6, 120.0, 121.5, 125.0, 126.0, 129.0, 129.3, 129.7, 131.4, 131.9, 132.7, 136.0, 138.4, 140.8, 145.1, 148.5, 151.5, 156.9, 159.2, 170.7; LCMS (electrospray) m/z 467 (M+H)⁺.

### 9-Methoxy-4-oxo-2-(4-(4-phenylpiperazin-1-yl)benzylamino)-4H-pyrido[1,2-a]pyrimidine-3-carbaldehyde (306)

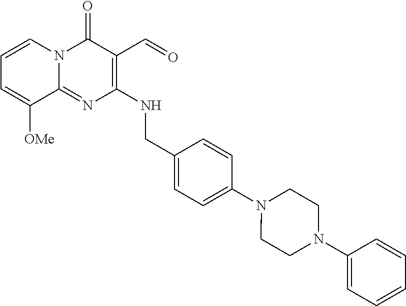 [SMILES: COc1cccn2c(=O)c(C=O)c(NCc3ccc(N4CCN(c5ccccc5)CC4)cc3)nc12]

Pale yellow solid (90%); mp=168.1-169.3° C.; ¹H NMR (400 MHz, CDCl₃) δ 3.28-3.38 (m, 8H), 3.99 (s, 3H), 4.80 (d, J=5.6 Hz, 2H), 6.82-6.90 (m, 2H), 6.93-7.02 (m, 5H), 7.27-7.34 (m, 4H), 8.51 (dd, J=1.2, 7.2 Hz, 1H), 9.95 (brs, 1H), 10.28 (s, 1H); LCMS (electrospray) m/z 469 (M+H)⁺.

### 3-(Hydroxymethyl)-9-methoxy-2-(4-(4-phenylpiperazin-1-yl)benzylamino)-4H-pyrido[1,2-a]pyrimidin-4-one (307)

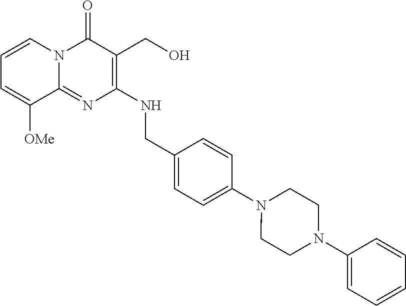 [SMILES: COc1cccn2c(=O)c(CO)c(NCc3ccc(N4CCN(c5ccccc5)CC4)cc3)nc12]

White solid (60%); mp=134.8-136.0° C.; ¹H NMR (400 MHz, CDCl₃) δ 2.00 (t, J=6.0 Hz, 1H), 3.99 (s, 3H), 4.75 (d, J=5.6 Hz, 2H), 4.80 (d, J=6.0 Hz, 2H), 5.98 (brt, J=5.6 Hz, 1H), 6.81-6.99 (m, 7H), 7.27-7.34 (m, 4H), 8.57 (dd, J=1.2, 7.2 Hz, 1H).

### 2-(4-(4-(4-Fluorophenyl)piperazin-1-yl)benzylamino)-9-methoxy-4-oxo-4H-pyrido[1,2-a]pyrimidine-3-carbaldehyde (308)

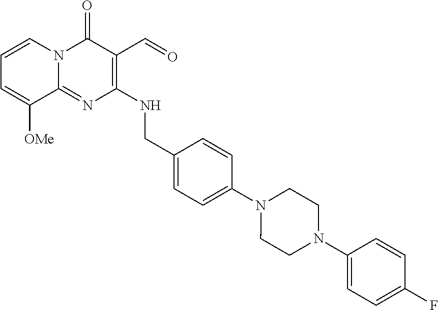 [SMILES: COc1cccn2c(=O)c(C=O)c(NCc3ccc(N4CCN(c5ccc(F)cc5)CC4)cc3)nc12]

Pale yellow solid (80%); mp=236.7-237.1° C.; ¹H NMR (400 MHz, DMSO-d₆) δ 3.19-3.21 (m, 4H), 3.24-3.26 (m, 4H), 3.94 (s, 3H), 4.69 (d, J=6.0 Hz, 2H), 6.95-7.09 (m, 7H), 7.29 (d, J=8.8 Hz, 2H), 7.39 (d, J=7.6 Hz, 1H), 8.40 (dd, J=1.2, 7.2 Hz, 1H), 9.74 (t, J=6.0 Hz, 1H), 10.07 (s, 1H).

### 2-(4-(4-(4-Fluorophenyl)piperazin-1-yl)benzylamino)-3-(hydroxymethyl)-9-methoxy-4H-pyrido[1,2-a]pyrimidin-4-one (309)

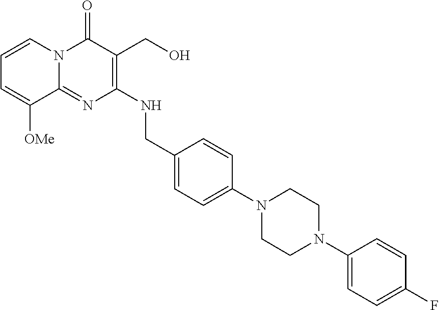 [SMILES: COc1cccn2c(=O)c(CO)c(NCc3ccc(N4CCN(c5ccc(F)cc5)CC4)cc3)nc12]

Pale yellow solid (88%); mp=136.1-137.8° C.; ¹H NMR (400 MHz, CDCl₃) δ 3.22-3.24 (m, 4H), 3.29-3.32 (m, 4H), 3.97 (s, 3H), 4.69 (d, J=5.6 Hz, 2H), 4.77 (brs, 2H), 6.19 (t, J=5.6 Hz, 1H), 6.75 (dd, J=7.2, 7.6 Hz, 1H), 6.87-6.99 (m, 7H), 7.30 (d, J=8.4 Hz, 2H), 8.46 (d, J=7.2 Hz, 1H); ¹³C NMR (100 MHz, CDCl₃) δ 44.8, 49.6, 50.5, 56.8, 56.9, 93.3, 111.7, 115.6, 115.8, 116.6, 118.3, 118.4, 120.0, 129.1, 130.6, 144.7, 148.0, 150.6, 150.8, 156.3, 157.0, 158.7, 159.2.

### 2-(4-Chlorobenzylamino)-9-methoxy-4-oxo-4H-pyrido[1,2-a]pyrimidine-3-carbaldehyde (310)

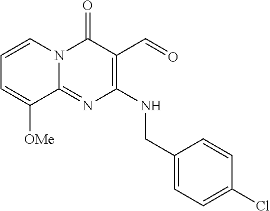 [SMILES: COc1cccn2c(=O)c(C=O)c(NCc3ccc(Cl)cc3)nc12]

Pale yellow solid (90%); mp=189.0-190.6° C.; ¹H NMR (400 MHz, CDCl₃) δ 3.98 (s, 3H), 4.83 (d, J=5.6 Hz, 2H), 6.85 (dd, J=7.2, 7.6 Hz, 1H), 7.02 (d, J=7.6 Hz, 1H), 7.28 (d, J=8.8 Hz, 2H), 7.34 (d, J=8.8 Hz, 2H), 8.52 (dd, J=1.2, 7.2 Hz, 1H), 10.02 (brs, 1H), 10.28 (s, 1H).

### 2-C4-Chlorobenzylamino)-3-(hydroxymethyl)-9-methoxy-4H-pyrido[1,2-a]pyrimidin-4-one (311)

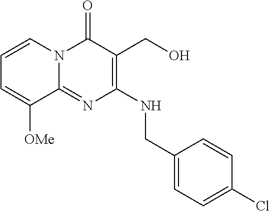 [SMILES: COc1cccn2c(=O)c(CO)c(NCc3ccc(Cl)cc3)nc12]

White solid (40%); mp=198.8-200.0° C.; ¹H NMR (400 MHz, DMSO-d₆) δ 3.89 (s, 3H), 4.54 (d, J=5.2 Hz, 2H), 4.67 (d, J=6.0 Hz, 2H), 4.77 (t, J=5.2 Hz, 1H), 6.95 (dd, J=7.2, 7.6 Hz, 1H), 7.18 (dd, J=1.2, 7.6 Hz, 1H), 7.27 (brt, J=6.0 Hz, 1H), 7.34 (d, J=8.8 Hz, 2H), 7.43 (d, J=8.4 Hz, 2H), 8.40 (dd, J=1.2, 7.2 Hz, 1H); LCMS (electrospray) m/z 346 (M+H)⁺.

### 2-(4-(4-(4-Fluorophenyl)piperazin-1-yl)phenylamino)-9-methoxy-4-oxo-4H-pyrido[1,2-a]pyrimidine-3-carbaldehyde (312)

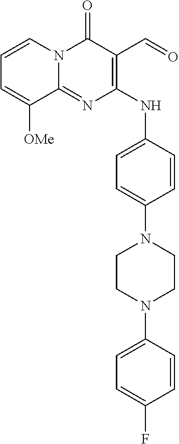 [SMILES: COc1cccn2c(=O)c(C=O)c(Nc3ccc(N4CCN(c5ccc(F)cc5)CC4)cc3)nc12]

Dark orange solid (91%); mp=277.8-278.5° C.; ¹H NMR (400 MHz, CDCl₃) δ 3.28-3.34 (m, 8H), 4.00 (s, 3H), 6.88-7.05 (m, 8H), 7.80 (d, J=8.8 Hz, 2H), 8.55 (dd, J=1.2, 6.4 Hz, 1H), 10.32 (s, 1H), 11.69 (s, 1H).

### 2-(4-(4-(4-Fluorophenyl)piperazin-1-yl)phenylamino)-3-(hydroxymethyl)-9-methoxy-4H-pyrido[1,2-a]pyrimidin-4-one (313)

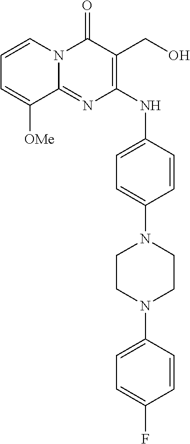 [SMILES: COc1cccn2c(=O)c(CO)c(Nc3ccc(N4CCN(c5ccc(F)cc5)CC4)cc3)nc12]

Pale yellow solid (84%); mp=>335° C. (decomp.); ¹H NMR (400 MHz, DMSO-d6) δ 3.23 (brs, 8H), 3.93 (s, 3H), 4.69 (d, J=5.2 Hz, 2H), 5.19 (t, J=5.2 Hz, 1H), 6.97-7.10 (m, 7H), 7.25 (d, J=6.8 Hz, 1H), 7.69 (d, J=9.2 Hz, 2H), 8.46 (dd, J=0.8, 6.8 Hz, 1H), 8.50 (brs, 1H); LCMS (electrospray) m/z 476 (M+H)⁺.

### 2-(3-(4-(4-Fluorophenyl)piperazin-1-yl)phenylamino)-9-methoxy-4-oxo-4H-pyrido[1,2-a]pyrimidine-3-carbaldehyde (314)

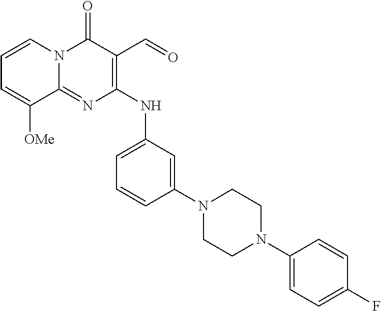 [SMILES: COc1cccn2c(=O)c(C=O)c(Nc3cccc(N4CCN(c5ccc(F)cc5)CC4)c3)nc12]

Yellow solid (91%); mp=227.5-228.3° C.; ¹H NMR (400 MHz, CDCl₃) δ 3.30 (brs, 4H), 3.46 (brs, 4H), 3.99 (s, 3H), 6.78 (s, 1H), 6.93-7.13 (m, 6H), 7.24-7.28 (m, 2H), 8.07 (s, 1H), 8.58 (d, J=7.2 Hz, 1H), 10.33 (s, 1H), 11.77 (s, 1H); LCMS (electrospray) m/z 474 (M+H)⁺.

### 2-(3-(4-(4-Fluorophenyl)piperazin-1-yl)phenylamino)-3-(hydroxymethyl)-9-methoxy-4H-pyrido[1,2-a]pyrimidin-4-one (315)

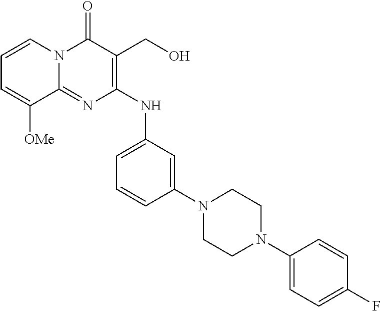 [SMILES: COc1cccn2c(=O)c(CO)c(Nc3cccc(N4CCN(c5ccc(F)cc5)CC4)c3)nc12]

Yellow solid (56%); mp=201.1-201.7° C.; ¹H NMR (400 MHz, DMSO-d6) δ 3.27-3.36 (m, 8H), 3.95 (s, 3H), 4.72 (d, J=5.2 Hz, 2H), 5.34 (t, J=5.2 Hz, 1H), 6.64 (dd, J=2.0, 8.0 Hz, 1H), 6.86 (dd, J=1.2, 7.6 Hz, 1H), 7.03-7.17 (m, 6H), 7.31 (d, J=8.0 Hz, 1H), 8.23 (s, 1H), 8.48 (dd, J=0.8, 6.8 Hz, 1H), 8.64 (s, 1H); LCMS (electrospray) m/z 476 (M+H)⁺.

### (9-Methoxy-4-oxo-2-(quinolin-6-ylamino)-4H-pyrido[1,2-a]pyrimidin-3-yl)methyl methyl carbonate (316)

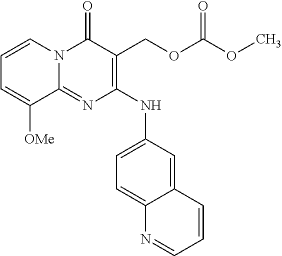 [SMILES: COC(=O)OCc1c(Nc2ccc3ncccc3c2)nc2c(OC)cccn2c1=O]

White solid; ¹H NMR (400 MHz, CDCl₃) δ 3.49 (s, 3H), 4.07 (s, 3H), 4.88 (s, 2H), 6.96 (dd, J=7.2 Hz, 7.2 Hz, 1H), 7.00 (d, J=7.2 Hz, 1H), 7.37 (dd, J=4.4 Hz, 8.0 Hz, 1H), 7.86 (dd, J=2.8 Hz, 8.8 Hz, 1H), 8.05 (d, J=9.2 Hz, 1H), 8.11 (d, J=9.2 Hz, 1H), 8.52 (s, 1H), 8.59 (d, J=2.4 Hz, 1H), 8.66 (dd, J=1.6 Hz, 6.8 Hz, 1H), 8.80 (dd, J=1.6 Hz, 4.4 Hz, 1H); LCMS (electrospray) m/z (M+H)⁺ 407.

### 3-(Hydroxymethyl)-9-methoxy-2-((4-(4-(4-(trifluoromethoxy)phenoxy)piperidin-1-yl)benzyl)amino)-4H-pyrido[1,2-a]pyrimidin-4-one (317)

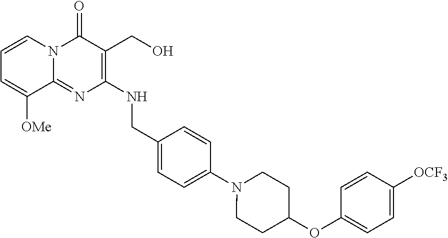 [SMILES: COc1cccn2c(=O)c(CO)c(NCc3ccc(N4CCC(Oc5ccc(OC(F)(F)F)cc5)CC4)cc3)nc12]

White solid; ¹H NMR (400 MHz, CDCl₃) δ 1.90-1.96 (m, 2H), 2.07-2.12 (m, 2H), 3.06-3.12 (m, 2H), 3.46-3.51 (m, 2H), 3.99 (s, 3H), 4.41-4.45 (m, 1H), 4.73 (d, J=5.6 Hz, 2H), 4.80 (s, 2H), 5.59 (brs, 1H), 6.80 (dd, J=7.2 Hz, 7.2 Hz, 1H), 6.89-6.93 (m, 5H), 7.13 (d, J=8.4 Hz, 2H), 7.30 (d, J=8.4 Hz, 2H), 8.56 (d, J=7.2 Hz, 1H); LCMS (electrospray) m/z (M+H)⁺ 571.

### 9-Methoxy-4-oxo-2-((4-(4-(4-(trifluoromethoxy)phenoxy)piperidin-1-yl)benzyl)amino)-4H-pyrido[1,2-a]pyrimidine-3-carbaldehyde (318)

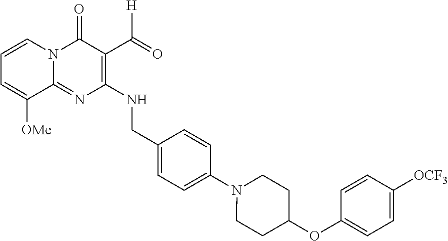 [SMILES: COc1cccn2c(=O)c(C=O)c(NCc3ccc(N4CCC(Oc5ccc(OC(F)(F)F)cc5)CC4)cc3)nc12]

White solid; ¹H NMR (400 MHz, CDCl₃) δ 1.93 (m, 2H), 2.09 (m, 2H), 3.10 (m, 2H), 3.45-3.50 (m, 2H), 3.99 (s, 3H), 4.43 (m, 1H), 4.79 (d, J=5.6 Hz, 2H), 6.84 (dd, J=7.2 Hz, 7.2 Hz, 1H), 6.89-6.93 (m, 4H), 7.01 (d, J=7.6 Hz, 1H), 7.13 (d, J=8.4 Hz, 2H), 7.30 (d, J=8.4 Hz, 2H), 8.52 (dd, J=1.2 Hz, 6.8 Hz, 1H), 9.93 (brs, 1H), 10.27 (s, 1H); LCMS (electrospray) m/z (M+H)⁺ 569.

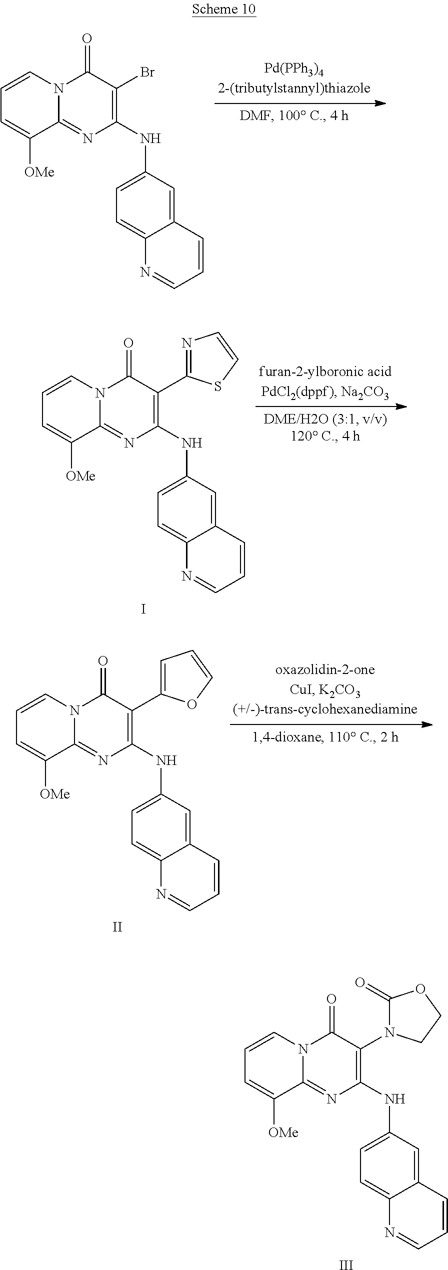

A mixture of 3-bromo-9-methoxy-2-(quinolin-6-ylamino)-4H-pyrido[1,2-a]pyrimidin-4-one (0.25 mmol), Pd(PPh₃)₄ (0.050 mmol) and 2-(tributylstannyl)thiazole (0.38 mmo) in DMF (1.5 mL) was stirred at 100° C. for 4 h. The solvent was concentrated under reduced pressure the resulting mixture was diluted with methylene chloride and washed with saturated Na₂CO₃ (aq.). The organic layer was washed with brine again, dried over MgSO₄ and concentrated in vacuo. The resulting crude residue was purified by flash column chromatography (MC:MeOH=50:1) and prep-HPLC (MC:MeOH=50:1) to give a target compound I.

To a solution of 3-bromo-9-methoxy-2-(quinolin-6-ylamino)-4H-pyrido[1,2-a]pyrimidin-4-one (0.25 mmol) in DME/H₂O (3:1, v/v, 1.3 mL) were added 2-furylboronic acid (0.30 mmol), PdCl₂(dppf) (7.56 umol) and Na₂CO₃ (0.50 mmol) and the resulting mixture was heated to 120° C. for 4 h. After reaction completion, the resulting mixture was diluted with methylene chloride and washed with saturated Na₂CO₃ (aq.). The organic layer was dried over MgSO₄ and concentrated in vacuo. The crude residue was purified by flash column chromatography (MC:MeOH=50:1) and prep-HPLC (MC:MeOH=50:1) to give a target compound II.

CuI (6.30 umol), K₂CO₃ (0.25 mmol) and oxazolidinone (0.13 mmol) were placed to the reaction flask filled with N₂ and then, a solution of 3-bromo-9-methoxy-2-(quinolin-6-ylamino)-4H-pyrido[1,2-a]pyrimidin-4-one (0.25 mmol) and (+/−)-trans-cyclohexanediamine (0.013 mmol) was added at rt. The reaction mixture was stirred at 110° C. for 2 h, the resulting mixture was diluted with methylene chloride and washed with saturated Na₂CO₃ (aq.). The organic layer was dried over MgSO₄ and concentrated in vacuo. The crude residue was purified by flash column chromatography (MC:MeOH=50:1 to 30:1) and prep-HPLC (MC:MeOH=30:1) to give a target compound III.

### 9-Methoxy-2-(quinolin-6-ylamino)-3-(thiazol-2-yl)-4H-pyrido[1,2-a]pyrimidin-4-one (319)

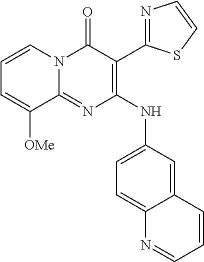 [SMILES: COc1cccn2c(=O)c(-c3nccs3)c(Nc3ccc4ncccc4c3)nc12]

yellow solid (31%); mp=237.1-238.2° C.; ¹H NMR (400 MHz, CDCl₃) δ 4.10 (s, 3H), 6.98 (dd, J=7.2, 7.6 Hz, 1H), 7.04 (dd, J=1.2, 7.6 Hz, 1H), 7.37 (d, J=4.4 Hz, 1H), 7.39 (d, J=3.6 Hz, 1H), 7.96 (d, J=3.6 Hz, 1H), 8.06-8.11 (m, 2H), 8.13 (dd, J=1.6, 8.4 Hz, 1H), 8.75 (dd, J=1.2, 7.2 Hz, 1H), 8.82 (dd, J=1.6, 4.4 Hz, 1H), 8.91 (d, J=1.6 Hz, 1H), 13.5 (s, 1H); LCMS (electrospray) m/z 402 (M+H)⁺.

### 3-(Furan-2-yl)-9-methoxy-2-(quinolin-6-ylamino)-4H-pyrido[1,2-a]pyrimidin-4-one (320)

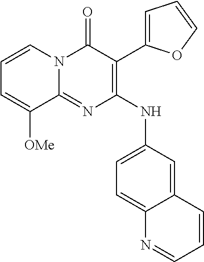 [SMILES: COc1cccn2c(=O)c(-c3ccco3)c(Nc3ccc4ncccc4c3)nc12]

Dark yellow solid (41%); mp=199.1-200.8° C.; ¹H ¹H NMR (400 MHz, CDCl₃) δ 4.07 (s, 3H), 6.66 (dd, J=1.6, 3.6 Hz, 1H), 6.95-6.97 (m, 2H), 7.36 (dd, J=4.4, 8.4 Hz, 1H), 7.44 (d, J=3.6 Hz, 1H), 7.61 (d, J=1.6 Hz, 1H), 7.86 (dd, J=2.4, 9.2 Hz, 1H), 8.05 (d, J=9.2 Hz, 1H), 8.09 (d, J=8.4 Hz, 1H), 8.71 (dd, J=2.4, 6.8 Hz, 1H) 8.78 (d, J=2.4 Hz, 1H), 8.80 (dd, J=1.6, 4.4 Hz, 1H), 9.38 (s, 1H); ¹³C NMR (100 MHz, CDCl₃) δ 57.0, 90.3, 110.8, 111.5, 112.3, 113.1, 116.4, 119.6, 121.6, 125.3, 129.3, 129.9, 135.8, 137.8, 140.1, 142.6, 145.4, 148.9, 149.0, 151.4, 152.9, 155.4.

### 3-(9-Methoxy-4-oxo-2-(quinolin-6-ylamino)-4H-pyrido[1,2-a]pyrimidin-3-yl)oxazolidin-2-one (321)

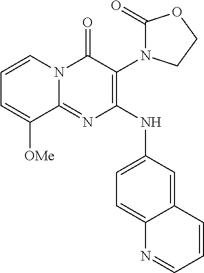 [SMILES: COc1cccn2c(=O)c(N3CCOC3=O)c(Nc3ccc4ncccc4c3)nc12]

White solid (62%); mp=276.9-277.8° C.; ¹H NMR (400 MHz, CDCl₃+CD₃OD) δ 3.93 (s, 3H), 3.96 (t, J=5.2 Hz, 2H), 4.41 (t, J=5.2 Hz, 2H), 6.98 (dd, J=1.2, 7.6 Hz, 1H), 7.08 (dd, J=7.2, 7.6 Hz, 1H), 7.44 (dd, J=4.4, 8.4 Hz, 1H), 8.10 (dd, J=2.4, 9.2 Hz, 1H), 8.20 (d, J=9.2 Hz, 1H), 8.24-8.26 (m, 2H), 8.72 (dd, J=1.2, 7.2 Hz, 1H), 8.87 (dd, J=1.6, 4.4 Hz, 1H); LCMS (electrospray) m/z 404 (M+H)⁺.

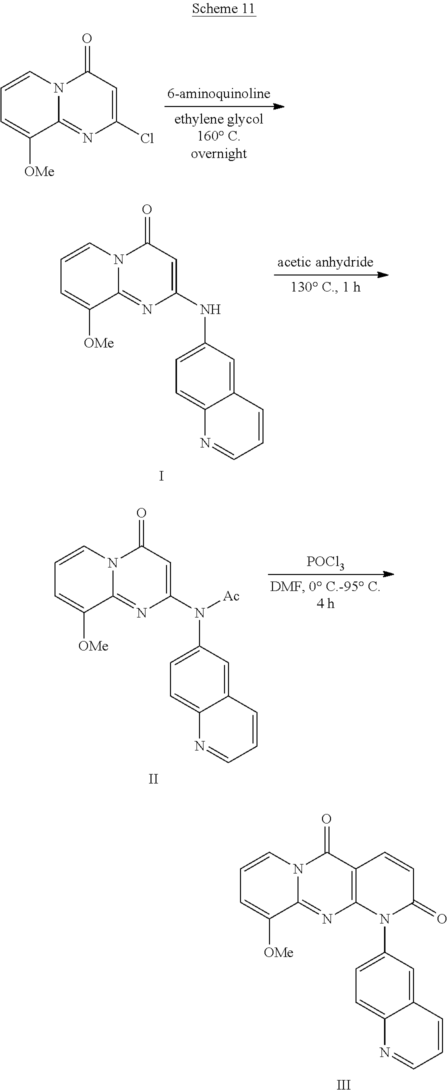

A mixture of 2-chloro-9-methoxy-4H-pyrido[1,2-a]pyrimidin-4-one (2.23 mmol), 6-aminoquinoline (0.011 mol) and ethylene glycol (12 mL) was stirred at 160° C. for overnight. The reaction mixture was poured to the water and extracted with methylene chloride several times. The organic layer was dried over MgSO₄ and concentrated in vacuo. The resulting solid was purified by flash column chromatography (MC:MeOH=30:1) and then resulting residue was dissolved with MC. At that time, the generating white solid was filtered and dried to give a target compound I.

Compound I (0.53 mmol) was suspended in acetic anhydride (3 mL) and the mixture was heated at 130° C. for an hour. After reaction completion, the reaction mixture was poured to water and extracted with methylene chloride several times. The crude residue was dissolved with MeOH and unsoluble solid was filtered and dried in vacuo to give a target compound II.

POCl₃ (0.67 mmol) was added dropwise with stirring to DMF (2 mL) which was contained in reaction flask with an ice-bath under N₂. The resulting solution was stirred at rt for 30 min, then a solution of compound II (0.44 mmol) in DMF (1 mL) was added and the reaction mixture was heated at 95° C. for 4 h. After reaction completion, the mixture was poured to ice and stirred for 10 min. At that time, the generating solid was filtered and dried in vacuo to give a target compound III.

### 9-Methoxy-2-(quinolin-6-ylamino)-4H-pyrido[1,2-a]pyrimidin-4-one (322)

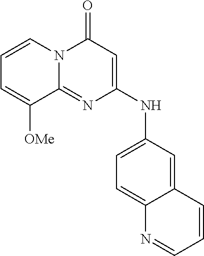 [SMILES: COc1cccn2c(=O)cc(Nc3ccc4ncccc4c3)nc12]

Brown solid; mp=259.3-262.1° C.; ¹H NMR (400 MHz, CDCl₃) δ 4.02 (s, 3H), 6.14 (s, 1H), 6.87 (dd, J=7.2, 7.6 Hz, 1H), 6.96 (dd, J=0.8, 7.6 Hz, 1H), 7.38-7.41 (m, 2H), 7.58 (dd, J=2.4, 8.8 Hz, 1H), 7.84 (d, J=2.4 Hz, 1H), 8.07 (d, J=8.8 Hz, 1H), 8.60 (dd, J=0.8, 7.2 Hz, 1H), 8.83 (dd, J=1.2, 4.0 Hz, 1H); LCMS (electrospray) m/z 319 (M+H)⁺.

### N-(9-Methoxy-4-oxo-4H-pyrido[1,2-a]pyrimidin-2-yl)-N-(quinolin-6-yl)acetamide (323)

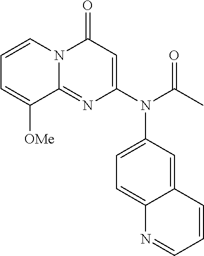 [SMILES: COc1cccn2c(=O)cc(N(C(C)=O)c3ccc4ncccc4c3)nc12]

White solid (83%); ¹H NMR (400 MHz, CDCl₃) δ 2.45 (s, 3H), 3.96 (s, 3H), 6.27 (s, 1H), 7.04-7.10 (m, 2H), 7.42 (dd, J=4.0, 8.0 Hz, 1H), 7.63 (dd, J=2.4, 8.8 Hz, 1H), 7.77 (d, J=2.4 Hz, 1H), 8.11 (d, J=8.4 Hz, 1H), 8.16 (d, J=8.8 Hz, 1H), 8.65 (dd, J=2.4, 6.8 Hz, 1H), 8.94 (dd, J=1.6, 4.0 Hz, 1H).

### 10-Methoxy-1-(quinolin-6-yl)-1H-dipyrido[1,2-a:2′,3′-d]pyrimidine-2,5-dione (324)

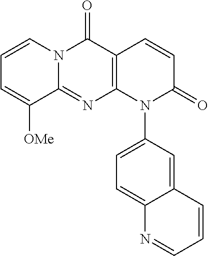 [SMILES: COc1cccn2c(=O)c3ccc(=O)n(-c4ccc5ncccc5c4)c3nc12]

White solid (45%); mp=313.0-315.0° C.; ¹H NMR (400 MHz, DMSO-d6) δ 3.58 (s, 3H), 6.56 (d, J=9.6 Hz, 1H), 7.19 (dd, J=7.2, 7.6 Hz, 1H), 7.30 (d, J=7.6 Hz, 1H), 7.62 (dd, J=4.4, 8.4 Hz, 1H), 7.68 (dd, J=2.0, 8.8 Hz, 1H), 7.99 (d, J=2.0 Hz, 1H), 8.13 (d, J=8.8 Hz, 1H), 8.19 (d, J=9.6 Hz, 1H), 8.46 (d, J=8.0 Hz, 1H), 8.56 (d, J=7.2 Hz, 1H), 9.01 (dd, J=1.6, 4.4 Hz, 1H).

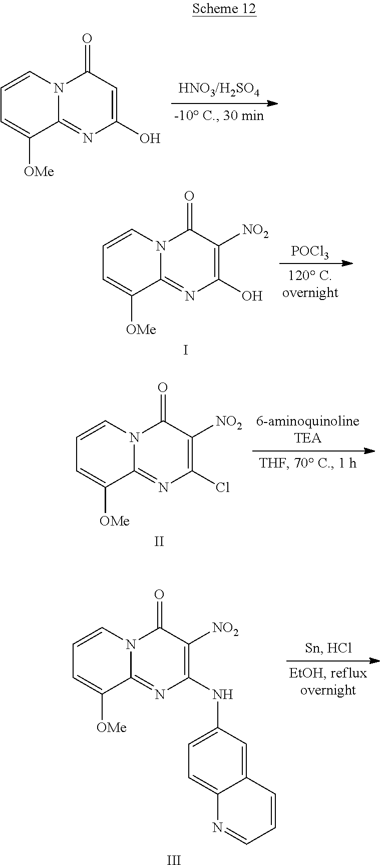

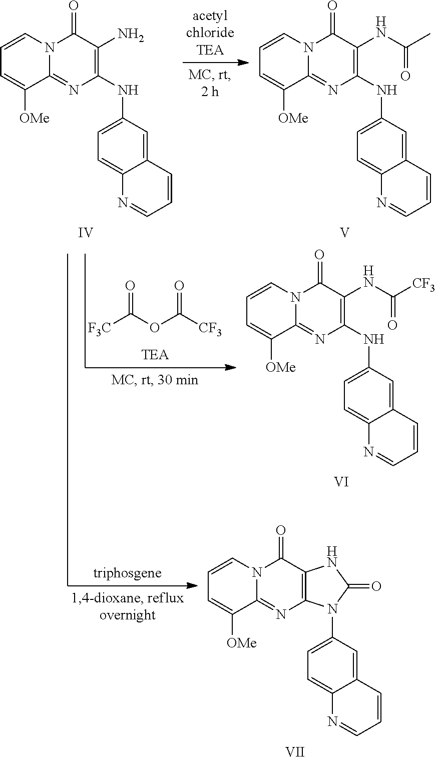

A mixture of nitric acid (0.4 mL) and H₂SO₄ (0.3 mL) was added to a solution of 2-hydroxy-9-methoxy-4H-pyrido[1,2-a]pyrimidin-4-one (1.56 mmol) in H₂ SO₄ (2 mL) at −10° C. and the resulting mixture was stirred for 30 min at the same temperature. After reaction completion, the reaction mixture was poured into the ice and the generating solid was filtered and dried to give a target compound I (yellow solid, 54%).

A mixture of compound I (0.42 mmol) and POCl₃ (2 mL) was stirred at 120° C. After overnight, the reaction mixture was poured into the ice and stirred for 10 min. The generating solid which is a mixture of starting material and desired product was filtered and then the crude mixture was purified by flash column chromatography (MC:MeOH=50:1) to give a target compound II (yellow solid, 45%).

To a stirred suspension of compound II (0.18 mmol) in THF (1 mL) were added 6-aminoquinoline (0.26 mmol) and TEA (0.53 mmol). The reaction mixture was stirred for an hour at 70° C. After reaction completion, the solvent was removed under reduced pressure and the resulting residue was dissolved with MeOH. At that time, the generating solid was filtered and dried to give a target compound III.

To a stirred suspension of compound III (0.083 mmol) in EtOH (1 mL) were added tin (30 mg) and a few drop of HCl (conc.). The reaction mixture was stirred for overnight at reflux temperature. The solvent was removed under reduced pressure and the resulting crude residue was purified by flash column chromatography (MC:MeOH=20:1) to give a target compound IV.

To a solution of compound IV (0.11 mmol) in MC (1 mL) were added acetyl chloride (0.17 mmol) and TEA (0.33 mmol) and the reaction mixture was stirred for 2 h at rt. The solvent was removed under reduced pressure and the resulting residue was purified by flash column chromatography (MC:MeOH=30:1 to 20:1) to give a target compound V.

To a solution of compound IV (0.090 mmol) in MC (1 mL) was added trifluoroacetic anhydride (0.099 mmol) at room temperature and the reaction mixture was stirred for 30 min. After reaction completion, the reaction was quenched with H₂O and extracted with methylene chloride twice. The resulting residue was purified by flash column chromatography (MC:MeOH=30:1) to give a target compound VI.

A mixture of compound IV (0.015 mmol) and triphosgene (0.017 mmol) in 1,4-dioxane (1 mL) was stirred for overnight at reflux temperature. The solvent was concentrated under reduced pressure and the resulting residue was purified by flash column chromatography (MC:MeOH=30:1) to give a target compound VII.

### 9-Methoxy-3-nitro-2-(quinolin-6-ylamino)-4H-pyrido[1,2-a]pyrimidin-4-one (325)

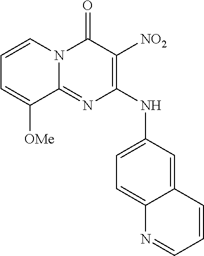 [SMILES: COc1cccn2c(=O)c([N+](=O)[O-])c(Nc3ccc4ncccc4c3)nc12]

Yellow solid (72%); mp=278.1-279.7° C.; ¹H NMR (400 MHz, DMSO-d6) δ 4.02 (s, 3H), 7.23 (dd, J=4.0, 7.6 Hz, 1H), 7.52-7.55 (m, 2H), 7.97 (d, J=8.8 Hz, 1H), 8.08 (d, J=9.2 Hz, 1H), 8.25 (d, J=8.0 Hz, 1H), 8.50 (d, J=7.2 Hz, 1H), 8.83-8.86 (m, 2H), 10.9 (s, 1H); LCMS (electrospray) m/z 364 (M+H)⁺.

### 3-Nitro-2-(quinolin-6-ylamino)-4H-pyrido[1,2-a]pyrimidin-4-one (326)

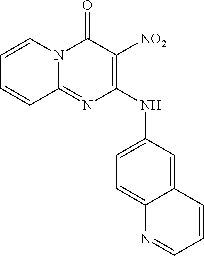 [SMILES: O=c1c([N+](=O)[O-])c(Nc2ccc3ncccc3c2)nc2ccccn12]

Dark yellow solid (74%); mp=288-289° C.; ¹H NMR (400 MHz, DMSO-d6) δ 7.30 (dd, J=6.8, 6.8 Hz, 1H), 7.52-7.56 (m, 2H), 8.03-8.09 (m, 3H), 8.38-8.43 (m, 2H), 8.86 (d, J=2.8 Hz, 1H), 8.920 (d, J=6.8 Hz, 1H), 10.85 (brs, 1H); LCMS (electrospray) m/z 334 (M+H)⁺.

### 3-Amino-9-methoxy-2-(quinolin-6-ylamino)-4H-pyrido[1,2-a]pyrimidin-4-one (327)

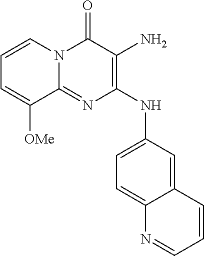 [SMILES: COc1cccn2c(=O)c(N)c(Nc3ccc4ncccc4c3)nc12]

Yellow solid (72%); mp=>236.6° C. (decomp.); ¹H NMR (400 MHz, DMSO-d6) δ 4.03 (s, 3H), 4.76 (s, 2H), 7.05-7.07 (m, 2H), 7.44 (dd, J=4.4, 8.4 Hz, 1H), 7.92 (d, J=8.8 Hz, 1H), 8.01 (dd, J=2.4, 8.8 Hz, 1H), 8.15 (d, J=8.4 Hz, 1H), 8.42 (dd, J=2.8, 8.4 Hz, 1H), 8.61 (brs, 1H), 8.71 (dd, J=1.6, 4.4 Hz, 1H), 8.85 (d, J=2.4 Hz, 1H); ¹³C NMR (100 MHz, CDCl₃) δ 56.6, 107.5, 110.4, 113.0, 113.2, 117.4, 121.6, 124.1, 128.7, 128.9, 134.8, 134.9, 139.2, 141.4, 144.0, 147.8, 150.8, 151.6; LCMS (electrospray) m/z 334 (M+H)⁺.

### N-(9-Methoxy-4-oxo-2-(quinolin-6-ylamino)-4H-pyrido[1,2-a]pyrimidin-3-yl)acetamide (328)

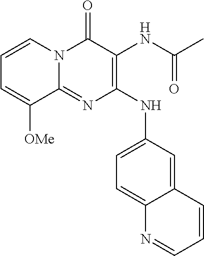 [SMILES: COc1cccn2c(=O)c(NC(C)=O)c(Nc3ccc4ncccc4c3)nc12]

Yellow solid (29%); mp=254.9-255.9° C. (decomp.); 'H NMR (400 MHz, DMSO-d6) δ 2.10 (s, 3H), 4.04 (s, 3H), 7.16 (dd, J=7.2, 7.2 Hz, 1H), 7.36 (d, J=7.2 Hz, 1H), 7.61 (dd, J=3.6, 8.4 Hz, 1H), 8.00 (d, J=9.2 Hz, 1H), 8.23 (d, J=9.2 Hz, 1H), 8.40-8.42 (m, 1H), 8.47 (d, J=7.2 Hz, 1H), 8.85-8.86 (m, 2H), 8.89 (brs, 1H), 9.07 (brs, 1H); LCMS (electrospray) m/z 376 (M+H)⁺.

### 2,2,2-Trifluoro-N-(9-methoxy-4-oxo-2-(quinolin-6-ylamino)-4H-pyrido[1,2-a]pyrimidin-3-yl)acetamide (329)

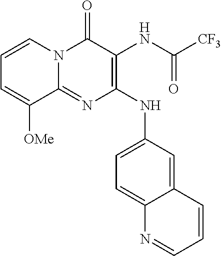 [SMILES: COc1cccn2c(=O)c(NC(=O)C(F)(F)F)c(Nc3ccc4ncccc4c3)nc12]

White solid (29%); mp=>310° C. (decomp.); ¹H NMR (400 MHz, CDCl₃+CD₃OD) δ 4.00 (s, 3H), 6.96-7.03 (m, 2H), 7.34 (dd, J=4.0, 8.0 Hz, 1H), 7.89 (dd, J=2.4, 9.2 Hz, 1H), 7.97 (d, J=9.2 Hz, 1H), 8.08 (d, J=8.0 Hz, 1H), 8.40 (d, J=2.4 Hz, 1H), 8.48 (dd, J=1.6, 6.8 Hz, 1H), 8.68 (dd, J=1.6, 4.0 Hz, 1H); LCMS (electrospray) m/z 430 (M+H)⁺.

### 5-Methoxy-3-(quinolin-6-yl)pyrido[1,2-a]purine-2,10 (1H,3H)-dione (330)

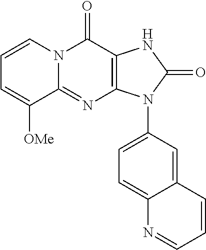 [SMILES: COc1cccn2c(=O)c3[nH]c(=O)n(-c4ccc5ncccc5c4)c3nc12]

Pale yellow solid (71%); mp=>350° C. (decomp.); ¹H NMR (400 MHz, CDCl₃+CD₃OD) δ 3.93 (s, 3H), 6.98 (dd, J=1.2, 7.6 Hz, 1H), 7.06 (dd, J=7.2, 7.6 Hz, 1H), 7.48 (dd, J=4.0, 8.4 Hz, 1H), 8.16 (dd, J=2.4, 9.2 Hz, 1H), 8.24-8.32 (m, 3H), 8.71 (dd, J=1.2, 7.2 Hz, 1H), 8.89 (s, 1H); LCMS (electrospray) m/z 360 (M+H)⁺.

### 3-(Quinolin-6-yl)pyrido[1,2-a]purine-2,10 (1H,3H)-dione (331)

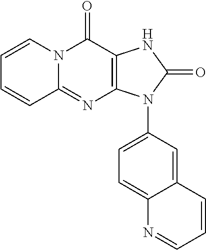 [SMILES: O=c1[nH]c2c(=O)n3ccccc3nc2n1-c1ccc2ncccc2c1]

Brown solid (41%); mp=>350° C. (decomp.); ¹H NMR (400 MHz, DMSO-d6) δ 7.32-7.35 (m, 1H), 7.61-7.65 (m, 2H), 7.82-7.86 (m, 1H), 8.02 (dd, J=2.4, 9.2 Hz, 1H), 8.19 (d, J=9.2 Hz, 1H), 8.29 (d, J=2.4 Hz, 1H), 8.48 (d, J=8.8 Hz, 1H), 8.98 (dd, J=1.6, 4.4 Hz, 1H), 9.08 (d, J=7.2 Hz, 1H); LCMS (electrospray) m/z 330 (M+H)⁺.

### 1-Methyl-3-(quinolin-6-yl)pyrido[1,2-a]purine-2,10 (1H,3H)-dione (332)

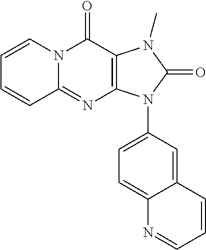 [SMILES: Cn1c(=O)n(-c2ccc3ncccc3c2)c2nc3ccccn3c(=O)c21]

White solid (71%); mp=276.1-276.7° C.; ¹H NMR (400 MHz, CDCl₃) δ 3.88 (s, 314), 7.15-7.19 (m, 1H), 7.47 (dd, J=4.0, 8.0 Hz, 1H), 7.60-7.70 (m, 2H), 8.10 (dd, J=2.4, 8.8 Hz, 1H), 8.24-8.31 (m, 3H), 8.98 (s, 1H), 9.15-9.18 (m, 1H); LCMS (electrospray) m/z 344 (M+H)⁺.

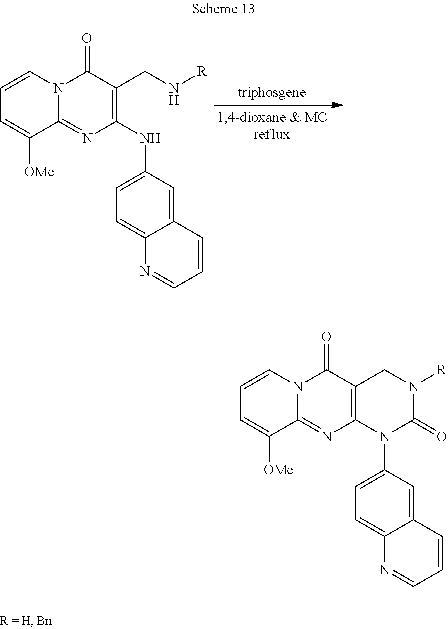

### 3-Benzyl-10-methoxy-1-(quinolin-6-yl)-3,4-dihydro-1H-pyrido[1,2-a]pyrimido[4,5-d]pyrimidine-2,5-dione (333)

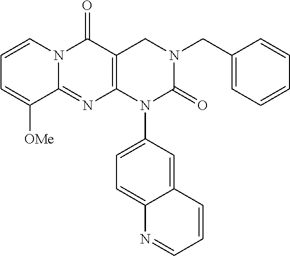 [SMILES: COc1cccn2c(=O)c3c(nc12)N(c1ccc2ncccc2c1)C(=O)N(Cc1ccccc1)C3]

White solid (24%); mp=282.9-284.5° C.; ¹H NMR (400 MHz, DMSO-d6) δ 3.59 (s, 3H), 4.40 (s, 2H), 4.70 (s, 2H), 7.14 (dd, J=7.2, 7.6 Hz, 1H), 7.24 (dd, J=1.2, 8.0 Hz, 1H), 7.30-7.43 (m, 5H), 7.56 (dd, J=4.0, 8.0 Hz, 1H), 7.68 (dd, J=2.4, 9.2 Hz, 1H), 7.69 (d, J=2.4 Hz, 1H), 8.05 (d, J=9.2 Hz, 1H), 8.40 (dd, J=1.2, 7.6 Hz, 1H), 8.47 (dd, J=1.2, 7.2 Hz, 1H), 8.95 (dd, J=1.2, 4.0 Hz, 1H); LCMS (electrospray) m/z 464 (M+H)⁺.

### 10-Methoxy-1-(quinolin-6-yl)-3,4-dihydro-1H-pyrido[1,2-a]pyrimido[4,5-d]pyrimidine-2,5-dione (334)

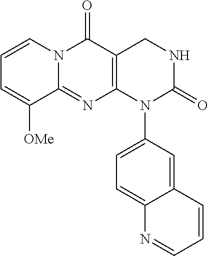 [SMILES: COc1cccn2c(=O)c3c(nc12)N(c1ccc2ncccc2c1)C(=O)NC3]

Pale yellow solid; ¹H NMR (400 MHz, DMSO-d6) δ 3.59 (s, 3H), 4.39 (s, 2H), 7.15 (dd, J=7.2, 8.0 Hz, 1H), 7.24 (d, J=8.0 Hz, 1H), 7.58 (dd, J=4.4, 8.0 Hz, 1H), 7.65 (dd, J=2.0, 7.2 Hz, 1H), 7.79 (s, 1H), 7.93 (s, 1H), 8.04 (d, J=8.8 Hz, 1H), 8.42 (d, J=8.0 Hz, 1H), 8.50 (d, J=7.2 Hz, 1H), 8.96 (d, J=4.0 Hz, 1H); LCMS (electrospray) m/z 374 (M+H)⁺.

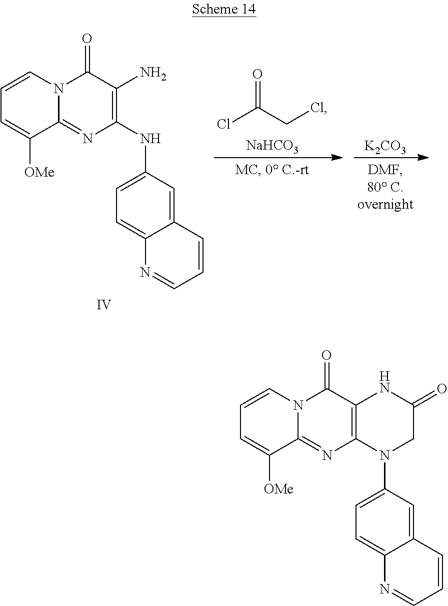

To a suspension of compound IV (0.036 mmol) and NaHCO₃ (0.11 mmol) in MC (500 uL) was added 2-chloroacetyl chloride (0.039 mmol) under ice bath. After the addition, the reaction mixture was warmed to room temperature and stirred for an hour. The reaction solvent was concentrated under reduced pressure, the resulting residue was dissolved with DMF and then K₂CO₃ (0.050 g, 0.36 mmol) was followed to the reaction flask. The resulting mixture was heated to 80° C. for overnight, the solvent was removed under reduced pressure and the crude residue was dissolved with MeOH. The unsoluble solid was filtered and dried to give a target compound.

### 6-Methoxy-4-(quinolin-6-yl)-3,4-dihydro-1H-pyrido[2,1-b]pteridine-2,11-dione (335)

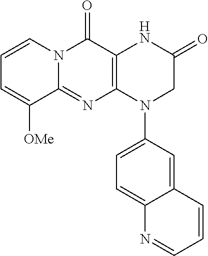 [SMILES: COc1cccn2c(=O)c3c(nc12)N(c1ccc2ncccc2c1)CC(=O)N3]

Pale yellow solid (45%); mp=315.3-317.3° C.; ¹H NMR (400 MHz, CDCl₃+CD₃OD) δ 3.76 (s, 3H), 4.56 (s, 2H), 6.83 (d, J=7.2 Hz, 1H), 6.92 (dd, J=7.2, 7.6 Hz, 1H), 7.40 (dd, J=4.4, 8.4 Hz, 1H), 7.59 (d, J=2.4 Hz, 1H), 7.90 (dd, J=2.4, 9.2 Hz, 1H), 8.00 (d, J=9.2 Hz, 1H), 8.15 (d, J=8.4 Hz, 1H), 8.44 (dd, J=1.2, 7.2 Hz, 1H), 8.75 (dd, J=1.6, 4.4 Hz, 1H); LCMS (electrospray) m/z 374 (M+H)⁺.

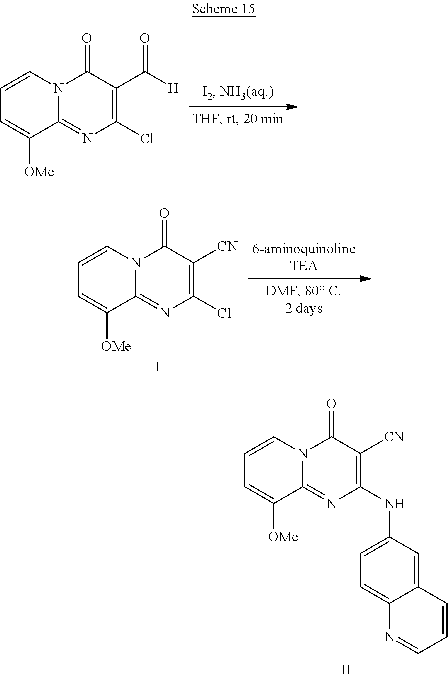

To a stirred suspension of 2-chloro-9-methoxy-4-oxo-4H-pyrido[1,2-a]pyrimidine-3-carbaldehyde (2.10 mmol) and I₂ (2.30 mmol) in THF (5 mL) was added ammonia water (10 mL). The resulting mixture was stirred for 20 min at room temperature. After reaction completion, unsoluble solid was filtered and washed with H₂O and MeOH. The resulting residue was purified by flash column chromatography (MC:MeOH=50:1) to give a target compound I (pale yellow solid, 53%).

To a solution of compound I (0.21 mmol) in DMF (2 mL) were added 6-aminoquinoline (0.23 mmol) and TEA (0.32 mmol). The reaction mixture was stirred for 2 days at 80° C. After reaction completion, the solvent was removed under reduced pressure and the resulting residue was dissolved with MeOH. At that time, the generating solid was filtered and dried to give a target compound II.

### 9-Methoxy-4-oxo-2-(quinolin-6-ylamino)-4H-pyrido[1,2-a]pyrimidine-3-carbonitrile (336)

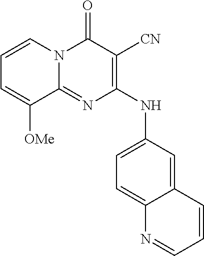 [SMILES: COc1cccn2c(=O)c(C#N)c(Nc3ccc4ncccc4c3)nc12]

Pale brown solid (45%); mp=273-274° C.; ¹H NMR (400 MHz, DMSO-d6) δ 3.97 (s, 3H), 7.22 (dd, J=7.2, 7.6 Hz, 1H), 7.49-7.53 (m, 2H), 7.96 (d, J=9.2 Hz, 1H), 8.11 (dd, J=2.4, 9.2 Hz, 1H), 8.24 (dd, J=1.2, 8.4 Hz, 1H), 8.46 (dd, J=1.2, 7.2 Hz, 1H), 8.58 (d, J=2.4 Hz, 1H), 8.18 (dd, J=1.2, 4.4 Hz, 1H), 9.90 (s, 1H); LCMS (electrospray) m/z 344 (M+H)⁺.

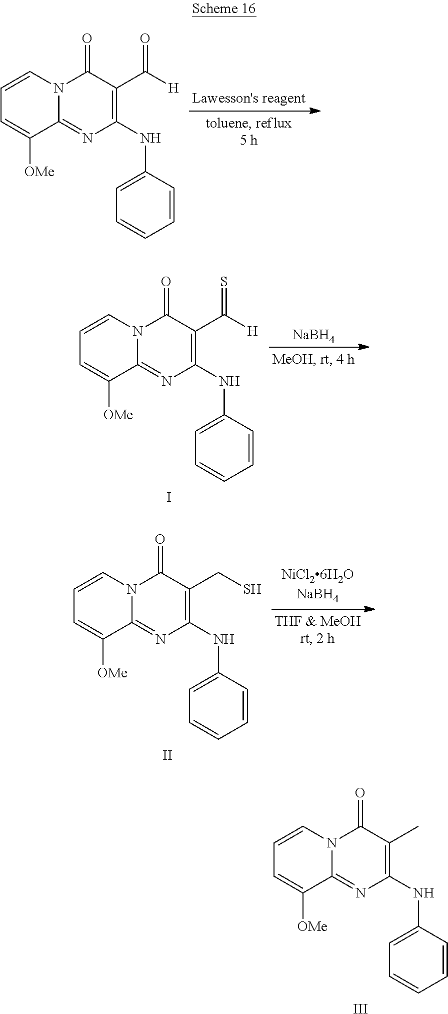

To a stirred solution of 9-methoxy-4-oxo-2-(phenylamino)-4H-pyrido[1,2-a]pyrimidine-3-carbaldehyde (0.68 mmol) in toluene (8 mL) was added Lawesson's reagent (0.81 mmol). The resulting mixture was stirred for 5 h at reflux temperature. After reaction completion, the reaction solvent was removed under reduced pressure and the resulting residue was purified by flash column chromatography (MC:MeOH=100:1) to give a target compound I (orange solid, 37%).

To a stirred solution of compound I (0.23 mmol) in MeOH (2 mL) was added sodium borohydride (0.34 mmol) and the reaction mixture was stirred for 4 h. The reaction was quenched with H₂O and the organic solvent was removed under reduced pressure. Unsoluble solid under aqueous condition was filtered, washed with water and dried. The crude residue was purified by flash column chromatography (MC:MeOH=50:1) to give a target compound II (pale yellow solid, 51%).

To a stirred solution of compound II (0.064 mmol) and nickel chloride hexahydrate (0.12 mmol) in a mixture of MeOH and THF (3:1 ratio, v/v) was added sodium borohydride (0.79 mmol) under ice bath. The reaction temperature was allowed to room temperature and the reaction mixture was stirred for 2 h. After reaction completion, the reaction was quenched with H₂O and the solvent was removed under reduced pressure. The black residue was dissolved with MeOH and unsoluble solid was filtered off using of cellite. The filtrate was concentrated under reduced pressure and the crude residue was purified by flash column chromatography (MC:MeOH=50:1) to give a target compound III (pale yellow solid).

### 9-Methoxy-4-oxo-2-(phenylamino)-4H-pyrido[1,2-a]pyrimidine-3-carbothialdehyde (337)

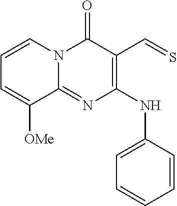 [SMILES: COc1cccn2c(=O)c(C=S)c(Nc3ccccc3)nc12]

Orange solid (37%); ¹H NMR (400 MHz, CDCl₃) δ 4.02 (s, 3H), 6.90 (dd, J=7.2, 7.6 Hz, 1H), 7.07 (d, J=7.6 Hz, 1H), 7.16-7.19 (m, 1H), 7.37-7.41 (m, 2H), 7.94-7.96 (m, 2H), 8.58 (d, J=7.2 Hz, 1H), 13.85 (s, 1H); LCMS (electrospray) m/z 312 (M+H)⁺, 344 (M+Na)⁺.

### 3-(Mercaptomethyl)-9-methoxy-2-(phenylamino)-4H-pyrido[1,2-a]pyrimidin-4-one (338)

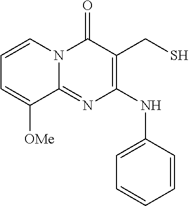 [SMILES: COc1cccn2c(=O)c(CS)c(Nc3ccccc3)nc12]

Pale yellow solid (51%); ¹H NMR (400 MHz, CDCl₃) δ 3.97 (s, 3H), 4.23 (s, 2H), 6.74 (dd, J=7.2, 7.6 Hz, 1H), 6.89 (dd, J=1.2, 7.6 Hz, 1H), 7.07-7.10 (m, 1H), 7.32-7.36 (m, 2H), 7.71 (s, 1H), 7.82-7.84 (m, 2H), 8.46 (dd, J=1.2, 7.2 Hz, 1H); LCMS (electrospray) m/z 314 (M+H)⁺.

### 9-Methoxy-3-methyl-2-(phenylamino)-4H-pyrido[1,2-a]pyrimidin-4-one (339)

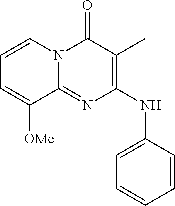 [SMILES: COc1cccn2c(=O)c(C)c(Nc3ccccc3)nc12]

Pale yellow solid; ¹H NMR (400 MHz, CDCl₃) δ 2.21 (s, 3H), 3.99 (s, 3H), 6.49 (s, 1H), 6.85-6.90 (m, 2H), 7.03 (dd, J=7.2, 7.2 Hz, 1H), 7.31-7.35 (m, 2H), 7.69-7.71 (m, 2H), 8.62 (dd, J=2.4, 6.0 Hz, 1H); LCMS (electrospray) m/z 282 (M+H)⁺.

### 9-Methoxy-4-oxo-2-(quinolin-6-ylamino)-4H-pyrido[1,2-a]pyrimidine-3-carbothialdehyde (340)

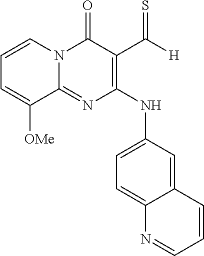 [SMILES: COc1cccn2c(=O)c(C=S)c(Nc3ccc4ncccc4c3)nc12]

Yellow solid (32%); mp=>250° C. (decomp.); ¹H NMR (400 MHz, CDCl₃) δ 4.08 (s, 3H), 6.95 (dd, J=7.2, 7.6 Hz, 1H), 7.13 (d, J=7.6 Hz, 1H), 7.40 (dd, J=4.0, 7.6 Hz, 1H), 8.07-8.14 (m, 3H), 8.60 (dd, J=1.2, 7.2 Hz, 1H), 8.78 (s, 1H), 8.86 (s, 1H), 11.6 (s, 1H); LCMS (electrospray) m/z 363 (M+H)⁺.

### 3-(Mercaptomethyl)-9-methoxy-2-(quinolin-6-ylamino)-4H-pyrido[1,2-a]pyrimidin-4-one (341)

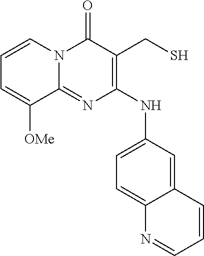 [SMILES: COc1cccn2c(=O)c(CS)c(Nc3ccc4ncccc4c3)nc12]

Pale yellow solid (46%); ¹H NMR (400 MHz, DMSO-d6) δ 3.99 (s, 3H), 4.35 (s, 2H), 7.01 (dd, J=7.2, 7.6 Hz, 1H), 7.29 (d, J=7.6 Hz, 1H), 7.47 (dd, J=4.4, 8.4 Hz, 1H), 7.91 (d, J=9.2 Hz, 1H), 8.14-8.20 (m, 2H), 8.38 (d, J=7.2 Hz, 1H), 8.77-8.80 (m, 2H), 8.86 (s, 1H).

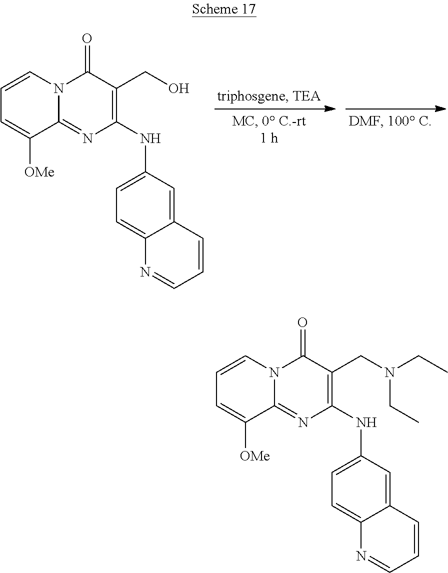 [SMILES: COc1cccn2c(=O)c(CO)c(Nc3ccc4ncccc4c3)nc12 ; CCN(CC)Cc1c(Nc2ccc3ncccc3c2)nc2c(OC)cccn2c1=O]

To a stirred solution of alcohol (0.14 mmol) and TEA (1.44 mmol) in methylene chloride (2 mL) was added triphosgene (0.21 mmol) slowly under ice bath. The reaction mixture was allowed to room temperature and stirred for an hour. The solvent was removed under reduced pressure, the resulting mixture was dissolved with DMF (2 mL) and stirred at 100° C. for 2 h. After reaction completion, the reaction mixture was concentrated and the resulting residue was purified by flash column chromatography (MC:MeOH=30:1) to give a target compound.

### 3-((Diethylamino)methyl)-9-methoxy-2-(quinolin-6-ylamino)-4H-pyrido[1,2-a]pyrimidin-4-one (342)

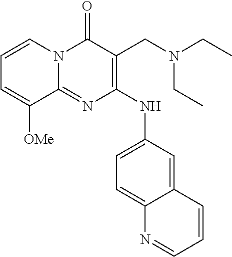 [SMILES: CCN(CC)Cc1c(Nc2ccc3ncccc3c2)nc2c(OC)cccn2c1=O]

Pale yellow solid; ¹H NMR (400 MHz, CDCl₃) δ 1.16 (t, J=7.2 Hz, 6H), 2.67 (q, J=7.2 Hz, 4H), 3.93 (s, 2H), 4.08 (s, 3H), 6.90-7.00 (m, 2H), 7.33 (dd, J=4.4, 8.4 Hz, 1H), 7.74 (dd, J=2.4, 9.2 Hz, 1H), 8.01 (d, J=9.2 Hz, 1H), 8.09 (d, J=8.0 Hz, 1H), 8.62-8.69 (m, 2H), 8.76 (d, J=2.4 Hz, 1H), 11.50 (s, 1H); LCMS (electrospray) m/z 404 (M+H)⁺.

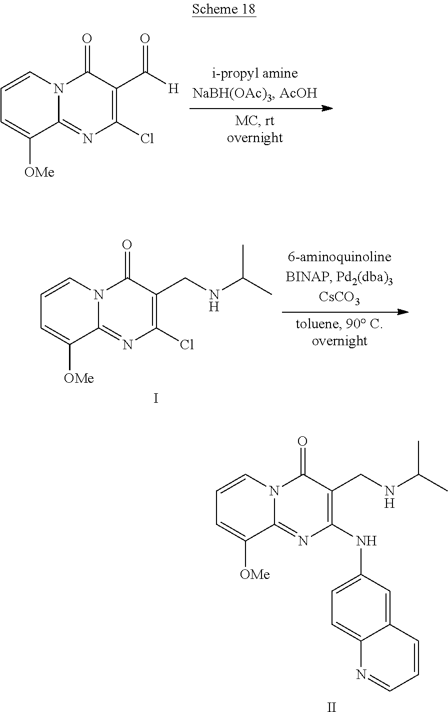

To a stirred solution of aldehyde (0.84 mmol) in methylene chloride (4 mL) were added i-propylamine (0.92 mmol), NaBH(OAc)₃ (1.68 mmol) and acetic acid (90 uL). The reaction mixture was stirred for overnight. After reaction completion, water was added and then the mixture was extracted with MC. Aqueous phase was titrated with saturated Na₂CO₃ solution until pH 8 and then extracted with methylene chloride several times. The organic phase was dried over MgSO₄ and concentrated in vacuo. The crude residue was purified by flash column chromatography (MC:MeOH=10:1) to give a target compound I.

To a stirred solution of compound I (0.18 mmol) in toluene (2 mL) were added 6-aminoquinoline (0.20 mmol), (R)-(+)-2,2′-bis(diphenylphosphino)-1,1′-binaphthyl (0.018 mmol), tris(dibenzylideneacetone) dipalladium(0) (8.9 umol) and cessium carbonate (0.27 mmol). The reaction mixture was heated to 90° C. for overnight. The solvent was removed under reduced pressure and the resulting residue was purified by flash column chromatography (MC:MeOH=50:1 to 20:1) to give a target compound II.

### 3-((Isopropylamino)methyl)-9-methoxy-2-(quinolin-6-ylamino)-4H-pyrido[1,2-a]pyrimidin-4-one (343)

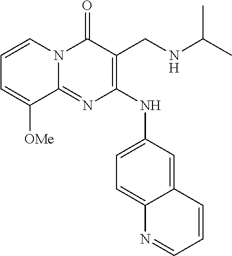 [SMILES: COc1cccn2c(=O)c(CNC(C)C)c(Nc3ccc4ncccc4c3)nc12]

Pale yellow solid; ¹H NMR (400 MHz, CDCl₃) δ 1.32 (d, J=6.0 Hz, 6H), 2.01-2.04 (m, 1H), 3.46-3.48 (m, 1H), 4.03 (s, 3H), 4.23 (s, 2H), 6.88-6.91 (m, 2H), 7.32 (dd, J=4.0, 8.4 Hz, 1H), 7.85-8.05 (m, 2H), 8.06 (d, J=7.2 Hz, 1H), 8.56-8.62 (m, 2H), 8.76 (dd, J=1.6, 4.0 Hz, 1H), 10.91 (s, 1H); LCMS (electrospray) m/z 390 (M+H)⁺.

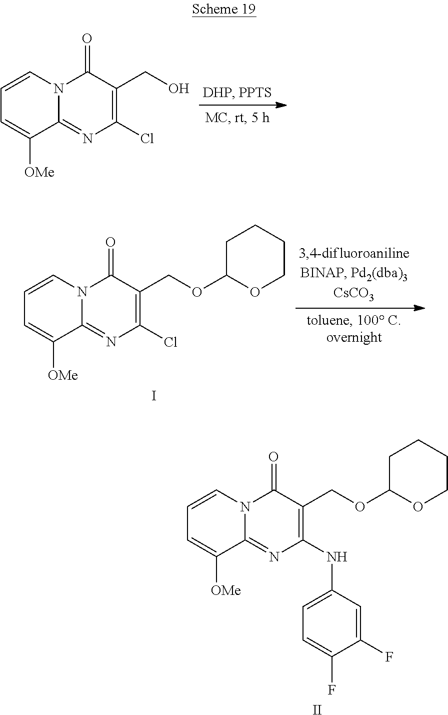

To a stirred solution of alcohol (2.08 mmol) in methylene chloride (10 mL) were added 3,4-dihydro-2H-pyran (4.16 mmol) and pyridinium-p-toluene sulfonate (3.12 mmol) and the reaction mixture was stirred for 5 h at room temperature. The reaction mixture was washed with saturated NaHCO₃ (aq.) and the organic phase was washed with brine again. The organic layer was dried over MgSO₄, concentrated in vacuo and the resulting crude residue was purified by flash column chromatography (MC:MeOH=50:1) to give a target compound I.

To a stirred solution of compound I (0.61 mmol) in toluene (3 mL) were added 3,4-difluoroaniline (0.68 mmol), (R)-(+)-2,2′-bis(diphenylphosphino)-1,1′-binaphthyl (0.062 mmol), tris(dibenzylideneacetone) dipalladium(0) (0.031 mmol) and cessium carbonate (0.92 mmol). The reaction mixture was heated to 100° C. for overnight. The solvent was removed under reduced pressure and the resulting residue was purified by flash column chromatography (MC:MeOH=100:1) to give a target compound II.

### 2-(3,4-Difluorophenylamino)-9-methoxy-3-((tetrahydro-2H-pyran-2-yloxy)methyl)-4H-pyrido[1,2-a]pyrimidin-4-one (344)

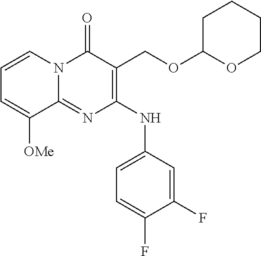 [SMILES: COc1cccn2c(=O)c(COC3CCCCO3)c(Nc3ccc(F)c(F)c3)nc12]

White solid; ¹H NMR (400 MHz, CDCl₃) δ 1.56-1.62 (m, 4H), 1.81-1.90 (m, 2H), 3.61-3.67 (m, 1H), 4.02 (s, 3H), 4.05-4.09 (m, 1H), 4.71-4.72 (m, 1H), 4.77 (d, J=12.0 Hz, 1H), 5.14 (d, J=12.0 Hz, 1H), 6.90 (dd, J=6.8, 7.6 Hz, 1H), 6.96 (dd, J=1.2, 7.6 Hz, 1H), 7.05-7.12 (m, 1H), 7.19-7.22 (m, 1H), 8.09-8.14 (m, 1H), 8.47 (s, 1H), 8.62 (dd, J=1.6, 7.2 Hz, 1H); LCMS (electrospray) m/z 418 (M+H)⁺.

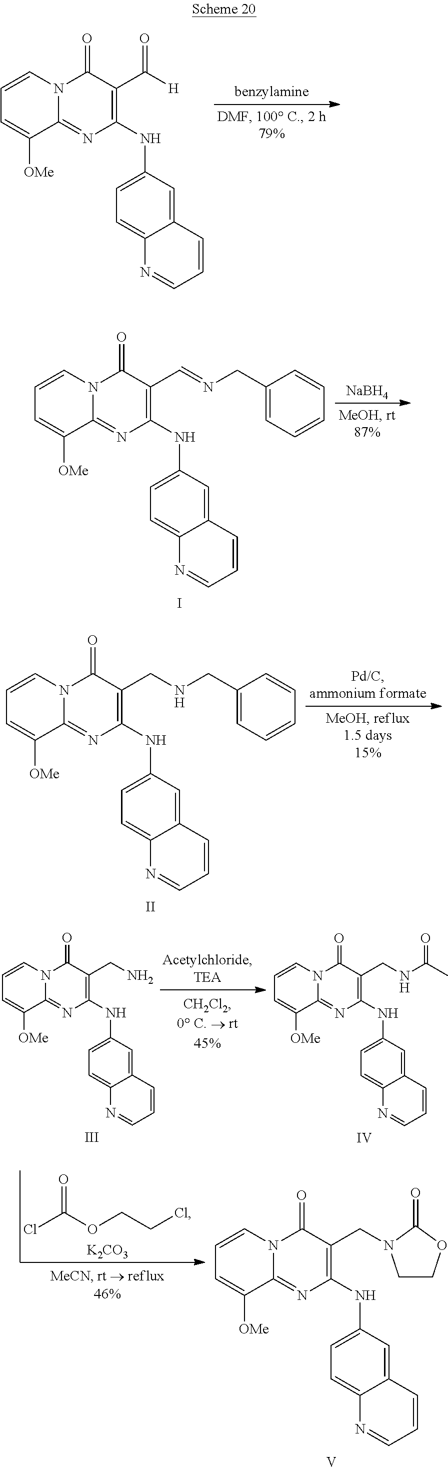

### General Procedure for the Synthesis of I

To a stirred solution of 9-methoxy-4-oxo-2-(quinolin-6-ylamino)-4H-pyrido[1,2-a]pyrimidine-3-carbaldehyde (0.52 mmol) in dimethylfomamide (2.5 mL) was added benzylamine (0.57 mmol). The reaction mixture was stirred at 100° C. for overnight. After cooling, the reaction mixture was concentrated in vacuo. The residue was purified by flash column chromatography to give I

### General Procedure for the Synthesis of II

To a stirred solution of I (0.16 mmol) in methanol (1.0 mL) was added sodium borohydride (0.24 mmol) at 0° C. The reaction mixture was stirred at room temperature for 3 hours. After reaction was completed, H₂O (1.0 mL) was added. The mixture was diluted with dichloromethane (10 mL) and washed with H₂O. The organic layer was dried over anhydrous MgSO₄ and concentrated in vacuo. The crude product was purified by flash column chromatography to give II.

### General Procedure for the Synthesis of III

To a stirred solution of II (0.068 mmol) in methanol (0.5 mL) was added ammonium formate (0.14 mmol) and Pd/C (0.068 mmol). The reaction mixture was stirred at reflux for overnight. After reaction was completed, filtered off and concentrated in vacuo. The crude product was purified by flash column chromatography to give III.

### General Procedure for the Synthesis of IV

To a stirred solution of III (0.144 mmol) in dichloromethane (0.5 mL) was added acetyl chloride (0.16 mmol) and triethylamine (0.22 mmol). The reaction mixture was stirred at room temperature for 1 hour. After reaction was completed, the mixture was diluted with dichloromethane (10 mL) and washed with H₂O (10 ml). The organic layer was dried over anhydrous MgSO₄ and concentrated in vacuo. The crude product was purified by flash column chromatography to give IV.

### General Procedure for the Synthesis of V

To a stirred solution of IV (0.11 mmol) in acetonitrile (0.5 mL) was added 2-chloroethyl-chloroformate (0.13 mmol) and Potassium carbonate (0.27 mmol). The reaction mixture was stirred at reflux for overnight. After reaction was completed, filtered off and concentrated in vacuo. The crude product was purified by flash column chromatography to give V.

### 3-(Aminomethyl)-9-methoxy-2-(quinolin-6-ylamino)-4H-pyrido[1,2-a]pyrimidin-4-one (345)

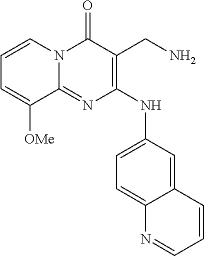 [SMILES: COc1cccn2c(=O)c(CN)c(Nc3ccc4ncccc4c3)nc12]

Pale yellow solid; ¹H NMR (400 MHz, DMSO-d₆) δ 4.00 (s, 3H), 4.10 (s, 2H), 7.11 (dd, J=7.2 Hz, 7.2 Hz, 1H), 7.31 (d, J=7.6 Hz, 1H), 7.45 (dd, J=4.0 Hz, 8.4 Hz, 1H), 7.92 (d, J=8.8 Hz, 1H), 8.06 (dd, J=2.4 Hz 9.2 Hz, 1H), 8.17 (d, J=8.0 Hz, 1H), 8.48 (d, J=6.4 Hz, 1H), 8.72 (d, J=6.4 Hz, 1H), 8.78 (d, J=2.4 Hz, 1H) ¹³C NMR (100 MHz, DMSO-d₆) δ 35.4, 57.5, 93.2, 1136.6, 114.2, 115.8, 119.2, 122.3, 125.6, 129.2, 129.5, 135.9, 139.2, 143.9, 144.9, 148.9, 151.4, 156.6, 157.8; LCMS (electrospray) m/z (M+H)⁺ 348.

### N-((9-Methoxy-4-oxo-2-(quinolin-6-ylamino)-4H-pyrido[1,2-a]pyrimidin-3-yl)methyl)acetamide (346)

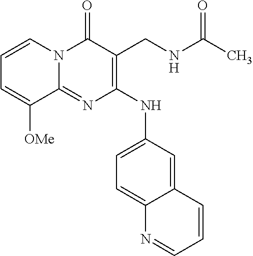 [SMILES: COc1cccn2c(=O)c(CNC(C)=O)c(Nc3ccc4ncccc4c3)nc12]

Pale yellow solid; ¹H NMR (400 MHz, DMSO-d₆) δ 1.92 (s, 3H), 4.02 (s, 3H), 4.35 (d, J=6.0 Hz, 2H), 7.12 (dd, J=7.2 Hz, 1H), 7.33 (d, J=7.2 Hz, 1H), 7.46 (dd, J=4.4 Hz, 8.4 Hz, 1H), 7.94 (d, J=9.2 Hz, 1H), 8.00 (dd, J=2.4 Hz, 9.2 Hz, 1H), 8.18 (d, J=7.6 Hz, 1H), 8.49 (d, J=6.8 Hz, 1H), 8.72 (d, J=2.8 Hz, 1H), 8.85 (d, J=2.4 Hz, 1H), 9.00 (s, 1H), 10.36 (s, 1H); ¹³C NMR (100 MHz, DMSO-d₆) δ 22.7, 34.1, 57.6, 95.1, 113.8, 114.36, 115.0, 119.4, 122.4, 124.8, 129.3, 129.8, 135.9, 139.3, 144.0, 144.9, 148.9, 151.4, 156.2, 158.2, 173.0; LCMS (electrospray) m/z (M+H)⁺ 390.

### N-((9-Methoxy-4-oxo-2-(quinolin-6-ylamino)-4H-pyrido[1,2-a]pyrimidin-3-yl)methyl)isobutyramide (347)

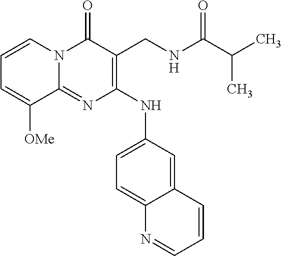 [SMILES: COc1cccn2c(=O)c(CNC(=O)C(C)C)c(Nc3ccc4ncccc4c3)nc12]

Pale yellow solid; ¹H NMR (400 MHz, DMSO-d₆) δ 0.95 (d, J=6.8 Hz, 6H), 3.32-3.34 (m, 1H), 3.97 (s, 3H), 4.34 (d, J=6.4 Hz, 2H), 7.08 (dd, J=7.6 Hz, 7.6 Hz, 1H), 7.28 (d, J=8.0 Hz, 1H), 7.40 (dd, J=4.0 Hz, 8.0 Hz, 1H), 7.88-7.94 (m, 2H), 8.12 (d, J=8.4 Hz, 1H), 8.45 (dd, J=1.2 Hz, 7.2 Hz, 1H), 8.67 (dd, J=1.6 Hz, 4.0 Hz, 1H), 8.81 (t, J=6.0 Hz, 1H), 8.87 (d, J=1.6 Hz, 1H), 10.1 (s, 1H); LCMS (electrospray) m/z (M+H)⁺ 418.

### Isobutyl ((9-methoxy-4-oxo-2-(quinolin-6-ylamino)-4H-pyrido[1,2-a]pyrimidin-3-yl)methyl)carbamate (348)

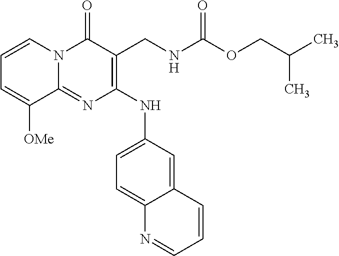 [SMILES: COc1cccn2c(=O)c(CNC(=O)OCC(C)C)c(Nc3ccc4ncccc4c3)nc12]

Pale yellow solid; ¹H NMR (400 MHz, CDCl₃) δ 0.89 (d, J=6.4 Hz, 6H), 1.87-1.94 (m, 1H), 3.94 (d, J=6.8 Hz, 2H), 4.05 (s, 3H), 4.54 (d, J=6.8 Hz, 2H), 5.72 (t, J=6.8 Hz, 1H), 6.91 (dd, J=7.2 Hz, 7.2 Hz, 1H), 6.97 (d, J=7.6 Hz, 1H), 7.34 (dd, J=4.4 Hz, 8.0 Hz, 1H), 8.05 (d, J=1.2 Hz, 2H), 8.10 (dd, J=1.6 Hz, 8.0 Hz, 1H), 8.61 (dd, J=1.6 Hz, 7.2 Hz, 1H), 8.69 (s, 1H), 8.78 (dd, J=1.6 Hz, 4.4 Hz, 1H), 9.69 (s, 1H); LCMS (electrospray) m/z (M+H)⁺ 448.

### N-((9-Methoxy-4-oxo-2-(quinolin-6-ylamino)-4H-pyrido[1,2-a]pyrimidin-3-yl)methyl)benzamide (349)

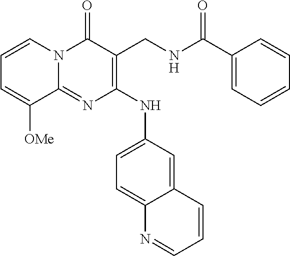 [SMILES: COc1cccn2c(=O)c(CNC(=O)c3ccccc3)c(Nc3ccc4ncccc4c3)nc12]

Pale yellow solid; ¹H NMR (400 MHz, CDCl₃) δ 4.05 (s, 3H), 4.82 (d, J=6.4 Hz, 2H), 6.90 (dd, J=7.2 Hz, 7.2 Hz, 1H), 6.96 (d, J=7.6 Hz, 1H), 7.31 (s, 1H), 7.35 (dd, J=4.0 Hz, 8.0 Hz, 1H), 7.44 (dd, J=7.2 Hz, 2H), 7.50 (d, J=7.2 Hz, 1H), 7.85 (d, J=7.2 Hz, 2H), 8.08 (d, J=9.2 Hz, 1H), 8.12 (d, J=8.4 Hz, 1H), 8.22 (dd, J=2.4 Hz, 9.2 Hz, 1H), 8.59 (d, J=7.2 Hz, 1H), 8.71 (d, J=2.0 Hz, 1H), 8.79 (d, J=2.8 Hz, 1H), 10.27 (s, 1H)); LCMS (electrospray) m/z (M+H)⁺ 452.

### Benzyl ((9-methoxy-4-oxo-2-(quinolin-6-ylamino)-4H-pyrido[1,2-a]pyrimidin-3-yl)methyl)carbamate (350)

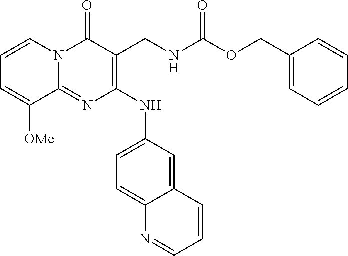 [SMILES: COc1cccn2c(=O)c(CNC(=O)OCc3ccccc3)c(Nc3ccc4ncccc4c3)nc12]

Pale yellow solid; ¹H NMR (400 MHz, CDCl₃) δ 4.05 (s, 3H), 4.56 (d, J=6.8 Hz, 2H), 5.84 (t, J=7.2 Hz, 1H), 6.87 (dd, J=7.2 Hz, 7.2 Hz, 1H), 6.95 (d, J=6.0 Hz, 1H), 7.27-7.31 (m, 3H), 7.34-7.37 (m, 3H), 7.99-8.06 (m, 2H), 8.10 (d, J=7.2 Hz, 1H), 8.58 (d, J=6.0 Hz, 1H), 8.66 (d, J=1.6 Hz, 1H), 8.78 (dd, J=1.6 Hz, 4.4 Hz, 1H), 9.59 (s, 1H)); LCMS (electrospray) m/z (M+H)⁺ 482.

### Phenyl ((9-methoxy-4-oxo-2-(quinolin-6-ylamino)-4H-pyrido[1,2-a]pyrimidin-3-yl)methyl)carbamate (351)

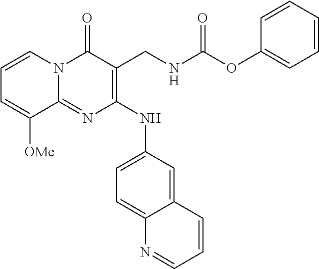 [SMILES: COc1cccn2c(=O)c(CNC(=O)Oc3ccccc3)c(Nc3ccc4ncccc4c3)nc12]

Pale yellow solid; ¹H NMR (400 MHz, CDCl₃) δ 4.06 (s, 3H), 4.64 (d, J=7.2 Hz, 2H), 6.10 (brs, 1H), 6.92-6.96 (m, 1H), 7.00 (d, J=7.2 Hz, 1H), 7.11 (d, J=8.4 Hz, 2H), 7.22 (d, J=8.0 Hz, 1H), 7.32-7.38 (m, 3H), 8.00 (s, 2H), 8.06 (d, J=8.4 Hz, 1H), 8.60 (s, 1H), 8.64 (d, J=6.8 Hz, 1H), 8.78 (d, J=4.4 Hz, 1H), 9.39 (s, 1H); LCMS (electrospray) m/z (M+H)⁺ 468.

### Isopropyl ((9-methoxy-4-oxo-2-(quinolin-6-ylamino)-4H-pyrido[1,2-a]pyrimidin-3-yl)methyl)carbamate (352)

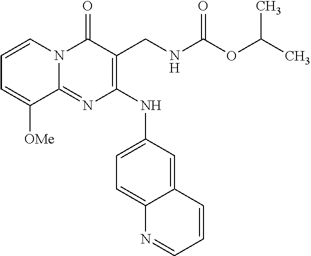 [SMILES: COc1cccn2c(=O)c(CNC(=O)OC(C)C)c(Nc3ccc4ncccc4c3)nc12]

Pale yellow solid; ¹H NMR (400 MHz, CDCl₃) δ 1.24 (d, J=4.8 Hz, 6H), 4.06 (s, 3H), 4.53 (d, J=6.8 Hz, 2H), 5.00-5.03 (m, 1H), 5.54 (brs, 1H), 6.90-6.99 (m, 2H), 7.26-7.34 (m, 1H), 8.05 (s, 2H), 8.11 (d, J=8.4 Hz, 1H), 8.61 (d, J=8.4 Hz, 1H), 8.71 (s, 1H), 8.78 (s, 1H), 9.75 (s, 1H); LCMS (electrospray) m/z (M+H)⁺ 434.

### 3-((9-Methoxy-4-oxo-2-(quinolin-6-ylamino)-4H-pyrido[1,2-a]pyrimidin-3-yl)methyl)oxazolidin-2-one (353)

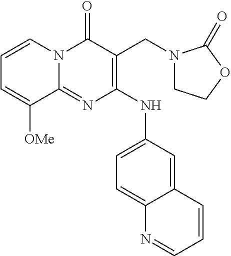 [SMILES: COc1cccn2c(=O)c(CN3CCOC3=O)c(Nc3ccc4ncccc4c3)nc12]

White solid; ¹H NMR (400 MHz, DMSO-d₆) δ 3.61 (t, J=8.4 Hz, 2H), 4.04 (s, 3H), 4.30 (t, J=8.4 Hz, 2H), 4.53 (s, 2H), 7.18 (d, J=7.6 Hz, 1H), 7.38 (d, J=7.2 Hz, 1H), 7.49 (dd, J=4.0 Hz, 8.4 Hz, 1H), 7.96 (s, 2H), 8.21 (d, J=7.6 Hz, 1H), 8.51 (d, J=7.2 Hz, 1H), 8.76 (d, J=2.8 Hz, 1H), 8.88 (s, 1H), 9.21 (s, 1H); LCMS (electrospray) m/z (M+H)⁺ 418.

### 9-Methoxy-3-((methylamino)methyl)-2-(quinolin-6-ylamino)-4H-pyrido[1,2-a]pyrimidin-4-one (354)

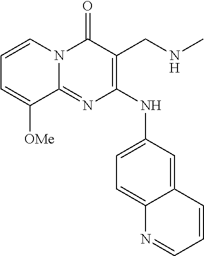 [SMILES: CNCc1c(Nc2ccc3ncccc3c2)nc2c(OC)cccn2c1=O]

White solid; ¹H NMR (400 MHz, DMSO-d₆) δ 2.85 (s, 3H), 4.01 (s, 3H), 7.15 (dd, J=7.6 Hz, 1H), 7.40 (d, J=8.0 Hz, 1H), 7.48 (dd, J=4.0 Hz, 8.4 Hz, 1H), 7.92 (s, 2H), 8.17 (d, J=8.4 Hz, 1H), 8.47 (d, J=7.2 Hz, 1H), 8.76 (d, J=1.6 Hz, 1H), 8.89 (s, 1H), 9.75 (d, J=4.0 Hz, 1H), 13.2 (s, 1H); LCMS (electrospray) m/z (M+H)⁺ 362.

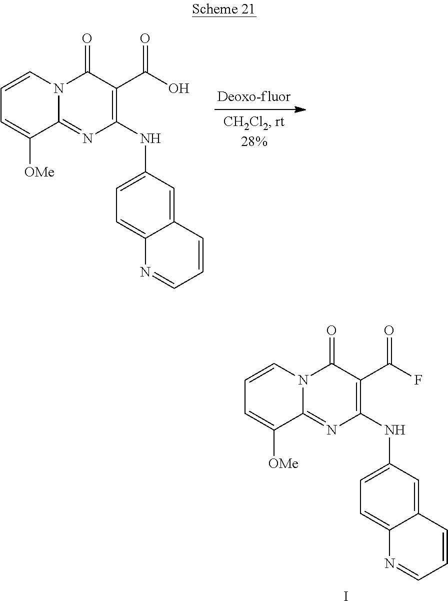

### General Procedure for the Synthesis of I

To a stirred solution of 9-methoxy-4-oxo-2-(quinolin-6-ylamino)-4H-pyrido[1,2-a]pyrimidine-3-carboxylic acid (0.27 mmol) in dichloromethane (1.0 mL) was added Deoxo-Fluor™ (0.30 mmol). The reaction mixture was stirred at room temperature for overnight. The resulting mixture was diluted with dichloromethane (10 mL) and washed with saturated NaHCO₃ solution (10 mL). The organic layer was dried over anhydrous MgSO₄, filtered and concentrated in vacuo. The crude product was purified by flash column chromatography to give I.

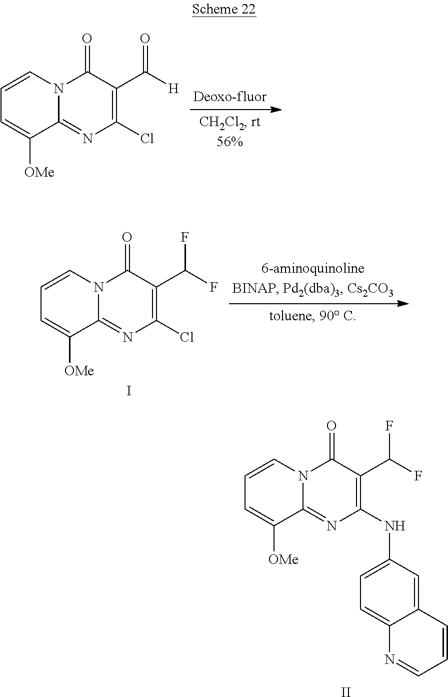

### General Procedure for the Synthesis of I

To a stirred solution of 2-chloro-9-methoxy-4-oxo-4H-pyrido[1,2-a]pyrimidine-3-carbaldehyde (0.84 mmol) in dichloromethane (4.0 mL) was added Deoxo-Fluor™ (1.26 mmol). The reaction mixture was stirred at room temperature for overnight. The resulting mixture was diluted with dichloromethane (20 mL) and washed with saturated NaHCO₃ solution (20 mL). The organic layer was dried over anhydrous MgSO₄, filtered and concentrated in vacuo. The crude product was purified by flash column chromatography to give I.

### General Procedure for the Synthesis of II

To a stirred solution of I (1.15 mmol) in toluene (5.0 mL) was added 6-aminoquinoline (1.15 mmol), 2,2′-bis(diphenylphosphino)-1,1′-binaphthyl (0.11 mmol), tris(dibenzylideneacetone) dipalladium(0) (0.05) and cessium carbonate (1.72 mmol). The reaction mixture was stirred at 90° C. for overnight. After reaction was completed, filtered off and concentrated in vacuo. The crude product was purified by flash column chromatography to give II.

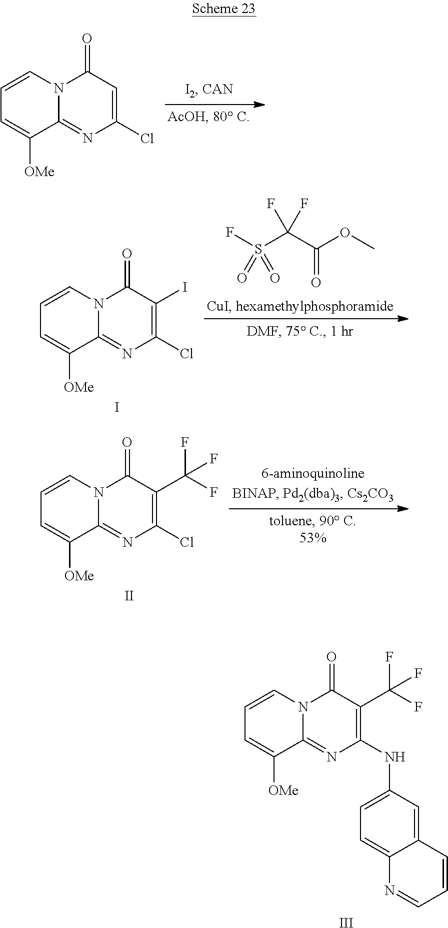

### General Procedure for the Synthesis of I

To a stirred solution of 2-chloro-9-methoxy-4H-pyrido[1,2-a]pyrimidin-4-one (2.37 mmol) in acetic acid (10.0 mL) was added cerium ammonium nitrate (1.18 mmol) and iodine (1.42 mmol). The reaction mixture was stirred at room temperature for overnight. The resulting mixture was concentrated in vacuo. The crude product was purified by flash column chromatography to give I.

### General Procedure for the Synthesis of II

To a solution of I (0.74 mmol) in dimethylformamide (5.0 mL) was added methyl-difluoro(fluorosulfonyl)acetate (5.55 mmol), CuI (1.11 mmol) and hexamethyl phosphoramide (2.0 mL). The reaction mixture was stirred at 75° C. for 1 hour. After reaction was completed, dichloromethane (30 mL) and saturated ammonium chloride solution (30 mL) were added. The organic layer was dried over anhydrous MgSO₄, filtered and concentrated in vacuo. The crude product was purified by flash column chromatography to give II.

### General Procedure for the Synthesis of III

To a solution of II (0.21 mmol) in toluene (1.0 mL) was added 6-aminoquinoline (0.21 mmol), 2,2′-bis(diphenylphosphino)-1,1′-binaphthyl (0.02 mmol), tris(dibenzylideneacetone) dipalladium(0) (0.01 mmol) and cessium carbonate (0.63 mmol). The reaction mixture was stirred at 90° C. for overnight. After reaction was completed, filtered off and concentrated in vacuo. The crude product was purified by flash column chromatography to give III.

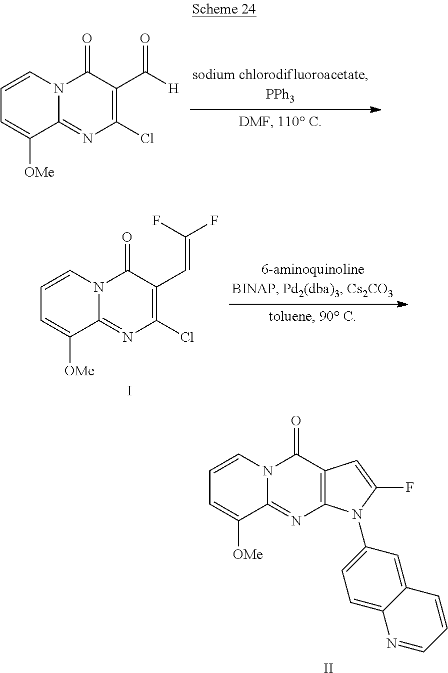

### General Procedure for the Synthesis of I

To a stirred solution of 2-chloro-9-methoxy-4-oxo-4H-pyrido[1,2-a]pyrimidine-3-carbaldehyde (2.09 mmol) in dimethylformamide (10.0 mL) was added sodium chlorodifluoroacetate (3.13 mmol) and triphenylphosphine (3.13 mmol). The reaction mixture was stirred at 115° C. for 1 hour. The resulting mixture was concentrated in vacuo. The crude product was purified by flash column chromatography to give I.

### General Procedure for the Synthesis of II

To a stirred solution of I (0.11 mmol) in toluene (1.0 mL) was added 6-aminoquinoline (0.13 mmol), 2,2′-bis(diphenylphosphino)-1,1′-binaphthyl (0.01 mmol), tris(dibenzylideneacetone) dipalladium(0) (0.005 mmol) and cessium carbonate (0.33 mmol). The reaction mixture was stirred at 90° C. for overnight. After reaction was completed, filtered off and concentrated in vacuo. The crude product was purified by flash column chromatography to give II.

### 9-Methoxy-4-oxo-2-(quinolin-6-ylamino)-4H-pyrido[1,2-a]pyrimidine-3-carbonyl fluoride (355)

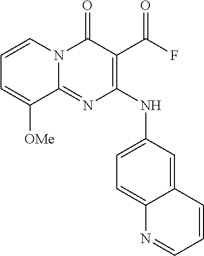 [SMILES: COc1cccn2c(=O)c(C(=O)F)c(Nc3ccc4ncccc4c3)nc12]

White solid; ¹H NMR (400 MHz, DMSO-d₆) δ 4.00 (s, 3H), 7.21 (dd, J=7.2 Hz, 1H), 7.48-7.54 (m, 2H), 7.93-8.00 (m, 2H), 8.21 (d, J=8.4 Hz, 1H), 8.47 (d, J=6.8 Hz, 1H), 8.81 (d, J=4.0 Hz, 1H), 8.87 (s, 1H), 10.43 (s, 1H)

### 3-(Difluoromethyl)-9-methoxy-2-(quinolin-6-ylamino)-4H-pyrido[1,2-a]pyrimidin-4-one (356)

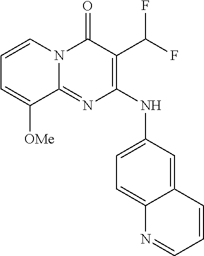 [SMILES: COc1cccn2c(=O)c(C(F)F)c(Nc3ccc4ncccc4c3)nc12]

White solid; ¹H NMR (400 MHz, CDCl₃) δ 4.06 (s, 3H), 7.00 (dd, J=7.2 Hz, 7.2 Hz, 1H), 7.09 (dd, J=1.2 Hz, 8.0 Hz, 1H), 7.37 (t, J=54.4 Hz, 1H, due to F₂), 7.40 (dd, J=4.4 Hz, 8.0 Hz, 1H), 7.71 (brs, 1H), 7.91 (dd, J=2.4 Hz, 9.2 Hz, 1H), 8.07 (d, J=9.2 Hz, 1H), 8.11 (d, J=8.4 Hz, 1H), 8.56 (d, J=2.4 Hz, 1H), 8.61 (dd, J=1.6 Hz, 6.8 Hz, 1H), 8.83 (d, J=2.8 Hz, 1H) ¹⁹F NMR (376 MHz, DMSO-d₆); 8-114.35 (d, J=54.1 Hz, 2F); LCMS (electrospray) m/z (M+H)⁺ 369.

### 9-Methoxy-2-(methyl(quinolin-6-yl)amino)-4-oxo-4H-pyrido[1,2-a]pyrimidine-3-carbaldehyde (357)

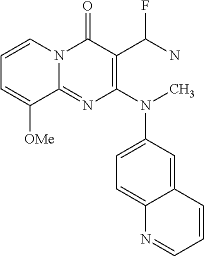 [SMILES: COc1cccn2c(=O)c(C(N)F)c(N(C)c3ccc4ncccc4c3)nc12]

White solid; ¹H NMR (400 MHz, CDCl₃) δ 3.78 (s, 3H), 4.00 (s, 3H), 6.98 (dd, J=7.6 Hz, 7.6 Hz, 1H), 7.10 (dd, J=1.2 Hz, 7.6 Hz, 1H), 7.35 (dd, J=4.4 Hz, 8.4 Hz, 1H), 7.56 (d, J=2.4 Hz, 1H), 7.64 (dd, J=2.4 Hz, 9.2 Hz, 1H), 8.02-8.06 (m, 2H), 8.63 (dd, J=1.6 Hz, 7.2 Hz, 1H), 8.84 (dd, J=1.6 Hz, 4.4 Hz, 1H), 9.90 (s, 1H); LCMS (electrospray) m/z (M+H)⁺ 361.

### 9-Methoxy-2-(quinolin-6-ylamino)-3-(trifluoromethyl)-4H-pyrido[1,2-a]pyrimidin-4-one (358)

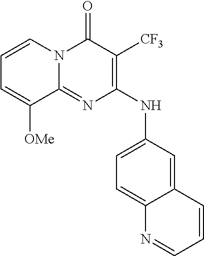 [SMILES: COc1cccn2c(=O)c(C(F)(F)F)c(Nc3ccc4ncccc4c3)nc12]

White solid; ¹H NMR (400 MHz, CDCl₃) δ 4.00 (s, 3H), 6.97 (d, J=7.2 Hz, 1H), 7.05 (d, J=7.2 Hz, 1H), 7.22 (s, 1H), 7.37 (s, 1H), 7.70 (s, 1H), 7.82 (d, J=8.0 Hz, 1H), 8.03-8.08 (m, 2H), 8.48 (s, 1H), 8.59 (d, J=5.6 Hz, 1H), 8.81 (s, 1H)

### 2-Fluoro-9-methoxy-1-(quinolin-6-yl)pyrido[1,2-a]pyrrolo[2,3-d]pyrimidin-4(1H)-one (359)

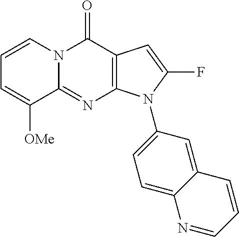 [SMILES: COc1cccn2c(=O)c3cc(F)n(-c4ccc5ncccc5c4)c3nc12]

White solid; ¹H NMR (400 MHz, DMSO-d₆) δ 3.91 (s, 3H), 6.36 (d, J=17.6 Hz, 1H, due to F), 7.13 (dd, J=7.2 Hz, 7.2 Hz, 1H), 7.30 (d, J=7.2 Hz, 1H), 7.47 (dd, J=4.0 Hz, 8.8 Hz, 1H), 7.51 (dd, J=1.2 Hz, 9.2 Hz, 1H), 7.89 (d, J=8.8 Hz, 1H), 8.18-8.21 (m, 1H), 8.39-8.40 (m, 1H), 8.73-8.76 (m, 2H); ¹⁹F NMR (376 MHz, DMSO-d₆); δ −93.82 (d, J=17.2 Hz, 1F); LCMS (electrospray) m/z (M+H)⁺ 369.

### Example 8

### Additional Studies on Pyridopyrimidinone Compounds

Table 3 shows the minimal inhibitory concentration (MIC) of one representative compound, 133, on different Mycobacterial species. While it has no effect on the fast growing Mycobacterium smegmatis mc², it was able to inhibit typical laboratory strains such as H37Rv, H37Ra and BCG Pasteur with an MIC of 2 μM. More importantly, the antimicrobial activity of 133 was also tested against clinical isolates strains of mycobacteria. The MIC values for multi-drug-resistant (MDR-TB) and extensive-drug-resistant (XDR-TB) isolates strains were within the micromolar range.

To address the issue of toxicity, compound 133 was tested on a panel of seven cell lines derived from different body tissue. Cells were incubated with increasing amounts of compound and cell viability was assessed with resazurin after 5 days of co-incubation. Percentage of cytotoxicity was determined by taking as a reference the resofurin fluorescence measured by DMSO containing wells. The concentration where fifty percent of the cells died was defined as the Minimal Toxic Concentration (MTC₅₀). Compound 133 showed no cytotoxicity for all tested cell lines up to 100 μM (Table 3). The selectivity index, which consists of the ratio between antitubercular activity and cytotoxicity was therefore above 50 for both extracellular and intracellular mycobacteria suggesting this series of compounds to be promising new anti-tuberculosis drugs.

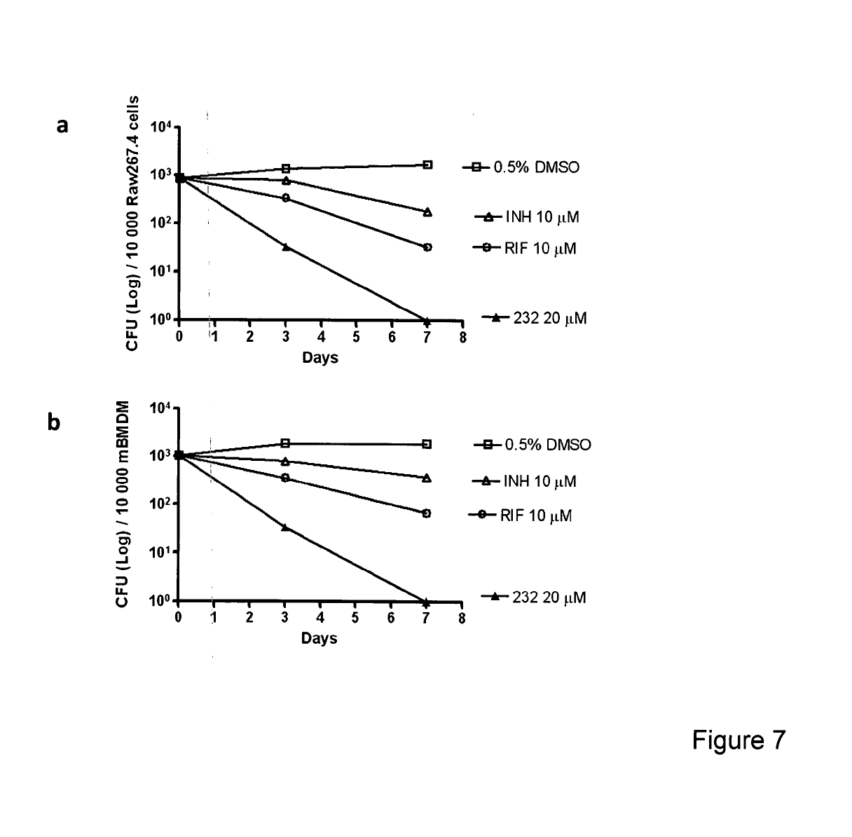

The effect of this series of compounds on primary macrophages was further determined. Host cells that had priory been incubated with compound 232 harbored fewer bacteria compared to DMSO control and were more abundant at day 5 after infection as shown in FIG. 7. Similar data were obtained for compound 133 (data not shown). Conventional CFU determination was then performed seven days after infection to quantify the remaining bacterial load. A ten-fold decrease in the number of CFUs, similar to that seen with INH, was observed for both compounds on both human and mouse cells (FIG. 7). This confirms the potency of this series of compounds.

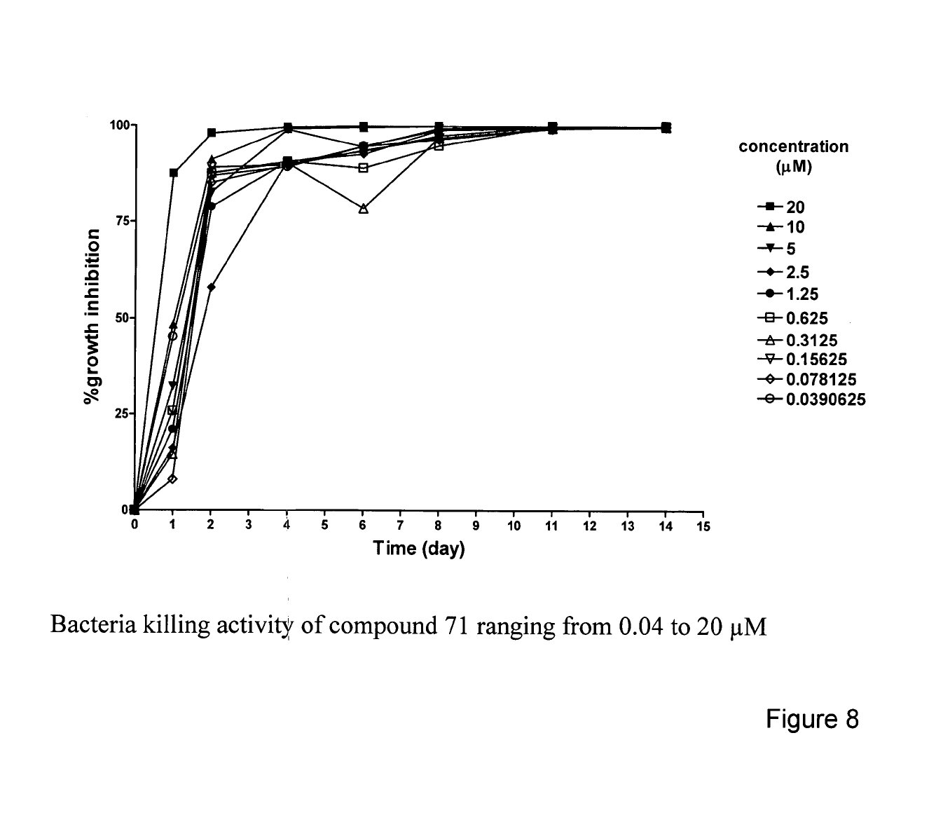

The bacteria killing activity of compound 71 was determined using the CFU counting method. Two-fold serial dilutions of compound 71 were incubated aerobically with H37Rv for 21 days at 37° C. FIG. 8 shows the bacteria growth inhibition rate in the presence of compound 71.

At concentrations ranging from 0.04 to 20 μM, compound 71 strongly inhibited the growth of M. tuberculosis. After only 2 days of incubation, it showed fast bacteria killing activity even at the lowest concentration. This further confirms the potency of this series of compounds.

Mutation frequency of M. tuberculosis H37Rv was determined for compound 264. Increasing numbers of bacteria grew on 7H10 agar medium supplemented with different concentrations of compound. After a 6-week growth, colonies were counted in order to evaluate the proportion of spontaneous mutational frequency (Table 4). Compound 264 gave frequencies of resistance of 3.4×10⁻⁶ and 8×10⁻⁶ at 0.4 and 0.8 μg/ml, respectively, and 2×10⁻⁸ at both 1.6 μg/ml and 3.2 μg/ml. Accordingly, spontaneous mutational rate was calculated to be 7×10⁻⁷.

Overall, these values are better than the frequency of mutation observed for INH (2.9×10⁻⁶). These results, therefore, demonstrate that this class of compounds result in a low frequency of mutation.

One of the current challenges for TB drug discovery is the identification of compounds that are active against persistent bacteria. Although the location and state of latent bacteria remains a matter of debate, one commonly shared hypothesis for mycobacterial persistence is that M. tuberculosis bacilli are able to survive in macrophages for prolonged periods of time and, unlike other bacteria, are able to actively replicate. The intraphagosomal profile of M. tuberculosis is complex; a large variety of genes are over-expressed and timely regulated and are also dependent on environmental factors. Altogether, this makes the identification of one specific tubercle factor that could be selected as the ideal target difficult. Consequently, non-target cell-based assays are a critical tool in the search of intracellular M. tuberculosis inhibitors.

Investigation of bacillus growth inhibitors within macrophages has long been limited due to cumbersome CFU plating, slow bacillus growth, safety requirements and difficulties in setting-up appropriate infection conditions. As a consequence, this approach was always used as a secondary assay after the initial selection of compounds that are active on in vitro extracellular growth. With the advent of automated confocal microscopy, the above mentioned limitations could be readdressed and the inventors show the feasibility of large scale compound screening. It was decided to perform suspension macrophage batch infection in order to minimize the steps and to meet safety requirements. To this end, careful attention was paid to the removal of the extracellular non-phagocytosed mycobacteria. The centrifugation conditions used during the wash steps were set up in order to recover only the infected cells and discard most of the extracellular bacteria. By microscopy the inventors confirmed that unbound mycobacteria represented less than 10% of the total bacterial load (data not shown). Mycobacteria are able to grow independently of host cells and consequently any remaining extracellular bacilli would greatly compromise the validity of the inventors' model. To this end, an additional amikacin treatment step was added to the protocol to further eliminate any remaining mycobacteria. Thus with the optimized protocol, there is almost no non-phagocytosed mycobacteria left by the time compound is added. The obtained results also demonstrate that it is specifically the effect on the intracellular mycobacteria that is being measured with compound treatment. Indeed, the inventors observed a weak inhibition with rifampin, an antibiotic that is known to poorly penetrate cells. The 50-fold reproducible decrease in MIC for rifampin in the intracellular assay compared to the in vitro growth assay proved that the targeted bacteria are not extracellular. Otherwise no difference would have been seen in MIC between the two assays. Similarly, compounds able to inhibit mycobacterial growth in the phenotypic cell-based assay, but not the in vitro growth assay were also identified. In addition, the fact that the compounds are mixed with previously infected cells should decrease the chance for the identification of primary infection inhibitors. However, such compounds may still be identified as blockers of neighboring cell infection.

Compared to a conventional CFU-plating method, the microscopy based detection of fluorescent bacteria is not sufficiently sensitive to distinguish between dead and live bacilli as the GFP signal is stable for several days. Indeed, at a high concentration of INH, rifampin or active compound, there is always 10% of the cells that appear to be infected, which is similar to the initial infection ratio. Surprisingly, no CFU could be recovered after plating such samples. Owing to the fact that latent bacilli are able to recover growth (Cho et al., 2007), the microscopy-detected bacilli must be dead bacilli rather than latent bacilli. Thus, the inventors' assay detects compounds that interfere with bacilli growth within macrophages.

As it is well established and confirmed (FIG. 1a), macrophages are able to support high bacterial loads which end up encompassing a large part of the cell cytoplasm and eventually lead to macrophage cell death. It is obvious when M. tuberculosis is the infectious agent compared to BCG (Bacille Calmette-Guerin), which even at high MOI fails to induce much cytotoxicity (data not shown). Taking this into account, it was decided to set the data acquisition at day 5 post-infection when the cell number in the DMSO samples had significantly decreased relative to the antibiotically protected controls. Thus, monitoring cell number was an additional parameter enabling the inventors to confirm the compound's antibacterial activity.

Unlike direct fluorescence based assays, analysis for image-based assays proved to be much more variable. Several parameters that are inherent to the biology of the assay partially explain the lower Z′-values that are usually accepted for HTS validation. The remaining fluorescent dead bacilli do not have much of an impact on the Z′-value, rather the variability in the infection ratio for the DMSO controls seems to account for the discrepancy. Also of importance is the fact that, upon infection, the macrophages had a tendency to migrate which in turn led to a heterogeneous set of images (FIG. 2a). However, the aim of the primary screen was to identify compounds fully active at a concentration of 20 μM. Thus, for this purpose, a positive Z′ for the infection ratio (INH/DMSO) was considered an acceptable value. The best proof of the validity of the hit selection according to the present invention comes from the subsequent serial dilution analysis, whereby almost 100% of the hits were confirmed. For each of the hits, a nicely fitted dose-response curve for the infection ratio was obtained as well as for the non-toxic compound in terms of cell number. Again, cell number brought an additional confirmation of the results that is totally independent of green fluorescence emission and GFP expression.

Obviously compounds found to be active against both intracellular and in vitro M. tuberculosis growth are the most promising. The best inhibitors isolated from this library have an inhibitory activity within the same range as INH. Further structure activity relationship studies will contribute to determine if their activity could be improved. In the course of another study using this phenotypic cell-based model, MIC down to the ng/mL scale was obtained for compounds with known in vitro antibacterial efficacy showing that compounds with a lower MIC than INH can be identified by the assay according to the present invention (data not shown). Of utmost interest are the compounds that are active only in the intracellular bacteria assay as they are likely to have a new mechanism of action independent of the infecting strain suggesting that they may also be active on the non-curable multi-drug-resistant (MDR)-strains.

Taken together, the above results show that monitoring M. tuberculosis growth with automated fluorescence microscopy is highly robust and reliable and that this method enables fast selection of potent anti-TB compounds.

### REFERENCES

Abadie, V., Badell, E., Douillard, P., Ensergueix, D., Leenen, P. J., Tanguy, M., Fiette, L., Saeland, S., Gicquel, B., and Winter, N. (2005). Neutrophils rapidly migrate via lymphatics after Mycobacterium bovis BCG intradermal vaccination and shuttle live bacilli to the draining lymph nodes. Blood 106, 1843-1850.
Andries, K., Verhasselt, P., Guillemont, J., Gohlmann, H. W., Neefs, J. M., Winkler, H., Van Gestel, J., Timmerman, P., Zhu, M., Lee, E., et al. (2005). A diarylquinoline drug active on the ATP synthase of Mycobacterium tuberculosis. Science 307, 223-227.
Arain, T. M., Resconi, A. E., Singh, D. C., and Stover, C. K. (1996). Reporter gene technology to assess activity of antimycobacterial agents in macrophages. Antimicrob Agents Chemother 40, 1542-1544.
Brodin, P., Majlessi, L., Marsollier, L., de Jonge, M. I., Bottai, D., Demangel, Cl., Hinds, J., Neyrolles, O., Butcher, P. D., Leclerc, C., Coles, S. T., Brosch, R., (2006). Dissection of ESAT-6 system 1 of Mycobacterium tuberculosis and impact on immunogenicity and virulence. Infect Immun 74, 88-98.
Cho, S. H., Warit, S., Wan, B., Hwang, C. H., Pauli, G. F., and Franzblau, S. G. (2007). Low-oxygen-recovery assay for high-throughput screening of compounds against nonreplicating Mycobacterium tuberculosis. Antimicrob Agents Chemother 51, 1380-1385.
Cremer, I., Dieu-Nosjean, M. C., Marechal, S., Dezutter-Dambuyant, C., Goddard, S., Adams, D., Winter, N., Menetrier-Caux, C., Sautes-Fridman, C., Fridman, W. H., and Mueller, C. G. (2002). Long-lived immature dendritic cells mediated by TRANCE-RANK interaction. Blood 100, 3646-3655.
Fenistein, D., Lenseigne, B., Christophe, T., Brodin, P., and Genovesio, A. (2008). A fast fully automated cell segmentation algorithm for high throughput and high content screening. Cytometry part A, 73, 958-64.
Houben, E. N., Nguyen, L., and Pieters, J. (2006). Interaction of pathogenic mycobacteria with the host immune system. Curr Opin Microbiol 9, 76-85.
Lenaerts, A. J., Hoff, D., Aly, S., Ehlers, S., Andries, K., Cantarero, L., Orme, I. M., and Basaraba, R. J. (2007). Location of persisting mycobacteria in a Guinea pig model of tuberculosis revealed by r207910. Antimicrob Agents Chemother 51, 3338-3345.
Lipinski, C. A., Lombardo, F., Dominy, B. W., and Feeney, P. J. (2001). Experimental and computational approaches to estimate solubility and permeability in drug discovery and development settings. Advanced Drug Delivery Reviews 46, 3-26.
Neyrolles, O., Hernandez-Pando, R., Pietri-Rouxel, F., Fornes, P., Tailleux, L., Barrios Payan, J. A., Pivert, E., Bordat, Y., Aguilar, D., Prevost, M. C., et al. (2006). Is adipose tissue a place for Mycobacterium tuberculosis persistence? PLoS ONE 1, e43.
Rohde, K. H., Abramovitch, R. B., and Russell, D. G. (2007). Mycobacterium tuberculosis invasion of macrophages: linking bacterial gene expression to environmental cues. Cell Host Microbe 2, 352-364.
Salomon, J. A., Lloyd-Smith, J. O., Getz, W. M., Resch, S., Sanchez, M. S., Porco, T. C., and Borgdorff, M. W. (2006). Prospects for advancing tuberculosis control efforts through novel therapies. PLoS Med 3, e273.
Schnappinger, D., Ehrt, S., Voskuil, M. I., Liu, Y., Mangan, J. A., Monahan, I. M., Dolganov, G., Efron, B., Butcher, P. D., Nathan, C., and Schoolnik, G. K. (2003). Transcriptional Adaptation of Mycobacterium tuberculosis within Macrophages: Insights into the Phagosomal Environment. J Exp Med 198, 693-704.
Van Rie, A., and Enarson, D. (2006). XDR tuberculosis: an indicator of public-health negligence. Lancet 368, 1554-1556.

TABLE 1

| Compound | QUM (μM) | QIM (μm) |
|---|---|---|
| 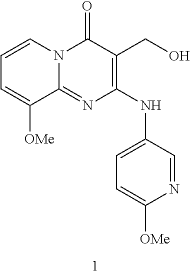 [SMILES: COc1ccc(Nc2nc3c(OC)cccn3c(=O)c2CO)cn1] | ++ | + |
| 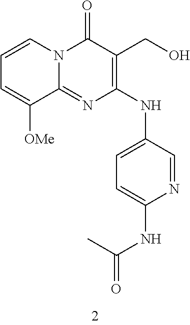 [SMILES: COc1cccn2c(=O)c(CO)c(Nc3ccc(NC(C)=O)nc3)nc12] | ++ | + |
| 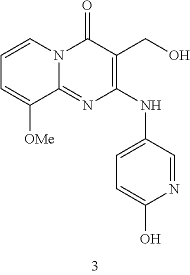 [SMILES: COc1cccn2c(=O)c(CO)c(Nc3ccc(O)nc3)nc12] | + | + |
| 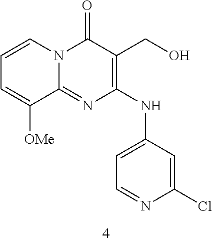 [SMILES: COc1cccn2c(=O)c(CO)c(Nc3ccnc(Cl)c3)nc12] | ++ | +++ |
| 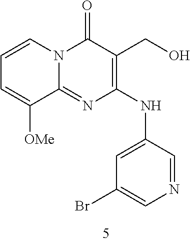 [SMILES: COc1cccn2c(=O)c(CO)c(Nc3cncc(Br)c3)nc12] | +++ | +++ |
| 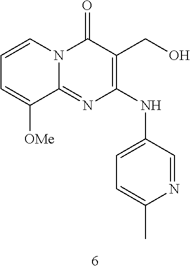 [SMILES: COc1cccn2c(=O)c(CO)c(Nc3ccc(C)nc3)nc12] | ++ | + |
| 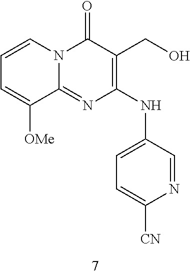 [SMILES: COc1cccn2c(=O)c(CO)c(Nc3ccc(C#N)nc3)nc12] | ++ | + |
| 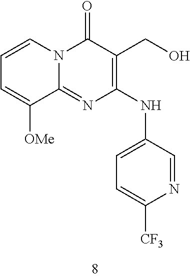 [SMILES: COc1cccn2c(=O)c(CO)c(Nc3ccc(C(F)(F)F)nc3)nc12] | ++ | + |
| 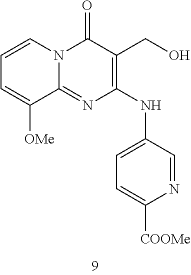 [SMILES: COC(=O)c1ccc(Nc2nc3c(OC)cccn3c(=O)c2CO)cn1] | + | + |
| 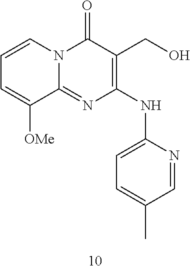 [SMILES: COc1cccn2c(=O)c(CO)c(Nc3ccc(C)cn3)nc12] | + | + |
| 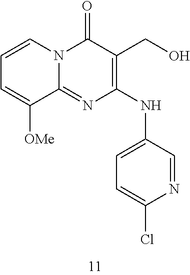 [SMILES: COc1cccn2c(=O)c(CO)c(Nc3ccc(Cl)nc3)nc12] | ++ | + |
| 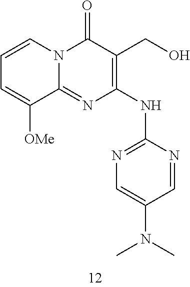 [SMILES: COc1cccn2c(=O)c(CO)c(Nc3ncc(N(C)C)cn3)nc12] | + | + |
| 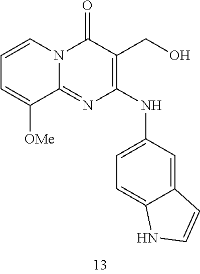 [SMILES: COc1cccn2c(=O)c(CO)c(Nc3ccc4[nH]ccc4c3)nc12] | +++ | ++ |
| 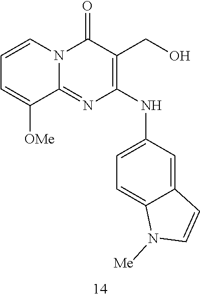 [SMILES: COc1cccn2c(=O)c(CO)c(Nc3ccc4c(ccn4C)c3)nc12] | ++ | + |
| 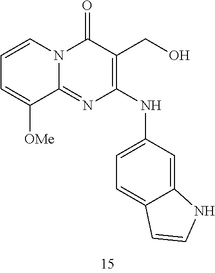 [SMILES: COc1cccn2c(=O)c(CO)c(Nc3ccc4cc[nH]c4c3)nc12] | ++ | + |
| 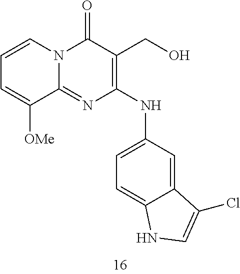 [SMILES: COc1cccn2c(=O)c(CO)c(Nc3ccc4[nH]cc(Cl)c4c3)nc12] | ++ | ++ |
| 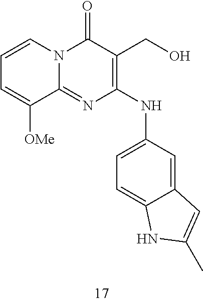 [SMILES: COc1cccn2c(=O)c(CO)c(Nc3ccc4[nH]c(C)cc4c3)nc12] | ++ | + |
| 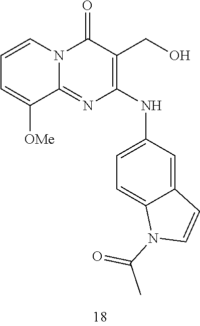 [SMILES: COc1cccn2c(=O)c(CO)c(Nc3ccc4c(ccn4C(C)=O)c3)nc12] | ++ | + |
| 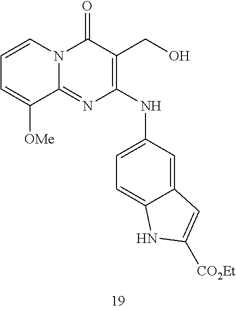 [SMILES: CCOC(=O)c1cc2cc(Nc3nc4c(OC)cccn4c(=O)c3CO)ccc2[nH]1] | ++ | + |
| 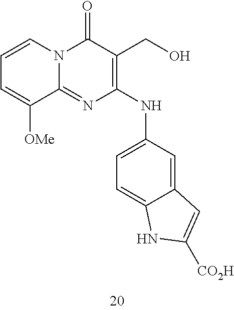 [SMILES: COc1cccn2c(=O)c(CO)c(Nc3ccc4[nH]c(C(=O)O)cc4c3)nc12] | + | + |
| 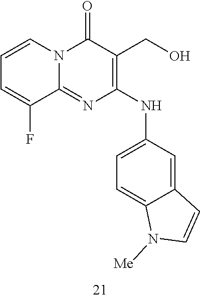 [SMILES: Cn1ccc2cc(Nc3nc4c(F)cccn4c(=O)c3CO)ccc21] | + | + |
| 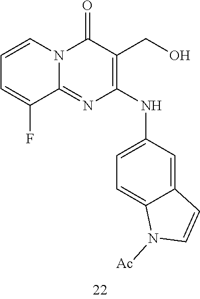 [SMILES: CC(=O)n1ccc2cc(Nc3nc4c(F)cccn4c(=O)c3CO)ccc21] | + | + |
| 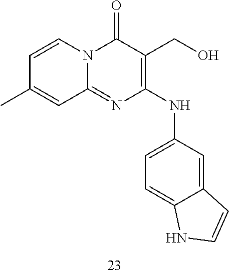 [SMILES: Cc1ccn2c(=O)c(CO)c(Nc3ccc4[nH]ccc4c3)nc2c1] | ++ | ++ |
| 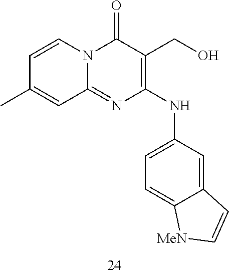 [SMILES: Cc1ccn2c(=O)c(CO)c(Nc3ccc4c(ccn4C)c3)nc2c1] | + | + |
| 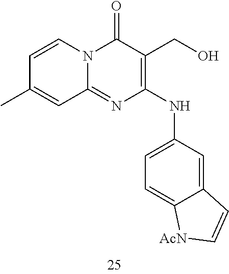 | ++ | ++ |
| 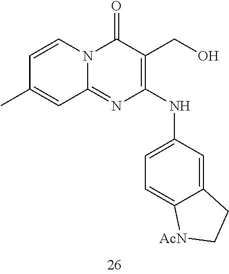 | + | + |
| 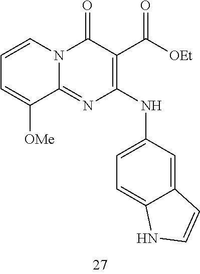 [SMILES: CCOC(=O)c1c(Nc2ccc3[nH]ccc3c2)nc2c(OC)cccn2c1=O] | + | + |
| 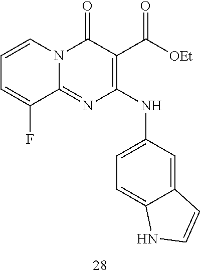 [SMILES: CCOC(=O)c1c(Nc2ccc3[nH]ccc3c2)nc2c(F)cccn2c1=O] | + | + |
| 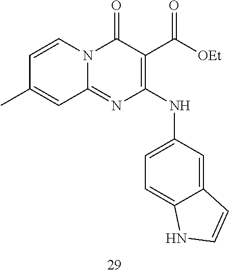 [SMILES: CCOC(=O)c1c(Nc2ccc3[nH]ccc3c2)nc2cc(C)ccn2c1=O] | + | + |
| 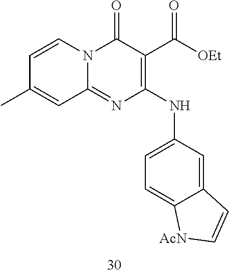 | + | + |
| 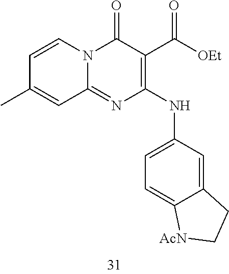 | + | + |
| 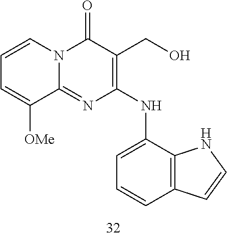 [SMILES: COc1cccn2c(=O)c(CO)c(Nc3cccc4cc[nH]c34)nc12] | ++ | ++ |
| 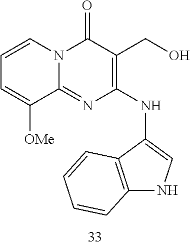 [SMILES: COc1cccn2c(=O)c(CO)c(Nc3c[nH]c4ccccc34)nc12] | ++ | + |
| 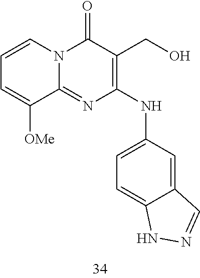 [SMILES: COc1cccn2c(=O)c(CO)c(Nc3ccc4[nH]ncc4c3)nc12] | ++ | ++ |
| 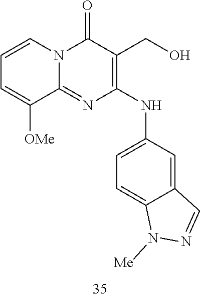 [SMILES: COc1cccn2c(=O)c(CO)c(Nc3ccc4c(cnn4C)c3)nc12] | ++ | + |
| 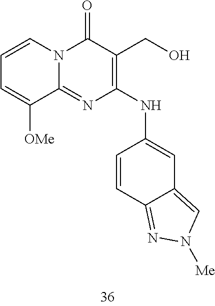 [SMILES: COc1cccn2c(=O)c(CO)c(Nc3ccc4nn(C)cc4c3)nc12] | ++ | + |
| 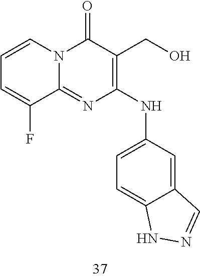 [SMILES: O=c1c(CO)c(Nc2ccc3[nH]ncc3c2)nc2c(F)cccn12] | ++ | ++ |
| 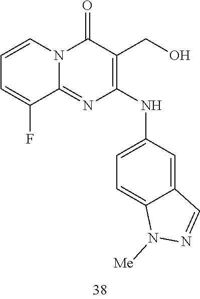 [SMILES: Cn1ncc2cc(Nc3nc4c(F)cccn4c(=O)c3CO)ccc21] | ++ | + |
| 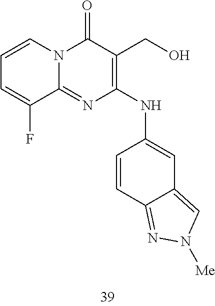 [SMILES: Cn1cc2cc(Nc3nc4c(F)cccn4c(=O)c3CO)ccc2n1] | + | + |
| 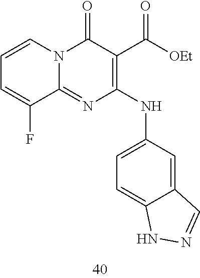 [SMILES: CCOC(=O)c1c(Nc2ccc3[nH]ncc3c2)nc2c(F)cccn2c1=O] | + | + |
| 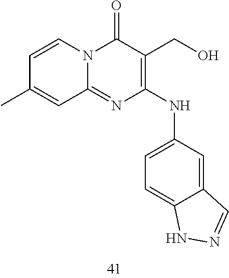 [SMILES: Cc1ccn2c(=O)c(CO)c(Nc3ccc4[nH]ncc4c3)nc2c1] | ++ | + |
| 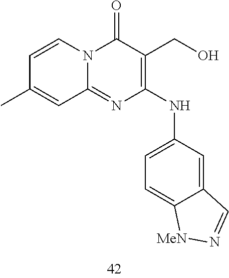 [SMILES: Cc1ccn2c(=O)c(CO)c(Nc3ccc4c(cnn4C)c3)nc2c1] | + | + |
| 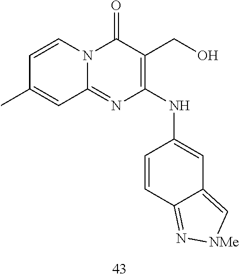 [SMILES: Cc1ccn2c(=O)c(CO)c(Nc3ccc4nn(C)cc4c3)nc2c1] | + | + |
| 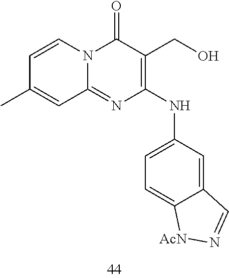 | ++ | ++ |
| 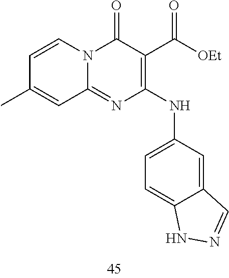 [SMILES: CCOC(=O)c1c(Nc2ccc3[nH]ncc3c2)nc2cc(C)ccn2c1=O] | + | + |
| 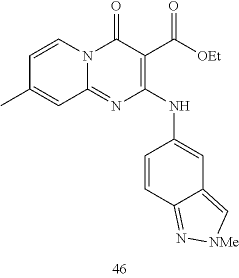 [SMILES: CCOC(=O)c1c(Nc2ccc3nn(C)cc3c2)nc2cc(C)ccn2c1=O] | + | + |
| 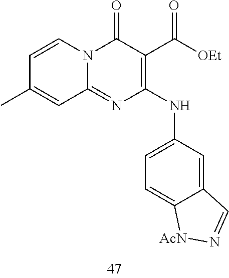 | + | + |
| 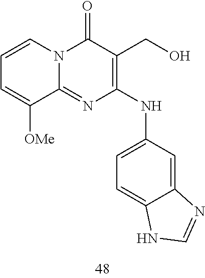 [SMILES: COc1cccn2c(=O)c(CO)c(Nc3ccc4[nH]cnc4c3)nc12] | ++ | + |
| 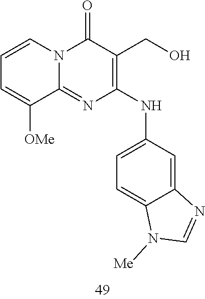 [SMILES: COc1cccn2c(=O)c(CO)c(Nc3ccc4c(c3)ncn4C)nc12] | + | + |
| 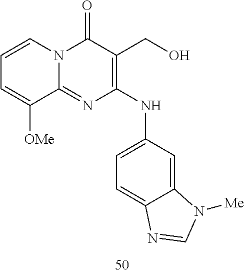 [SMILES: COc1cccn2c(=O)c(CO)c(Nc3ccc4ncn(C)c4c3)nc12] | ++ | + |
| 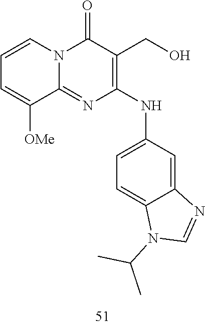 [SMILES: COc1cccn2c(=O)c(CO)c(Nc3ccc4c(c3)ncn4C(C)C)nc12] | + | + |
| 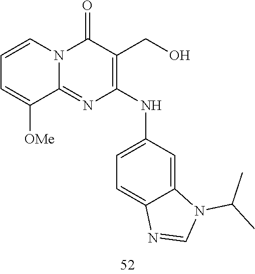 [SMILES: COc1cccn2c(=O)c(CO)c(Nc3ccc4ncn(C(C)C)c4c3)nc12] | ++ | + |
| 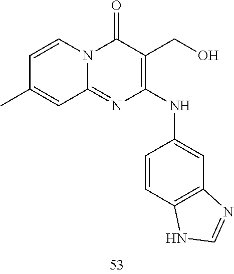 [SMILES: Cc1ccn2c(=O)c(CO)c(Nc3ccc4[nH]cnc4c3)nc2c1] | ++ | + |
| 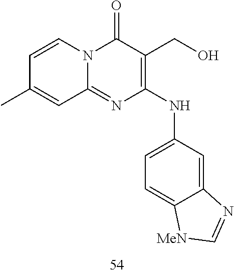 [SMILES: Cc1ccn2c(=O)c(CO)c(Nc3ccc4c(c3)ncn4C)nc2c1] | + | + |
| 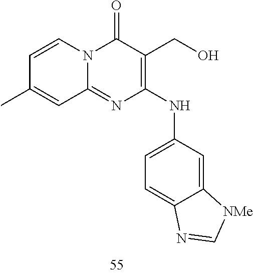 [SMILES: Cc1ccn2c(=O)c(CO)c(Nc3ccc4ncn(C)c4c3)nc2c1] | + | + |
| 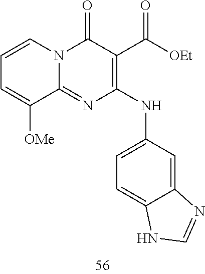 [SMILES: CCOC(=O)c1c(Nc2ccc3[nH]cnc3c2)nc2c(OC)cccn2c1=O] | + | + |
| 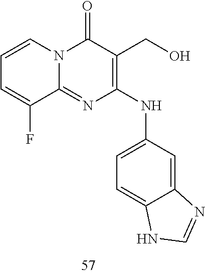 [SMILES: O=c1c(CO)c(Nc2ccc3[nH]cnc3c2)nc2c(F)cccn12] | + | + |
| 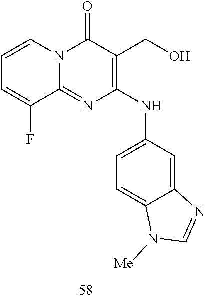 [SMILES: Cn1cnc2cc(Nc3nc4c(F)cccn4c(=O)c3CO)ccc21] | + | + |
| 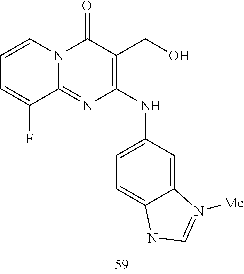 | ++ | + |
| 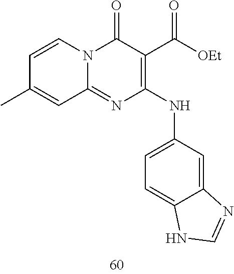 [SMILES: CCOC(=O)c1c(Nc2ccc3[nH]cnc3c2)nc2cc(C)ccn2c1=O] | + | + |
| 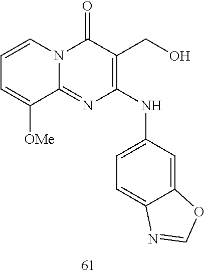 [SMILES: COc1cccn2c(=O)c(CO)c(Nc3ccc4ncoc4c3)nc12] | +++ | ++ |
| 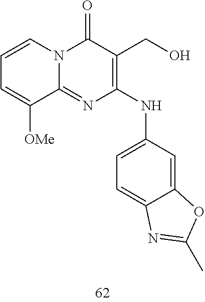 [SMILES: COc1cccn2c(=O)c(CO)c(Nc3ccc4nc(C)oc4c3)nc12] | ++ | ++ |
| 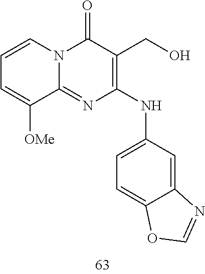 [SMILES: COc1cccn2c(=O)c(CO)c(Nc3ccc4ocnc4c3)nc12] | ++ | ++ |
| 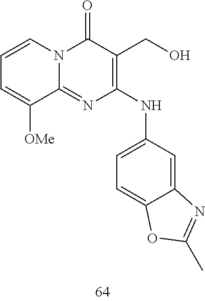 [SMILES: COc1cccn2c(=O)c(CO)c(Nc3ccc4oc(C)nc4c3)nc12] | ++ | + |
| 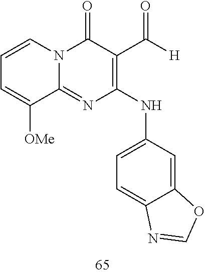 [SMILES: COc1cccn2c(=O)c(C=O)c(Nc3ccc4ncoc4c3)nc12] | +++ | ++ |
| 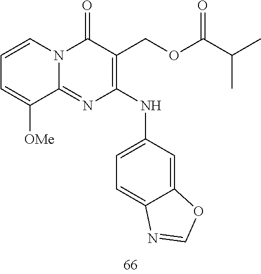 [SMILES: COc1cccn2c(=O)c(COC(=O)C(C)C)c(Nc3ccc4ncoc4c3)nc12] | ++ | ++ |
| 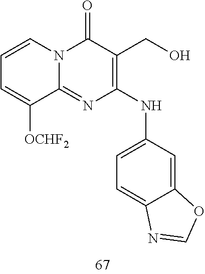 [SMILES: O=c1c(CO)c(Nc2ccc3ncoc3c2)nc2c(OC(F)F)cccn12] | ++ | + |
| 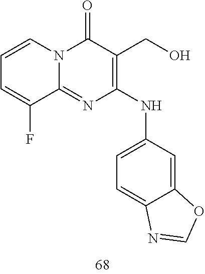 [SMILES: O=c1c(CO)c(Nc2ccc3ncoc3c2)nc2c(F)cccn12] | ++ | + |
| 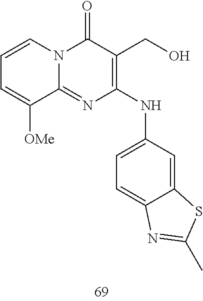 [SMILES: COc1cccn2c(=O)c(CO)c(Nc3ccc4nc(C)sc4c3)nc12] | ++ | ++ |
| 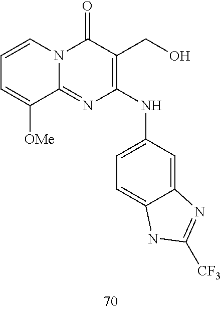 [SMILES: COc1cccn2c(=O)c(CO)c(Nc3ccc4[nH]c(C(F)(F)F)nc4c3)nc12] | + | + |
| 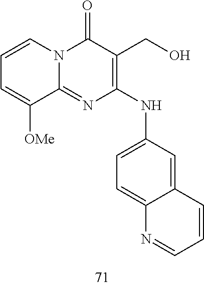 [SMILES: COc1cccn2c(=O)c(CO)c(Nc3ccc4ncccc4c3)nc12] | +++ | ++ |
| 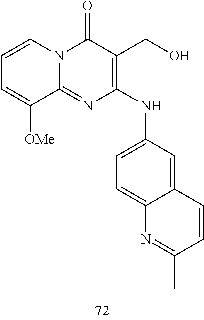 [SMILES: COc1cccn2c(=O)c(CO)c(Nc3ccc4nc(C)ccc4c3)nc12] | ++ | + |
| 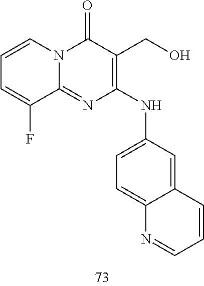 [SMILES: O=c1c(CO)c(Nc2ccc3ncccc3c2)nc2c(F)cccn12] | ++ | + |
| 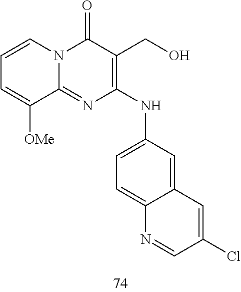 [SMILES: COc1cccn2c(=O)c(CO)c(Nc3ccc4ncc(Cl)cc4c3)nc12] | +++ | ++ |
| 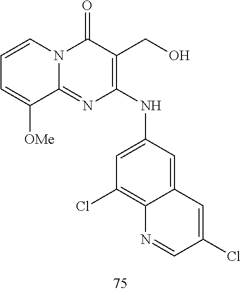 [SMILES: COc1cccn2c(=O)c(CO)c(Nc3cc(Cl)c4ncc(Cl)cc4c3)nc12] | + | + |
| 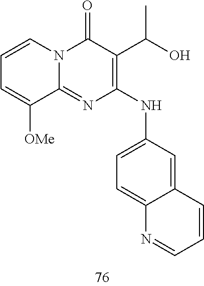 [SMILES: COc1cccn2c(=O)c(C(C)O)c(Nc3ccc4ncccc4c3)nc12] | + | + |
| 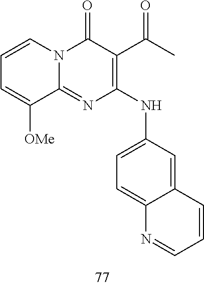 [SMILES: COc1cccn2c(=O)c(C(C)=O)c(Nc3ccc4ncccc4c3)nc12] | + | + |
| 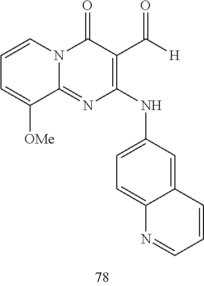 [SMILES: COc1cccn2c(=O)c(C=O)c(Nc3ccc4ncccc4c3)nc12] | +++ | +++ |
| 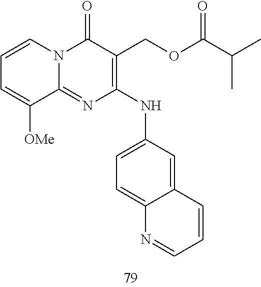 [SMILES: COc1cccn2c(=O)c(COC(=O)C(C)C)c(Nc3ccc4ncccc4c3)nc12] | ++ | ++ |
| 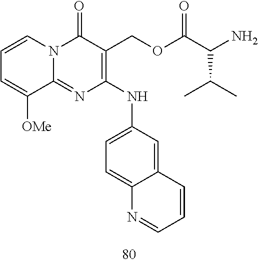 | ++ | + |
| 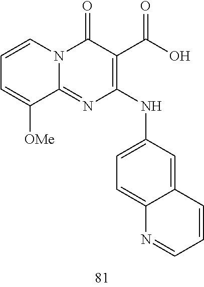 [SMILES: COc1cccn2c(=O)c(C(=O)O)c(Nc3ccc4ncccc4c3)nc12] | ++ | + |
| 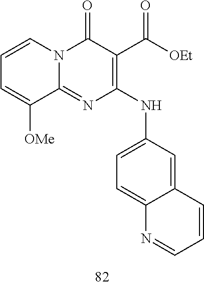 [SMILES: CCOC(=O)c1c(Nc2ccc3ncccc3c2)nc2c(OC)cccn2c1=O] | + | + |
| 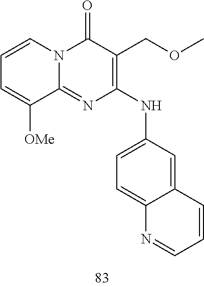 [SMILES: COCc1c(Nc2ccc3ncccc3c2)nc2c(OC)cccn2c1=O] | ++ | ++ |
| 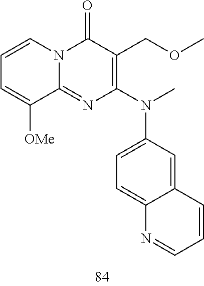 [SMILES: COCc1c(N(C)c2ccc3ncccc3c2)nc2c(OC)cccn2c1=O] | + | + |
| 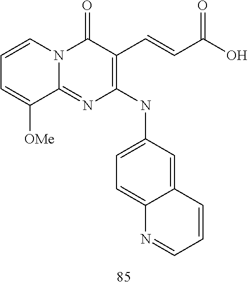 [SMILES: COc1cccn2c(=O)c(/C=C/C(=O)O)c(Nc3ccc4ncccc4c3)nc12] | + | + |
| 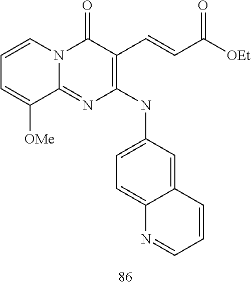 [SMILES: CCOC(=O)/C=C/c1c(Nc2ccc3ncccc3c2)nc2c(OC)cccn2c1=O] | + | + |
| 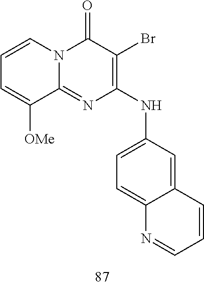 [SMILES: COc1cccn2c(=O)c(Br)c(Nc3ccc4ncccc4c3)nc12] | nd | nd |
| 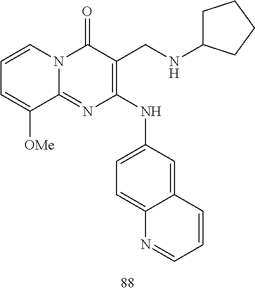 [SMILES: COc1cccn2c(=O)c(CNC3CCCC3)c(Nc3ccc4ncccc4c3)nc12] | ++ | + |
| 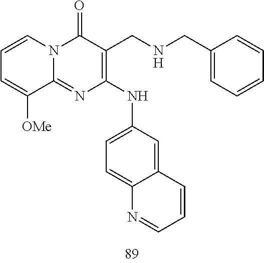 [SMILES: COc1cccn2c(=O)c(CNCc3ccccc3)c(Nc3ccc4ncccc4c3)nc12] | + | + |
| 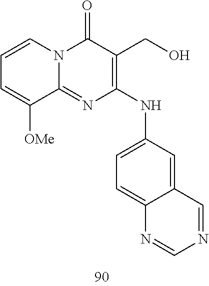 [SMILES: COc1cccn2c(=O)c(CO)c(Nc3ccc4ncncc4c3)nc12] | ++ | + |
| 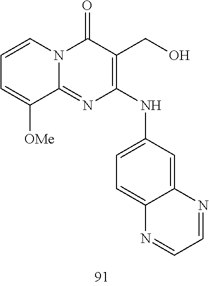 [SMILES: COc1cccn2c(=O)c(CO)c(Nc3ccc4nccnc4c3)nc12] | ++ | + |
| 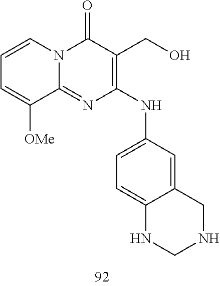 [SMILES: COc1cccn2c(=O)c(CO)c(Nc3ccc4c(c3)CNCN4)nc12] | + | + |
| 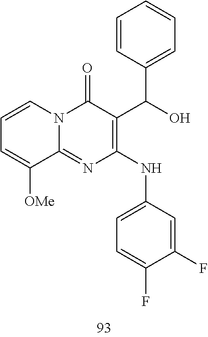 [SMILES: COc1cccn2c(=O)c(C(O)c3ccccc3)c(Nc3ccc(F)c(F)c3)nc12] | + | + |
| 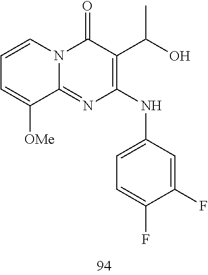 [SMILES: COc1cccn2c(=O)c(C(C)O)c(Nc3ccc(F)c(F)c3)nc12] | + | + |
| 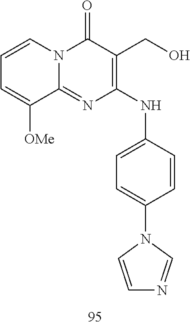 [SMILES: COc1cccn2c(=O)c(CO)c(Nc3ccc(-n4ccnc4)cc3)nc12] | + | + |
| 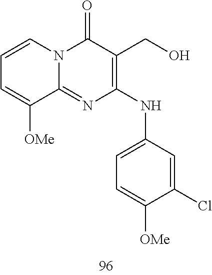 [SMILES: COc1ccc(Nc2nc3c(OC)cccn3c(=O)c2CO)cc1Cl] | ++ | + |
| 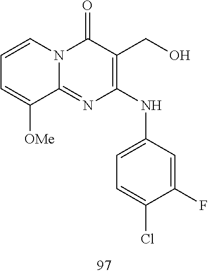 [SMILES: COc1cccn2c(=O)c(CO)c(Nc3ccc(Cl)c(F)c3)nc12] | +++ | ++ |
| 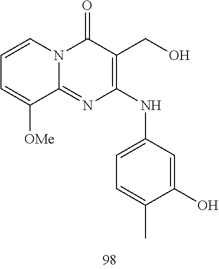 [SMILES: COc1cccn2c(=O)c(CO)c(Nc3ccc(C)c(O)c3)nc12] | ++ | + |
| 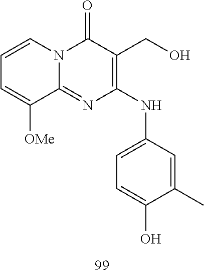 [SMILES: COc1cccn2c(=O)c(CO)c(Nc3ccc(O)c(C)c3)nc12] | + | + |
| 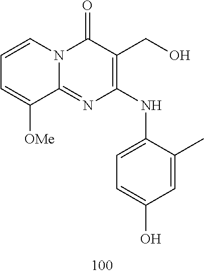 [SMILES: COc1cccn2c(=O)c(CO)c(Nc3ccc(O)cc3C)nc12] | ++ | + |
| 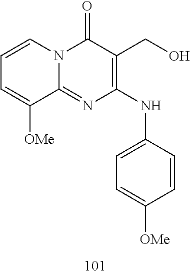 [SMILES: COc1ccc(Nc2nc3c(OC)cccn3c(=O)c2CO)cc1] | ++ | + |
| 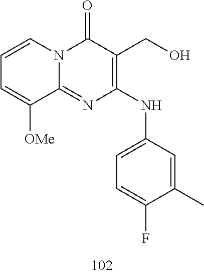 [SMILES: COc1cccn2c(=O)c(CO)c(Nc3ccc(F)c(C)c3)nc12] | +++ | ++ |
| 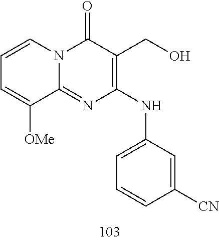 [SMILES: COc1cccn2c(=O)c(CO)c(Nc3cccc(C#N)c3)nc12] | +++ | ++ |
| 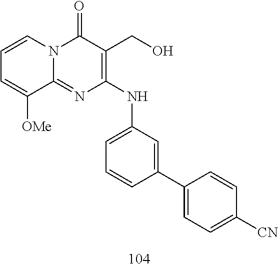 [SMILES: COc1cccn2c(=O)c(CO)c(Nc3cccc(-c4ccc(C#N)cc4)c3)nc12] | +++ | + |
| 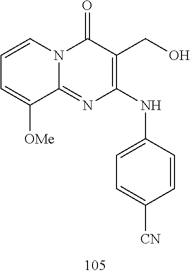 [SMILES: COc1cccn2c(=O)c(CO)c(Nc3ccc(C#N)cc3)nc12] | +++ | + |
| 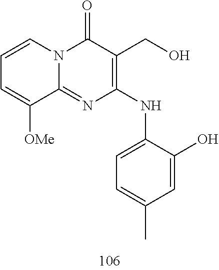 [SMILES: COc1cccn2c(=O)c(CO)c(Nc3ccc(C)cc3O)nc12] | ++ | + |
| 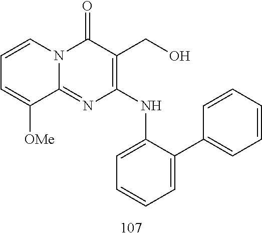 [SMILES: COc1cccn2c(=O)c(CO)c(Nc3ccccc3-c3ccccc3)nc12] | + | + |
| 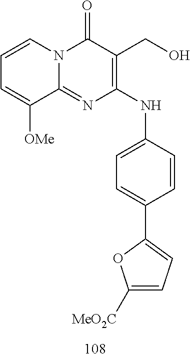 [SMILES: COC(=O)c1ccc(-c2ccc(Nc3nc4c(OC)cccn4c(=O)c3CO)cc2)o1] | + | + |
| 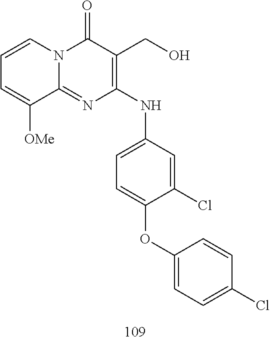 [SMILES: COc1cccn2c(=O)c(CO)c(Nc3ccc(Oc4ccc(Cl)cc4)c(Cl)c3)nc12] | + | + |
| 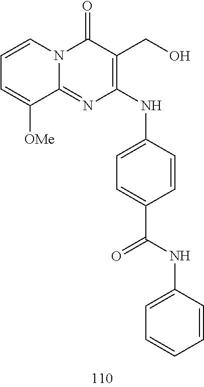 [SMILES: COc1cccn2c(=O)c(CO)c(Nc3ccc(C(=O)Nc4ccccc4)cc3)nc12] | + | + |
| 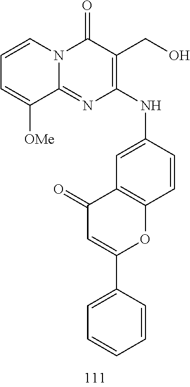 [SMILES: COc1cccn2c(=O)c(CO)c(Nc3ccc4oc(-c5ccccc5)cc(=O)c4c3)nc12] | + | + |
| 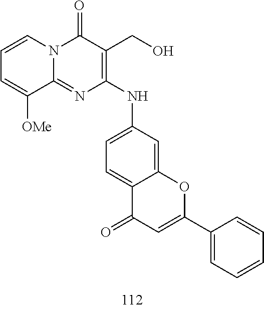 [SMILES: COc1cccn2c(=O)c(CO)c(Nc3ccc4c(=O)cc(-c5ccccc5)oc4c3)nc12] | + | + |
| 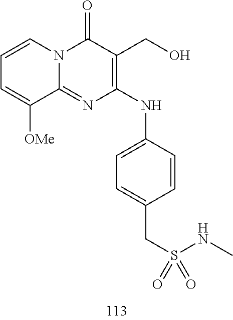 [SMILES: CNS(=O)(=O)Cc1ccc(Nc2nc3c(OC)cccn3c(=O)c2CO)cc1] | + | + |
| 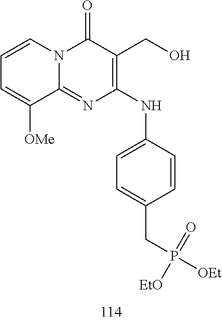 [SMILES: CCOP(=O)(Cc1ccc(Nc2nc3c(OC)cccn3c(=O)c2CO)cc1)OCC] | + | + |
| 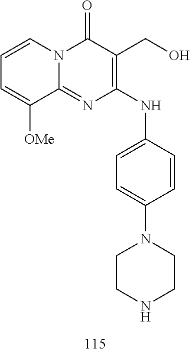 [SMILES: COc1cccn2c(=O)c(CO)c(Nc3ccc(N4CCNCC4)cc3)nc12] | + | + |
| 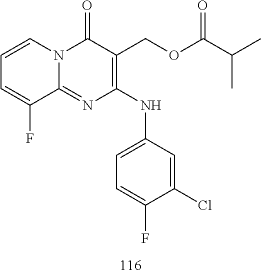 [SMILES: CC(C)C(=O)OCc1c(Nc2ccc(F)c(Cl)c2)nc2c(F)cccn2c1=O] | ++ | ++ |
| 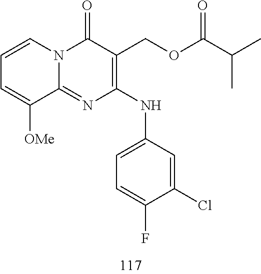 [SMILES: COc1cccn2c(=O)c(COC(=O)C(C)C)c(Nc3ccc(F)c(Cl)c3)nc12] | +++ | ++ |
| 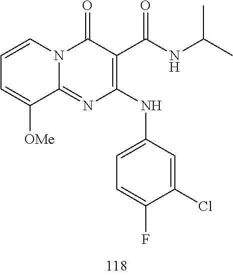 [SMILES: COc1cccn2c(=O)c(C(=O)NC(C)C)c(Nc3ccc(F)c(Cl)c3)nc12] | + | + |
| 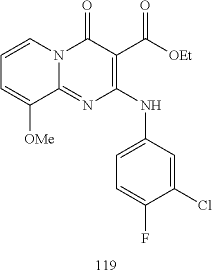 [SMILES: CCOC(=O)c1c(Nc2ccc(F)c(Cl)c2)nc2c(OC)cccn2c1=O] | + | + |
| 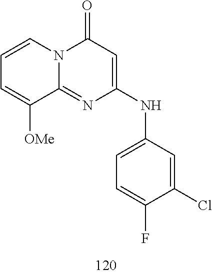 [SMILES: COc1cccn2c(=O)cc(Nc3ccc(F)c(Cl)c3)nc12] | + | + |

Activity range: +++ indicates <1 uM, ++ indicates between 1-20 uM, + indicates >20 uM
nd: not determined

TABLE 2

| Compound | QIM (μM) | QUM (μM) |
|---|---|---|
| 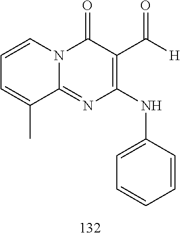 [SMILES: Cc1cccn2c(=O)c(C=O)c(Nc3ccccc3)nc12] | + | ++ |
| 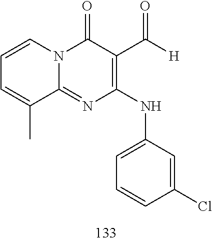 [SMILES: Cc1cccn2c(=O)c(C=O)c(Nc3cccc(Cl)c3)nc12] | ++ | +++ |
| 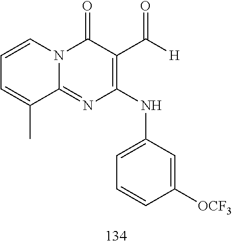 [SMILES: Cc1cccn2c(=O)c(C=O)c(Nc3cccc(OC(F)(F)F)c3)nc12] | + | ++ |
| 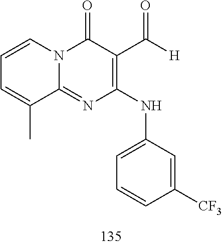 [SMILES: Cc1cccn2c(=O)c(C=O)c(Nc3cccc(C(F)(F)F)c3)nc12] | + | ++ |
| 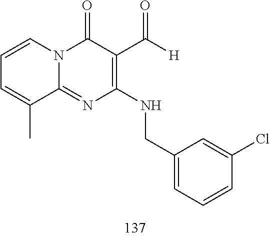 [SMILES: Cc1cccn2c(=O)c(C=O)c(NCc3cccc(Cl)c3)nc12] | + | + |
| 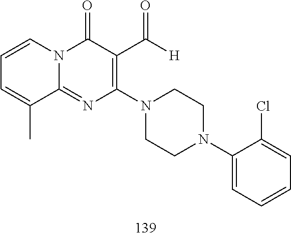 [SMILES: Cc1cccn2c(=O)c(C=O)c(N3CCN(c4ccccc4Cl)CC3)nc12] | + | + |
| 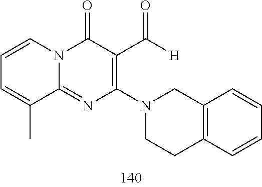 [SMILES: Cc1cccn2c(=O)c(C=O)c(N3CCc4ccccc4C3)nc12] | + | + |
| 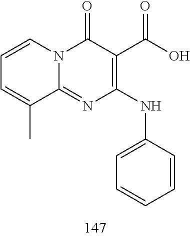 [SMILES: Cc1cccn2c(=O)c(C(=O)O)c(Nc3ccccc3)nc12] | + | + |
| 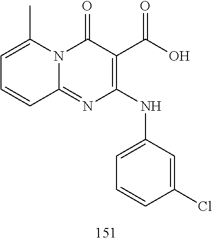 [SMILES: Cc1cccc2nc(Nc3cccc(Cl)c3)c(C(=O)O)c(=O)n12] | ++ | + |
| 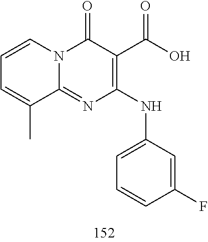 [SMILES: Cc1cccn2c(=O)c(C(=O)O)c(Nc3cccc(F)c3)nc12] | + | + |
| 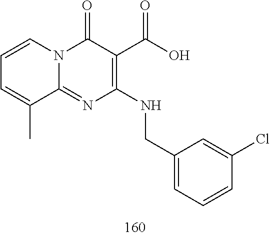 [SMILES: Cc1cccn2c(=O)c(C(=O)O)c(NCc3cccc(Cl)c3)nc12] | + | + |
| 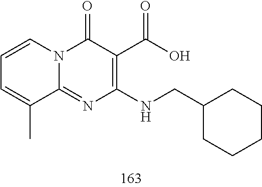 [SMILES: Cc1cccn2c(=O)c(C(=O)O)c(NCC3CCCCC3)nc12] | + | + |
| 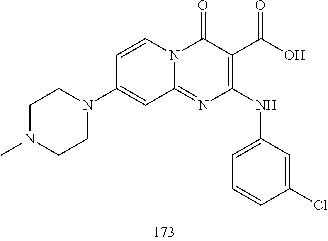 [SMILES: CN1CCN(c2ccn3c(=O)c(C(=O)O)c(Nc4cccc(Cl)c4)nc3c2)CC1] | + | + |
| 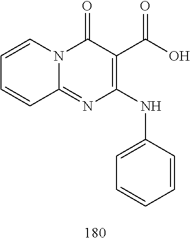 [SMILES: O=C(O)c1c(Nc2ccccc2)nc2ccccn2c1=O] | + | + |
| 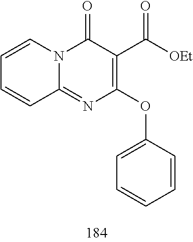 [SMILES: CCOC(=O)c1c(Oc2ccccc2)nc2ccccn2c1=O] | + | + |
| 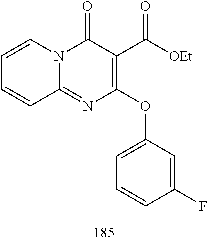 [SMILES: CCOC(=O)c1c(Oc2cccc(F)c2)nc2ccccn2c1=O] | + | ++ |
| 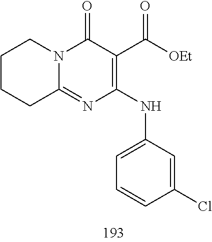 [SMILES: CCOC(=O)c1c(Nc2cccc(Cl)c2)nc2n(c1=O)CCCC2] | + | + |
| 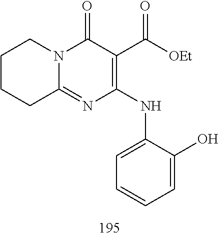 [SMILES: CCOC(=O)c1c(Nc2ccccc2O)nc2n(c1=O)CCCC2] | + | + |
| 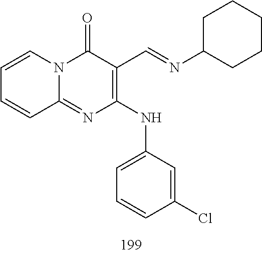 [SMILES: O=c1c(/C=N/C2CCCCC2)c(Nc2cccc(Cl)c2)nc2ccccn12] | + | ++ |
| 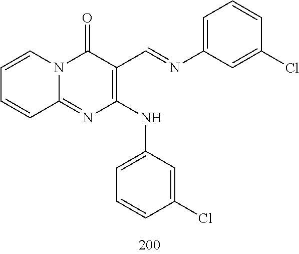 [SMILES: O=c1c(/C=N/c2cccc(Cl)c2)c(Nc2cccc(Cl)c2)nc2ccccn12] | + | + |
| 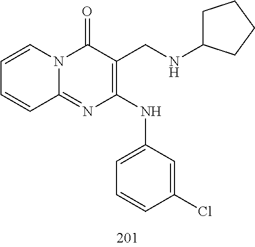 [SMILES: O=c1c(CNC2CCCC2)c(Nc2cccc(Cl)c2)nc2ccccn12] | + | + |
| 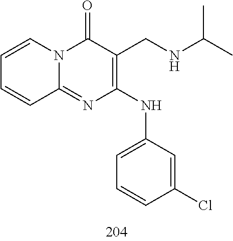 [SMILES: CC(C)NCc1c(Nc2cccc(Cl)c2)nc2ccccn2c1=O] | + | + |
| 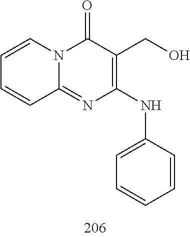 [SMILES: O=c1c(CO)c(Nc2ccccc2)nc2ccccn12] | +++ | +++ |
| 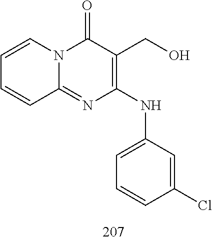 [SMILES: O=c1c(CO)c(Nc2cccc(Cl)c2)nc2ccccn12] | +++ | +++ |
| 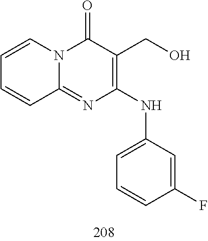 [SMILES: O=c1c(CO)c(Nc2cccc(F)c2)nc2ccccn12] | +++ | +++ |
| 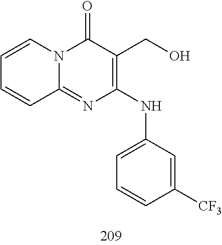 [SMILES: O=c1c(CO)c(Nc2cccc(C(F)(F)F)c2)nc2ccccn12] | +++ | +++ |
| 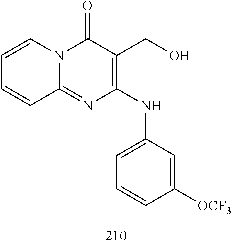 [SMILES: O=c1c(CO)c(Nc2cccc(OC(F)(F)F)c2)nc2ccccn12] | +++ | +++ |
| 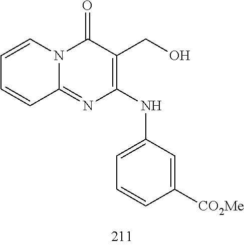 [SMILES: COC(=O)c1cccc(Nc2nc3ccccn3c(=O)c2CO)c1] | + | +++ |
| 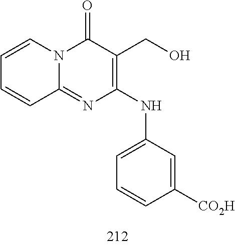 [SMILES: O=C(O)c1cccc(Nc2nc3ccccn3c(=O)c2CO)c1] | + | + |
| 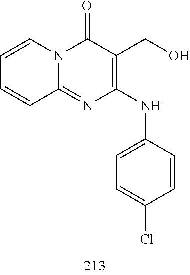 [SMILES: O=c1c(CO)c(Nc2ccc(Cl)cc2)nc2ccccn12] | + | +++ |
| 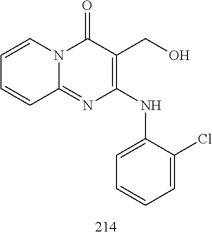 [SMILES: O=c1c(CO)c(Nc2ccccc2Cl)nc2ccccn12] | + | ++ |
| 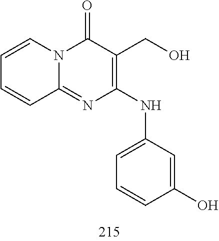 [SMILES: O=c1c(CO)c(Nc2cccc(O)c2)nc2ccccn12] | + | + |
| 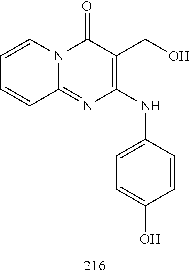 [SMILES: O=c1c(CO)c(Nc2ccc(O)cc2)nc2ccccn12] | + | + |
| 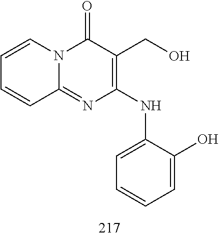 [SMILES: O=c1c(CO)c(Nc2ccccc2O)nc2ccccn12] | + | +++ |
| 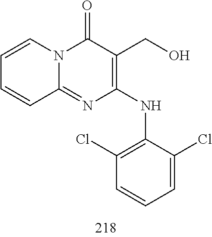 [SMILES: O=c1c(CO)c(Nc2c(Cl)cccc2Cl)nc2ccccn12] | + | + |
| 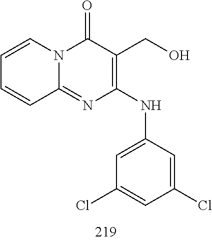 [SMILES: O=c1c(CO)c(Nc2cc(Cl)cc(Cl)c2)nc2ccccn12] | + | + |
| 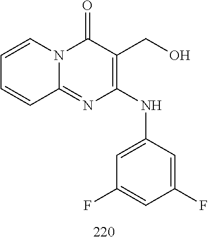 [SMILES: O=c1c(CO)c(Nc2cc(F)cc(F)c2)nc2ccccn12] | ++ | +++ |
| 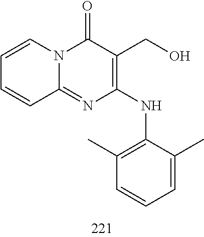 [SMILES: Cc1cccc(C)c1Nc1nc2ccccn2c(=O)c1CO] | + | + |
| 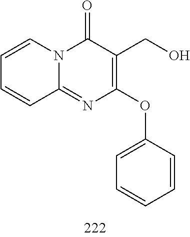 [SMILES: O=c1c(CO)c(Oc2ccccc2)nc2ccccn12] | + | + |
| 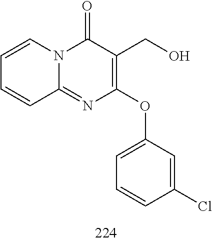 [SMILES: O=c1c(CO)c(Oc2cccc(Cl)c2)nc2ccccn12] | + | + |
| 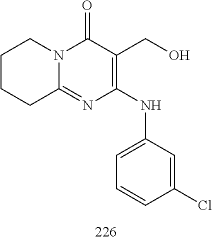 [SMILES: O=c1c(CO)c(Nc2cccc(Cl)c2)nc2n1CCCC2] | + | + |
| 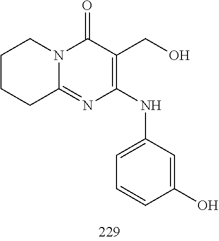 [SMILES: O=c1c(CO)c(Nc2cccc(O)c2)nc2n1CCCC2] | + | + |
| 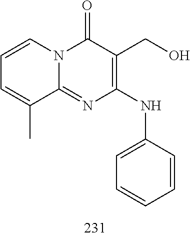 [SMILES: Cc1cccn2c(=O)c(CO)c(Nc3ccccc3)nc12] | +++ | +++ |
| 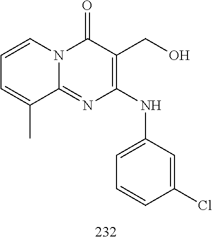 [SMILES: Cc1cccn2c(=O)c(CO)c(Nc3cccc(Cl)c3)nc12] | +++ | ++ |
| 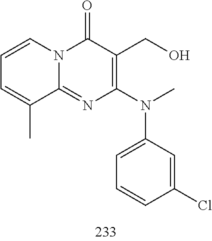 [SMILES: Cc1cccn2c(=O)c(CO)c(N(C)c3cccc(Cl)c3)nc12] | + | + |
| 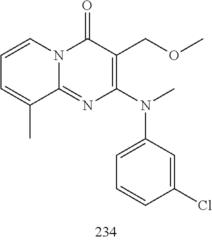 [SMILES: COCc1c(N(C)c2cccc(Cl)c2)nc2c(C)cccn2c1=O] | + | + |
| 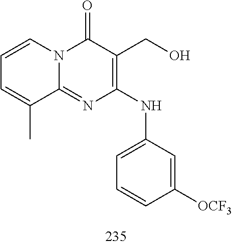 [SMILES: Cc1cccn2c(=O)c(CO)c(Nc3cccc(OC(F)(F)F)c3)nc12] | +++ | +++ |
| 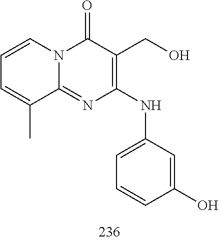 [SMILES: Cc1cccn2c(=O)c(CO)c(Nc3cccc(O)c3)nc12] | ++ | +++ |
| 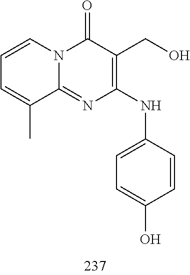 [SMILES: Cc1cccn2c(=O)c(CO)c(Nc3ccc(O)cc3)nc12] | + | +++ |
| 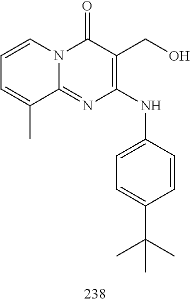 [SMILES: Cc1cccn2c(=O)c(CO)c(Nc3ccc(C(C)(C)C)cc3)nc12] | + | + |
| 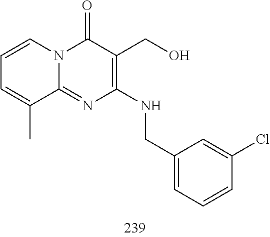 [SMILES: Cc1cccn2c(=O)c(CO)c(NCc3cccc(Cl)c3)nc12] | + | + |
| 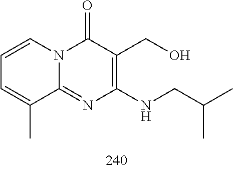 [SMILES: Cc1cccn2c(=O)c(CO)c(NCC(C)C)nc12] | ++ | ++ |
| 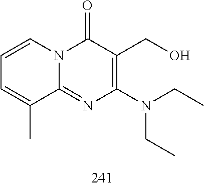 [SMILES: CCN(CC)c1nc2c(C)cccn2c(=O)c1CO] | + | + |
| 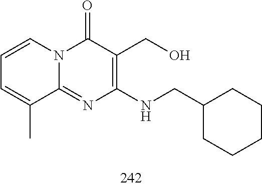 [SMILES: Cc1cccn2c(=O)c(CO)c(NCC3CCCCC3)nc12] | + | + |
| 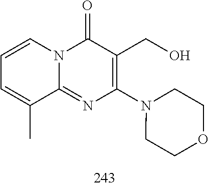 [SMILES: Cc1cccn2c(=O)c(CO)c(N3CCOCC3)nc12] | + | + |
| 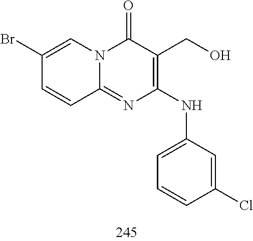 [SMILES: O=c1c(CO)c(Nc2cccc(Cl)c2)nc2ccc(Br)cn12] | + | ++ |
| 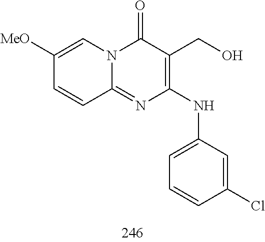 [SMILES: COc1ccc2nc(Nc3cccc(Cl)c3)c(CO)c(=O)n2c1] | + | + |
| 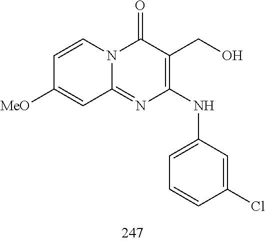 [SMILES: COc1ccn2c(=O)c(CO)c(Nc3cccc(Cl)c3)nc2c1] | + | +++ |
| 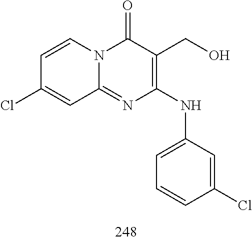 [SMILES: O=c1c(CO)c(Nc2cccc(Cl)c2)nc2cc(Cl)ccn12] | + | +++ |
| 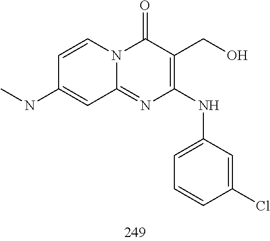 [SMILES: CNc1ccn2c(=O)c(CO)c(Nc3cccc(Cl)c3)nc2c1] | + | + |
| 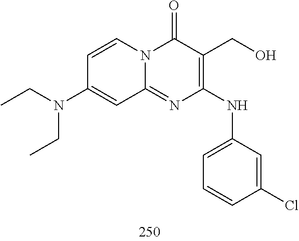 [SMILES: CCN(CC)c1ccn2c(=O)c(CO)c(Nc3cccc(Cl)c3)nc2c1] | + | + |
| 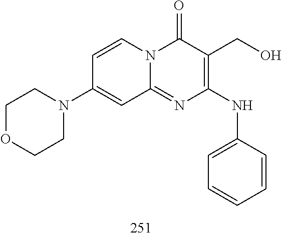 [SMILES: O=c1c(CO)c(Nc2ccccc2)nc2cc(N3CCOCC3)ccn12] | + | + |
| 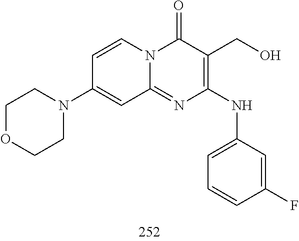 [SMILES: O=c1c(CO)c(Nc2cccc(F)c2)nc2cc(N3CCOCC3)ccn12] | + | + |
| 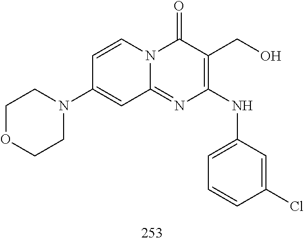 [SMILES: O=c1c(CO)c(Nc2cccc(Cl)c2)nc2cc(N3CCOCC3)ccn12] | + | + |
| 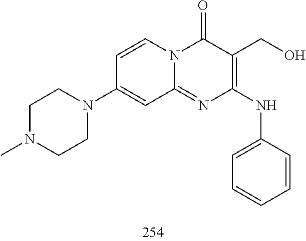 [SMILES: CN1CCN(c2ccn3c(=O)c(CO)c(Nc4ccccc4)nc3c2)CC1] | + | + |
| 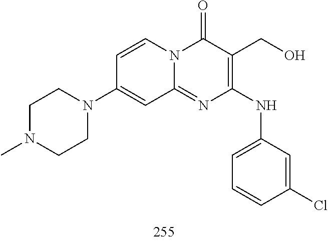 [SMILES: CN1CCN(c2ccn3c(=O)c(CO)c(Nc4cccc(Cl)c4)nc3c2)CC1] | + | + |
| 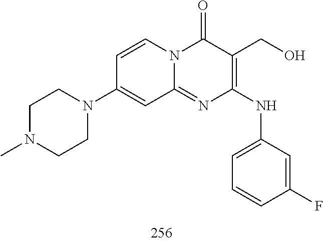 [SMILES: CN1CCN(c2ccn3c(=O)c(CO)c(Nc4cccc(F)c4)nc3c2)CC1] | + | + |
| 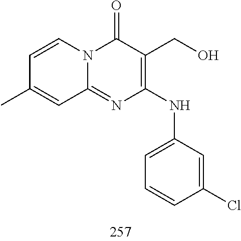 [SMILES: Cc1ccn2c(=O)c(CO)c(Nc3cccc(Cl)c3)nc2c1] | +++ | +++ |
| 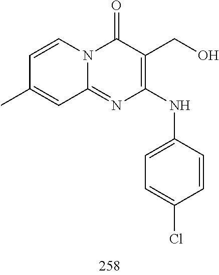 [SMILES: Cc1ccn2c(=O)c(CO)c(Nc3ccc(Cl)cc3)nc2c1] | +++ | +++ |
| 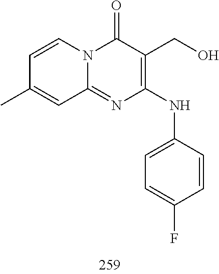 [SMILES: Cc1ccn2c(=O)c(CO)c(Nc3ccc(F)cc3)nc2c1] | ++ | +++ |
| 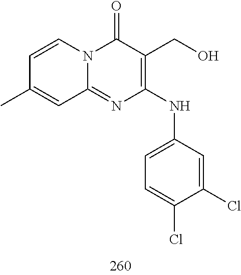 [SMILES: Cc1ccn2c(=O)c(CO)c(Nc3ccc(Cl)c(Cl)c3)nc2c1] | + | ++ |
| 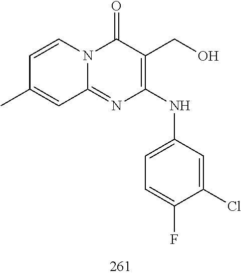 [SMILES: Cc1ccn2c(=O)c(CO)c(Nc3ccc(F)c(Cl)c3)nc2c1] | +++ | +++ |
| 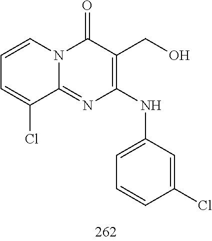 [SMILES: O=c1c(CO)c(Nc2cccc(Cl)c2)nc2c(Cl)cccn12] | + | + |
| 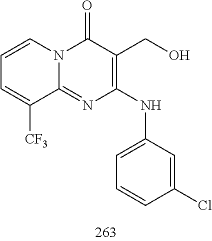 [SMILES: O=c1c(CO)c(Nc2cccc(Cl)c2)nc2c(C(F)(F)F)cccn12] | + | + |
| 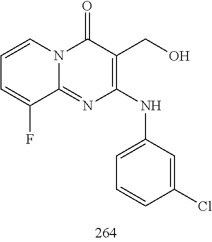 [SMILES: O=c1c(CO)c(Nc2cccc(Cl)c2)nc2c(F)cccn12] | ++ | +++ |
| 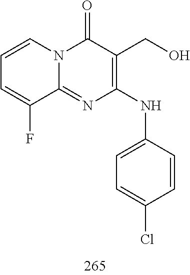 [SMILES: O=c1c(CO)c(Nc2ccc(Cl)cc2)nc2c(F)cccn12] | +++ | +++ |
| 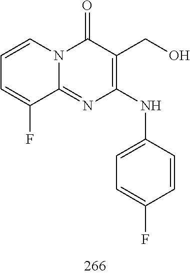 [SMILES: O=c1c(CO)c(Nc2ccc(F)cc2)nc2c(F)cccn12] | + | +++ |
| 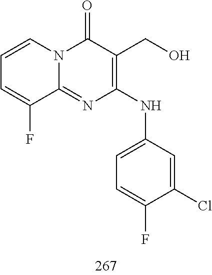 [SMILES: O=c1c(CO)c(Nc2ccc(F)c(Cl)c2)nc2c(F)cccn12] | +++ | +++ |
| 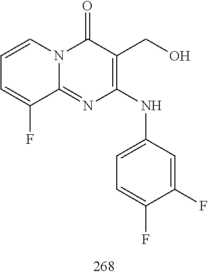 [SMILES: O=c1c(CO)c(Nc2ccc(F)c(F)c2)nc2c(F)cccn12] | + | ++ |
| 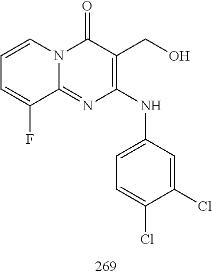 [SMILES: O=c1c(CO)c(Nc2ccc(Cl)c(Cl)c2)nc2c(F)cccn12] | ++ | ++ |
| 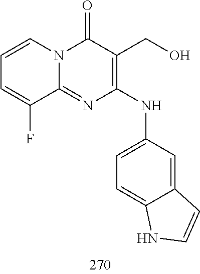 [SMILES: O=c1c(CO)c(Nc2ccc3[nH]ccc3c2)nc2c(F)cccn12] | +++ | +++ |
| 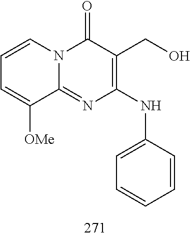 [SMILES: COc1cccn2c(=O)c(CO)c(Nc3ccccc3)nc12] | + | +++ |
| 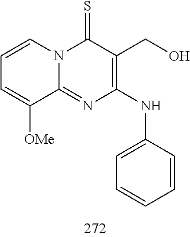 [SMILES: COc1cccn2c(=S)c(CO)c(Nc3ccccc3)nc12] | + | + |
| 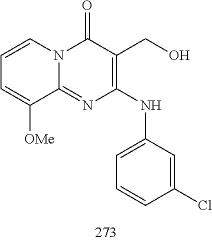 [SMILES: COc1cccn2c(=O)c(CO)c(Nc3cccc(Cl)c3)nc12] | +++ | +++ |
| 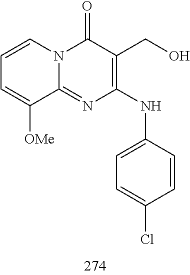 [SMILES: COc1cccn2c(=O)c(CO)c(Nc3ccc(Cl)cc3)nc12] | + | +++ |
| 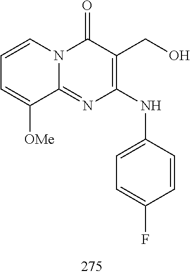 [SMILES: COc1cccn2c(=O)c(CO)c(Nc3ccc(F)cc3)nc12] | + | +++ |
| 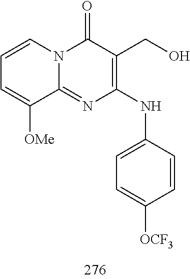 [SMILES: COc1cccn2c(=O)c(CO)c(Nc3ccc(OC(F)(F)F)cc3)nc12] | + | ++ |
| 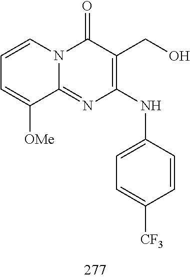 [SMILES: COc1cccn2c(=O)c(CO)c(Nc3ccc(C(F)(F)F)cc3)nc12] | + | ++ |
| 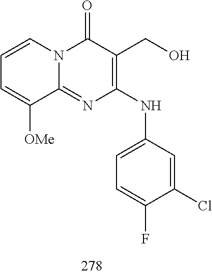 [SMILES: COc1cccn2c(=O)c(CO)c(Nc3ccc(F)c(Cl)c3)nc12] | +++ | +++ |
| 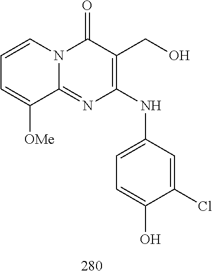 [SMILES: COc1cccn2c(=O)c(CO)c(Nc3ccc(O)c(Cl)c3)nc12] | + | +++ |
| 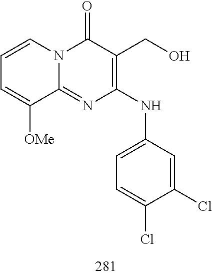 [SMILES: COc1cccn2c(=O)c(CO)c(Nc3ccc(Cl)c(Cl)c3)nc12] | + | +++ |
| 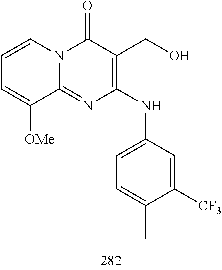 [SMILES: COc1cccn2c(=O)c(CO)c(Nc3ccc(C)c(C(F)(F)F)c3)nc12] | + | + |
| 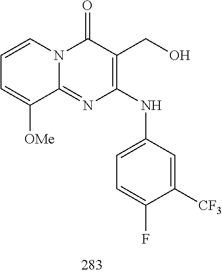 [SMILES: COc1cccn2c(=O)c(CO)c(Nc3ccc(F)c(C(F)(F)F)c3)nc12] | + | + |
| 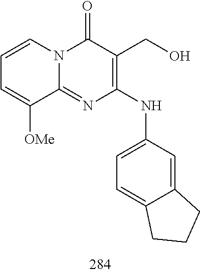 [SMILES: COc1cccn2c(=O)c(CO)c(Nc3ccc4c(c3)CCC4)nc12] | + | +++ |
| 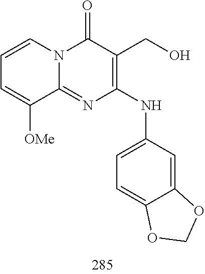 [SMILES: COc1cccn2c(=O)c(CO)c(Nc3ccc4c(c3)OCO4)nc12] | + | +++ |
| 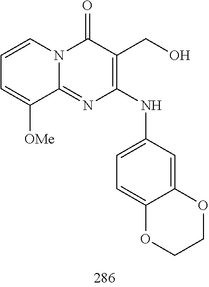 [SMILES: COc1cccn2c(=O)c(CO)c(Nc3ccc4c(c3)OCCO4)nc12] | + | +++ |
| 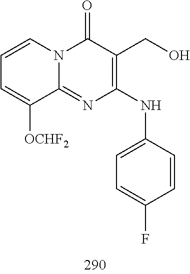 [SMILES: O=c1c(CO)c(Nc2ccc(F)cc2)nc2c(OC(F)F)cccn12] | + | + |
| 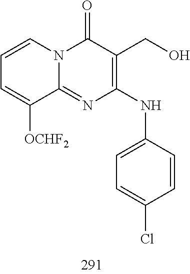 [SMILES: O=c1c(CO)c(Nc2ccc(Cl)cc2)nc2c(OC(F)F)cccn12] | + | + |
| 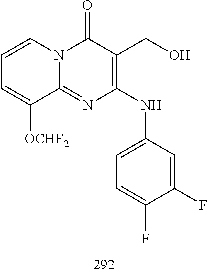 [SMILES: O=c1c(CO)c(Nc2ccc(F)c(F)c2)nc2c(OC(F)F)cccn12] | + | + |
| 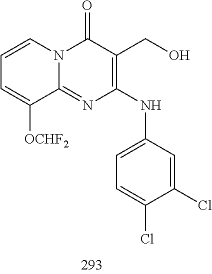 [SMILES: O=c1c(CO)c(Nc2ccc(Cl)c(Cl)c2)nc2c(OC(F)F)cccn12] | + | + |
| 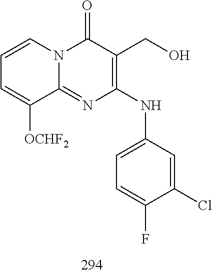 [SMILES: O=c1c(CO)c(Nc2ccc(F)c(Cl)c2)nc2c(OC(F)F)cccn12] | + | + |
| 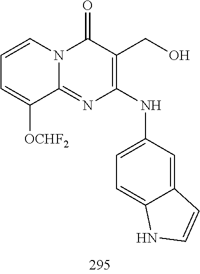 [SMILES: O=c1c(CO)c(Nc2ccc3[nH]ccc3c2)nc2c(OC(F)F)cccn12] | ++ | +++ |
| 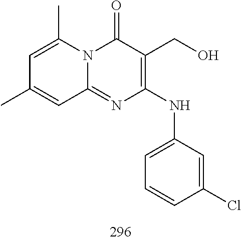 [SMILES: Cc1cc(C)n2c(=O)c(CO)c(Nc3cccc(Cl)c3)nc2c1] | + | + |
| 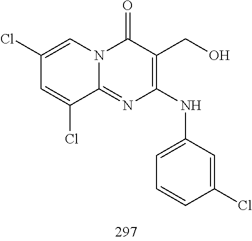 [SMILES: O=c1c(CO)c(Nc2cccc(Cl)c2)nc2c(Cl)cc(Cl)cn12] | + | + |
| 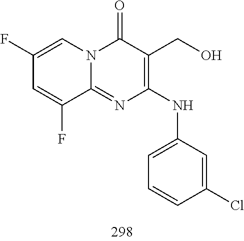 [SMILES: O=c1c(CO)c(Nc2cccc(Cl)c2)nc2c(F)cc(F)cn12] | + | + |
| 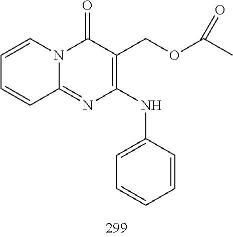 [SMILES: CC(=O)OCc1c(Nc2ccccc2)nc2ccccn2c1=O] | ++ | +++ |
| 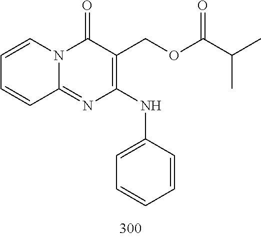 [SMILES: CC(C)C(=O)OCc1c(Nc2ccccc2)nc2ccccn2c1=O] | ++ | +++ |
| 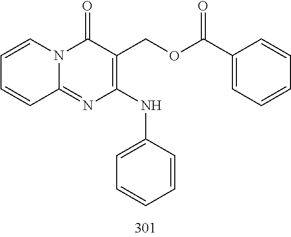 [SMILES: O=C(OCc1c(Nc2ccccc2)nc2ccccn2c1=O)c1ccccc1] | ++ | +++ |
| 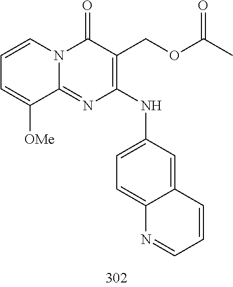 [SMILES: COc1cccn2c(=O)c(COC(C)=O)c(Nc3ccc4ncccc4c3)nc12] | +++ | +++ |
| 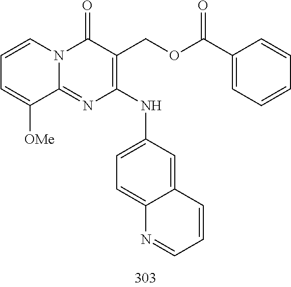 [SMILES: COc1cccn2c(=O)c(COC(=O)c3ccccc3)c(Nc3ccc4ncccc4c3)nc12] | +++ | +++ |
| 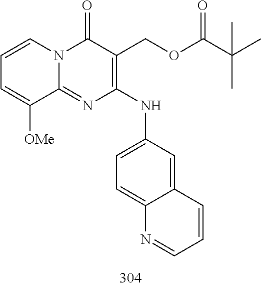 [SMILES: COc1cccn2c(=O)c(COC(=O)C(C)(C)C)c(Nc3ccc4ncccc4c3)nc12] | +++ | +++ |
| 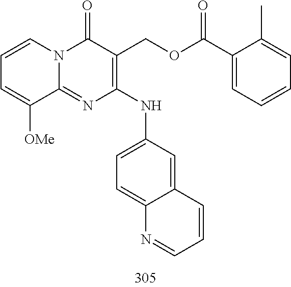 [SMILES: COc1cccn2c(=O)c(COC(=O)c3ccccc3C)c(Nc3ccc4ncccc4c3)nc12] | +++ | +++ |
| 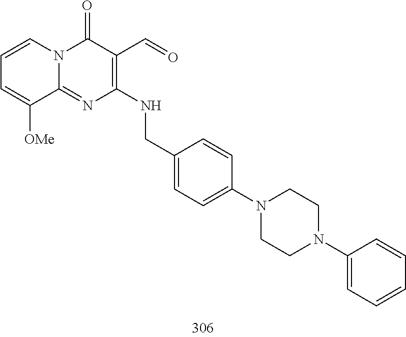 [SMILES: COc1cccn2c(=O)c(C=O)c(NCc3ccc(N4CCN(c5ccccc5)CC4)cc3)nc12] | + | + |
| 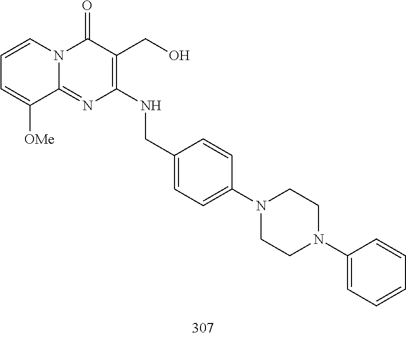 [SMILES: COc1cccn2c(=O)c(CO)c(NCc3ccc(N4CCN(c5ccccc5)CC4)cc3)nc12] | + | + |
| 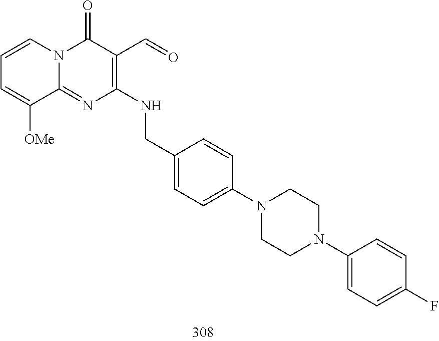 [SMILES: COc1cccn2c(=O)c(C=O)c(NCc3ccc(N4CCN(c5ccc(F)cc5)CC4)cc3)nc12] | + | + |
| 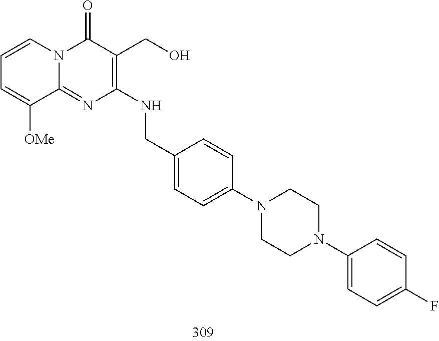 [SMILES: COc1cccn2c(=O)c(CO)c(NCc3ccc(N4CCN(c5ccc(F)cc5)CC4)cc3)nc12] | + | + |
| 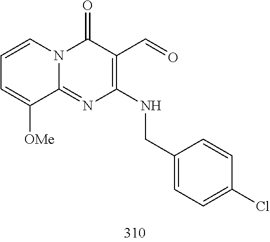 [SMILES: COc1cccn2c(=O)c(C=O)c(NCc3ccc(Cl)cc3)nc12] | + | + |
| 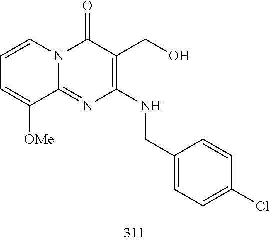 [SMILES: COc1cccn2c(=O)c(CO)c(NCc3ccc(Cl)cc3)nc12] | + | + |
| 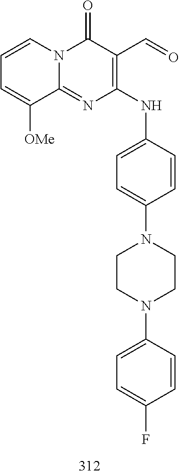 [SMILES: COc1cccn2c(=O)c(C=O)c(Nc3ccc(N4CCN(c5ccc(F)cc5)CC4)cc3)nc12] | + | + |
| 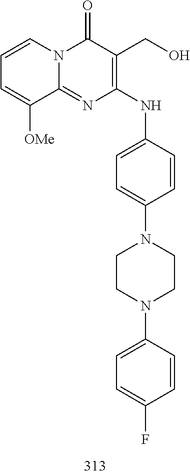 [SMILES: COc1cccn2c(=O)c(CO)c(Nc3ccc(N4CCN(c5ccc(F)cc5)CC4)cc3)nc12] | + | + |
| 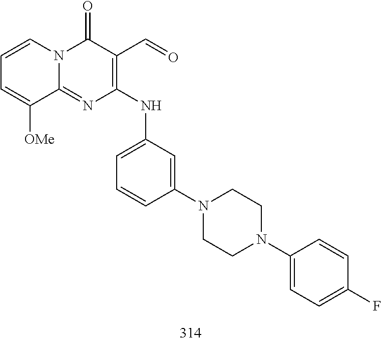 [SMILES: COc1cccn2c(=O)c(C=O)c(Nc3cccc(N4CCN(c5ccc(F)cc5)CC4)c3)nc12] | + | + |
| 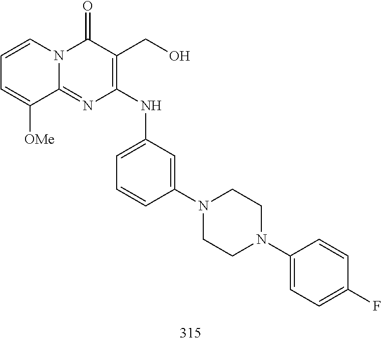 [SMILES: COc1cccn2c(=O)c(CO)c(Nc3cccc(N4CCN(c5ccc(F)cc5)CC4)c3)nc12] | + | + |
| 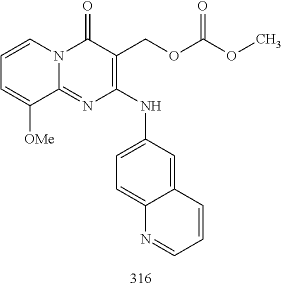 [SMILES: COC(=O)OCc1c(Nc2ccc3ncccc3c2)nc2c(OC)cccn2c1=O] | ++ | ++ |
| 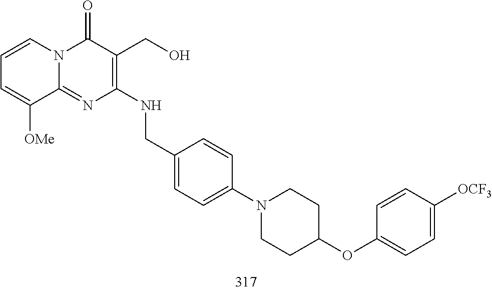 [SMILES: COc1cccn2c(=O)c(CO)c(NCc3ccc(N4CCC(Oc5ccc(OC(F)(F)F)cc5)CC4)cc3)nc12] | + | + |
| 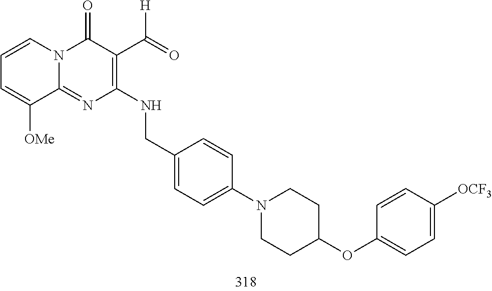 [SMILES: COc1cccn2c(=O)c(C=O)c(NCc3ccc(N4CCC(Oc5ccc(OC(F)(F)F)cc5)CC4)cc3)nc12] | + | + |
| 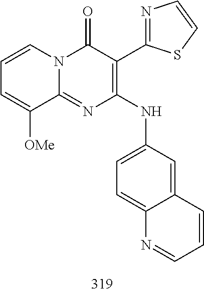 [SMILES: COc1cccn2c(=O)c(-c3nccs3)c(Nc3ccc4ncccc4c3)nc12] | + | + |
| 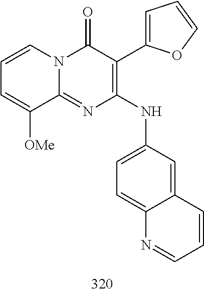 [SMILES: COc1cccn2c(=O)c(-c3ccco3)c(Nc3ccc4ncccc4c3)nc12] | + | + |
| 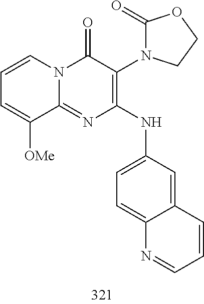 [SMILES: COc1cccn2c(=O)c(N3CCOC3=O)c(Nc3ccc4ncccc4c3)nc12] | + | + |
| 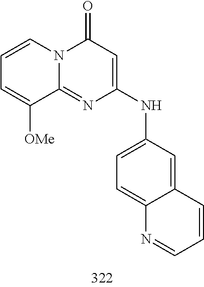 [SMILES: COc1cccn2c(=O)cc(Nc3ccc4ncccc4c3)nc12] | + | + |
| 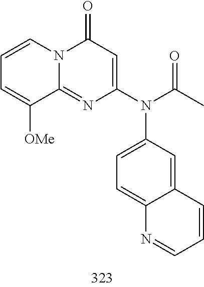 [SMILES: COc1cccn2c(=O)cc(N(C(C)=O)c3ccc4ncccc4c3)nc12] | + | + |
| 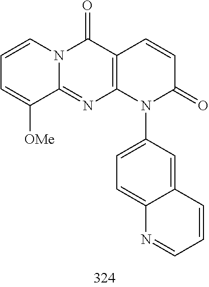 [SMILES: COc1cccn2c(=O)c3ccc(=O)n(-c4ccc5ncccc5c4)c3nc12] | ++ | ++ |
| 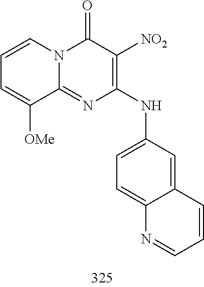 [SMILES: COc1cccn2c(=O)c([N+](=O)[O-])c(Nc3ccc4ncccc4c3)nc12] | + | + |
| 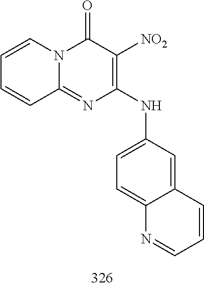 [SMILES: O=c1c([N+](=O)[O-])c(Nc2ccc3ncccc3c2)nc2ccccn12] | + | + |
| 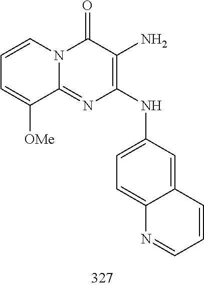 [SMILES: COc1cccn2c(=O)c(N)c(Nc3ccc4ncccc4c3)nc12] | + | + |
| 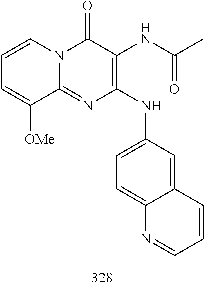 [SMILES: COc1cccn2c(=O)c(NC(C)=O)c(Nc3ccc4ncccc4c3)nc12] | + | + |
| 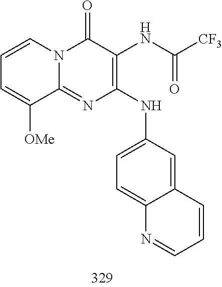 [SMILES: COc1cccn2c(=O)c(NC(=O)C(F)(F)F)c(Nc3ccc4ncccc4c3)nc12] | + | + |
| 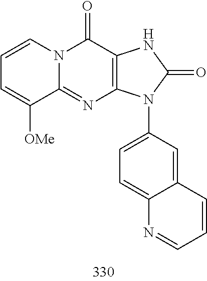 [SMILES: COc1cccn2c(=O)c3[nH]c(=O)n(-c4ccc5ncccc5c4)c3nc12] | + | + |
| 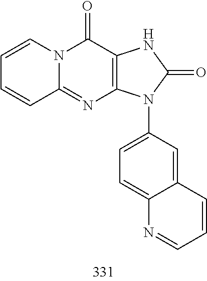 [SMILES: O=c1[nH]c2c(=O)n3ccccc3nc2n1-c1ccc2ncccc2c1] | + | + |
| 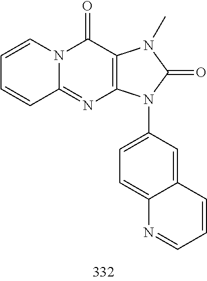 [SMILES: Cn1c(=O)n(-c2ccc3ncccc3c2)c2nc3ccccn3c(=O)c21] | + | + |
| 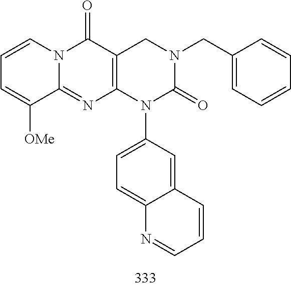 [SMILES: COc1cccn2c(=O)c3c(nc12)N(c1ccc2ncccc2c1)C(=O)N(Cc1ccccc1)C3] | + | + |
| 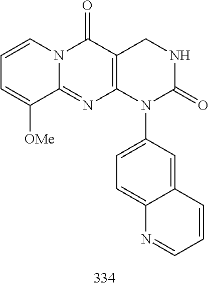 [SMILES: COc1cccn2c(=O)c3c(nc12)N(c1ccc2ncccc2c1)C(=O)NC3] | + | + |
| 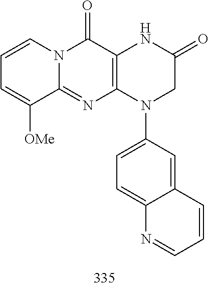 [SMILES: COc1cccn2c(=O)c3c(nc12)N(c1ccc2ncccc2c1)CC(=O)N3] | + | + |
| 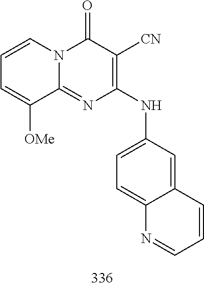 [SMILES: COc1cccn2c(=O)c(C#N)c(Nc3ccc4ncccc4c3)nc12] | + | + |
| 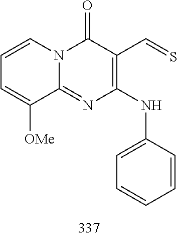 [SMILES: COc1cccn2c(=O)c(C=S)c(Nc3ccccc3)nc12] | ++ | +++ |
| 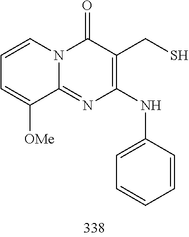 [SMILES: COc1cccn2c(=O)c(CS)c(Nc3ccccc3)nc12] | + | + |
| 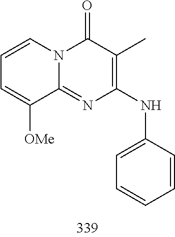 [SMILES: COc1cccn2c(=O)c(C)c(Nc3ccccc3)nc12] | + | + |
| 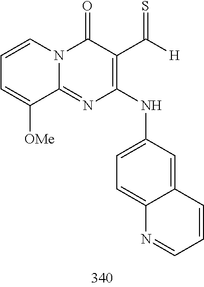 [SMILES: COc1cccn2c(=O)c(C=S)c(Nc3ccc4ncccc4c3)nc12] | +++ | +++ |
| 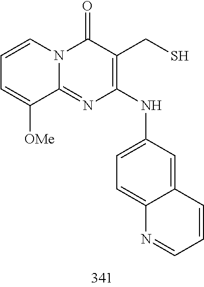 [SMILES: COc1cccn2c(=O)c(CS)c(Nc3ccc4ncccc4c3)nc12] | ++ | ++ |
| 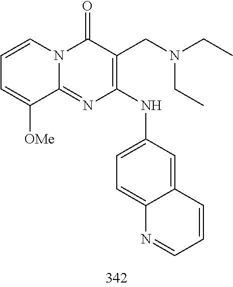 [SMILES: CCN(CC)Cc1c(Nc2ccc3ncccc3c2)nc2c(OC)cccn2c1=O] | + | + |
| 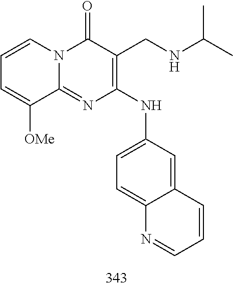 [SMILES: COc1cccn2c(=O)c(CNC(C)C)c(Nc3ccc4ncccc4c3)nc12] | + | + |
| 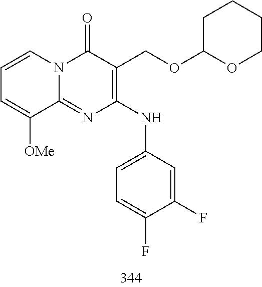 [SMILES: COc1cccn2c(=O)c(COC3CCCCO3)c(Nc3ccc(F)c(F)c3)nc12] | + | + |
| 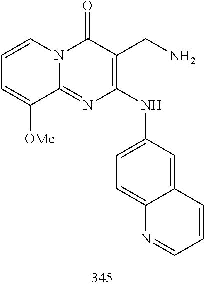 [SMILES: COc1cccn2c(=O)c(CN)c(Nc3ccc4ncccc4c3)nc12] | + | + |
| 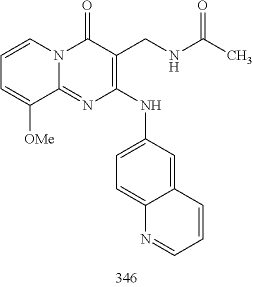 [SMILES: COc1cccn2c(=O)c(CNC(C)=O)c(Nc3ccc4ncccc4c3)nc12] | + | + |
| 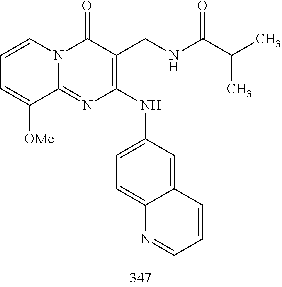 [SMILES: COc1cccn2c(=O)c(CNC(=O)C(C)C)c(Nc3ccc4ncccc4c3)nc12] | + | + |
| 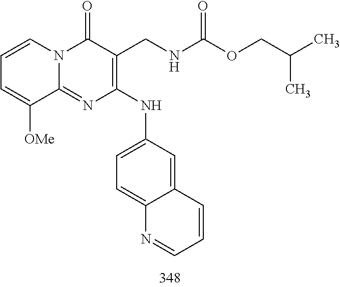 [SMILES: COc1cccn2c(=O)c(CNC(=O)OCC(C)C)c(Nc3ccc4ncccc4c3)nc12] | + | + |
| 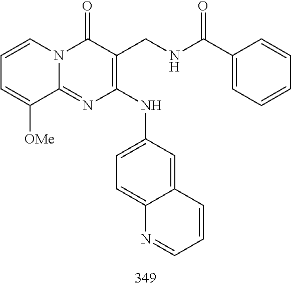 [SMILES: COc1cccn2c(=O)c(CNC(=O)c3ccccc3)c(Nc3ccc4ncccc4c3)nc12] | + | + |
| 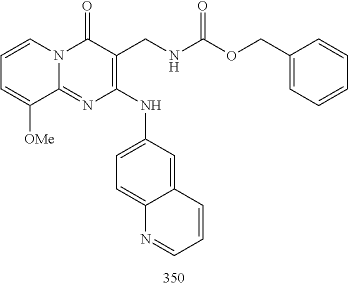 [SMILES: COc1cccn2c(=O)c(CNC(=O)OCc3ccccc3)c(Nc3ccc4ncccc4c3)nc12] | + | + |
| 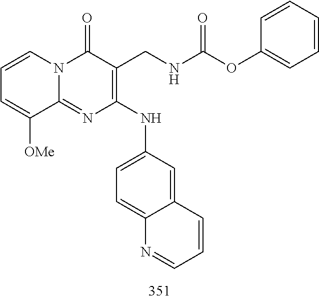 [SMILES: COc1cccn2c(=O)c(CNC(=O)Oc3ccccc3)c(Nc3ccc4ncccc4c3)nc12] | + | + |
| 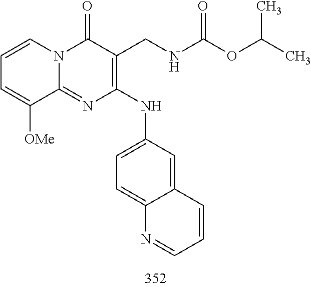 [SMILES: COc1cccn2c(=O)c(CNC(=O)OC(C)C)c(Nc3ccc4ncccc4c3)nc12] | + | + |
| 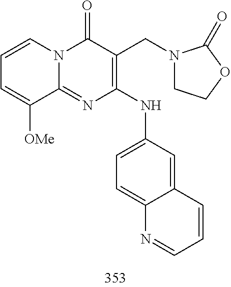 [SMILES: COc1cccn2c(=O)c(CN3CCOC3=O)c(Nc3ccc4ncccc4c3)nc12] | + | + |
| 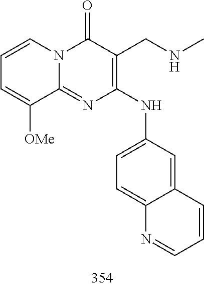 [SMILES: CNCc1c(Nc2ccc3ncccc3c2)nc2c(OC)cccn2c1=O] | + | + |
| 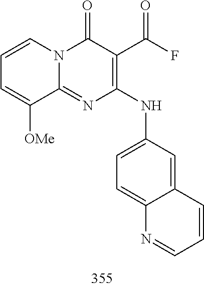 [SMILES: COc1cccn2c(=O)c(C(=O)F)c(Nc3ccc4ncccc4c3)nc12] | ++ | ++ |
| 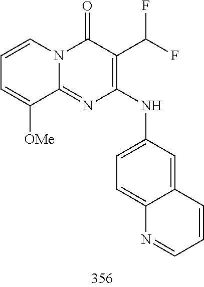 [SMILES: COc1cccn2c(=O)c(C(F)F)c(Nc3ccc4ncccc4c3)nc12] | +++ | +++ |
| 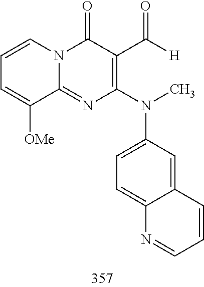 [SMILES: COc1cccn2c(=O)c(C=O)c(N(C)c3ccc4ncccc4c3)nc12] | + | + |
| 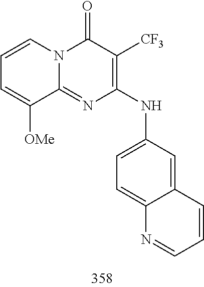 [SMILES: COc1cccn2c(=O)c(C(F)(F)F)c(Nc3ccc4ncccc4c3)nc12] | + | + |
| 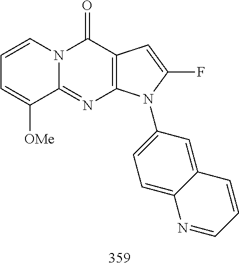 [SMILES: COc1cccn2c(=O)c3cc(F)n(-c4ccc5ncccc5c4)c3nc12] | + | + |

Activity range: +++ indicates <5 uM, ++ indicates between 5-20 uM, + indicates >20 uM

TABLE 3
Cytotoxicity
Compounds 133

| Host Cells | Range of MTC₅₀ (μM) |
|---|---|
| SK-N-SH -Brain | >100 |
| HepG2-Hepatocytes | >100 |
| MRC5- Lung | >100 |
| BJ- Skin | >100 |
| HEK293- Kidney | >100 |
| Jurkat -T-cell | >100 |
| THP-1 - Monocytes | >100 |
| Primary BMDM | >100 |
| Primary human macrophages | >100 |

Antibacterial activity & Specificity

| | | | | Range of MICs |
|---|---|---|---|---|
| Mycobacterium | | | | for multiple |
| Strains/Isolates | Type | Origin | Number | strains (μM) |
| M. tuberculosis | Drug | Sputum | 2 | 5->20 |
| clinical isolates¹ | Sensitive | Tissue | 2 | 2.5-5 |
| | RIF^{R} | Sputum | 1 | 2.5 |
| | | Tissue | 1 | 1.2 |
| | INH^{R} RIF^{R} | Sputum | 3 | 0.3-1.2 |
| | Strep^{R} | Tissue | 1 | 1.2 |
| | XDR | Sputum | 4 | 0.6-2.5 |
| | | Tissue | 5 | 0.3-5 |
| | MDR | Sputum | 3 | 0.3-1.2 |
| | | Tissue | 1 | 1.2 |
| M. tuberculosis | H37Rv | | | 2 |
| laboratory | H37Ra | | | 2 |
| strains | BCG Pasteur- | | | 2 |
| | Tokyo | | | |
| M. smegmatis | mc² 155 | | | >100 |

| | |
|---|---|
| Gram-negative | |
| Acinetobacter baumannii, Escherichia coli, Enterobacter cloacae, | NE |
| E. aerogenes, Klebsiella oxytoca, Pseudomonas aeruginosa, | |
| Salmonella enteridis, Vibrio mimicus | |
| Gram-positive | |
| Staphylococcus aureus, S. epidermis, S. capitis, S. xylosus, | NE |
| Micrococcus luteus, Listeria innocua, Lactobacillus gallinarum, | |
| group G Streptococcus, Streptococcus agalactiae, S. pyogenes, | |
| Enterococcus faecalis, E. faecium, E. gallinarum, Bacillus pumilus | |
| Corynebacterium | |
| C. striatum | NE |
| C. jeikeium | |
| Fungi | |
| Candida albicans, C. glabrata, C. parapsilosis | NE |

INH: Isoniazid, RIF: Rifampin, Strep: Streptomycin,
^{R}resistant.
¹The clinical isolates were isolated either from resected lung tissue or sputum specimen, which were collected from active tuberculosis in-patients from the National Masan Tuberculosis Hospital during October 2003 to March 2007.
NE: No effect up to 100 μg/mL equivalent to 320 μM. The antimicrobial spectrum was performed on clinical isolates from CHU d'Angers, France.

TABLE 4

| | Concentration | Bacteria inoculum (CFU) | | | Frequency of |
|---|---|---|---|---|---|
| Compound | (μg/ml) | 10⁶ | 10⁷ | 10⁸ | resistance |
| 264 | 0.4 | — | 37 | 306 | 3.4 × 10⁻⁶ |
| | 0.8 | — | 5 | 117 | 8 × 10⁻⁶ |
| | 1.6 | — | — | 22 | 2 × 10⁻⁸ |
| | 3.2 | — | — | 2 | 2 × 10⁻⁸ |
| INH-control | 10 | — | 4 | 18 | 2.9 × 10⁻⁶ |

—: no colonies

1. A compound selected from the group consisting of:
A) a compound having the general formula I: 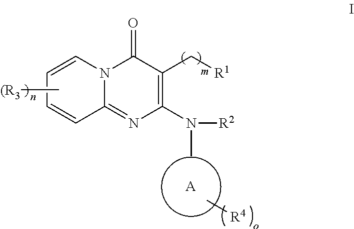
wherein
m is 0, 1, 2, or 3;
n is 1, 2, 3, or 4;
o is 1, 2, 3, or 4;
A is C₅-C₁₂ heteroaryl;
R¹ is selected from the group consisting of hydrogen, halogen, C₁-C₁₀ alkyl, C₃-C₁₀ cycloalkyl, C₂-C₁₀ alkenyl, C₃-C₁₀ cycloalkenyl, C₃-C₁₅ cycloalkylalkoxy, C₃-C₁₅ cycloalkylalkyl, hydroxyl, haloalkyl, oxo, —OR⁵, —OC(O)R⁵, —OC(O)N(R⁵)₂, —C(O)OR⁵, —C(O)R⁵, —C(O)N(R⁵)₂, —CN, —NO₂, —NH₂, —N(R⁵)₂, —N(R⁵)C(O)R⁵, —N(R⁵)C(O)N(R⁵)₂, —OR⁵HetA, —OR⁵N(R⁵)₂, —C(O)N(R⁵)R⁵HetA, —C(O)N(R⁵)HetA, —C(O)HetA, —C(O)N(R⁵)R⁵S(O)₂R⁵; SH, C(S)H, —S(O)₂N(R⁵)₂, —S(O)₂R⁵, —N(R⁵)C(O)R⁵SR⁵, —N(R⁵)R⁵S(O)₂R⁴, —N(R⁵)S(O)₂R⁵, aryl, benzyl, heteroaryl, and heterocyclyl, any of which is optionally substituted;
R² is selected from the group consisting of hydrogen, halogen, C₁-C₁₀ alkyl, C₃-C₁₀ cycloalkyl, C₂-C₁₀ alkenyl, C₃-C₁₀cycloalkenyl, C₃-C₁₅ cycloalkylalkyl, —NH₂, —N(R⁶)₂, —C(O)R⁶, —C(O)OR⁶, —C(O)N(R⁶)₂, —S(O)R⁶, —S(O)₂R⁶, —S(O)₂N(R⁶)₂, aryl, benzyl, heteroaryl, and heterocyclyl, or R¹ and R² are connected with each other to make a five or six membered cyclic or heterocyclic ring, any of which is optionally substituted;
R³ is selected from the group consisting of hydrogen, halogen, C₁-C₁₀ alkyl, C₃-C₁₀ cycloalkyl, hydroxyl, —OR⁶, —CN, —NO₂, —NH₂, —N(R⁶)C(O)R⁶, —C(O)R⁶, —C(O)OR⁶, C(O)N(R⁶)₂, —S(O)R⁶, —S(O)₂R⁶, —S(O)₂N(R⁶)₂, aryl, benzyl, heteroaryl, and heterocyclyl, or two groups of R³ are connected with each other to make a five or six membered cyclic or heterocyclic ring, any of which is optionally substituted;
R⁴ is independently, at each occurrence, selected from the group consisting of hydrogen, halogen, C₁-C₁₀ alkyl, C₃-C₁₀ cycloalkyl, hydroxyl, —OR⁶, —CN, —NO₂, —NH₂, —N(R⁶)C(O)R⁶, —C(O)R⁶, —C(O)OR⁶, —C(O)N(R⁶)₂, —S(O)R⁶, —S(O)₂R⁶, —S(O)₂N(R⁶)₂, aryl, benzyl, heteroaryl, and heterocyclyl, or two groups of R⁴ are connected with each other to make a five or six membered cyclic or heterocyclic ring, any of which is optionally substituted;
R⁵ and R⁶ are independently, at each occurrence, selected from the group consisting of hydrogen, C₁-C₁₀ alkyl, C₃-C₁₀ cycloalkyl, C₇-C₁₀ alkenyl, C₃-C₁₀ cycloalkenyl, C₂-C₁₀ alkynyl, C₁-C₁₀ haloalkyl, aryl, benzyl, heteroaryl, and heterocyclyl, any of which is optionally substituted; and
HetA is heteroaryl;
and pharmaceutically acceptable salts thereof;
B) a compound having the general formula II: 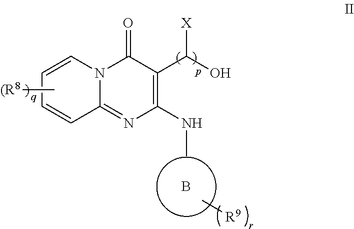
wherein
p is 0, 1, 2, or 3;
q is 1, 2, 3, or 4;
r is 1, 2, 3, or 4;
X is alkyl or aryl;
B is C₅-C₁₂ aryl;
R⁸ is selected from the group consisting of hydrogen, halogen, C₁-C₁₀ alkyl, C₃-C₁₀ cycloalkyl, hydroxyl, —OR¹⁰, —CN, —NO₂, —NH₂, —N(R¹⁰)C(O)R¹⁰, —C(O)R¹⁰, —C(O)—OR¹⁰, —C(O)N(R¹⁰)₂, —S(O)R¹⁰, —S(O)₂R¹⁰, —S(O)₂N(R¹⁰)₂, aryl, benzyl, heteroaryl, and heterocyclyl, or two groups of R⁸ are connected with each other to make a five or six membered cyclic or heterocyclic ring, any of which is optionally substituted;
R⁹ is selected from the group consisting of hydrogen, halogen, C₁-C₁₀ alkyl, C₃-C₁₀ cycloalkyl, C₂-C₁₀ alkenyl, C₃-C₁₀ cycloalkenyl, C₃-C₁₅ cycloalkylalkoxy, C₃-C₁₅ cycloalkylalkyl, hydroxyl, oxo, —OR¹¹, —OC(O)R¹¹, —OC(O)N(R¹¹)₂, —C(O)OR¹¹, —C(O)R¹¹, —C(O)N(R¹¹)₂, —CN, —NO₂, —NH₂, —N(R¹¹)₂, —N(R¹¹)C(O)R¹¹, —N(R¹¹)C(O)N(R¹¹)₂, —OR¹¹HetA, —OR¹¹N(R¹¹)₂, —C(O)N(R¹¹)R¹¹HetA, —C(O)N(R¹¹)HetA, —C(O)HetA, —C(O)N(R¹¹)R¹¹—S(O)₂R¹¹, —S(O)₂N(R¹¹)₂, —S(O)₂R¹¹, —N(R¹¹)C(O)R¹¹SR¹¹, —N(R¹¹)R¹¹S(O)₂R¹¹, —N(R¹¹)—S(O)₂R¹¹, —R¹¹P(O)(OR¹¹)2, aryl, benzyl, heteroaryl, and heterocyclyl, or two groups of R⁹ are connected with each other to make a five or six membered cyclic or heterocyclic ring, any of which is optionally substituted;
R¹⁰ and R¹¹ are independently, at each occurrence, selected from the group consisting of hydrogen, C₁-C₁₀ alkyl, C₃-C₁₀ cycloalkyl, C₂-C₁₀ alkenyl, C₃-C₁₀ cycloalkenyl, C₂-C₁₀ alkynyl, C₁-C₁₀ haloalkyl, aryl, benzyl, heteroaryl, and heterocyclyl, any of which is optionally substituted;
HetA is heteroaryl;
and pharmaceutically acceptable salts thereof;
C) a compound having the general formula VIII: 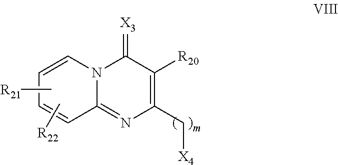
wherein
m is 0, 1, 2, or 3;
X₃ is selected from the group consisting of CH₂, O, S and NH;
X₄ is selected from the group consisting of halide, alkyl, OR₂₃, SR₂₄ and NR₂₅R₂₆;
R₂₀ is selected from the group consisting of acyl, alkoxy, alkyl, alkylamino, alkylcarboxylic acid, arylcarboxylic acid, alkylcarboxylic alkylester, alkylene, alkylether, alkylhydroxy, alkylthio, alkynyl, amido, amino, aryl, arylalkoxy, arylamino, arylthio, carboxylic acid, cyano, cycloalkyl, carboxylic acid, ester, halo, haloalkoxy, haloalkyl, haloalkylether, heteroaryl, heteroarylamino, heterocycloalkyl and hydrogen, any of which is optionally substituted;
R₂₁ and R₂₂ are each independently selected from the group consisting of alkoxy, alkyl, alkylamino, alkylene, alkylether, alkylthio, alkynyl, amido, amino, aryl, arylether, arylalkoxy, arylamino, arylthio, carboxy, cyano, cycloalkyl, ester, halo, haloalkoxy, haloalkyl, heteroaryl, heteroarylamino, heterocycloalkyl, hydroxyl, hydrogen, nitro, thio, sulfonate, sulfonyl and sulfonylamino, any of which is optionally substituted;
R₂₃ is selected from the group consisting of acyl, alkyl, alkylamino, alkylene, alkynyl, aryl, arylalkoxy, arylamino, arylthio, carboxy, cycloalkyl, ester, ether, haloalkyl, heteroaryl heteroarylamino, heterocycloalkyl, hydrogen, thio, sulfonate, and sulfonylamino, any of which is optionally substituted;
R₂₄ is selected from the group consisting of alkyl, alkylaryl, alkylene, alkynyl, aryl, cycloalkyl, ester, halo, haloalkyl, heteroaryl, heterocycloalkyl, and hydrogen, any of which is optionally substituted; and
R₂₅ and R₂₆ are each independently selected from the group consisting of acyl, alkyl, aminoalkyl, alkylene, alkylthio, alkynyl, aryl, arylalkoxy, arylamino, arylthio, carboxy, cycloalkyl, ester, ether, halo, haloalkoxy, haloalkyl, haloalkylether, heteroaryl, heteroarylamino, heterocycloalkyl and hydrogen, any of which is optionally substituted;
and pharmaceutically acceptable salts thereof; and
D) a compound having the general formula VIIIa: 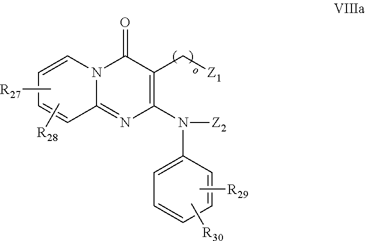
wherein
o is 0, 1, 2, or 3:
Z₁ and Z₂ are each independently selected from the group consisting of hydrogen, halogen, C₁-C₁₀ alkyl, C₃-C₁₀ cycloalkyl, C₂-C₁₀ alkenyl, C₃-C₁₀ cycloalkenyl, C₃-C₁₅ cycloalkylalkoxy, C₃-C₁₅ cycloalkylalkyl, hydroxyl, haloalkyl, oxo, —OR³¹, —OC(O)R³¹, —OC(O)N(R³¹)₂, —C(O)OR³¹, —C(O)R³¹, —C(O)N(R³¹)₂, —CN, —NO₂, —NH₂, —N(R³¹)₂, —N(R³¹)C(O)R³¹, —N(R³¹)C(O)N(R³¹)₂, —OR³¹HetA, —OR³¹N(R³¹)₂, —C(O)N(R³¹)R³¹HetA, —C(O)N(R³¹)HetA, —C(O)HetA, —C(O)N(R³¹)R³¹S(O)₂R³¹; SH, C(S)H, —S(O)₂N(R³¹)₂, —S(O), R³¹, —N(R³¹)C(O)R³¹SR³¹, —N(R³¹)R³¹S(O)₂R³¹,—N(R³¹)S(O)₂R³¹, aryl, benzyl, heteroaryl, and heterocyclyl, or two groups of Z₁ and Z₂ are connected with each other to make a five or six membered cyclic, heterocyclic or heteroaryl ring, any of which is optionally substituted;
R₂₇ and R₂₈ are each independently selected from the group consisting of alkoxy, alkyl, alkylamino, alkylene, alkylether, alkylthio, alkynyl, amido, amino, aryl, arylether, arylalkoxy, arylamino, arylthio, carboxy, cyano, cycloalkyl, ester, halo, haloalkoxy, haloalkyl, heteroaryl, heteroarylamino, heterocycloalkyl, hydroxyl, hydrogen, nitro, thio, sulfonate, sulfonyl and sulfonylamino, any of which is optionally substituted;
R₂₉ and R₃₀ are each independently selected from the group consisting of alkoxy, alkyl, alkylamino, alkylene, alkylether, alkylthio, alkynyl, amido, amino, aryl, arylether, arylalkoxy, arylamino, arylthio, carboxy, cyano, cycloalkyl, ester, halo, haloalkoxy, haloalkyl, heteroaryl, heteroarylamino, heterocycloalkyl, hydroxyl, hydrogen, nitro, thio, sulfonate, sulfonyl and sulfonylamino, or R₂₉ and R₃₀ are connected with each other to make a five or six membered cyclic, heterocyclic, aryl, or heteroaryl ring, any of which is optionally substituted;
R³¹ is independently, at each occurrence, selected from the group consisting of hydrogen, C₁-C₁₀ alkyl, C₃-C₁₀ cycloalkyl, C₂-C₁₀ alkenyl, cycloalkenyl, C₂-C₁₀ alkynyl, C₁-C₁₀ haloalkyl, aryl, benzyl, heteroaryl, and heterocyclyl, any of which is optionally substituted;
and pharmaceutically acceptable salts thereof.

2. The compound, according to claim 1, having the general formula II: 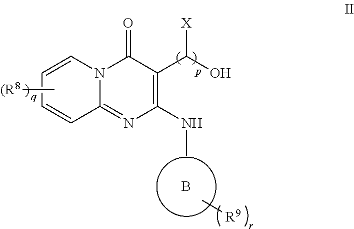
wherein
p is 0, 1, 2, or 3;
q is 1, 2, 3, or 4;
r is 1, 2, 3, or 4;
X is alkyl or aryl;
B is C₅-C₁₂ aryl;
R⁸ is selected from the group consisting of hydrogen, halogen, C₁-C₁₀ alkyl, C₃-C₁₀ cycloalkyl, hydroxyl, —OR¹⁰, —CN, —NO₂, —NH₂, —N(R¹⁰)C(O)R¹⁰, —C(O)R¹⁰, —C(O)—OR¹⁰, —C(O)N(R¹⁰)₂, —S(O)R¹⁰, —S(O)₂R¹⁰, —S(O)₂N(R¹⁰)₂, aryl, benzyl, heteroaryl, and heterocyclyl, or two groups of R⁸ are connected with each other to make a five or six membered cyclic or heterocyclic ring, any of which is optionally substituted;
R⁹ is selected from the group consisting of hydrogen, halogen, C₃-C₁₀ alkyl, C₃-C₁₀ cycloalkyl, C₂-C₁₀ alkenyl, C₃-C₁₀ cycloalkenyl, C₃-C₁₅ cycloalkylalkoxy, C₃-C₁₅ cycloalkylalkyl, hydroxyl, oxo, —OR¹¹, —OC(O)R¹¹, —OC(O)N(R¹¹)₂, —C(O)OR¹¹, —C(O)R¹¹, —C(O)N(R¹¹)₂, —CN, —NO₂, —N(R¹¹)₂, —N(R¹¹)C(O)R¹¹, —N(R¹¹)C(O)N(R¹¹)₂, —OR¹¹HetA, —OR¹¹N(R¹¹)₂, —C(O)N(R¹¹)R¹¹HetA, —C(O)N(R¹¹)HetA, —C(O)HetA, —C(O)N(R¹¹)R¹¹—S(O)₂R¹¹, —S(O)₂N(R¹¹)₂, —S(O)₂R¹¹, —N(R¹¹)C(O)R¹¹SR¹¹, —N(R¹¹)R¹¹S(O)₂R¹¹, —N(R¹¹)—S(O)₂R¹¹, —R¹¹P(O)(OR¹¹)₂, aryl, benzyl, heteroaryl, and heterocyclyl, or two groups of R⁹ are connected with each other to make a five or six membered cyclic or heterocyclic ring, any of which is optionally substituted;
R¹⁰ and R¹¹ are independently, at each occurrence, selected from the group consisting of hydrogen, C₁-C₁₀ alkyl, C₃-C₁₀ cycloalkyl, C₂-C₁₀ alkenyl, C₃-C₁₀ cycloalkenyl, C₂-C₁₀ alkynyl, C₁-C₁₀ haloalkyl, aryl, benzyl, heteroaryl, and heterocyclyl, any of which is optionally substituted;
HetA is heteroaryl;
and pharmaceutically acceptable salts thereof.

3. The compound, according to claim 1, having the general formula VIII: 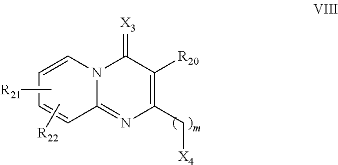
wherein
m is 0, 1, 2, or 3;
X₃ is selected from the group consisting of CH₂, O, S and NH;
X₄ is selected from the group consisting of halide, alkyl, OR₂₃, SR₂₄ and NR₂₅R₂₆;
R₂₀ is selected from the group consisting of acyl, alkoxy, alkyl, alkylamino, alkylcarboxylic acid, arylcarboxylic acid, alkylcarboxylic alkylester, alkylene, alkylether, alkylhydroxy, alkylthio, alkynyl, amido, amino, aryl, arylalkoxy, arylamino, arylthio, carboxylic acid, cyano, cycloalkyl, carboxylic acid, ester, halo, haloalkoxy, haloalkyl, haloalkylether, heteroaryl, heteroarylamino, heterocycloalkyl and hydrogen, any of which is optionally substituted;
R₂₁ and R₂₂ are each independently selected from the group consisting of alkoxy, alkyl, alkylamino, alkylene, alkylether, alkylthio, alkynyl, amido, amino, aryl, arylether, arylalkoxy, arylamino, arylthio, carboxy, cyano, cycloalkyl, ester, halo, haloalkoxy, haloalkyl, heteroaryl, heteroarylamino, heterocycloalkyl, hydroxyl, hydrogen, nitro, thio, sulfonate, sulfonyl and sulfonylamino, any of which is optionally substituted;
R₂₃ is selected from the group consisting of acyl, alkyl, alkylamino, alkylene, alkynyl, aryl, arylalkoxy, arylamino, arylthio, carboxy, cycloalkyl, ester, ether, haloalkyl, heteroaryl, heteroarylamino, heterocycloalkyl, hydrogen, thio, sulfonate, and sulfonylamino, any of which is optionally substituted;
R₂₄ is selected from the group consisting of alkyl, alkylaryl, alkylene, alkynyl, aryl, cycloalkyl, ester, halo, haloalkyl, heteroaryl, heterocycloalkyl, and hydrogen, any of which is optionally substituted; and
R₂₅ and R₂₆ are each independently selected from the group consisting of acyl, alkyl, aminoalkyl, alkylene, alkylthio, alkynyl, aryl, arylalkoxy, arylamino, arylthio, carboxy, cycloalkyl, ester, ether, halo, haloalkoxy, haloalkyl, haloalkylether, heteroaryl, heteroarylamino, heterocycloalkyl and hydrogen, any of which is optionally substituted;
and pharmaceutically acceptable salts thereof.

4. The compound, according to claim 1, having the general formula VIIIa: 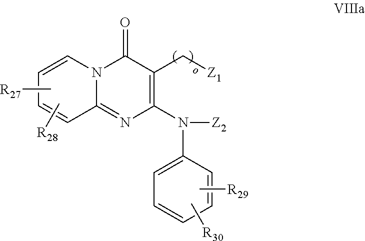
wherein
o is 0, 1, 2, or 3;
Z₁ and Z₂ are each independently selected from the group consisting of hydrogen, halogen, C₁-C₁₀ alkyl, C₃-C₁₀ cycloalkyl, C₂-C₁₀ alkenyl, C₃-C₁₀ cycloalkenyl, C₃-C₁₅ cycloalkylalkoxy, C₃-C₁₅ cycloalkylalkyl, hydroxyl, haloalkyl, oxo, —OR³¹, —OC(O)R³¹, —OC(O)N(R³¹)₂, —C(O)OR³¹, —C(O)R³¹, —C(O)N(R³¹)₂, —CN, —NO₂, —NH₂, —N(R³¹)₂, —N(R³¹)C(O)R³¹, —N(R³¹)C(O)N(R³¹)₂, —OR³¹HetA, —OR³¹N(R³¹)₂, —C(O)N(R³¹)R³¹HetA, —C(O)N(R³¹)HetA, —C(O)HetA, —C(O)N(R³¹)R³¹S(O)₂R³¹; SH, C(S)H, —S(O)₂N(R³¹)₂, —S(O)₂R³¹, —N(R³¹)C(O)R³¹SR³¹, N(R³¹)R³¹S(O)₂R³¹, —N(R³¹)S(O)₂R³¹, aryl, benzyl, heteroaryl, and heterocyclyl, or two groups of Z₁ and Z₂ are connected with each other to make a five or six membered cyclic, heterocyclic or heteroaryl ring, any of which is optionally substituted;
R₂₇ and R₂₈ are each independently selected from the group consisting of alkoxy, alkyl, alkylamino, alkylene, alkylether, alkylthio, alkynyl, amido, amino, aryl, arylether, arylalkoxy, arylamino, arylthio, carboxy, cyano, cycloalkyl, ester, halo, haloalkoxy, haloalkyl, heteroaryl, heteroarylamino, heterocycloalkyl, hydroxyl, hydrogen, nitro, thio, sulfonate, sulfonyl and sulfonylamino, any of which is optionally substituted;
R₂₉ and R₃₀ are each independently selected from the group consisting of alkoxy, alkyl, alkylamino, alkylene, alkylether, alkylthio, alkynyl, amido, amino, aryl, arylether, arylalkoxy, arylamino, arylthio, carboxy, cyano, cycloalkyl, ester, halo, haloalkoxy, haloalkyl, heteroaryl, heteroarylamino, heterocycloalkyl, hydroxyl, hydrogen, nitro, thio, sulfonate, sulfonyl and sulfonylamino, or two groups of R₂₉ and R₃₀ are connected with each other to make a five or six membered cyclic, heterocyclic, aryl, or heteroaryl ring, any of which is optionally substituted;
R³¹ is independently, at each occurrence, selected from the group consisting of hydrogen, C₁-C₁₀ alkyl, C₃-C₁₀ cycloalkyl, C₂-C₁₀ alkenyl, C₃-C₁₀ cycloalkenyl, C₂-C₁₀ alkynyl, C₁-C₁₀ haloalkyl, aryl, benzyl, heteroaryl, and heterocyclyl, any of which is optionally substituted;
and pharmaceutically acceptable salts thereof

5. The compound according to claim 1 and having one of the formulas 1-124 as shown in Example 6, or one of the formulas 125-359 as shown in Example 7.

6. The compound according to claim 1 and having one of the formulas listed in Table 1 or 2.

7-8. (canceled)

9. A pharmaceutical composition comprising a compound according to claim 1 and a pharmaceutically acceptable diluent, carrier or excipient.

10. A method of treatment of a bacterial infection, said method comprising the application of a pharmaceutically suitable amount of a compound according to claim 1.

11. The method according to claim 10, wherein the bacterial infection is Tuberculosis.

12. The compound, according to claim 1, having the general formula I: 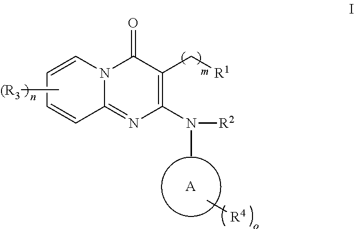
wherein
m is 0, 1, 2, or 3;
n is 1, 2, 3, or 4;
o is 1, 2, 3, or 4;
A is C₅-C₁₂ heteroaryl;
R¹ is selected from the group consisting of hydrogen, halogen, C₁-C₁₀ alkyl, C₃-C₁₀ cycloalkyl, C₂-C₁₀ alkenyl, C₃-C₁₀ cycloalkenyl, C₃-C₁₅ cycloalkylalkoxy, C₃-C₁₅ cycloalkylalkyl, hydroxyl, haloalkyl, oxo, —OR⁵, —OC(O)R⁵, —OC(O)N(R⁵)₂, —C(O)OR⁵, —C(O)R⁵, —C(O)N(R⁵)₂, —CN, —NO₂, —NH₂, —N(R⁵)₂, —N(R⁵)C(O)R⁵, —N(R⁵)C(O)N(R⁵)₂, —OR⁵HetA, —OR⁵N(R⁵)₂, —C(O)N(R⁵)R⁵HetA, —C(O)N(R⁵)HetA, —C(O)HetA, —C(O)N(R⁵)R⁵S(O)₂R⁵; SH, C(S)H, —S(O)₂N(R⁵)₂, —S(O)₂R⁵, —N(R⁵)C(O)R⁵SR⁵, —N(R⁵)R⁵S(O)₂R⁴, —N(R⁵)S(O)₂R⁵, aryl, benzyl, heteroaryl, and heterocyclyl, any of which is optionally substituted;
R² is selected from the group consisting of hydrogen, halogen, C₁-C₁₀ alkyl, C₃-C₁₀ cycloalkyl, C₂-C₁₀ alkenyl, C₃-C₁₀ cycloalkenyl, C₃-C₁₅ cycloalkylalkyl, —NH₂, —N(R⁶)₂, —C(O)R⁶, —C(O)OR⁶, —C(O)N(R⁶)₂, —S(O)R⁶, —S(O)₂R⁶, —S(O)₂N(R⁶)₂, aryl, benzyl, heteroaryl, and heterocyclyl, or R¹ and R² are connected with each other to make a five or six membered cyclic or heterocyclic ring, any of which is optionally substituted;
R³ is selected from the group consisting of hydrogen, halogen, C₁-C₁₀ alkyl, C₃-C₁₀ cycloalkyl, hydroxyl, —OR⁶, —CN, —NO₂, —N(R⁶)C(O)R⁶, —N(R⁶)C(O)R⁶, —C(O)R⁶, —C(O)OR⁶, —C(O)N(R⁶)₂, —S(O)R⁶, —S(O)₂R⁶, —S(O)₂N(R⁶)₂, aryl, benzyl, heteroaryl, and heterocyclyl, or two groups of R³ are connected with each other to make a five or six membered cyclic or heterocyclic ring, any of which is optionally substituted;
R⁴ is independently, at each occurrence, selected from the group consisting of hydrogen, halogen, C₁-C₁₀ alkyl, C₃-C₁₀ cycloalkyl, hydroxyl, —OR⁶, —CN, —NO₂, —NH₂, —N(R⁶)C(O)R⁶, —C(O)R⁶, —C(O)OR⁶, —C(O)N(R⁶)₂, —S(O)R⁶, —S(O)₂R⁶, —S(O)₇N(R⁶)₂, aryl, benzyl, heteroaryl, and heterocyclyl, or two groups of R⁴ are connected with each other to make a five or six membered cyclic or heterocyclic ring, any of which is optionally substituted;
R⁵ and R⁶ are independently, at each occurrence, selected from the group consisting of hydrogen, C₁-C₁₀ alkyl, C₃-C₁₀ cycloalkyl, C₂-C₁₀ alkenyl, C₃-C₁₀ cycloalkenyl, C₂-C₁₀ alkynyl, C₁-C₁₀ haloalkyl, aryl, benzyl, heteroaryl, and heterocyclyl, any of which is optionally substituted; and
HetA is heteroaryl;
and pharmaceutically acceptable salts thereof.

13. The compound, according to claim 5, which has one of formula 4, 5, 13, 61, 65, 71, 74, 78, 97, 102, 103, 104, 105, or 117 as shown in Table 1 and FIG. 8.

14. The compound, according to claim 5, which has one of formula 132-135, 137, 139-140, 147, 151-152, 160, 163, 173, 180, 184-185, 193, 195, 199-201, 204, 206-222, 224, 226, 229, 231-243, 245-278, 280-286, 290-305, 316, 324, 337, 340, 341, 355 and 356 as shown in Table 2.

15. The compound, according to claim 5, which has one of formula 4, 5, 13, 61, 65, 71, 74, 78, 97, 102-105, 117, 133, 206-210, 220, 231, 232, 235, 236, 257-259, 261, 264, 265, 267, 270, 273, 278, 295, 299-305, 337, 340 and 356 as shown in Tables 1-4 and FIG. 7.
